(19) United States
(12) Patent Application Publication
(10) Pub. No.: US 2026/0351402 A1
(43) Pub. Date: Nov. 5, 2026

(54) NETWORK-BASED MEDICAL APPARATUS CONTROL AND DATA MANAGEMENT SYSTEMS

(71) Applicant: Abiomed, Inc., Danvers, MA (US)

(72) Inventor: Dawn Bardot, Danvers, MA (US)

(73) Assignee: Abiomed, Inc., Danvers, MA (US)

(21) Appl. No.: 19/573,168

(22) Filed: Mar. 20, 2026

Related U.S. Application Data
(63) Continuation of application No. 17/882,271, filed on Aug. 5, 2022, now abandoned.
Application No. 17/882,271 is a continuation of application No. PCT/US2022/011506, filed on Jan. 6, 2022.
Application No. PCT/US2022/011506 is a continuation of application No. 17/484,760, filed on Sep. 24, 2021, now abandoned.
(60) Provisional application No. 63/134,951, filed on Jan. 7, 2021.

(51) Int. Cl. G16H 40/67; G06N 20/00; G16H 10/60; G16H 40/40

(52) U.S. Cl. CPC G16H 40/67; G06N 20/00; G16H 10/60; G16H 40/40

(57) ABSTRACT

Provided are new methods for controlling the operation of medical devices and related systems. Devices collect and relay sensor data via a secure internet connection in a streaming manner, but can also collect such data locally as cache data. When certain events occur, the devices also relay collected cache data. Device data is received by a network data system that analyzes the data and controls operation of the devices and other network devices based on such analysis. The medical devices can comprise multiple zones performing different data collection functions and being subject to different data relay processes. The system can segregate protected health information from commercial users that are allowed to access certain analytical data. In aspects, the system interacts with third party databases, e.g., customer relationship management systems, in generating analytical data. In aspects, machine learning processes are employed to improve the operation of such systems.

### CROSS REFERENCE TO RELATED APPLICATIONS

This application claims priority to currently co-pending U.S. patent application Ser. No. 17/484,760, entitled NETWORK-BASED MEDICAL APPARATUS CONTROL AND DATA MANAGEMENT SYSTEMS, filed Sep. 24, 2021, which claims the benefit of U.S. Provisional Patent Application No. 63/134,951, entitled SECURE MULTI-FUNCTIONAL MEDICAL APPARATUS CONTROL AND DATE MANAGEMENT SYSTEMS, filed Jan. 7, 2021. This application incorporates by reference the entirety of, these above-referenced priority applications.

### FIELD OF THE INVENTION

This invention relates to medical devices configured for use in specialized medical device data networks, systems for controlling such devices, and networks formed between such devices and systems.

### BACKGROUND OF THE INVENTION

Medical devices play an increasingly important role in health care. The WHO estimates the world market includes 2 million different kinds of devices distributed in over 22,000 categories. Modern medical devices collect information from sensors, implement different levels of therapy/therapeutic support, or both. Such devices may display relevant information or provide alerts to users, such as health care providers (“HCPs”). For example, US20200288988 (Abiomed, Inc.) discloses a system that measures flow in a blood vessel having a catheter-based heart pump inserted therein, without relying on measurements of electric current drawn by a motor that drives the heart pump, in which the pump is coupled to a signal generator that generates a signal indicative of pump turbine speed, reflecting speed of fluid flow in the blood vessel, and is coupled with other components that calculate blood flow rate from turbine rotational speed.

Increasingly, medical device data is made available to connected computer networks/systems, such as hospital information systems and electronic medical records (“EMRs”). However, electronic data systems in the market currently operate with a limited number of users, process limited types of data, or performed limited analyses or operations based on such data, limiting the adoption of such systems. Nonetheless, growing sophistication has been proposed for managing and using data collected from networks of medical devices (sometimes referred to as the “internet of medical things” (abbreviated “IoMT”)).

As described herein, the development of comprehensive, effective, medical device data management and control systems, which can bring together, manage, and utilize both physical components and data components of a complex, integrated, modern medical treatment environment, in a safe, compliant, and effective manner, usable by various users that interact with such systems, requires the application of significant inventive ingenuity. Such inventive systems and related methods are provided herein.

### CONSTRUCTION, TERMS, AND ACRONYMS

This section offers guidelines for reading this disclosure. The intended audience for this disclosure (“readers”) are persons having ordinary skill in the practice of technologies discussed or used herein. Readers may also be called “skilled persons,” and such technologies called “the art.” Terms such as “understood,” “known,” and “ordinary meaning,” refer to the general knowledge of skilled persons.

The term “uncontradicted” means not contradicted by this disclosure, logic, or plausibility based on knowledge of skilled persons.

Disclosed here are several different but related exemplary aspects of the invention (“aspects”) (“cases,” “facets,” or “embodiments”). The invention encompasses all aspects, as described individually and as can be arrived at by any combination of such individual aspects. The breadth and scope of the invention should not be limited by any exemplary embodiments. No language in this disclosure should be construed as indicating any element/step is essential to the practice of the invention unless such a requirement is explicitly stated. Uncontradicted, any aspect(s) can be combined with any other aspect(s).

Uncontradicted, all technical/scientific terms used here generally have the same meanings as commonly understood by skilled persons, regardless of any narrower examples or descriptions provided here (including any term introduced initially in quotations). However, aspects characterized by the inclusion of elements, steps, etc., associated with specific descriptions provided here are distinct embodiments of the invention. Uncontradicted, disclosure of any aspect using known terms, which terms are narrowed by example or otherwise in this disclosure, implicitly discloses related aspects in which such terms are alternatively interpreted using the broadest reasonable interpretation of skilled persons.

Uncontradicted, “or” means “and/or” here, regardless of any occasional inclusion of “and/or” (e.g., phrases such as “A, B, or C” and “A, B, and/or C” simultaneously disclose aspects including (1) all of A, B, and C; (2) A and C; (3) A and B; (4) B and C; (5) only A; (6) only B; and (7) only C (and also support sub-groupings, such as “A or B,” “A or C,” etc.)).

Uncontradicted, “also” means “also or alternatively.” Uncontradicted, “here,” and “herein” mean “in this disclosure”. The term “i.a.” (“ia” or “ia”) means “inter alia” or “among other things.” “Also known as” is abbreviated “aka” or “AKA” (and can be to signify terms are synonyms even if the terms are not known in the art). “Elsewhere” means “elsewhere herein.”

For conciseness, symbols are used where appropriate. E.g., “&” is used for “and,” & “~” for “about.” Symbols such as <and > are given their ordinary meaning (e.g., “≤” means “less than or equal to” & “≥” means “greater than or equal to”). A slash “/” can represent “or” (“A/B” means “A or B”) or identify synonyms of an element, as will be clear from context.

The inclusion of “(s)” after an element or a step indicates that ≥1 of such an element is present, step performed, and the like. E.g., “element(s)” means both 1 element or ≥2 elements, with the understanding that each thereof is an independent aspect of the invention.

Use of the abbreviation “etc.” (or “et cetera”) in association with a list of elements/steps means any or all suitable combinations of the recited elements/steps or any known equivalents of such recited elements/steps for achieving the function(s) of such elements/steps that are known in the art. Terms such as “and combinations,” or “or combinations” regarding listed elements/steps means combinations of any or all such elements/steps. “Suitability” signifies element(s)/step(s) acceptable or appropriate for performing a particular function/achieving particular state(s)/outcome(s), and typically means effective, practical, and non-deleterious/harmful to the intended function/characteristics/results.

Uncontradicted, heading(s) (e.g., “Construction, Terms, and Acronyms”) and subheadings are included for convenience and do not limit the scope of any aspect(s). Uncontradicted, aspect(s), step(s), or element(s) described under one heading can apply to other aspect(s) or step(s)/element(s) here.

Ranges of values are used to represent each value falling within such range that are within an order of magnitude of the smallest endpoint of the range without having to explicitly write each value of the range. E.g., a recited range of 1-2 implicitly discloses each of 1.0, 1.1, 1.2, . . . 1.9, and 2.0 and 10-100 implicitly discloses each of 10, 11, 12, . . . 98, 99, and 100). Uncontradicted, all ranges include the range's endpoints, regardless of how a range is described. E.g., “between 1-5” includes 1 and 5 in addition to 2, 3, and 4 (and all numbers between such numbers within an order of magnitude of such endpoints, e.g., 1.0, 1.1, . . . 4.9, and 5.0). For the avoidance of doubt, any number within a range, regardless of the order of magnitude of the number, is covered by the range (e.g., a range of 2-20 covers 18.593).

Terms of approximation (e.g., “about,” “~,” or “approximately”) are used (1) to refer to a set of related values or (2) where a precise value is difficult to define (e.g., due to limits of measurement). Uncontradicted, all exact values provided here simultaneously/implicitly disclose corresponding approximate values and vice versa (e.g., disclosure of “about 10” provides explicit support for the use of 10 exactly in such aspect/description). Ranges described with approximate value(s) include all values encompassed by each approximate endpoint, regardless of presentation (e.g., “about 10-20” has the same meaning as “about 10-about 20”). The scope of value(s) encompassed by an approximate term typically depends on the context of the disclosure, criticality or operability, statistical significance, understanding in the art, etc. In the absence of guidance here or in the art, terms such as “about” should be interpreted as +/−10% of the indicated value(s).

Lists of aspects, elements, steps, and features are sometimes employed for conciseness. Unless indicated, each member of each list should be viewed as an independent aspect. Each aspect defined by any individual member of a list can have, and often will have, nonobvious properties vis-a-vis aspects characterized by other members of the list.

Uncontradicted, the terms “a” and “an” and “the” and similar referents encompass both the singular and the plural form of the referenced element, step, or aspect. Uncontradicted, terms in the singular implicitly convey the plural and vice versa herein (in other words, disclosure of an element/step implicitly discloses corresponding use of such/similar elements/steps and vice versa). Hence, e.g., a passage regarding an aspect including X step supports a corresponding aspect including several X steps. Uncontradicted, any mixed use of a referent such as “a” in respect of one element/step or characteristic and “one or more of” with respect to another element/step or characteristic in a paragraph, sentence, aspect, or claim, does not change the meaning of such referents. Thus, for example, if a paragraph describes a composition comprising “an X” and “one or more Ys,” the paragraph should be understood as providing disclosure of “one or more Xs” and “one or more Ys.”

Uncontradicted, “significant” and “significantly” mean results/characteristics that are statistically significant using ≥1 appropriate test(s)/trial(s) in the given context (e.g., p≤0.05/0.01). “Detectable” means measurably present/different using known detection tools/techniques. The acronym “DOS” (or “DoS”) means “detectable(ly) or significant(ly).” Uncontradicted, as suggested above, the term “suitable” generally means appropriate, acceptable, or in contexts sufficient, or providing at least generally or substantially all of an intended function, without causing or imparting significant negative/detrimental impact.

Uncontradicted, for any value here that is not accompanied by a unit of measurement (e.g., a weight of 50 or a length of 20), any previously provided unit for the same element/step or the same type of element/step will apply, or, in cases where no such disclosure exists, the unit most commonly used in association with such an element/step in the art applies.

Uncontradicted, the terms “including,” “containing,” “comprising,” and “having” mean “including, but not limited to” or “including, without limitation.” Uncontradicted, use of terms such as comprising and including regarding elements/steps means including any detectable number or amount of an element or including any detectable performance of a step/number of steps (with or without other elements/steps).

For conciseness, description of an aspect “comprising” or “including” an element, with respect to a collection/whole (e.g., a device/composition), simultaneously implicitly provides support for any detectable amount/number or ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the whole/collection being made up of the element, or essentially all of the whole/collection being made up of the element (i.e., that the collection consists essentially of the referenced element). Similarly, a method described as including a step with respect to an effect/outcome implicitly provides support for the referenced step providing ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the effect/outcome, representing ≥~1%, ≥~5%, ≥~10%, ≥~20%, ≥~25%, ≥~33%, ≥~50%, ≥~51%, ≥~66%, ≥~75%, ≥~90%, ≥~95%, ≥~99%, or ~100% of the steps/effort performed, or both. Explicit listing of percentages of elements/steps in connection with aspects does not limit or contradict such implicit disclosure.

Uncontradicted, terms such as “comprising” when used in connection with a step of a method provide implicit support for performing the step once, ≥2 times, or until an associated function/effect is achieved.

The term “one” means a single type, single iteration/copy/thing, of a recited element or step, or both. For example, “one” component of a device/composition can mean one type of element (which may be present in numerous copies, as in the case of an ingredient in a device/composition), one unit of the element, or both. Similarly, “one” component, a “single” component, or the “only component” of a system typically means 1 type of element (which may be present in numerous copies), 1 instance/unit of the element, or both. Further, “one” step of a method typically means performing one type of action (step), one iteration of a step, or both.

The term “some” means ≥2 copies/instances or ≥5% of a listed collection/whole is, or is made up of, an element. Regarding methods, some means ≥5% of an effect, effort, or both, is made up of or is attributable to a step (e.g., as in “some of the method is performed by step Y”) or indicates a step is performed ≥2 times (e.g., as in “step X is repeated some number of times”). “Predominately,” “most,” or “mostly,” means detectably >50% (e.g., mostly comprises, predominately includes, etc., mean>50%) (e.g., a system that mostly includes element X is composed of >50% of element X). The term “generally” means ≥75% (e.g., generally consists of, generally associated with, generally comprises, etc., means ≥75%) (e.g., a method that generally consists of step X means that 75% of the effort or effect of the method is attributable to step X). “Substantially” or “nearly” means ≥95% (e.g., nearly all, substantially consists of, etc., mean≥95%) (e.g., a collection that nearly entirely is made up of element X means that at least 95% of the elements in the collection are element X).

Uncontradicted, any aspect described with respect to an optionally present element(s)/step(s) also provides implicit support for corresponding aspect(s) in which one, some, most, generally all, nearly all, essentially all, or all of such element(s) are lacking/step(s) not performed, in respect of the relevant aspect. E.g., disclosure of a system comprising element X implicitly also supports a system lacking element X. Uncontradicted, changes to tense or presentation of terms (e.g., using “comprises predominately” in place of “predominately comprises”) do not change the meaning of the corresponding term/phrase.

Uncontradicted, all methods provided here can be performed in any suitable order regardless of presentation (e.g., a method comprising steps A, B, and C, can be performed in the order C, B, and A; B and A and C simultaneously, etc.). Uncontradicted, elements of a device/composition can be assembled in any suitable manner by any suitable method. In general, any methods and materials similar or equivalent to those described here can be used in the practice of embodiments. Uncontradicted, the use of ordinal numbers such as “first,” “second,” “third,” and so on is to distinguish respective elements rather than to denote a particular order of elements. Uncontradicted, any elements, steps, components, or features of aspects and all variations thereof, etc., are within the scope of the invention.

Elements associated with a function can be described as “means for” performing a function in a composition/device/system or a “step for” performing a part of a method, and parts of this disclosure refer to “equivalents,” which means equivalents known in the art for achieving a referenced function associated with disclosed mean(s)/step(s). However, no element of this disclosure or claim should be interpreted as limited to a “means-plus-function” construction unless such intent is clearly indicated by the use of the terms “means for” or “step for.” Terms such as “configured to” or “adapted to” do not indicate “means-plus-function” interpretation, but, rather, describe element(s)/step(s) configured to, designed to, selected to, or adapted to achieve a certain performance, characteristic, property, etc., using teachings provided here or in the art.

All references (e.g., publications, patent applications, and patents) cited herein are hereby incorporated by reference as if each reference were individually and specifically indicated to be incorporated by reference and set forth in its entirety herein. Uncontradicted, any suitable principles, methods, or elements of such references (collectively “teachings”) can be combined with or adapted to aspects. However, citation/incorporation of patent documents is limited to the technical disclosure thereof and does not reflect any view regarding the validity, patentability, etc., thereof. In the event of any conflict between this disclosure and the teachings of such documents, the content of this disclosure controls regarding aspects of the invention. Numerous references are cited here to concisely incorporate known information and aid skilled persons in putting aspects into practice. While efforts have been made to include the most relevant references for such purposes, readers will understand that not every aspect of every cited reference will apply to every aspect of the invention.

### ADDITIONAL TERMS, CONCEPTS, AND ACRONYMS

The following description of certain terms and acronyms is provided to assist readers in understanding the invention. Additional acronyms may be only provided in other parts of this disclosure and acronyms that are well known in the art may not be provided here.

Uncontradicted, “improved” herein means detectably or significantly better, such as being increased with respect to a favorable outcome, characteristic, etc., and reduced with respect to a negative outcome, characteristic, etc., or with respect to time required, cost/energy required, and the like. Uncontradicted, terms such as “enhanced,” “improved,” and the like are used synonymously here. Uncontradicted, “causing” means directly causing or indirectly causing. E.g., “causing” an engine of a system/device to perform an analysis can be performed by selecting to operate the system, upon which the engine may thereafter automatically either repeatedly, periodically, in response to command(s), or in response to the existence of detected (typically preprogrammed) condition(s).

A “system” of the invention comprises an electronic device/collection of devices that is composed of interrelated/interacting components/devices (including, e.g., separate networked device(s)/component(s)), including (1) memory component(s) comprising computer-readable media including preprogrammed instructions for carrying out Function(s) and (2) a computer/processing unit capable of reading/executing such instructions causing Function(s) to be performed. Systems of the invention are often also called network data systems (“NDSs”) or, wherein the NDS controls operation of networked devices, a MAC-DMS, to distinguish the system from references to other systems (e.g., systems contained in an NDS, an MA, or OND, etc.). Readers will be able to identify where the term system is used to describe an NDS from context.

Persons/entities that access/utilize an NDS, directly or through a network device are called “users.” In aspects, users are characterized by, i.a., the “class” of role/profession to which they belong (e.g., administrators, researchers, or HCPs). E.g., an “Administrator” is one type of user. Specifically, an Administrator is an individual, team, or organization, typically associated with the owner of an NDS, that is responsible for managing, building, or maintaining one or more Functions/Modules or the system.

Systems/NDSs receive subject-related data from one or more, typically several, subject-associated medical apparatuses that networked with the NDS. A medical apparatus (“MA”) is an electronic medical device comprising hardware and that facilitates or performs task(s) related to the diagnosis, treatment, prevention, alleviation, or cure of a disease or condition in subjects (including symptoms thereof), such as human patients; comprises or is associated with sensors that detect patient-relevant data, device data, or both; and that can directly or indirectly relay such data to an NDS.

MAs often diagnose, treat, or otherwise sense or modulate the condition of “subjects.” A “subject” can be any type of a mammal (e.g., companion animals). In aspects, subjects comprise or are human patients. In aspects, patients are patients diagnosed as suffering from one or more critical (potentially life threatening) conditions, such as a brain condition, heart condition, lung condition, or other organ/system condition. Uncontradicted, “subject” and “patient” implicitly support one another here.

A “group” or “network” of medical apparatuses (MAs) means any collection of one or more types of MAs associated by one or more characteristics, such as being all MAs networked with an NDS, being all MAs in a location (site, facility group, metropolitan area, region (e.g., a county or similar division), state/province, interstate/interprovince region (e.g., New England), country, international region (e.g., the EU), or continent, or belonging to an Entity. An “Entity” is an organization (e.g., a corporation or other company) recognized as having legal rights under the laws of one or more countries or international organizations. Entities are “independent” if they are recognized as separate entities under applicable law. Typically, independent Entities are not under common control/ownership.

MAs are computerized medical devices (e.g., medical devices comprising computer(s)). Terms such as “computing unit,” “computer,” and the like, typically mean a device comprising physical computer-readable media and a processor that processes (“reads”) information in such media. The media can comprise informative data and also functional information (modifiable/programmable computer executable instructions (CEI)). Such instructions comprise specialized Engines and perform Functions. Processor(s) read CEI causing such Function(s) to be performed (e.g., generating “output”)). In aspects, a “computer” or “computerized device,” such as an “other network device” (“OND”), can be, e.g., a mobile computing device (e.g., a smartphone), a desktop computer, a laptop computer, a tablet device, etc. Other network components (e.g., systems) comprise more complicated processing or memory components, as described elsewhere. Functional data structures used in systems/networks can comprise an interface (a set of operations that the data structure supports), an implementation (including an internal representation of a data structure, definition of code/algorithms used in the operations of the data structure, or both), or a combination thereof.

Computerized devices in communication with each other form a data network (“network”). Typically, any reference to a “network” herein is in reference to a network that, in operation, includes an NDS and a number of MAs, optionally with other components (e.g., ONDs, CRM databases, etc.). Such networks are an aspect of the invention. In cases, the term network is used to describe other networks (e.g., networks/groups of MAs). Readers will be able to determine when the term network is used to refer to a network other than a network comprising an NDS and MAs.

Components of a network (e.g., of an NDS:MA network) in operation typically interact/communicate a recurring basis (typically a regular or continuous basis), usually using common communication protocols over digital interconnections for the purpose of sharing data, functions, or other resources. Networks typically comprise physical components, e.g., routers, switches, and the like, described elsewhere.

Components of networks are sometimes described as “clients” and “servers.” Client and server devices/components/Units are generally remote from each other and typically interact through a communication/data network. The relationship of client and server typically arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated in a user device, e.g., a result of user interaction, can likewise by relayed to a server.

Although system components can be/comprise computers, typically systems/NDSs have memory and processing capabilities that far exceed those of typical general-purpose laptops, mobile phones, etc., as well as comprising specialized instructions/systems for performing the particular processes described herein. In aspects, system components comprise remote/virtual computing units, such as cloud memory or cloud processing units. Other network devices (ONDs), however, can, in aspects, comprise laptops, mobile phones, and the like.

The computer units of NDSs, MAs, and other network components perform various Functions. A “Function” or “function” is a computer-implemented action performed by an NDS or other component of a network based on both preprogrammed computer readable and executed instruction(s), e.g., in response to input(s). A Function also can describe the result of step(s) of a method. The step(s) of such methods/elements of such Function(s) can comprise algorithms, methods, and the like described below. In general, a Function can be carried out by both Unit(s)/component(s) of an NDS/device and steps of a related method.

An “engine” (“Engine” or “data engine”) typically is a computer/processor-executable software program/application, which is configured to perform specified Function(s), based on computer-executable/readable instructions (“CEI”) contained in a memory (and that make up the Engine). Typically, an Engine processes input(s) and generate output(s). An Engine also can be implemented on computer(s) in one or more locations. In aspects, multiple components of an Engine are installed and running on one or more computer(s); multiple instances of an engine are installed and running one or more computers; or both. The operation of an Engine performs a Function and a Function can be performed by an Engine. Such corresponding aspects are implicitly described by any explicit description of either an Engine or Function (e.g., description of a system/component comprising an Engine for performing Function(s) implicitly discloses a method including performance of the Function as step(s)). An Engine that receives User input and provides output, i.a., to an end User can be called an “application.” Engines can make up part of, or also be described as, “programs,” code,” or “algorithms.” Engines/programs can encoded/written in any form of programming language (code), including compiled or interpreted languages, or declarative or procedural languages; and it can be deployed in any form, including as a standalone program or as a module, component, subroutine, or other unit suitable for use in a computing environment. A program may, but need not, correspond to a file in a file system. A program/Engine can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program/Engine can be deployed to be executed on one computer or on multiple computers that are located at one site or distributed across multiple sites and interconnected by a data communication network. Programs/engines also can be described as “instructions” or “computer-implemented instructions,” “processor-implemented instructions”, “computer-readable instructions,” “computer implemented data engines,” etc.

The term “Module” or “module” typically refers to a combination of mechanical/electrical or otherwise physical/non-software component(s) of a computer system (e.g., a network interface card, processor(s), etc.) and Engine(s) that together perform Function(s). Uncontradicted, a term such as Module implicitly discloses a corresponding combination of “a processor and an Engine” comprising computer readable instructions for performing the indicated Functions. Uncontradicted, use of the term “Engine” or “engine” in any aspect implicitly discloses a corresponding aspect where referenced Function(s) are performed by a Module, and vice versa. Thus, uncontradicted, any disclosed “Engine” or “engine” implicitly supports a corresponding module comprising physical components (such as processor(s) and memory component(s)).

The term “Unit” or “unit” typically refers to structural element(s) of a computer device, system, or component that is either an engine or module and, uncontradicted, implicitly discloses and, thus, can be substituted with either a corresponding engine or module.

The term “controller” can be applied to any engine, unit, module, device, component, system, etc., which controls the operation of another device, engine, system, unit, etc., directly or indirectly, even if not always described as such. Thus, e.g., an engine in a system that generates an application that controls the operation of a networked device can be also described as a controller of such other device. Engines, processors, and other elements of systems and devices also can be described as “components” of such systems, devices, and the like. Engines also can comprise component parts such as algorithms, sub-programs, etc.

The step(s) of Function(s) or Function(s) of Engine(s) are sometimes described here using pseudocode/algorithms. For conciseness, pseudocode is sometimes presented on a single line using indicators in the order of (1) Arabic numerals, (a) lower case letters, (I) upper case Roman numerals, (A) upper case letters, and (i) lower case roman numerals to provide hierarchy. E.g., pseudocode for the algorithm:

| START | |
|---|---|
| (1) | DECLARE A = 10 |
| (2) | INPUT B |
| (3) | IF B > 0 |

| | |
|---|---|
| (a) | WHILE A ≤ 10 |

| (I) | IF A ≠ 0 |
|---|---|
| | (A) DECLARE C = A * B |
| (II) | ELSE |
| (A) | PRINT C |

| | |
|---|---|
| (b) | DECREMENT A (A = A−1) |

END

Can be written START, (1) DECLARE A = 10, (2) INPUT B, (3) IF B > 0: (a) WHILE A ≤ 10: (I)
IF A ≠ 0: (A) DECLARE C = A * B, (II) ELSE: (A) PRINT C, (b) DECREMENT A (A =−1),
and END.

The way in which an engine is encoded depends on the features of the computer/system that carries out the Function and selection of human readable instructions provided to the system/computer allowing for manual modification and control of the Function. Each engine is/acts as a specialized device comprising physical media comprising physically encoded process/computer executable instructions for performing specific steps providing specialized technical effects when read/executed by processor(s). Such technical effects include transforming data in a manner that cannot be reasonably be carried out by persons (e.g., by simultaneously sorting and displaying portions of such data to different, independent types of stakeholders), improving the efficiency of systems (e.g., the speed of control of medical devices, the speed of delivery of device data, etc.), or control operation of medical devices for treatment.

“Input” typically means data that is provided to an NDS, device, etc., from any external source (e.g., an associated medical device, a user providing information to a networked device, an external database, or a combination thereof (“CT”)). An “output” can be any device or component for displaying or relaying data to a user or other device/component of a device/system/network, or applying activities based on data (e.g., control of a medical apparatus). In cases, output that is not purely data is referred to as an output application.

An “interface” in the context of data input/output can be any suitable interface between a user and a computer, a network, or a system (e.g., an NDS of the invention). In aspects, the term “interface” is used to refer a user interface implemented in the form of a web page or similar media that can be viewed/navigated on several devices via the internet (e.g., using standard web browser(s)) (a “web interface” or “software interface”). Such an interface typically comprises a preprogrammed layout specific to the user, user class, or both. A device that displays/accesses an interface, such as a specialized web interface, can be considered a device of a network. In aspects, devices that access a network comprise fixed/specialized interface hardware. As such, network devices are sometimes referred to as devices/interfaces; however, uncontradicted, any disclosure of one such aspect implicitly discloses the other and vice versa.

Terms such as “data” and “information” are used interchangeably here and are only limited to a specific form when so indicated by explicit definition/statement or clear context. Interrelated data can be described as a “Record” or “record.” Records will often have attributes (characteristics) and values (measurements/attributes). Records can be in structured, semi-structured, or unstructured form, depending on context, as discussed infra. Collections of records can be called “datasets.” Collections of datasets in memory can be called “data repositories.”

A “schema” typically is an organization of data typically generated by application of constraints/structures to a collection of less ordered data to form a new ordered structured data collection from “underlying” data. Other collections of data, such as data repositories, are discussed elsewhere. A “representation” is a similar data construct, but typically used specifically with respect to data that is specifically designed for or associated with instructions for display on a graphical user interface or for other visual output (e.g., print). Representations also can provide information concerning objects, systems, devices, processes, etc., which exist in a structured/hierarchical or otherwise interacting or connected relationship (e.g., a grouping of medical apparatuses or data therefrom, which apparatuses are associated with a particular hospital, geographic area, class of users, Independent Entity, etc.).

Data that is generated and transmitted in networks/systems of the invention can be characterized as functional data, comprising CEI (e.g., instructions for a processor to cause an MA to change settings relating to treatment of a patient) or nonfunctional data (e.g., sensor measurements). Uncontradicted, data can be relayed, transmitted, etc. through any suitable mode/mechanism and terms such as relayed, transmitted, conveyed, etc. are used interchangeably unless otherwise specified. The terms upload and download can be used in relation to the direction of data flow to (into) and from (out of) a referenced system, device, or other source.

Electronic information relayed between components or over a network is typically relayed in packets, which can comprise other information such as identification data, source data, destination data, size data, persistence information (time to live (TTL)), composition data (e.g., flags), etc., as known in the art and discussed elsewhere. Packets can be organized into “streams.” Data also can be relayed via frames/chunks, or other known units/forms. Use of such terms can, accordingly, be considered exemplary herein, such forms being typically interchangeable or replaceable by data in other formats suitable for data transmission, storage, or both.

Terms such as “authorized” or “authorization” describe, i.a., data or Function(s) configured to be accessible only by certain users, types of users (classes), devices, or CT, and typically are subject to one or more Functions designed to restrict access to such data/Function(s) (e.g., firewalls, authorization protocols such as password access restrictions, biometric access restrictions, and other forms of identification restrictions).

A “regulation” means a law, treaty, regulation, rule, guidance, code of conduct, standard, ruling, or similar directive (e.g., that applies to the application of healthcare, diagnosis of disease, or the like). A “Regulatory Authority” (RA) is an Entity that promulgates, oversees, or enforces Regulations. Terms such as “regulatory requirements” (abbreviated “RRs”) have the same meaning as “regulations.”

Terms such as “machine learning” (“ML”) and “artificial intelligence” (“AI”) mean any suitable method/system in which one or more Functions applies one or more machine learning models to information, typically resulting in computer modification of the Function by the operation of the machine learning model(s). ML/AI methods can involve Administrators, either in terms of training, supervision, or both.

Generally, any method described herein can be adapted to provide a corresponding system/NDS and vice versa. Accordingly, disclosure of any method simultaneously implicitly discloses a corresponding system that can carry out the indicated Functions and a disclosure of an NDS that comprises Engines/Modules implicitly discloses a corresponding method that comprises the steps for performing Functions corresponding to the Modules. Thus, uncontradicted, terms like “system” implicitly mean a corresponding “system or (i.e., and/or) method”, and terms like “method” likewise implicitly disclose a corresponding “method or (i.e., and/or) system.”

The following table lists further acronyms that are frequently used in this disclosure and provides a description of the related elements:

TABLE 1
Select Acronyms (not in alphabetical order)

| Acronym | Term | Brief Description |
|---|---|---|
| MA | Medical Apparatus | A medical device that relays data comprising sensor |
| | | data to a system/NDS. |
| MAG | MA group | A group of MAs related in respect(s) (e.g., being |
| | | controlled by the same Independent Entity). |
| MZMA | Multi-Zone MA | An MA comprising ≥2 operating zones that are |
| | | subject to separate security units/rules or different |
| | | regulatory status, have different communication |
| | | capabilities, perform different MA functions, etc. |
| MA-D | MA-data | Data generated by an MA and relayed to an NDS. |
| NDS | Network Data System | A system of the invention that receives MA-D from |
| | | networked MAs, analyzes such data, and delivers |
| | | output based the analysis to networked MAs/ONDs. |
| MAC-DMS | MA control(ling) & | An NDS that in operation controls the operation of |
| | data management | MAs in one or more way(s), such as changing |
| | system | application of medical/diagnostic functions. |
| NDS-AD | NDS Analytical Data | Information generated by an NDS based on, i.a., MA-D. |
| | | Also called “analytical data” or “analysis.” |
| CDSS | Clinical Decision | A unit/engine that is not regulated as SaMD and that |
| | Support System (or | analyzes data (e.g., data within EMRs) and performs |
| | CDS System) | tasks (e.g., providing prompts, reminders, or |
| | | information) to assist HCPs in implementing clinical |
| | | protocols, guidelines, etc. |
| DR | Data Repository | A discrete collection of stored/persistent data, e.g., a |
| | | database (DB), data lake (DL), enhanced data lake, |
| | | data warehouse, etc. |
| MLM | Machine Learning | An Engine/Unit that applies ML processes, models, |
| | Module | or analysis to NDS Function(s). |
| NTCRM | Non-Transitory | Media comprising CEI that is mostly, generally, or |
| | Computer Readable | entirely not transient in nature (“transient” meaning |
| | Medium | not be reusable, transferrable, or both). |
| CEI | Computer-Executable | Instructions (e.g., code) stored in a PTRCRM and |
| | Instructions | executed (ran) by a processor/computer. |
| PTRCRM | Physical, | Computer-readable media that is physical, |
| | Transferrable, and | transferrable, and reproducible. E.g., transferable & |
| | Reproducible | reproducible signals with physical effects that execute |
| | Computer Readable | Function(s), flash memory, RAM, or NTCRM, but |
| | Medium | excludes transient signals. |
| HCP | Health Care Provider | A person involved with providing health care, often |
| | | through use of an MA. |
| PHI | Personal/protected | Information concerning patient(s) that is subject to |
| | health information | one or more patient privacy-related law(s)/RR(s) |
| | | (e.g., US HIPAA). |
| SAMD/ | Software as a Medical | Software intended to be used for medical purpose(s) |
| SaMD | Device | that is subject to RR(s) |
| EMR | Electronic Medical | Electronic record comprising patient-specific |
| | Record | medical-related data. |
| IEs | Independent Entities | Entities that are legally separate (e.g., that neither |
| | | control nor is under common control with another |
| | | referenced Entity) |
| SMAD | Streaming MA-D | MA data relayed in a streaming manner from network |
| | | component(s) |
| MA-CD | MA cache data | MA-D stored, at least initially, in computer memory |
| | | of an MA |
| SUMAD | Semi-unstructured | MA-D that can be characterized as semi-unstructured |
| | MA-D | data |
| SDP | Streaming Data | A NDS component that receives and analyzes SMAD |
| | Processor/Engine/or | separate from other Engines (e.g., an Analytical |
| | Unit | Unit/Engine) |
| SO | System | Entity(ies) that own or otherwise is/are in control of |
| | Owner/Operator | the NDS. |
| ONDI | Other Network Device | Interactive computerized devices or web/network |
| | (OND)/Interface | interfaces that receive NDS-AD from the NDS, other |
| | | than MAs. Uncontradicted, ONDI is used |
| | | interchangeable with OND. |
| CRMS | Customer relationship | Computer systems, typically managed by an Entity, |
| | management (CRM) | specifically configured to perform functions relating |
| | system | to managing interactions with customers of |
| | | goods/services, e.g., Salesforce ™ CRM. |
| S/F and | Step/Function | Step(s) of methods and Function(s) of Units/Engines |
| S(s)/F(s) | | of NDSs/computers |
| U/F | Unit(s)/Function(s) | Unit(s) of NDSs or devices (e.g., MAs) or Functions |
| | | performed in methods they carry out. |

In cases, a description of some of these acronyms is repeated in the following portions of the disclosure to aid readability.

### SUMMARY OF THE INVENTION

The systems, methods, and devices/apparatuses disclosed herein have many attributes and aspects including, but not limited to, those set forth in, e.g., described or referenced in, this Summary of the Invention (“Summary”). This Summary is not intended to be all-inclusive, and the scope of the invention is not limited to or limited by the aspects, features, elements, or embodiments provided in this Summary. Rather, this Summary is provided to illustrate the nature of the invention by providing a description of aspects that illustrate some of the key features and characteristics of such systems, methods, and devices. Any aspects of devices/systems or methods described in this section can be combined with any other aspect in this or any other section.

The volume of patent disclosures related to ideas for medical device data management systems reflects the fact that the development of comprehensive, effective, medical device data management and control systems, which can bring together, manage, and utilize both physical components and data components of a complex, integrated, modem medical treatment environment, in a safe, compliant, and effective manner, usable by various users that interact with such systems, will require the application of significant inventive ingenuity. Such inventive systems and related methods are provided herein.

In one aspect, the invention provides computer-implemented methods for controlling operation of medical apparatuses and other devices in a data network comprising (1) providing a data network, the data network comprising (a) a collection of remote-controllable, selectively operable, and internet-connected medical apparatuses, each of the medical apparatuses comprising (I) one or more sensors that detect one or more patient-associated physiological states of any patient operationally associated with the medical apparatus that convert information regarding such one or more physiological states to processor-readable medical apparatus data (MA-D), the MA-D, (II) an output engine that selectively relays data via secure internet data communications, (III) a medical apparatus memory component that selectively stores MA-D and comprises processor-executable instructions for analyzing and relaying MA-D and evaluating the connection between the medical apparatus and the network, (IV) a medical apparatus processor that executes the instructions in the medical apparatus memory component, (b) a medical apparatus controller and data management system (“MAC-DMS”) comprising (I) a MAC-DMS processor that reads computer readable instructions to analyze data and perform functions, (II) a streaming data processing engine, (III) a cache MA-D processing engine, (IV) an analytical engine, and (V) a MAC-DMS memory component that comprises processor-executable instructions and one or more data repositories, the one or more data repositories comprising an enhanced data lake that stores at least some of the MA-D and at least some of the analytical data separately and under different access conditions; (2) causing each operating medical apparatus to repeatedly collect sensor data from the patient; (3) causing each operating medical apparatus to automatically and repeatedly assess whether a secure network connection is available, and (a) when a secure and stable network connection is available, automatically relaying data comprising MA-D to the MAC-DMS or (b) when a secure and stable network connection is not available, (I) storing MA-D in the medical apparatus memory component as cache MA-D until a secure and stable network connection becomes available and (II) when a secure and stable network connection becomes available, relaying the cache MA-D to the MAC-DMS via secure internet data communication; (4) causing the MAC-DMS processor to use the cache MA-D processor to (a) receive cache MA-D when cache MA-D is relayed from a medical apparatus to the MAC-DMS and (b) determine whether the received cache MA-D is suitable for analysis by the analytical engine, storage in at least one of the one or more data repositories, or both; (5) causing the MAC-DMS processor to use the analytical engine to perform one or more analytical functions on MA-D stored in the enhanced data lake upon request from a user, upon occurrence of a preprogrammed condition, or both, and (5) causing the MAC-DMS processor to relay one or more outputs via secure internet communication to one or more medical apparatuses, the one or more outputs comprising (a) analytical data output, (b) one or more output applications comprising instructions that control the operation of (I) one or more medical apparatus functions in one or more of the medical apparatuses of the data network, (II) one or more other network device functions in one or more of the other network devices of the data network, or (III) both (I) and (II), or (c) a combination of (a) and (b). In more particular aspects, such methods are further characterized by (1) the MA-D comprises MA-D that complies with a pre-established semi-structured data format; (2) the system/MAC-DMS comprises an output engine that selectively relays data via secure internet data communications; (3) the streaming data processing engine automatically and continuously receives and processes MA-D relayed from the medical apparatuses and performs an initial analysis on received streaming MA-D to determine if one or more preprogrammed conditions are present in the streaming MA-D and, if such one or more conditions are present, acting as a controller of one or more of the medical apparatuses, one or more other network devices, or both by performing one or more of a preprogrammed limited set of initial functions, which initial functions comprise relaying instructions for controlling the operation of one or more medical apparatuses, one or more other network computerized devices, or both; (4) the analytical engine performing one or more analytical functions at least partially based on analytical data stored in the enhanced data lake upon request from a user, upon occurrence of a preprogrammed condition, or both; or (5) the network comprises at least 3 computerized other network devices, each other network device (a) comprising (I) a network device processor, (II) a network device memory component, and (III) a remote controllable graphical user interface, and (b) being associated with a user assigned to at least one class of users, wherein classes of users comprise (I) health care providers authorized to access patient protected health information and (II) commercial users that are subject to restrictions on the receipt of patient protected health information, wherein the method further comprises delivering output to at least one other one of the other network devices.

In another aspect, the invention provides systems/NDSs (such as a MAC-DMS) that comprise (1) a streaming data processing Unit that automatically and continuously receives and processes MA-D relayed from a number of medical apparatuses that the NDS has permission to receive MA-D from and that performs an initial analysis on received streaming MA-D to determine if one or more preprogrammed conditions are present in the streaming MA-D and, if such one or more conditions are present, acting as a controller of one or more of the medical apparatuses, one or more other network devices, or both by performing one or more of a preprogrammed limited set of initial functions, which initial functions comprise relaying instructions for controlling the operation of one or more medical apparatuses networked with the NDS, one or more other network computerized devices networked with the NDS, or both; (2) an NDS memory component that comprises processor-executable instructions and one or more data repositories, the one or more data repositories comprising an enhanced data lake that automatically stores at least some of the MA-D and further stores at least some of the analytical data separately from, and under different access conditions than, the MA-D and that comprises (a) governance rules for sorting and managing stored data, (b) pre-determined data formatting standards applicable to at least some of the stored data, or (c) both (a) and (b); (3) an analytical engine that performs one or more analytical functions at least partially based on analytical data stored in the enhanced data lake upon request from a user, upon occurrence of a preprogrammed condition, or both; (4) a cache MA-D processing engine that analyzes cache MA-D when received by a medical apparatus and determines if the cache MA-D should be stored in the MAC-DMS memory component, analyzed by the analytical engine, or both; and (5) an analytical data controller that relays one or more outputs via secure internet communication to one or more medical apparatuses, one or more other network computerized devices, or both, the one or more outputs comprising (a) analytical data output; (b) one or more output applications comprising instructions that control the operation of (I) one or more medical apparatus functions in one or more of the networked medical apparatuses, (II) one or more other network device functions in one or more of the other network devices networked with the NDS, or (III) both (I) and (II).

In another aspect, the invention provides internet-based data networks comprising (1) a number of remote-controllable, selectively operable, and internet-connected medical apparatuses, each of the medical apparatuses comprising (a) one or more sensors that detect one or more patient-associated physiological states and convert information regarding such one or more physiological states to processor-readable medical apparatus data (MA-D), the MA-D comprising MA-D that complies with a pre-established semi-structured data format, (b) an output engine that selectively relays data via secure internet data communications, (c) a medical apparatus memory component that selectively stores MA-D and comprises processor-executable instructions for one or more medical apparatus computer implemented data engines, (d) a medical apparatus processor that executes the instructions in the medical apparatus memory component, and (e) a network status engine that (I) automatically repeatedly checks for availability of a secure and stable internet connection when in operation, (II) when a secure and stable internet connection is present, relays at least some of the MA-D via such secure and stable internet connection to one or more intended recipient internet connected devices or systems, (III) when a secure and stable internet connection is not present causes at least some of the MA-D to be stored in the medical apparatus memory component as cache MA-D until a secure and stable internet connection is established or reestablished and thereafter relays at least some of the cache MA-D to the one or more recipient internet connected devices or systems; (2) a medical apparatus controller and data management system (“MAC-DMS”) comprising (a) a system processor that reads computer readable instructions to analyze data and perform functions; (b) a streaming data processing engine/Engine that automatically and continuously receives and processes MA-D relayed from the medical apparatuses and performs an initial analysis on received streaming MA-D to determine if one or more preprogrammed conditions are present in the streaming MA-D and, if such one or more conditions are present, acts as a controller of one or more of the medical apparatuses by performing one or more of a preprogrammed limited set of initial functions, which initial functions comprise relaying instructions for controlling the operation of one or more medical apparatuses; (c) a MAC-DMS memory component that comprises processor-executable instructions and one or more data repositories, the one or more data repositories comprising an enhanced data lake that automatically stores at least some of the MA-D and further stores at least some of the analytical data separately from, and under different access conditions than, the MA-D; (d) an analytical engine that performs one or more analytical functions at least partially based on analytical data stored in the enhanced data lake upon request from a user, upon occurrence of a preprogrammed condition, or both; (e) a cache MA-D processing engine that analyzes cache MA-D when received by a medical apparatus and determines if the cache MA-D should be stored in the MAC-DMS memory component, analyzed by the analytical engine, or both; and (f) a network device controller (analytical data controller) that relays one or more outputs via secure internet communication to one or more medical apparatuses, one or more other network computerized devices, or both, the one or more outputs comprising (I) analytical data output; (II) one or more output applications comprising instructions that control the operation of one or more medical apparatus functions in one or more of the medical apparatuses of the data network; or (III) both (I) and (II).

In another aspect, the invention provides internet-based data networks comprising the features described in the preceding paragraph and further comprising at least 3 other computerized network devices (“ONDs”), each other network device (a) comprising (I) a network device processor, (II) a network device memory component, and (III) a remote controllable graphical user interface, and (b) being associated with a user assigned to at least one class of users, wherein classes of users comprise (I) health care providers authorized to access patient protected health information and (II) commercial users that are subject to restrictions on the receipt of patient protected health information, wherein the streaming data processing engine optionally relays output to the one or more other network devices when a condition is detected by the streaming data processing engine, the MAC-DMS relays output application based on or comprising analytical data to the ONDs, or both.

In another aspect, the invention provides networks according to either or both of the preceding two paragraphs wherein the NDS/MAC-DMS has access to a customer relationship management operated by an entity that is independent of the one or more medical apparatus-associated entities and the MAC-DMS associated entity and that comprises (a) customer relationship management information comprising information concerning sales of medical apparatuses to the one or more medical apparatus-associated entities and (b) a component for relaying the customer relationship management information to the MAC-DMS processor, wherein the MAC-DMS processor combines the customer relationship management information with analytical data and relays the combined information to other network devices of commercial users.

In another aspect, the invention provides internet-based data networks comprising (1) a number of remote-controllable, selectively operable, and internet-connected medical apparatuses, each of the medical apparatuses comprising (a) one or more sensors that detect one or more patient-associated physiological states and convert information regarding such one or more physiological states to processor-readable medical apparatus data (MA-D), the MA-D comprising MA-D that complies with a pre-established semi-structured data format, (b) an output engine that selectively relays data via secure internet data communications, (c) a medical apparatus memory component that selectively stores MA-D and comprises processor-executable instructions for one or more medical apparatus computer implemented data engines, (d) a medical apparatus processor that executes the instructions in the medical apparatus memory component, (e) a network status engine that (I) automatically repeatedly checks for availability of a secure and stable internet connection when in operation, (II) when a secure and stable internet connection is present, relays at least some of the MA-D via such secure and stable internet connection to one or more intended recipient internet connected systems, (III) when a secure and stable internet connection is not present, causes at least some of the MA-D to be stored in the medical apparatus memory component as cache MA-D until a secure and stable internet connection is established or reestablished and thereafter relays at least some of the cache MA-D to the one or more recipient internet connected devices or systems; and (2) a medical apparatus controller and data management system (“MAC-DMS”) comprising (a) a MAC-DMS processor that reads computer readable instructions to analyze data and perform functions, (b) a streaming data processing engine that automatically and continuously receives and processes MA-D relayed from the medical apparatuses and performs an initial analysis on received streaming MA-D to determine if one or more preprogrammed conditions are present in the streaming MA-D and, if such one or more conditions are present, acts as a controller of one or more of the medical apparatuses, one or more other network devices, or both by performing one or more of a preprogrammed limited set of initial functions, which initial functions comprise relaying instructions for controlling the operation of one or more medical apparatuses, one or more other network computerized devices, or both, (c) a MAC-DMS memory component that comprises processor-executable instructions for one or more MAC-DMS-implemented engines and one or more data repositories, the one or more data repositories comprising (I) an enhanced data lake comprising a plurality of data lake management zones each data lake management zone being associated with different data lake management zone access rules, (II) a first relational database, and (III) a second relational database, (d) one or more MA-D memory ingestion engines that analyzes MA-D and records MA-D to one or more data lake management zones based on one more preprogrammed criteria, (e) an analytical engine that performs one or more analytical functions at least partially based on analytical data stored in the enhanced data lake upon request from a user, upon occurrence of a preprogrammed condition, or both, (f) an analytical data memory ingestion engine that analyzes analytical data based on one or more preprogrammed conditions and based on such preprogrammed conditions records (I) analytical data generated directly from MA-D in the first relational database, (II) analytical data generated from data contained in the enhanced data lake in the second relational database, and (III) analytical data in one or more data lake management zones of the enhanced data lake, (g) a data repository inspection engine that collects information concerning data being ingested into the first relational database, data being ingested into the second relational database, or both, and relays such information to one or more graphical user interfaces accessible by one or more system administrators, and (h) a network device controller that relays one or more outputs via secure internet communication to one or more medical apparatuses, one or more other network computerized devices, or both, the one or more outputs comprising (I) analytical data output; (II) one or more output applications comprising instructions that control the operation of (A) one or more medical apparatus functions in one or more of the medical apparatuses of the data network, (B) one or more other network device functions in one or more of the other network devices of the data network, or (C) both (A) and (B); or (III) a combination of (I) and (II).

In another aspect, the invention provides a method of treating a medical condition of a patient in a population of patients, the method comprising: (1) providing a data network comprising MAs and an NDS/MAC-DMS, each having the features described in the preceding aspects of this Section with respect to such elements, (2) causing each operating medical apparatus to repeatedly collect sensor data from the patient associated therewith; (3) causing each of the medical apparatuses to automatically and repeatedly assess whether a secure network connection is available, and (a) when a secure and stable network connection is available, automatically relaying data comprising MA-D in a substantially continuous manner via secure internet data communication to the MAC-DMS, or (b) when a secure and stable network connection is not available (I) storing MA-D in the medical apparatus memory component as cache MA-D until a secure and stable network connection becomes available and (II) when a secure and stable network connection becomes available, relaying the cache MA-D to the MAC-DMS via secure internet data communication; (3) causing the MAC-DMS processor to automatically use the streaming data processing engine to (a) receive the streaming MA-D relayed from the medical apparatuses, (b) perform an initial analysis on the relayed streaming MA-D received from each medical apparatus, and (c) perform one or more of a preprogrammed limited set of initial functions if the initial analysis identifies one or more preprogrammed conditions in the streaming relayed MA-D, which initial functions comprise relaying instructions for controlling the operation of one or more medical apparatuses, one or more other network devices, or both; (4) causing the MAC-DMS processor to use the cache MA-D processor to (a) receive cache MA-D when cache MA-D is relayed from a medical apparatus to the MAC-DMS and (b) determine whether the received cache MA-D is suitable for analysis by the analytical engine, storage in at least one of the one or more data repositories, or both; (5) causing the MAC-DMS processor to automatically store at least some of the MA-D in the enhanced data lake; (6) causing the MAC-DMS processor to use the analytical engine to perform one or more analyses on at least some of the analytical data stored in the enhanced data lake upon request from a user, upon occurrence of a preprogrammed condition, or both, wherein the analytical engine (a) performs an analysis using analytical data generated by MA-D from the medical apparatuses and in performing the analysis and (b) compares MA-D from each medical apparatus to (I) the previously collected MA-D analytical data, (II) preprogrammed standards, or (III) both (I) and (II); and (7) causing the MAC-DMS processor to relay one or more outputs via secure internet communication to one or more medical apparatuses, one or more other network devices, or both, the one or more outputs comprising (a) analytical data output relevant to the treatment of the patient relayed to (I) the medical apparatus associated with the patient, (II) another network device associated with a healthcare provided associated with the patient, or (III) both (I) and (II), (b) one or more output applications comprising instructions that control the operation of (I) one or more medical apparatus functions in one or more of the medical apparatuses of the data network, (II) one or more other network device functions in one or more of the other network devices of the data network that is associated with a healthcare provider associated with the patient, or (III) both (I) and (II). or (c) a combination of (a) and (b).

In still another aspect, the invention provides medical apparatuses (MAs) comprising (1) a high security zone comprising a collection of directly interoperating components, the high security zone components comprising (a) one or more therapeutic components that in operation apply medical treatment to a patient and are controllable through received electronic control instructions, (b) a high security zone memory component that comprises computer readable media comprising instructions for a plurality of computer-implemented instructions that control operation of one or more high security zone components, (c) a computer processor component that executes computer-implemented instructions contained in the high security zone memory component to send electronic instructions to the one or more therapeutic components to control operation of the one or more therapeutic components, (d) a communication component that (I) receives electronic communications from (i) the medium zone component and (ii) a local physical input, (II) comprises a security engine that analyzes communications received from the medium zone component to ensure that only communications that comply with one or more validation standards are allowed to control operation of high security zone components, and (III) relays electronic communication to (i) the medium zone component and (ii) a local medical apparatus output, (e) a physical anti-tampering system that limits access to the high security zone memory component to only authorized users, and wherein (f) the high security zone lacks any capability for direct internet communications; and (2) a medium security zone comprising a second collection of interoperative components, the medium security zone components comprising (a) one or more sensors that measure one or more physical conditions of(J) performance of one or more therapeutic components in the high security zone, (II) one or more physiological conditions of an associated patient, or (III) both (I) and (II) as medical apparatus data (MA-D), and convert such measurements into electronically transmittable data, (b) a medium security zone memory component that comprises (I) computer readable media comprising instructions for a plurality of computer-implemented instructions that control operation of one or more medium security zone components and (II) a component for storage of MA-D, and (c) a computer processor component that executes computer-implemented instructions contained in the medium security zone memory component including (I) a network status engine that evaluates if a secure and stable internet collection is available and (A) when such a connection is available (i) relays MA-D to one or more intended internet recipient devices and (ii) receives instructions relayed from one or more remote control devices via secure internet communications, or (B) when such a connection is not available, stores MA-D in the medium zone security memory component as cache data until such a connection is available and then relays the cache data to one or more intended recipient devices, and (II) an inter-apparatus communication engine that receives data from, and sends electronic instructions to, the therapeutic component.

### BRIEF DESCRIPTION OF THE DRAWINGS/FIGURES

Exemplary aspects of the invention are illustrated in the Figures provided with this disclosure. Such aspects shown in the Figures are briefly described in this section and in more detail elsewhere. Uncontradicted, features/steps illustrated in any figure may combined with element(s)/step(s) of any other figure(s). Figures should be generally viewed as component aspects of overall embodiment(s) of the invention, but with the understanding that not all illustrated features are necessary for each embodiment.

FIG. 1 depicts a simplified network connection between a medical apparatus (MA) and a Network Data System (NDS/System).

FIG. 2 is an overview of a simplified network of MAs and their relationship to an NDS according to one exemplary embodiment.

FIG. 3 is a simplified layout of a level of an MA network comprising several MA subnetworks identifiable by groups of MAs, and communication of data to an NDS according to exemplary aspects.

FIG. 4 is an overview of an exemplary process to address MA data collection in offline periods according to one aspect.

FIG. 5 provides a description of a scenario further illustrating the collection and possible processing of cache data according to one embodiment, with reference to exemplary physical components and locations.

FIG. 6 illustrates an MA/NDS network comprising an MA network, an NDS, and ONDIs according to exemplary NDSs/methods.

FIG. 7 provides an exemplary data management process within a network system memory unit according to an aspect.

FIG. 8 depicts the application of different data zones to an NDS data repository/memory unit (e.g., an enhanced data lake), per one aspect.

FIG. 9 provides an overview of an exemplary network comprising MA groups and an NDS, illustrating physical device components operating within the network, according to one embodiment.

FIG. 10 illustrates the flow and processing of data within an exemplary network of the invention.

FIG. 11 illustrates data processing methods employable in components of a multi-zone medical apparatus (MZMA).

FIG. 12 provides an overview of the components of an MZMA in a network with an NDS according to aspects and illustrating management of data flows through such components.

FIG. 13 illustrates an exemplary flow of data from an NDS into and through another exemplary MZMA.

FIG. 14 depicts select physical components of an MZMA and the control of data into and within an exemplary MZMA.

FIG. 15 illustrates exemplary physical components of an exemplary MZA and control of data flows between an MZMA and an NDS.

FIG. 16 depicts a secure process for updating the operating system or other software of parts of an MZMA via a staged data exchange with components of an MZMA and an NDS.

FIG. 17 illustrates a network comprising MAs in different operational states relaying and receiving functional data from an NDS.

FIG. 18 is an illustration of the relationship of different user entity interfaces that receive analytical data from an NDS (NDS-AD) through specialized user class displays comprising user class sorted NDS-AD.

FIG. 19 is a schematic that reflects the application of different but overlapping NDS architectures for different regions/countries.

FIG. 20 depicts an overview of the processing of MA data over time and according to time-dependent rules.

FIG. 21 illustrates the application of various user class-level and individual user level event alerts/alarms in an exemplary network.

FIG. 22 depicts an exemplary network including multiple machine learning modules (MLMs) managed by a master MLM.

FIG. 23 depicts flow of data into, within, and from a streaming data processor component of a system/NDS and the selective processing and analysis of streaming data before ingestion into NDS memory.

FIG. 24 is another exemplary network demonstrating flow of data through various parts of the network including two relational databases that form part of the NDS data repository, each of which being subject to data quality monitoring dashboards.

FIG. 25 is an example of a semi-unstructured dataset, including device performance data, which can be relayed from an MA to an NDS.

FIG. 26 is another exemplary dataset, including patient sensor data.

FIG. 27 is an additional illustrative semi-unstructured dataset including device (MA) alarm (performance) data.

FIG. 28 is a further exemplary semi-unstructured dataset including MA system/software information.

### DETAILED DESCRIPTION OF THE INVENTION

For convenience, principal features/parts (e.g., steps/functions of methods and Units of systems) are described in this section, individually, as well as sometimes in combination with other aspects, features, embodiments, elements, etc. As indicated elsewhere, any specific aspect, embodiment, function, feature, or unit can be combined with or applied to any other aspect, embodiment, etc., or, where suitable, any described step, aspect, element, etc., can substitute for any alternative aspect, step, feature, etc.

In one aspect, the invention provides systems (each, a network data system (“NDS”)) including modules, units, engines, or components, and methods comprising steps for performing the functions of such modules, units, engines, or components, for securely receiving, analyzing, storing, managing, and applying machine apparatus data (MA-D) from a number of MAs in a data network with the system/NDS, generating analytical data from such MA-D (NDS-AD), and relaying such NDS-AD or related outputs to MAs, other network devices (ONDs), or both, typically through secure internet communications. In aspects, such related outputs include output application(s) that control one or more aspects of MA operation, OND operation, or both (such that the NDS is a MAC-DMS). E.g., outputs can include registering alarms on MAs/ONDs, changing the graphical displays of MAs/ONDs, or changing therapeutic or diagnostic component(s) of MAs that interact with subjects. In aspects, MAs of the network include multi-zone MAs (MZMAs) that comprise separate components under separate performance, security, or communication settings from the other zone of the device, thereby promoting device security when the MZMA is communicating with the NDS via internet communications. In aspects, MAs collect cache data (MA-CD) under certain conditions, such as when internet communications are not available, and thereafter relay the MA-CD to the NDS, which determines whether the MA-CD should be stored in the data repository of the NDS, used in generating NDS-AD, or both.

In aspects, NDSs perform real time (“RT”) or near real time (“NRT”) Function(s) related to such MA-D and MAs, typically to facilitate, promote, or carry out medical treatment through or related to such MAs. In aspects, NDSs have access to group(s) of separately located and at least partially remotely controllable MAs and data therefrom. In aspects, methods comprise collecting data communicated from such MA group(s) (“MAG(s)”), storing the data from such MAG(s), analyzing or evaluating data communicated from such MAG(s) to generate analyses (NDS-AD), executing Function(s) based on such analyses. In aspects, Function(s) comprise communicating NDS-AD, instructions, or both, to network component(s), such as MA(s) in MAG(s), other device(s) in the network (ONDs), or both. MAs of a network typically are in recurring, and often substantially continuous communication with the system (NDS), which is typically facilitated through known or described secure internet-based communication methods.

In aspects, MAGs in a network with an NDS are under control of ≥two Independent Entities (IEs) (e.g., ≥3, ≥5, ≥7, ≥10, ≥12, ≥20, ≥30, ≥50, or ≥about 100 IEs), and the NDS comprises Functions for identifying communications from and relaying data to each specific MA in the network including identifying each Entity associated with each MA, executing MAG-specific instructions, segregating data based on MAG association, or a combination thereof (CT).

MAs collect data concerning the diagnosis or treatment of disease in subjects. For example, MAs can comprise sensor(s) that in operation collect sensor data (comprising measurements of physical phenomenon, such as device performance, patient physiological state, or both). Such “sensor data” is a component of data collected by an MA (i.e., MA data (or “MA-D”)). MAs typically also include (1) device/local memory (comprising physical, transferrable, and reproducible computer readable medium (PTRCRM)) and (2) a processor/processing function/unit for executing device CEIs, which can include functions concerning when the MA stores MA-D, relays/transmits MA-D, or both. MA-D that is at least sometimes stored in such MA local memory is also called “cache data,” even when such data is later relayed from an MA to an NDS.

MAs of a network can comprise device display unit(s) (comprising, e.g., graphical user interface(s) (GUI(s))) or other interfaces/output devices/components). In aspects, output of an NDS to an MA is dependent on, i.a., the class of user(s) associated with the MA.

MAs of a network typically comprise data relay unit(s) that transmit MA-D from the MA to the NDS. In aspects, in operation MA(s) collect(s) and relay(s) MA-D to an NDS sometimes, most of the time, generally all of the time, or substantially all of the time as streaming (or streaming and real-time) time MA data (as “streaming MA-D” or “SMAD).” Like other MA-D, SMAD can comprise MA sensor data, device performance data, device identification data, patient identification data, or a combination of any or all thereof). In aspects, cache data, when relayed, also is relayed as a data stream. In aspects, cache data is sent with real-time (RT) SMAD (“RT-SMAD”). In aspects, cache data is relayed separately from RT-SMAD.

MA-D can comprise semi-unstructured, structured, or unstructured data. In aspects, some, most, or at least generally all MA-D relayed by the MA comprises semi-unstructured data. In aspects, use of semi-unstructured data detectably or significantly enhances the speed of the NDS, uptime of the NDS, or both (e.g., by reducing incoming data load/processing burden).

In aspects, some, most, generally all, substantially all, essentially all, or all MA-D is sensor data. In aspects, MA-D in some, most, generally all, or all cases comprises additional data (non-sensor data). In aspects, MA-D comprises device status/performance data (data concerning one or more aspects of MA operation/operability, such as power status, pump speed, etc.). Such data can be referred to as “device data.” In aspects, most, generally all, substantially all, essentially all, or all MA-D is sensor data combined with device performance data or other device data. In aspects, some, most, generally all, or all device performance data provides indirect information concerning patient status (e.g., movements, such as rotations of a device or device component, device pressure, etc., may provide indirect information concerning one or more patient physiological parameters that are preprogrammed into an NDS/MA).

MAs of the network can security feature(s), such as an incoming data security unit for ensuring only authorized information can modify MA operation(s), physical anti-tampering detection or blocking measures, etc. Such components are known and discussed elsewhere.

In operation, MAs sometimes, most of the time, generally all the time, or substantially all the time relay data to an NDS/system. In aspects, in operation, most of the time, generally all of the time, or substantially all of the time MA(s) are in substantially continuous communication with an NDS via internet or other WAN/SDWAN connection.

An NDS typically will comprise (1) a NDS memory unit/component (“memory”) comprising PTRCRM comprising data repository(ies) (DR(s)) that (A) receive(s) and store(s) MA-D and (B) comprises NDS CEI (NCEI) including encoded/preprogrammed instructions for Functions (engines) that are carried out by (2) an NDS processor that reads/executes the NCEI. NDS(s) can further comprise components that can be characterized as, e.g., (3) an NDS data input unit/engine (e.g., an engine that selectively automatically receives data from MAs (and typically determines if MA-D received from each MA is SMAD, cache data, or both)), (4) NDS analytical unit(s)/engine(s) (e.g., an engine that analyzes MA-D and possibly other input(s) according to pre-programmed and programmable rules to generate analytical data (NDS-AD)), and uses such analysis to direct performance of NDS application(s)/function(s) (e.g., through controller function(s)), (5) a NDS data evaluation unit/engine that evaluates if MA-D is approved for use by the analytical functions or determines how MA-D can be used by such functions (and optionally one or more data improvement units that perform data harmonization, data cleaning, and data validation functions), (6) a NDS output engine/unit/processing system that automatically filters MA-D, analysis, or both, for delivery to MAs and to other network devices and interfaces (ONDIs), based on preprogrammed factors/rules, e.g., confidentiality and health care compliance rules, and (7) an NDS data relay unit/engine. An NDS data relay engine/unit typically securely relays over the internet (A) information to MAs that is specific to such MA, associated patient, or both, and typically is adaptable for receipt through a security unit of such MAs and (B) information comprising MA-D, analysis, or both, to one or more other network devices/interfaces (ONDIs). Optionally, an NDS can further comprise (8) a NDS cache data analytical function that comprises special instructions for identification of cache data, analysis of cache data, or handling of cache data, and, e.g., generates cache data NDS analyses based on application of functions to data stored in the NDS memory/DR(s). In aspects, cache data is used to supplement RT-SMAD; reconstruct a data stream where RT-MMAD is lacking/missing; validate incomplete, questionable, or otherwise impaired RT-SMAD; etc.

In aspects, some, most, generally all, or all MAs of a network are mobile MAs, which may from time-to-time lose network (e.g., internet) connectivity and, accordingly, have periods in which RT-SMAD (SMAD) cannot be relayed to the NDS. In aspects, analysis of both SMAD and cache data leads to a DOS improvement in subject care.

Data relayed to ONDIs can comprise, e.g., information from several MAs associated with ≥2 independent entities (IEs) or NDS-AD derived from MAs owned/controlled by ≥2 IEs. In aspects, such information can be combined with information external systems/data repositories, such as CRMs.

NDSs typically include processing capabilities/component(s) and unit(s)/engine(s) that allow for the concurrent receipt and processing of MA-D, and for relaying MA-D or NDS-AD to ONDIs, MAs, other devices/interfaces (e.g., accessible web portals), or a combination thereof, such as data tagging functions and massively parallel distributed and scalable cloud-based processing functions. Such capabilities also can allow for an NDS/method to be applied to multiple types of MAs, multiple patient conditions, multiple patient types, and multiple MA states, concurrently, expanding the applications of such NDSs/methods, and the robustness of the data in and derived by such systems (e.g., in concurrently collecting data from research class individuals/groups as well as patients in the clinic), and further allowing for national-level and even international-level application of such NDSs/methods. Facilitating step(s)/function(s) performed by NDSs/included in methods can also include data queueing and prioritization methods (applied on inbound/incoming MA-D, as discussed elsewhere).

Data in DRs of the NDS can be subject to multiple “levels” of identification, management, security, access, etc., based on data characteristics. Functional data/CEI of an NDS can be used to filter, redact, direct, and otherwise manage distribution of data generated or received by an NDS (e.g., excluding PHI from data provided to commercial users, such as device salespeople and for otherwise ensuring compliance with Regulatory Requirements (RRs)). In aspects, NDSs can concurrently deliver different sets of NDS-generated data (analytical data/analysis or NDS-AD), MA-D, output based on NDS-AD/MA-D, or a combination thereof, to MAs and various other network devices/systems, which, in aspects, include devices assigned to different users/classes. In aspects, the data relayed by the NDS varies depending on RRs and user needs/roles applicable to/associated with users of different roles/classes. In aspects, an NDS accommodates several individualized or class-level alarms/alerts set at a local/user level, NDS/class level, or both.

MAs of a network can comprise one or more protections against unauthorized modification or tampering. E.g., in aspects MAs comprise anti-tampering component(s)/systems (e.g., engines, sensors, etc.) that limit/inhibit or stop MA performance when unauthorized tampering is detected. In aspects, MAs comprise sensors, components, engines, systems, etc., which register or relay alarms (e.g., by registering a physical auditory, visual, or audiovisual alarm on an MA; by sending an alarm message to the NDS, ONDIs, or both; or a combination thereof).

In aspects, MAs can be characterized as “multi-zone MAs” (MZMAs), which are MAs that comprise 2 or more sets of components or systems having different levels of interactivity with the network, different levels of interaction with a patient, or different levels of device security (e.g., an MZMA can comprise first zone comprising a first therapeutic component, e.g., a component involved in critical care/life support functions, such as cardiovascular or pulmonary life support/treatment functions, that is subject to more restrictions in terms of incoming data than a second component which can be, e.g., involved in a different type of therapeutic application or a diagnostic/monitoring application).

MAs can be, in aspects, directly controlled by an NDS (a MAC-DMS) in one or more respects, such as the application of direct medical therapy, in displaying a course of treatment for HCPs, or for providing a recommended course of treatment for HCPs.

NDSs can comprise machine learning modules (MLMs), for each type of device, conditions treated, patient types, etc., and, in aspects, can further comprise master MLM(s) for managing multiple, overlapping MLMs for patient(s). In aspects, NDS/method have a relationship between data upload from MAs and performance of analytical functions, such as MLMs, e.g., such that (1) there are more, typically many more (e.g., ≥5, ≥10, or ≥25), data collection cycles contributing to an analyzable data collection performed in the period it takes to collect a sufficient data collection for performing the analytical function(s) and (2) there are step(s)/function(s) for re-starting the contribution of data collection cycles to the data collection in the event that there is a detected problem with anyone data collection cycle that cannot be repaired or otherwise compensated for by the NDS.

Additional aspects of these and other features of the systems, networks, and methods of the invention are described in particular detail in the following sections of this disclosure.

### A. Medical Apparatuses (MAs) and MA Groups (MAGs)

Networks can comprise group(s) (sometimes called networks) of multiple MAs, each MA group (“MAG”) comprising one or more type(s) of MA(s), and each type of MA typically being associated, in operation, with one or more type(s) of patients/subjects, type(s) of treatments/diagnosis/applications, or a combination thereof.

In aspects, a network comprises ≥5 MAs in communication with an NDS, and in aspects a network comprises ≥~20, ≥~100, ≥~200, ≥~500, ≥~1000, ≥~5000 MAs (MA units) (e.g., 100-1000 MAs, 100-10,000 MAs, or 100-100,000 MAs) in communication with an NDS (in ≥1, ≥2, ≥3, ≥5 or more MA types).

In aspects, a network comprises ≥2 MAGs (e.g., ≥3, ≥5, ≥7, ≥10, ≥12, ≥15, ≥20, ≥25, ≥35, or ≥50 MAGs), each MAG comprising e.g., ≥~5 MAs (e.g., ≥10, ≥20, ≥25, ≥50, or ≥100 MAs). In aspects, some, most, generally all, or all MAs of some, most, generally all, substantially all, or all MAGs are under the primary control of independent entities (IEs), distinct from each other and from the system owner/system operator. In aspects, a network comprises ≥5 MAGs (e.g., 5-100, 5-50, or 10-40 MAGs) that each are under primary control of separate independent entities (IEs) (with respect to each other and the NDS owner/operator (SO)). “Primary control” means final responsibility for the operation of the MA (e.g., by owning title to the MA, bearing the liability for its treatment/diagnosis of subject(s), or both).

In aspects, an SO is the owner of the NDS. In aspects, the SO is operated by an operator entity or individual. In either case, the SO can employ persons that manage aspects of the NDS, which can be referred to as “administrators,” “admins,” “system administrators,” etc. In aspects, one or more IEs associated with an MAG allows a SO to receive data from MAs, control aspects of MA operation, or both. In aspects, MAs, an NDS, or both, are provided with electronic contracting function(s) that comprise, e.g., means for establishing such access authorization. In aspects, such electronic contracting functions are programmable and updatable.

In other aspects, some, most, generally all, or all Entities that own MAs in the network maintain complete control over the MAs but are granted permission to use the NDS by the SO (and typically grant the SO right to access IE-associated PHI, such as MA sensor data, to allow the SO to operate the NDS).

In aspects, some, most, generally all, or all MAs in a network are MAs that were initially controlled by the SO (e.g., are MAs that were manufactured or sold/granted/transferred by or for the SO to current IE owners). In some such aspects, SO-imposed capabilities in terms of NDS interaction in the MAs are established prior to transfer of ownership (e.g., ability to collect or transmit data in one or more of the standards described herein) and maintained in most, generally all, substantially all, or all MAs in the network. Such aspects allow for specific configuration of elements (e.g., the format of most, generally all, or substantially all MA-D, NDS-AD, etc.). However, in other aspects, such functionality is also or alternatively achieved by IE grant(s) of permission to the SO to control aspects of MA operation. In aspects, components of operation are achieved by both measures (e.g., initial settings were set by the SO when it owned the device but have to be updated later based on IE permission).

The connection of MAs owned/managed by separate IEs (from each other) typically imposes additional data management requirements to ensure that confidentiality of MA-D obtained from MAs associated with each different IE are protected from access by users associated with other IEs (as well as not disclosed to another other unauthorized entity/individual). Accordingly, NDSs associated with such multiple IE-owned MAGs can comprise protocols at the MA and NDS level in which MA-D and NDS-AD derived from different IE MAGs are identified as associated with the applicable IE. Method of data “tagging” are known and described elsewhere. Such methods can comprise, e.g., addition of packet information to MA-D that identifies the source of MA at one or more levels (such as a specific device level, device type level, device functional status level, an IE level, or other levels such as a regional or national level). In aspects, engines, units, or functions applied/performed at the device level, system level, or both, also identify other levels of association that can define MAGs to which each MA belongs (e.g., a hospital treatment group, a hospital, a hospital system, a town, a metropolitan area, a county, a state/province, etc.).

In aspects, MA(s) in a network can be present as a stand-alone medical apparatus(es) (e.g., an independent medical device). In aspects, ≥2 MAs form a MAG that accounts for some of the MAs in the network. MAGs can be defined by location (e.g., a site), by a sub-network (e.g., an Entity MAG, such as a network of hospitals or clinics), a geographic location (e.g., an area MAG which can define a city, county, state, region, country, or continent), or e.g., a group of patients (e.g., a patient group comprising patients diagnosed with a shared condition). In aspects, MAGs are modifiable after initial creation, such as one or more MAs can be added or removed from a group, or one or more MAs can be transferred from one MAG to another. In aspects, an MA can be long to 2, 3, 5, or MAGs, e.g., where multiple MAG categories are accounted for in a network (e.g., an MA can belong to a MAG defined by MAs owned by IE X, MAs in region Y, or both where such MAGs overlap). In aspects, the characterization/grouping of MA(s) can fluctuate over time, in aspects in real time, such as groupings of MAs based on patient status, e.g., groups of patients sharing one or more physiological parameters in common, such parameter(s) changing over time and hence the grouping of associated MAs changing over time. In aspects, one or more MAs can belong to multiple collections at any single point in time, e.g., belonging to both a site group and a patient group. To identify an MA in MAG(s) an NDS can comprise CEI comprising protocols/rules for associating each MA into relevant MAG(s), based on, e.g., MA-D reflecting operation of the MA, associated subjects, location, IE-affiliation, MA type, etc. E.g., an MA can add such information to MA-D and an NDS can comprise CEI that specifically identifies such information. In aspects, such information is identifiable as a type of attribute/value pair in semi-unstructured MA-D.

In facets, each MA of a network is at least partially remotely controllable; that is, in some facets most, generally all, or each MA has ≥1 function, ≥about 2, ≥about 3, ≥about 4, or, e.g., ≥about 5 or more functions, that can be operated from a location that is remote/not local to the apparatus, e.g., from the network data system (NDS), other network interfaces/devices (e.g., a HCPs device, an IE administrator interface, or both, or other ONDI(s)).

In embodiments, some MAs of a network are classifiable as stationary devices or apparatuses (e.g., MAs that generally or substantially always operate in a single location once set up). In aspects, some, most, generally all, substantially all, or all MAs in a network are mobile medical devices. A mobile MA is an MA that is adapted to, configured to, and capable of ready movement in healthcare settings, such as in a hospital, in ambulance care, etc., e.g., being lightweight, being adapted for portability (e.g., by being a wheeled push device, a handheld device, a robotic mobile device, a vehicle-based device, etc.). In aspects, mobile MA(s) comprise wireless internet or other types of wireless internet access/telemetry capabilities (e.g., ability to transmit data over cellular networks, Bluetooth, etc.). In aspects, a network can comprise a combination of stationary and mobile MAs. In aspects, some, most, generally all or all of the mobile MAs mostly, generally always, or only relay MA-D to the NDS via a wireless secure internet communication protocol, such as Wi-Fi.

A network can include one or more types of MAs of any suitable type(s). In aspects, some, most, generally all, substantially all, or all of the MAs are critical care MAs. In aspect, the MAs support cardiovascular, pulmonary, nervous system, or gastrointestinal system or organ support such as heart support, lung support, liver support, kidney support, etc. In aspects, MAs comprise a heart pump. In aspect, MAs comprise extracorporeal membrane oxygenation (ECMO) devices. In aspects, MAs of a network comprise both ECMO and heart pump devices.

According to aspects, MAs comprise ≥2 heterogenous types of MAs. In aspects, an NDS determines the type of device each MA is associated with in the uploading of data from MAs, downloading of data to MAs, or both (e.g., by reading device-type identifying MA-D relayed by each MA-D). According to aspects, heterogeneous MAs can share≥1 characteristics in common, e.g., their primary medical purpose (e.g., cardiovascular or pulmonary support). In aspects, at least some, most, or generally all heterogenous MAs of a network provide distinct functions, such as support of different organ or physiological systems (e.g., cardiovascular system, pulmonary system, or nervous system support). In aspects, heterogeneous MAs can each provide MA-D related to 1 or more of the same measured elements, such as certain physiological parameter(s) (e.g., blood pressure, heart rate, etc.). According to aspects, heterogeneous MAs provide MA-D for shared physiological parameter(s) such that the MA-D originating from such devices is also at least partially in a shared/common format (e.g., comprising similarly formatted value/attribute pairs for blood pressure, oxygen level, heart rate, and the like, in semi-unstructured MA-D). According to alternative facets, heterogeneous MAs also or alternatively provide MA-D for shared physiological parameter(s) in different formats. In such cases, data harmonization can be performed in an NDS using known methods to “unify” MA-data from different types of MAs to generate combined NDS-AD. Such processes are discussed further elsewhere.

MAs can be associated with any type of medical procedure, medical workflow, and the like, e.g., can provide intervening or monitoring support for any medical condition. In aspects, an MA can be a component of an outpatient-related medical device. In aspects, an MA can be an apparatus designed for operation by trained medical personnel within an established medical facility such as a hospital or clinic. In aspects, one, some, most, generally all, or all of the MAs of a network of MAs is/are associated with a critical life support function such as supporting function of the cardiovascular system (heart), pulmonary system (lung), brain, or kidney(s). In certain facets, at least most of the MAs of a network are associated with a critical life support function (such as, e.g., supporting/treating the respiratory system, the cardiovascular system, or the nervous system). In aspects, an MA is an implantable medical device. In facets, an MA is a medical device that in operation is present within the cardiovascular system, pulmonary/respiratory system, brain, or kidney(s) of subject(s). In more particular aspects, one or more MA(s) is an implantable medical device implanted into the heart, such as a device similar to one of the known Impella® heart pump devices (Abiomed, Inc.). In other aspects, the MA is a device that comprises components that are internally positioned in subjects or that interface with internally implanted components but comprises external components, such as a Breethe OXY-1 System (Abiomed, Inc.). In aspects, a network comprises both heart pumps and ECMOs (e.g., Impella® and Breethe™ devices).

In facets, most, generally all, substantially all, or all MAs in a network are in substantially continuous communication with an NDS in operation. For example, in aspects, MAs in one or more group(s) in a network are in continuous communication with an NDS for ≥about 50%, ≥about 65%, ≥about 75%, ≥about 80%, ≥about 85%, ≥about 90%, ≥92.5%, ≥about 95%, ≥about 97%, ≥about 98%, or even ≥about 99% of time on average, on a daily, weekly, monthly, quarterly, or annual basis. In aspects, MAs are mobile MAs that, in operation, transmit RT-SMAD to an NDS via a wireless communication protocol≥about 50%, ≥~65%, ≥75%, ≥about 85%, ≥about 90%, ≥about 95%, or ≥about 97%, ≥~98%, or even ≥about 99% of the time, on average, on a daily, weekly, monthly, quarterly, or annual basis.

In aspects, “continuous communication” means, uploading, downloading, or both uploading and downloading an analyzable/actionable amount data less than every 1 minute (e.g., less than every 30 seconds, ≤~15 seconds, ≤~10 seconds, ≤~5 seconds, ≤~2 seconds, ≤~1 second, ≤~0.5 seconds, ≤~0.25 seconds, or ≤~0.1 seconds) in operation. “Substantially continuous” communication means achieving such levels of communication substantially all of the time the MA is in operation (i.e., at least 95% of the time when in operation). In aspects, substantially continuous or continuous communications comprise, generally consist of, consist essentially of, or consist of streaming communications (discussed elsewhere). An “analyzable/actionable” amount of data means an amount of data that is sufficient for performing one or more analytical functions/processes, one or more operative functions (e.g., controlling actions of one or more other components of the network, such as MA(s)), or both.

In exemplary aspects, MAs comprise at least two different MA types, the MA types comprising a first type of medical apparatus that performs pulmonary therapeutic task(s) and a second type of medical apparatus that performs one or more cardiovascular therapeutic task(s), wherein operation of the NDS comprises applying a machine learning module (“MLM”) on MA-D received from both the first type of medical apparatuses and the second type medical apparatuses to generate machine learning-generated NDS-AD. In aspects, machine learning NDS-AD comprises predicted, patient-specific, physiological parameters associated with the therapeutic tasks being performed by MA(s). In aspects, an NDS is preprogrammed to deliver such predicted patient-specific physiological parameters automatically or conditionally to MA(s) of one or both of the two different MA types, to other network devices (ONDs), or a combination thereof.

In aspects, users comprise research user(s), and one or more MA(s), OND(s), or both, in the data network are associated with one or more research user type(s). In such aspects, an NDS/MAC-DMS can, e.g., receive input from research user-associated MA(s), (b) the MAC-DMS can combine MA-D from at least some of the research user associated MA(s) with MA-D obtained from medical apparatuses associated with health care providers to form a mixed data set, and (c) the MAC-DMS can perform an analysis/analytical function on the mixed data set. In aspects, such mixed data or mixed data function/application (or research data/research data application output(s)) is/are specifically identified separately from other NDS-AD/output(s) in output. In aspects, such output is only provided to user devices associated with research class user(s) (e.g., researchers engaged in clinical trial(s)). In aspects, other user(s), such as practicing HCP(s), have the ability to opt in to receive outputs, analysis, etc., based on mixed data, based purely on research data, or both. Such identification can be performed through packet/data tagging, as known and described elsewhere herein. E.g., in aspects, an NDS comprises CEI for adding such tag(s) and MA(s)/OND(s) comprise CEI for identifying such tag(s).

### I. MA Subject/User Interaction Components

MAs can be characterized based on components that interact with users/subjects, such as in collecting data from subjects through sensors, collecting data from users through direct data input in the MA or an associated user interface, applying therapy to a subject, providing directions for therapy to a HCP, monitoring/diagnosing (detecting) conditions, etc.

### 1. Sensors

In aspects, some, most, generally all, or all MAs in the network comprise sensor(s), are associated with sensor(s), or both. Sensor(s) typically comprise or are device(s) or component(s) of device(s)/system(s) that sense condition(s) relating to a patient physiological state, device performance/state, other physical condition, or a combination thereof. Sensor(s) can, e.g., comprises sensing components/devices that detect subject-associated physiological state(s) and convert information regarding such physiological state(s) to processor-readable data (MA-D). As discussed elsewhere, various sensor technologies are known and applicable to aspects.

In aspects, one, some, most, generally all, or all sensor(s) of, or associated with, MAs or one or more type(s) of MA(s) are sensors that make contact with the body, such as wearable sensors. In aspects, one, some, most, generally all, or all sensor(s) of or associated with an MA are internal sensors, sensing conditions of a patient by placement in, e.g., a patient's organ, blood vessels, lungs, or other internal tissue/system.

A sensor that is associated with an MA can be a sensor that is separate from the MA but that relay(s) information about the MA, the patient the MA is associated with, or both, to the MA or to other device(s) or network(s) that users can review.

Sensor(s) can be any suitable sensor(s) known in the art and typically the nature of the sensor(s) associated with a subject will depend on the nature of the condition in the subject that is being sought to be prevented, treated, or diagnosed through the MA(s). Examples of sensors include oxygen saturation monitors, respiration monitors, temperature monitors, blood oxygen monitors and other oxygen level monitors, heart rate monitors, blood pressure monitors, arterial pressure monitors, brain activity monitors, response monitors (e.g., reaction monitors), capnography monitors, blood flow sensors, blood/tissue/urine glucose monitors, biosensors (e.g., pathogen biosensors), other urine monitors, other fluid monitors, position monitors, movement/motion monitors/accelerometers, electromyograms (EMGs), spirometers and other airflow monitors, electroencephalograms (EEGs), other electrical bio-signal monitors, gas exchange monitors, force sensors/inertial sensors, sleep monitors, skin condition monitors, electrocardiogram monitors, and any other physiological or health monitor known in the art. In aspects, sensor(s) can also sense ambient/environmental data. Examples of sensors are described in, e.g., Wilson CB. Sensors 2010. BMJ. 1999; 319(7220):1288. doi:10.1136/bmj.319.7220.1288 and Sergio L. Stevan, “Sensors for Health Monitoring,” Scholar Community Encyclopedia, accessed at https://encyclopedia.pub/2084. Sensors typically comprise computer interfaces, which convert sensor readings into measurement(s) that can be relayed to computer processors using typical device data relay standards or other data relay standards.

In aspects, MA(s) is/are classifiable as diagnostic MA(s), which primarily, substantially only, or solely interact with subjects through sensor(s) (e.g., ultrasound and MRI machines, PET and CT scanners, and x-ray machines, scopes, biopsy devices, or other devices involved with monitoring conditions, diagnosing conditions, or both, such as imaging devices (including, e.g., cameras), probes, and other sensor-based devices).

In aspects, MA(s) are therapeutic devices, comprising sensor(s) in addition to therapeutic component(s). In aspects, in therapeutic devices, one, some, most, generally all, substantially all, or all sensor(s) are associated with the provision/attempted provision of a therapeutic effect associated with device performance. In aspects, an MA will comprise ≥1 sensor that, when the MA-D is in operation, detects at least 1 condition in a patient. In aspects, each MA comprises ≥about 2, ≥about 3, ≥about 5, ≥about 8, or ≥about 10 sensors or more, such as ≥about 15 or ≥about 20 or more sensors, capable of detecting≥about 2, ≥about 3, ≥about 5, ≥about 8, ≥about 10 conditions (e.g., physiological parameters) or more, such as ≥about 15 or ≥about 20 or more conditions or parameters, e.g., ≥~30, ≥40, or ≥50 conditions/parameters.

### 2. Therapeutic Components

In aspects, MA(s) comprise(s) therapeutic component(s). “Therapeutic components” are MA component(s) associated with causing, promoting, or maintaining a therapeutic effect in a subject (in operation preventing, mitigating, treating, or curing disease(s) or condition(s)). In aspects, MA(s) are critical care devices, involved in treating or preventing a disease or condition that is related to sustaining life (e.g., preventing imminent risk of death), normal brain function, basic mobility/material quality of life, etc. Examples of such MAs include, e.g., medical ventilators, incubators, defibrillators, anesthetic machines, heart-lung machines, suction devices, pressure devices, lavage/flushing devices, ECMO devices, pumps (e.g., infusion pumps, heart pumps, etc.), biopsy devices, surgical devices (e.g., spinal surgery devices, heart surgery devices, etc.), shunts or other pressure-relieving devices, ventricular assist devices, pacemakers, tissue/organ or vessel/lumen temperature modifying devices, balloon pumps, other vessel/arterial opening/modulation devices, catheters, stents, syringe pumps, injection devices, sutures, valves, analgesia pumps, transfusion devices, organ function supplementation or replacement devices, circulation access devices (ports, lines, etc.), other circulation devices, etc., and dialysis machines. In aspects, therapeutic components modify parts of a subject's body through surgery or other body manipulation (e.g., medical lasers, incision devices, suction devices, punch biopsy and other biopsy devices, debriding devices, clamps, high frequency ultrasound, temperature modulation devices, etc.).

In aspects, therapeutic or diagnostic components, or both, are in part or entirely remote controlled, robotic or robotically-assisted, computer system-controlled, etc.

### 3. Operational Controls

In aspects, MAs comprise operational control(s) controlling≥1 aspect of MA operation. Such controls can include, e.g., controls for therapeutic component(s) (e.g., pump speed, flow rate, pressure, etc., depending on the therapeutic component), controls for diagnostic/monitoring component(s), or both. Operational controls can also include display controls, alarm/alert controls, or software/data controls (e.g., ports for downloading/transferring or uploading data, interactive interfaces, data inputting devices, touch screens, etc.). In aspects, most, generally all, or substantially all operational control(s) are controlled by aspects of an MA computer operating system/software (e.g., engine(s)). In aspects, some, most, or generally all operational control(s) of an MA are remote controllable operational controls. In aspects, some, most, or generally all of the operational control(s) are controlled by, i.a., NDS output(s).

### 4. MA Display/Output Unit(s)

MAs can comprise output unit(s), such as display unit(s). A display unit can be any suitable device/component for visually displaying information to users, such as a computer monitor/screen, etc. In aspects, display units can comprise or be interactive (e.g., touch screen devices). MA display units also can relay or receive information in audio format. In this respect, the term display can be construed as meaning “sensory output.” Some, most, generally all, or all of an MA display unit can sometimes, mostly, generally always, or always, be subject to remote control, specifically to NDS control.

In aspects, an MA display unit also can be separate from the MA. In this respect, an MA display unit can be supplemented or replaced by a web/software interface, and, accordingly, references to interfaces and display units often can be interchanged herein and, uncontradicted, considered to provide implicit disclosure of a corresponding aspect comprising such a substitution of term. An MA display unit can be a physical component of an MA. A display unit can display, i.a., NDS-AD and related information (e.g., recommendations for treatment, directions for treatment, vital sign predictions that can act as markers for assessing progress of a procedure, condition, etc., etc.). An MA output unit can comprise components for output, including audio output (instructions, alarms, or both), motion output, etc.

In aspects, MA display unit(s) can communicate some, most, generally all, or all conditions of device operation, patient conditions, as well as one, some, or several types of NDS-AD to users (e.g., ≥3, ≥4, ≥5, ≥7, ≥10, ≥12, ≥15, or ≥20 NDS-AD information features/data points). Examples of data features can be but may not be limited to character format (e.g., alpha, numeric, mixed alpha/numeric), coded and uncoded data, language (e.g., English, Spanish, French, German, Arabic, Korean, Chinese, Japanese, etc.), inclusion or exclusion of special characters such as slashes, dashes, asterisks, exponents (or, e.g., subscript and superscript characters), units, symbols, flow charts, diagrams, graphical data, tabular data, images, etc. NDS-AD can include recommended treatment steps, predicted vital sign or other sensor measurements (e.g., based on machine learning modules (MLMs)), system status, software update status, status of monitoring the MA-D by other users of the NDS, etc. MA display units can be a part of a combined input/display unit, such as in a graphical user interface (GUI).

### II. MA Computerized/Computer System Components

MAs comprise computerized components that can, i.a., receive sensor data and relay sensor data or other MA-D (e.g., device status/performance MA-D) to an NDS.

### 1. MA-MEMU/DM (MA Memory Unit(s))

MAs typically comprise memory component(s)/unit(s), systems, or components (which can be referred to as “MA-memory, “device memory” (“DM”), or MA-MEMU). MA-memory typically comprise CEI and other data (e.g., MA-D) contained in PTCRM. DM can store any suitable MA-D, including data received from sensor(s) over time, such as over the period of minutes, hours, days, weeks, months, or over a period of years, depending on memory size, demand, etc. In aspects, storage of data in DM occurs selectively, automatically, or conditionally automatically selectively (e.g., in response to preprogrammed condition(s), such as availability of secure and stable network connections for otherwise relaying data to the NDS), or a combination thereof. In aspects, DM also stores data from other input(s), such as data inputted to MA from a user, data from a subject associated EMR/EHR, internal device operations data, etc. In aspects, the DM is adapted to be capable of storing data generated outside of the MA, e.g., NDS-AD or other output delivered to the MA, such as recommended treatment step(s). DM also typically comprises CEI for the operation of software-controlled components of the MA, which can include engine(s) for controlling aspects of some, most, generally all, or at least substantially all therapeutic components, diagnostic components, display functions, alarm/alert functions, or data receipt/relay functions/operations of the MA. MA-memory/DM can be any suitable type of memory including, e.g., hard drive memory, flash memory, etc. The size of DM will vary with the data load of the MA (e.g., number of sensors, sensor operation rates, complexity of the MA's software/operating system, number of data points measured, etc.), data transmission protocols, MA usage conditions, etc. In aspects, DM will comprise, e.g., ≥512 MB, ≥1 GB, ≥2 GB, ≥4 GB, ≥8 GB, ≥10 GB, ≥15 GB, ≥20 GB, ≥50 GB, or ≥100 GB capacity. In aspects, DM is supplemented by local external hard drive/flash drive memory, local associated computer memory, or the like. In aspects, DM can comprise cloud memory separate from NDS memory. Sensor data or other MA-D contained in DM, even temporarily, can be characterized as “cache data.” Cache data is discussed elsewhere.

### 2. MA Processors

MAs also comprise processor unit(s)/processor(s), which execute device CEIs (MA-CEIs) stored in DM. MAs can comprise any suitable number of processor(s), each being any suitable type of processor(s) (e.g., processor unit(s) comprising a single processor or several processors working together). In aspects, MA processors is/are mostly, substantially only, or only contained in the MA. In aspects, at least some MA processing functions are performed outside of the MA. In aspects, MA(s) comprise ≥2 physically separated and independently operational processing units, at least one of which, in aspects, comprises a multi-processor processing unit (e.g., a multi-core processor).

In general, processors in MAs and other network device(s) can include any suitable type and number of switching elements (e.g., electronic circuits), which maintain states (e.g., binary states suitable for application of binary code machine language) or other suitable states (e.g., in the case of quantum computers, DNA computers, or other alternative computing platforms), with selective change of state functionality and means for reporting state (output), typically based on the logical combination of the states of 1 or more other switching elements, such as logic gates. These basic switching elements can be combined to create more complex logic circuits, including registers, adders-subtractors, arithmetic logic units, floating-point units, or the like. Examples of processor elements/processors include application specific integrated circuits (ASICs), digital signal processors (DSPs), neural network processors (NNPs), digital signal processing devices (DSPDs), programmable logic devices (PLDs), field programmable gate arrays (FPGAs), and CT. MAs and other network devices can comprise one, some, or several of such types of processor systems/components.

An MA processor can have any suitable processor capabilities or characteristics. In aspects where data processing demands on the MA are relatively limited (e.g., in devices with only one or a small number of sensors collecting simple data measurements and typically not collecting image data), a low powered processor can be used as one, some, most, or all of the MA processor(s). In aspects, some, most, generally all, or all MA(s) of a network comprise, mostly comprise, generally comprise, or only comprise such lower power/limited capacity processors, which can, in aspects, be advantageously associated with DOS better power performance (e.g., longer battery life, etc.). E.g., in aspects MAs can comprise a lower power processor which can have a microamp per megahertz (μA/MHz) rating of ≤~200, e.g., ≤~150, or ≤~100. In aspects, an MA processor can have an μA/MHz rating of ≤~70, e.g., ≤~50, or ≤~40 (e.g., by comprising an ARM processor characterized by a 35 μA/MHz performance). In aspects, battery life in such devices can on average, generally, or substantially exceed 3 years, such as being ≥~4 years, ≥~5 years, ≥~6 years, ≥~7 years, or ≥~8 years (e.g., ~10 years). In aspects, energy demands of such processors are about 2-10 volts, e.g., ~3-9 volts, ~3-6 volts, or ~3-5 volts. In aspects, processing speed of a lower powered MA processor is also relatively limited (e.g., operating at ~50 KHz to ~100 MHz, e.g., ~100 KHz-~50 MHz, ~250 kHz-~20 MHz, or ~500 KHz to ~10 MHz, e.g., ~1.5 MHz-~75 MHz, ~2 MHz-~50 MHz, or ~2.5-100 MHz). In aspects, processor(s) of some, most, generally all, or all MAs comprise microcontrollers, system on chip (SoC)/embedded processors, or both, as described elsewhere and in the art. In aspects, such processors can be considered secondary or peripheral processors, having less processing capability in one or more respects (e.g., processing speed) than primary processor unit(s). E.g., in aspects, a main MA processing function is responsible for all MA processor operations not performed by secondary/peripheral processors, such as microprocessors, SoCs, FGPAs, etc., which may be associated with only 1 zone/part of an MZMA. In aspects, most, generally all, or substantially all MA processor functions are performed through a single processor, which, in aspects, is a microprocessor. In aspects, MAs comprise ≥2, ≥3, or ≥4 separate processor components, with one component acting as a primary/main MA processor (which can be a multiple processor processing system or a single processor) and other components acting as specialized processors for certain data/functions (e.g., a microprocessor in a highly restricted component/part/zone of an MZMA). E.g., in aspects, an MA comprises a main processor, and 2 or 3 specialized processors, which can be, e.g., FGPAs, microprocessors, embedded processors, etc. In aspects, MAs comprise ≥1 high power processor (a processor that operates at speeds of greater than 100 MHz, typically greater than 250 MHz, and often greater than 500 MHz). In aspects, high power MA processors comprise graphical processing unit(s) (GPUs), e.g., NVIDIA/ATI GPUs. In aspects, MA processors are or comprise field programmable gate arrays (FPGAs), digital signal processors (DSPs), or application-specific integrated circuits (ASICs). In aspects, high power MA processors comprise multi-core (e.g., dual core, quad core, etc.)/parallel processing-capable architectures, such as Intel or Xeon multi-core processing units. In aspects, MA processor(s) comprise master processor(s) and slave processor(s), which both can be, in aspects, associated with a common bus. In aspects, operating software for a parallel processing-capable MA processor comprises OpenMP or another multiplatform shared-memory parallel programming API. In aspects, the number of cores in a multi-core MA processor is ≥~10, e.g., ≥~20, ≥~50, ≥~100, ≥~200, ≥~500, or ≥~1,000. In aspects, an MA processor can comprise coprocessor(s) (e.g., PCIe card(s)). In aspects, an MA processor can comprise CPU(s) and GPU(s), and in aspects a plurality of CPU core(s) and GPU core(s). Processor (e.g., CPU) cache sizes in MA processor can be, e.g., L1, L2, or L3 cache sizes, or larger caches (e.g., L4). Bus characteristics of MA processor can comprise PCI Express 1.0, 2.0, 3.0 etc. (e.g., AGP, PCI-X, VLB, etc.). An exemplary processing memory for a high-powered MA is, e.g., 32 GB (DDR2-667 FB-DIMIM memory) or 8 GB dual channel memory (e.g., DDR2 667/800), a memory controller (e.g., an Intel 5000X Chipset), but any other suitable configuration can be used.

CEI executed by MA/network device processor(s) and stored in CRM can comprise assembler instructions, instruction-set-architecture (ISA) instructions/CEI, machine instructions, machine dependent instructions, microcode, firmware instructions, state-setting data, configuration data for integrated circuitry, or either source code or object code written in any combination of one or more programming languages, including an object oriented programming language such as Smalltalk, C++, Java, Visual BASIC, Python, or the like, and procedural programming languages, such as the “C” programming language, database-focused programs (e.g., SQL), or similar or other suitable programming languages. In aspects relating to functions performed by MAs or other network components, computer readable program instructions/CEI can execute entirely on the applicable MA/device as a stand-alone software package, partly on an MA/network device and partly on a remote computer (e.g., in the NDS), or entirely on the remote computer or server. In the latter scenario, the remote computer can be connected to the other computer (e.g., the NDS) through any type of network, including a local area network (LAN) or a wide area network (WAN)/wide-area packet-switched network, or the connection can be made to an external computer (for example, through the Internet using an Internet Service Provider). In aspects, electronic circuitry including, for example, programmable logic circuitry, field-programmable gate arrays (FPGA), or programmable logic arrays (PLA) can execute CEI by utilizing state information of the CEI to personalize the electronic circuitry, to perform step(s)/Function(s) (S(s)/F(s)) of the method/NDS.

MA processor(s) typically process electrophysiological signals relayed from sensors. Where sensor data is complex (image data, waveform data, etc.) or demand load is otherwise high, the MA processor of an MA can utilize parallel processing, distributed processing, or both, and typically will include processing functions for protection against sensor error, device error, external events (e.g., power failure, power surge, etc.), etc. Examples of MA processing systems (e.g., parallel or distributed processors) and related principles, some of which may be adaptable to the practice of the invention, are described in, e.g., U.S. Pat. Nos. 5,464,435, 5,734,106, 6,185,460, 7,013,178, 7,758,567, U.S. Ser. No. 10/252,054, U.S. Ser. No. 10/499,854, U.S. Pat. No. 7,446,295, US20060009921, US20060206882, US20120226331, and U.S. Pat. No. 5,607,458.

### 3. MA Relay Component(s)/Unit(s)/Engine(s)

In operation, MAs of a network transmit device information comprising MA-D from the MA to the NDS/MAC-DMS. The components responsible for data relay from a MA can be characterized as a “relay unit,” (aka, a RELAYU or device data relay unit (DDRU)). An MA relay unit can comprise software, hardware, or software and hardware components and draw upon/utilize or comprise aspects of other units/components of the MA. MA relay unit(s) typically selectively, automatically, or selectively automatically transmit MA to the NDS.

In aspects, MA-D is transmitted from an MA relay unit to the NDS via the internet. Typically, a MA relay unit can operate selectively, automatically, or selectively automatically (e.g., attempting to relay data continuously or periodically when selected to do so). E.g., MAs may not relay data when certain conditions are met, such as when the MA is offline (no secure/stable network is available), being updated, tested, not in operation, etc. MA relay units typically relay MA-D or other MA data via secure internet data communication. In aspects, a MA relay unit will, whenever the MA processor determines that a secure and stable network connection to the NDS is available, relay MA-D or other MA data in a substantially continuous/streaming manner to the NDS. In aspects, if such a connection is not available, MA processing unit(s) execute CEI in DM causing the MA to evaluate if there is a stable and secure connection with the NDS/network. CEI in DM also can include instructions regarding how data is relayed, where it is relayed, when it is relayed, etc. In cases, an MA processing unit automatically and repeatedly, in operation, performs a Function/comprises a unit that operates according to the algorithm: START, (1) IF operational, (a) REPEAT UNTIL not operational, (I) SEND GET request for channel/socket to NDS, (II) OPEN/RECEIVE NDS response, (A) IF NDS response received, (i) CHECK for secure and reliable channel, (*) IF secure and reliable channel present, (#) SEND MA-D, (**) ELSE, (##) STORE Cache MA-D, (***) END IF, (B) ELSE, (i) STORE Cache MA-D, (C) END IF, and END.

In aspects, each operating MA automatically and repeatedly assesses whether a secure network connection is available, and (a) if a secure and stable network connection is available, automatically relay data comprising MA-D in a substantially continuous manner via secure internet data communication to the NDS/MAC-DMS; and (b) if a secure and stable network connection is not available (I) store MA-D in the medical apparatus memory unit as cache MA-D until a secure and stable network connection becomes available and (II) when a secure and stable network connection becomes available relaying the cache MA-D to the MAC-DMS via secure internet data communication. Techniques for assessing availability of a communication channel are known. A typical method is “pinging” of the server (here, the NDS) (e.g., via ICMP transfer methods known in the art). E.g., a system similar to, e.g., Microsoft's Network Connectivity Status Indicator (Windows) can be employed by attempting connection/pinging to the NDS on a regular basis. In aspects, a system such as XML/HTTP requests can be routinely relayed to the NDS using a formed message which the server will respond to with a status code computed by the server doing a number of checks to ensure that required NDS subsystems are online (e.g., streaming processing engine, etc.). Cloud-based server monitoring systems also are publicly available and can be used for monitoring cloud-based NDS servers (such as the Anturis service, CloudStats service, etc.), e.g., an NDS hosted on a leading commercial cloud platform such as Azure, AWS, or Google.

In aspects, some, most, generally all, essentially all, substantially all, or all the MA-relayed MA-D is semi-structured/semi-unstructured data. In aspects, some, most, generally all, essentially all, substantially all, or all of the MA-relayed MA-D conforms with a known/set format, which allows such data to be processed DOS faster by the NDS/MACMDS.

A MA relay unit can comprise or interact with/use direct communication media (e.g., physical connections facilitated by, e.g., communication cables such as coaxial cable lines, fiber optic lines, ethernet connections, etc.) or via wireless/remote communication means, such as Wi-Fi, Bluetooth, etc. In aspects, an MA relay unit will comprise a network interface, such as a NIC (network interface card/controller). In aspects, an MA relay unit can also or alternatively comprise or interact with a modem/router, such as a cable modem, DSL modem, router, switch, or the like. A MA relay unit can, accordingly, also comprise components such as a Wi-Fi transmitter/receiver module (aka a wireless transmitter).

Processor functions that can be considered a component of the MA relay unit can convert data into a transmission ready format, e.g., using a suitable protocol, e.g., TCP/IP, operating at an application protocol layer (e.g., FTP or WWW protocols), TCP layer, IP layer, and hardware layer (e.g., an ethernet card). Input units (discussed elsewhere) typically translate incoming data in a similar manner but in the “opposite direction.”

Suitable MA relay units/relay unit components are known. Accordingly, in aspects, a MA comprises means for relaying MA data/MA-D (meaning any suitable collection of relay unit components/systems described herein or equivalents in the art) and methods can comprise a step for relaying MA data/MA-D meaning employing any of the MA-D/MA data relay methods described herein or their equivalents.

Transmission ready data can be transmitted in any suitable form. In aspects, transmitted data is packaged and transmitted as packets, e.g., TCP/IP formatted packets. In aspects, other components of the network also sometimes, mostly, generally, essentially, substantially, or only communicate via packet communications. Accordingly, in aspects, the network can be characterized as a packet switched network (PSN). In aspects, most, generally all, substantially all, or all communications from MAs, in normal operation, are directed only to the NDS/MAC-DMS.

Data packets typically comprise header portions, comprising, e.g., data routing information, sequencing/relationship information, source information, packet data error detection/correction-relevant information (checksum, parity bits, or cyclic redundancy information), time to live/hop limit information, packet length information, packet priority information, payload information (relevant for queuing/prioritization), or any CT. Packets also typically include payload portions (comprising the primary or substantive relayed data, such as sensor data) and trailer portions (which can similarly include information relevant to error correction etc.). MA-relayed packet payload information typically will comprise MA-D including sensor information SMAD and other information, such as MA-D classifying/identifying information (device address, user class, device type, location information, patient/subject type, device status, or any CT) and other data classifying information (e.g., cache data/SMAD classification). In aspects, packet headers, payloads, or both, will comprise authentication information, enabling packet firewalls, filters, and other routing/screening/security units to operate, at least in part. E.g., packet information can include allowed IP addresses, allowed packet type, allowed port number, and other authentication-relevant information.

A MA relay unit can transmit data at any suitable speed. In aspects, the MA relay unit of some, most, generally all, or all MAs of the network can transmit using Gigabit Ethernet or Infiniband standards known in the art. In aspects, some, most, generally all, substantially all, or all MAs in a network relay data at 1 of these rate standards. In aspects, the MA relay unit transmits data on a continuous or near continuous basis as described elsewhere. In aspects, the MA relay unit transmit MA-data on a streaming basis, real-time basis, or both, most of the time, generally all the time, or at least substantially all the time.

In aspects, MA-D transmitted by an MA relay unit to an NDS is sensor data. In facets, some, most, generally all, or all of the MA-D relayed or transmitted by the MA relay unit in operation is real time data (RT-MA-D), such as real time sensor data, stored data, which may also be referred to as locally stored data (MA-CD/cache data), such as locally stored sensor data, or both RT-MA-D and MA-CD. In aspects, MA-D, e.g., SMAD and cache data, comprises structured data. In aspects, MA-D mostly, generally, or substantially only is unstructured data. In aspects, MA-D, relayed by most, generally all, substantially all, or all MAs on average, most of the time, or always, comprises both structured and unstructured data. In one facet, MA-D transmitted by an MA relay unit comprises images of MAs, the MA environment (e.g., the MA GUI), or both, and non-image sensor data. In aspects, an MA relay unit can be characterized as comprising or working with a wireless transmitter that can relay MA-D via Wi-Fi or similar wireless transmission mode.

### 4. MA Input Components)/Unit(s)/Engine(s)

MAs in a network receive data inputted into the MA, either through sensors, from the NDS, from local/direct input, from other devices/interfaces/sources (e.g., from an EMR), etc. The component(s) that handle input of data into an MA can be characterized as an MA input unit(s) (MA-INPU(s)). An MA input unit can comprise, e.g., physical components, such, as, e.g., network card(s) for receiving data, or, for example, other data receiving hardware, such as a modem. In aspects, such an MA input unit can comprise or interact with users via an interface or an input device, such as a keyboard, touch screen, voice interface, eye gazing/tracking interface, or the like. In aspects, an MA-INPU can be a digital interface receiving digitally transmitted data. In aspects, an MA input unit comprises or interacts with input devices (e.g., a touch screen or a keyboard input device, or a gesture-based or a voice input system/device). An MA input unit can comprise unit(s)/engine(s), e.g., software/operating system elements associated with conversion of received packets into device-usable data, such as instructions for device display, device control, device alarms/alerts, or any combination thereof (CT). In aspects, an MA input unit somewhat, mostly, generally, or entirely is composed of components that also act as an output unit (e.g., an MA can comprise network/interface card(s), modem(s), port(s), busses, etc., which serve as input/output component(s) for an MA). Input/output component(s)/system(s) of MAs or other devices of the network (including the NDS) can comprise engine(s)/components for encoding of data into appropriate communication media/medium (e.g., for inner-device or network communication), which will depend on communication channels (e.g., wi-fi vs. ethernet), recipient system(s)/capabilities, suitable communication language/code, etc. Engine(s) typically will ensure or apply, e.g., time communication for synchronization facilitation and other code (e.g., packet headers) of identification, data type, authorization, etc. An input engine/function typically is configured to routinely, automatically, or conditionally automatically, send acknowledgment message(s) to the NDS (e.g., in response to NDS pings or status queries). I/O systems in network devices/systems are typically adapted for packet switching communication, circuit switching communication, or both. In aspects, most, generally all, or all network communication is performed via packet switching protocols and I/O components/engines are adapted to apply/interpret packet switching protocols, such as statistical multiplexing. In other aspects, an MA input unit can comprise specialized engine(s) that handle one or more aspect(s) of the receipt of data, such as protocol(s) for processing received data packets.

### 5. MA Security Engine(s)/Unit(s)

In aspects, some, most, generally all, or all MAs in a network further comprise a system for maintaining data security (an MA Security Unit). An MA security unit (aka, an MA-SECURU) can associate with or be considered a part of an MA-input unit. An MA security unit can perform different function(s) relating to data security. Typically, such functions include firewall functions, which restrict data flow into the MA's software/operating system or memory to only certain authorized data inputs. MA Security Unit component(s)/Engine(s) can include/perform user authorization Functions/Engines (e.g., password or biometric authorization protections for logging into the MA or a CT such as a 2-factor authentication function). An MA Security Unit can comprise authentication for network access separate from or integrated with MA local access, which can comprise any such authentication method. Authentication information can be required to be partially or entirely updated at one or both levels upon the occurrence of event(s) (e.g., security breaches, lost authentication, etc.), based on passage of time, or both.

Authorization/user authentication components/functions that can be part of a security unit applied at a local (device) level, network level, or both can include, e.g., pre-shared key/device authentication/recognition, password/code authentication, biometric information, knowledge-based authorization, use of key fobs/devices or authentication applications (e.g., operated on mobile devices), behavioral/psychometric authentication, or any CT (e.g., 2-factor or three-factor authorization methods). Other authorization methods, principles, etc., adaptable to such aspects are described in, e.g., U.S. Pat. Nos. 5,684,951, 6,421,943, 5,832,209, 6,263,432, 7,904,956, 7,991,902, 5,999,711, 5,613,012, 5,742,756, 6,289,344, 6,594,759, 6,675,153, 6,711,681, EP1115074, U.S. Pat. No. 7,434,257, US20020032661, US20200286055, US20020184161, US20200329051, US20200267147, US20200311285, U.S. Pat. Nos. 6,910,041, 7,080,037, 7,685,173, 7,366,913, 7,178,163, 7,664,752, 8,365,254, 8,024,794, US20080183625, and U.S. Pat. No. 8,646,027.

In facets, each MA can comprise a mechanism for restricting the features and operating conditions of the MA which can be controlled remotely. In facets, each MA can comprise a mechanism for restricting user access to features of the MA based on a user profile. In embodiments, each MA can restrict data inputs (e.g., data received by the MA-INPU) based on one or more established security rules/protocols, and can further restrict data output (e.g., data sent by the MA relay unit based on one or more established security rules. In aspects, at least some, most, generally all, or all data coming into or exiting an MA is subject to ≥1 layer of data security. In aspects, each MA has an independent device security system. In aspects, a group of MAs can comprise a shared device security system. In further aspects, a network of MAs can comprise a shared device security system.

In aspects, the MA security system can identify user level of access to information contained in incoming MA-D data or analysis sent by an NDS relay unit and apply redaction or exclusion rules and data modifications to the information based on user/customer level of access. Such rules can work in place of or in association with NDS functions such as filtering functions, routing functions, NDS-security units, or any combination thereof.

In aspects, the MA-security unit comprises one or more physical components that are dedicated or at least materially, at least mostly, or generally dedicated to security functions. E.g., an MA security unit can comprise embedded processor(s) (or computers/sub-computers comprising processor(s) and memory—e.g., system-on-a-chip (“SoC”) devices/components, known in the art, or other multi-functional integrated circuits), which can perform various security-related Functions (e.g., running challenge and response authentication functions, firewall functions, or both). Such a device can comprise, e.g., code for an encryption engine, providing data encryption functions. Dedicated processors and other components (including most, generally all, or all MA processors) can be subject to physical security, e.g., shielding, such as a metal shield, and tamper dedication/mitigation functions/components, e.g., tamper sensors that erase data (e.g., authentication information, encryption keys, or most, generally all, or all data—e.g., by overwriting most, generally all, or all binary/machine data with 0s), when tampering is detected (or detected and not mitigated within a preprogrammed period by a user with sufficient credentials/authentication). Examples of such devices include 8-bit microcontrollers to 8-bit, 16-bit, or 32-bit embedded processors and MAs can comprise either, similar devices, or combinations of such devices, which can, i.a., perform security functions. Such devices can also comprise, e.g., 128 to 512 kb of flash code storage and from, e.g., 32 to 256 kb of high-speed SRAM. In aspects, an MA security unit can record access to an MA for later reference by, e.g., a user or an NDS Owner/Operator (SO).

In aspects, inclusion of embedded processors or microcontrollers in an MA detectably or significantly further reduces power expenditure, operating heat, or both, as compared to a comparable device in which such functions are performed by a primary MA processing unit (e.g., a MA microprocessor controlling most, generally all, or substantially all operations of MA computer and computer-controlled operations). In aspects, one, some, most, generally all, or all microcontrollers, embedded processors, SoCs, any combination thereof, etc. in the MA utilize(s) less than 10 watts of power in operation, e.g., ≤~7, ≤~5, or ≤~3 watts. In aspects, external processors (with respect to a primary MA processor), such as microprocessors, embedded processors, etc., exhibit an average power usage of less than 10, ≤~7, ≤~5, or ≤~3 watts.

Engine(s) encoded in relevant memory and executed by such processors can comprise encryption protocols, such as DES, triple DES, SHA-1, AES, and RSA encryption methods/engines (cryptographic accelerator(s)). Uncontradicted, disclosure of any aspect herein with respect to any such device (SoC, microprocessor, or other embedded processor or integrated circuit) implicitly provides support for such other components or other equivalent means in the art (e.g., in terms of functionality, structure, operational characteristics such as power usage, or any CT). In aspects, MAs comprise microcontroller(s) or similar devices, such as specialized embedded processors, SoCs, etc. In aspects, one, some, generally all, or all microcontrollers, embedded processors, SoCs, etc., are separate from a primary MA processor and have less processing power than the primary processor. In aspects, some, most, or generally all processors of an MA integrate analog components needed to control non-digital electronic systems that can be in the MA or associated with the MA.

In aspects, an MA can comprise/utilize, or an MA security unit can comprise or utilize, ≥1 microcontroller, such as, for example, ≥about 2, ≥about 3, ≥about 4, ≥about 5, or ≥about 10 microcontrollers or more, which can, i.a., perform 1 or more data protection functions. In aspects, such a microcontroller can, e.g., restrict data input to only recognizable data, such as, for example, data that meets established pre-defined identification threshold, indicating that the data is of an approved data type (for example, data is within an established expected range, is of an expected numeric or alpha character, comprises the appropriate units, is formatted in an established expected format, or the like). MAs can also comprise SoCs, microprocessors, embedded processors, etc., as part of an overall MA processing function, e.g., for initial detection or control of sensor(s), separate from security functions, or that at least mostly perform functions separate from security functions (on average or on most or generally all operating periods, e.g., days or years). Most, generally all, or all microcontrollers etc. in an MA provide real-time response to events in the associated/embedded system the microcontroller(s) are controlling. In aspects, some, most, generally all, or all microcontrollers or similar processors comprise an interrupt system that suspends microcontroller processing of a typical or current instruction sequence and begins an interrupt service routine (ISR, or “interrupt handler”) which will perform any processing required based on the source of the interrupt, before returning to the original instruction sequence. In aspects, some, most, generally all, or all functions related to the microcontroller's performance (microcontroller Engine(s)/Function(s)) are encoded/stored in the primary MA memory, rather than in separate microcontroller memory. In aspects, a microcontroller will execute code/Engines encoded both in microcontroller-associated standalone memory and in the primary MA memory. In aspects, most, generally all, substantially all, or all microcontrollers of most, generally all, or all MA(s) will comprise an analog-to-digital converter (ADC), a digital-to-analog converter (DAC) (e.g., a DAC that allows the processor to output analog signals or voltage levels), or a CT. In aspects, most, generally all, or all microcontrollers will have the ability to communicate in 1 or more digital formats with other components, e.g., via Inter-Integrated Circuit (I²C), Serial Peripheral Interface (SPI), Universal Serial Bus (USB), Ethernet, RS232, or a CT, or any similar protocols/means in the art. In aspects, most, generally all, or all microcontrollers of most, generally all, or all MAs will be programmable, and, in aspects, most, generally all, or all of the microcontrollers are programmable in an object-oriented language, such as a version of Python, Java, C, or the like. In aspects, most, generally all, or all microcontrollers comprise CMOS construction. In aspects, some, most, generally all, or all microcontrollers of most MAs or each MA of MAs in the network are RISC (reduced instruction set) or CISC microcontrollers. In aspects, some, most, or all microcontrollers of some, most, or all MAs of the network are special purpose computers, performing e.g., ≤5, ≤3, ≤2, or only 1 function (e.g., detection or measure of sensor data from a particular sensor, control of data flow into or within the MA, etc.) and typically runs a corresponding number of software applications. In aspects, one, some, most, generally all, essentially all, or all microcontrollers comprise dedicated input(s), a display unit (e.g., an LED display), or both.

An MA security unit can comprise one or more firewall functions. A “firewall” can be considered a security device, system, or component that monitors/analyzes data transmission/flows, such as network traffic. Like other data filters or data curators, a firewall filters incoming data flow (traffic), outgoing data flow/traffic, or both, typically based on a set of established/preprogrammed standards/rules. A firewall can be placed on a hardware level of a device/system, software level of a device/system, or both, to secure it from unauthorized/malicious traffic. Depending on the setup, a firewall can protect a single machine, groups of machines, system, or a whole network. Firewalls in this disclosure can comprise software firewalls (e.g., host firewalls) (e.g., performed by the primary microprocessor or other primary processing unit of the MA's computer system), hardware firewalls (e.g., appliance firewalls) (e.g., a microcontroller or embedded processor firewall, or a separate but associated device firewall), or both.

In aspects, the firewall function of a security unit will comprise 1 or more packet-filtering firewalls. In aspects such firewalls comprise protocols for checking packet data against access control list(s), and a protocol for dropping/blocking unauthorized relayed packets, passing authorized packets, or both. In aspects, most, generally all, or substantially all an MA firewall function is composed of packet filter(s), such as a stateless packet filter firewall. In aspects, a security system will comprise 1 or more other types of firewalls besides or in place of stateless packet filter firewall(s) (aka “packet firewalls”). Other types of firewalls known in the art and discussed elsewhere that can be employed in a security unit include stateful packet inspection (SPI), proxy server firewalls (e.g., applied to outgoing/relayed data transmitted over the internet), circuit level gateways, or any CT/combination. In aspects, a security unit comprises a deep packet inspection firewall, which analyzes some, most, generally all, substantially all, or all the payload content of some, most, generally all, substantially all, or all received packets, TCP handshakes, URL filtering, or any ACT. In aspects, a firewall of a security unit performs both surface and deep packet inspection, e.g., in an ordered fashion, based on analysis of the packets, or both. In aspects, the firewall function(s) also perform antivirus scanning/protection functions, spam filtering functions, application control functions, or a combination thereof. In aspects, a firewall function can terminate secure sockets layer (SSL), transport layer security (TLS) connections, or both.

In aspects, an MA security unit, also comprises an Intrusion Prevention NDSs (IPS) (e.g., that performs signature tracing and anomaly detection to prevent threats from entering data streams. In aspects, a security unit, such as an MA-SECURU, also or alternatively comprises a sandboxing function, isolating incoming code, executing it, and inspecting it, either regularly or based on triggers in other security unit(s)/engine(s)/function(s) (U/Fs).

Uncontradicted, aspects of MA security units discussed here can be applied to NDS security units (described below) and vice versa. E.g., characteristics of firewall components of an MA security unit also can be components of an NDS security unit.

In some facets, MA security unit(s) comprise(s) an anti-tampering detection function that sends a signal to the NDS if a prohibited tampering event occurs in an MA. In aspects, if such a signal is received by an NDS, the NDS can, in aspects, establish new communication rules associated with such an MA and can, further, in facets alert appropriate users, e.g., users local to the MA (e.g., via network access device(s) (NAD(s))) of the presence of a tampering event or take other actions as indicated elsewhere (e.g., locking down use of the system, erasing data, etc.).

### III. MULTI-ZONE MAs (MZMAs)

In aspects, at least some MAs in a network (or most, generally all, or all MAs in a network) comprise ≥2 components or collection of components/sub-devices that are subject to separate security protocols or security units (“zones”), perform different MA functions, are subject to different regulatory status, have different communication capabilities, etc. Such MAs can be characterized as “multi-zone” MAs (“MZMAs”) (or multi-component MAs (“MCMAs”)). Inter-communication or inter-connection of the zones/components of an MZMA can be achieved through any suitable component(s), methods, or means. In aspects, most, generally all, or all the physical components, software components, or both, of most, generally all, or all the zones/components of an MZMA are housed within a single hardware housing of an MA. In aspects, components of an MZMA communicate through direct cable/wire connections that relay data (e.g., using current versions of RS-232, RS-422, RS-485, or Ethernet protocols/standards). In aspects, data relay/communication between zones or zone components also or alternatively is managed through wireless communication methods known in the art (e.g., Bluetooth or light/laser data transmission). In aspects, the multiple components/zones of an MZMA share component(s)/function(s) (e.g., power or display functions, etc.).

In aspects, two or more zones of an MZMA can comprise separate, dedicated, and zone-specific processing functions or applications. In aspects, 2 or more zones of an MZMA can comprise different processing functions, be under the control of one or more different processors, utilize one or more different processors, or any or all thereof. As a non-limiting example, in one aspect, one or more zones of an MZMA can comprise one or more zone-specific microprocessors and one or more other zones can comprise a system on a chip (SoC). According to some aspects, zones can share processor(s) or processing function(s). In aspects, zones can both share processor(s) or processing function(s) while concurrently comprising separate, dedicated, and zone-specific processing functionality or applications, e.g., one or more processors can be shared and one or more processors can be zone-specific.

One, some, most, generally all, or all MAs in a network can comprise a more restricted zone/part and a less restricted zone/part. In aspects, the more restricted zone/part is associated with therapeutic component(s) (e.g., a critical care treatment component) and the less restricted zone/part is associated with subject monitoring, diagnosis, therapeutic component(s) that is/are less critical to patient safety/status, or any combination thereof (CT). In one exemplary aspect, an MZMA comprises a highly restricted therapeutic application component that provides a critical life support system treatment function, comprises physical anti-tampering protection, and comprises MA CEIs that are mostly, substantially only, or only locally modifiable. In aspects, only packets/data relating to control of the therapeutic component can be relayed from the NDS to the highly restricted zone/component. In aspects, packets/data relating to control of the therapeutic component or that otherwise are permitted to pass to the highly restricted zone/part of the MZMA are required to pass through ≥2 firewalls (e.g., a less restricted zone/overall MA firewall and a microcontroller security unit that controls communications between the zones of the MZMA). In aspects, a highly restricted part/zone will comprise anti-tampering detection/protection systems, user authorization systems, or both. In aspects, all data relayed from the highly restricted zone/component is relayed first to a lesser restricted zone/component, which in turns relays such MA-D to the NDS (e.g., where the lesser zone/component is the only part of the MA to be in communication with the internet). In embodiments, at least some of the MAs comprise a patient monitoring/diagnosis component that comprises a processing unit that can receive system update availability but also comprises MA CEIs that are mostly, are substantially only, or are only modifiable through a pull request (or confirmed request) sent to the NDS. In aspects, a highly restricted part of an MZMA will comprise backup functions that allow the MZMA to operate independently of lesser restrictive parts/zones of the MZMA, such as where the highly restrictive part is engaged in critical life support activities.

In aspects, most, generally all, or all parts/zones of an MZMA are associated with the same subject, e.g., a first zone treats a subject in a first manner and a second zone detects one or more types of device data, sensor data, or other data associated with the subject. In aspects, one or more parts of an MZMA are housed in a single unit housing. Zones of an MZMA can supporting perform therapeutic or diagnostic tasks supporting same physiological condition/system (e.g., the cardiovascular system). In aspects, zones of an MZMA focus on different conditions/subject systems. In aspects, zones of an MZMA are associated with monitoring or applying different medical device applications. In aspects, each part of an MZMA has a different regulatory status. E.g., a highly restricted part of an MZMA may be regulated as a Class II medical device or Class III/PMA medical device by US FDA and a less restricted part may be regulated as a Class I device (a lower level of regulation).

In aspects, a network comprises a number of MZMAs, each MZMA of the network comprising two or more distinct components contained in two or more distinct zones, each component (a) comprising a separate processor that processes at least some MA-D not processed by the processor of at least one other component and (b) (I) receiving information from different sensors, (II) performing different therapeutic/preventative or diagnostic medical tasks, or (III) receiving information from different sensors and performing different therapeutic/preventative or diagnostic medical tasks, wherein at least one component in each MZMA is subject to a different level of interaction with one or more other parts of the data network than at least one other component of the MZMA. E.g., component(s) in a first zone of an MZMA can be subject to more security and less direct or no direct communication with the data network, whereas component(s) in a second zone can be subject to direct communication normally or under certain conditions (e.g., relaying data out regularly, but receiving data in only when it passes a firewall or when it is in response to a request/pull signal sent from second zone MZMA component(s)).

In cases, at least one component of at least one of the MZMAs in a network is associated with application of a therapeutic/preventative medical task (uncontradicted, references to therapeutic and preventative tasks herein implicitly provide support for each other), and such at least one component of the MZMA is not in direct communication with the data network. In aspects, at least one component of at least one of the MZMAs in a network (1) is associated with application of a therapeutic medical task, a preventative task, or both, (2) is in communication with a NDS/MAC-DMS, and (c) only permits a pre-established amount of input from the NDS/MAC-DMS, wherein changes to the operating system, software, or approved forms of NDS/MAC-DMS input in the at least one component or associated zone of the MZMA requires local approval from an authorized operator of the MZMA.

Readers will recognize that MZMAs are an independent aspect of the invention (i.e., separate from aspects of the invention involving NDSs). In such a respect, the invention provides, e.g., a medical device comprising two or more zones that are subject to different levels of interaction with an associated data network, such as the internet or other network, wherein such ≥2 zones are subject to different rules/limitations in terms of interactivity with the network, typically wherein a zone associated with critical components of patient care is subject to restricted communication with the network/internet (e.g., as in such zone not being permitted to directly receive information from the external network/internet, and in some cases also to send information directly to the network/internet). In aspects, such an MZMA or restricted components thereof are only subject to manual updating/modification or updating over the network/internet via a push/request for such an update by an authorized user. In aspects, the most sensitive components of such a device are only subject to manual updating. In aspects, such an MZMA is subject to the various security components discussed in this disclosure (e.g., anti-tampering protections). Other features/aspects described herein in connection with MZMAs also can be incorporated in such MZMAs which, again, can, in aspects, operate independently of an MZMA (e.g., by networking with other servers, the internet, other medical devices, other computers, other networks, etc.).

### B. System(s) (NDSs) and Networks

One or more MA(s), typically group(s) of MA(s), and an NDS in operation form a network. As noted elsewhere, a network also can include or interface with other devices/components, e.g., a CRMS, other ONDIs, devices/systems hosting EMRs, etc. A network formed by MAs and an NDS can be a defined part of a broader network, such as the internet.

In aspects, an NDS can control one or more aspects of the operation of some, most, generally all, or all MAs of the network. Such an NDS can be characterized as a MA controlling and data management system (“MAC-DMS”). A MAC-DMS also can optionally control some or most operational aspects of some, most, generally all, or all ONDIs in the network. In general, and uncontradicted, any aspect described here with respect to a network/NDS also can be applied to a MAC-DMS and each disclosure of an NDS/network implicitly also discloses a corresponding aspect in which the referenced NDS/network is a MAC-DMS.

The components of networks can be interconnected through any suitable method/means. In aspects, most, generally all, or at least substantially all components of the network will be connected via internet communication. In aspects, certain parts of a network can be excluded from internet communication, e.g., parts of medical apparatuses (MAs) in the network can be excluded from internet communications, such as a therapeutic component involved in direct application of therapies to a patient (e.g., in a restricted zone of an MZMA). Components of a network can be distinguished/connected through points of connection (e.g., a node(s)). Nodes of a network that are subject to some level of control by the SO, IEs, or both, can include node(s) for an Entity that serve(s) as a connection point between Entity MA(s) (which can be characterized as an IE MAG) and the NDS), points of screening/filtering (e.g., firewalls and other security units at a local MA, NDS, or intervening level), or both. In aspects, communications between some, most, generally all, or all components of a network are encrypted (e.g., communicated via VPN or other form of encrypted tunnel). In aspects, some communications are not encrypted, but some data (e.g., PHI, device control application(s), etc.) are encrypted or subject to higher levels of encryption/security than other data relayed in the network between network devices.

In aspects, the network can be classified as a wide area network (WAN). In aspects, the network can be classified as or comprise a software defined WAN (SDWAN) or cloud-based WAN. In aspects, the WAN comprises a plurality of LANs (e.g., entity LANs, facility LANs, or LANs associated with classes of users or groups of users in classes, e.g., groups of researcher users associated with different research organization Entities).

In aspects, the owner/operator of the NDS/MAC-DMS (SO) is a different Entity from the owner of some, most, generally all, substantially all, or all of the MAs in the network. In aspects, an NDS can be characterized as having access to MAs or groups of MAs, e.g., through the grant of access for permission to receive or relay data from/to network MA(s) from the MA owner(s). In aspects, an NDS can comprise an electronic contracting function associated with the connection of MAs owned by IEs (regarding to the NDS owner), which presents, negotiates, executes, maintains, updates, and otherwise manages contractual terms for access of the NDS by the MAs (including terms relating to intellectual property, ownership and modification of software or data, confidentiality, patient safety, compliance with laws, liability, performance warranties, financial terms, etc.). In aspects, such electronic contracting functions are selectively or conditionally updatable (e.g., renewable when terms expire or device/NDS conditions change).

An NDS comprises components/systems/capabilities for handling incoming and outgoing communications with MAs and, optionally, ONDIs (e.g., can both receive and transmit data), and an NDS will also comprise data storage, and data processing capability(ies), as discussed elsewhere. In aspects, an NDS can comprise the ability to communicate to any one or more MAs, individually or as a group or as MA groups, such as, communicating to any 2 or more MAs at a time or to any 2 or more groups of MAs (MAGs) at a time. The NDS can comprise any suitable combination of hardware, software, etc. In aspects, most, generally all, substantially all, or all the NDS components are cloud-based resources, such as cloud-based memory, cloud-based processor functions, etc.

In aspects, NDSs relay of information to or MAC-DMS control the operation of MAs based on, i.a., NDS-AD (e.g., the comparison of NDS-AD to pre-programmed standards/rules or algorithms). NDS-AD can be used to generate control data, which can be characterized as output applications or instructions, as well as or alternatively to relaying informational NDS-AD, which also can be delivered to MAs and other network devices/interfaces (ONDIs). E.g., informational NDS-AD can include, e.g., predicted physiological data for a subject, which is generated by an NDS through analyzing MA-D of similarly situated subjects, optionally with reference to other medical data.

Control applications/controller(s) of an NDS can control MA displays, other sensory MA functions (e.g., by playing alarms), or control therapeutic or diagnostic components of MAs/MA parts/components. As such, NDSs can be characterized as MA controlling and data management system (“MAC-DMSs”). Although the term NDS is often used here, it will be understood that in common aspects NDSs also can be classified as MAC-DMSs and readers will understand that the use of the term NDS here implicitly discloses corresponding aspects in which the NDS is a MAC-DMS.

### I. NDS Components

To better illustrate possible components of NDS(s) and their operation, several select components are discussed in the following sections.

### 1. NDS Memory Systems/Component(s)Unit(s)

An NDS comprises one or more memory unit(s) (“memory” or “NDS-MEMU”) that can maintain MA-D, NDS-AD, and CEI for operation of the NDS. An NDS memory unit can comprise any suitable type of PTRCRM contained in one or more structures (≥1 drives, media banks, etc.). In aspects, most, generally all, or all NDS memory is organized into data repository(ies) (DR(s)). In aspects, an NDS comprises one or more query-able (queryable)/searchable DR(s) comprising MA-D, NDS-AD, optionally inputs from other input sources (e.g., connected CRMS(s)), or any combination thereof. In aspects, an NDS may comprise ≥2 types of memory/DRs that are functionally distinct, physically distinct, or both. E.g., in aspects an NDS will comprise a memory unit that is associated with a Streaming Data Processor (SDP) that is operationally or physically separated from a primary memory of the NDS, e.g., by being separate from most, generally all, or all DR(s) of the NDS. The portion of NDS memory that comprises most or all of the DRs can be described as the “primary memory” of the NDS. As discussed below, an NDS memory unit/system, such as an NDS primary memory, can be divided into different physical components, different functional zones/areas, or both.

In aspects, some, generally all, or all an NDS memory unit (sometimes referred to as NDS-MEMU) comprises a network-attached storage (NAS) infrastructure, such as a clustered NAS infrastructure (comprising, e.g., NAS pods, each comprising several separate storage devices, typically connected to a master NAS device, forming an interconnected memory device/media). In aspects, some, most, generally all, or all an NDS memory unit is based on a cloud computing platform/paradigm (e.g., DR/data store as a Service/Storage (DaaS) Platform or an Infrastructure as a Service (IaaS) or Platform as a Service (PaaS) Platform, comprising processing and possibly other functions in addition to memory and memory-supportive functions only). In aspects, the cloud based NDS memory is based on a distributed system, scalable on demand (or automatically) system, or both. In aspects, distributed file systems that form some, most, generally all, or all of the NDS memory store data across many separate servers. In some facets, most, generally all, or substantially all the NDS memory is not distributed. In aspects, NDS memory and related NDS Input Unit Functions comprise redundant storage of data in a distributed memory system.

Hardware components of an NDS memory unit (or the memory unit of other devices of a network) can use any suitable type of memory media. In aspects, memory will comprise dynamic RAM or Flash memory. In aspects, memory comprises disk-based storage memory or a hybrid structure disk-based storage and DRAM/flash memory (e.g., using disk storage for “colder” data with DRAM or flash being used for “hotter” data).

In general, memory unit(s), such as NDS memory, may include firmware, kernel(s), and/or applications. A kernel can be an operating system, including modules for memory management, scheduling and management of processes, input/output, and communication, or device drivers that allow the operating system to communicate with the hardware modules/components (e.g., memory units, networking interfaces, ports, and buses) of the relevant computing device/system. Applications may be one or more user-space software programs, such as web browsers or email clients, as well as any software libraries used by these programs. Memory may also store data used by these and other programs and applications.

Data storage/memory unit(s) of an NDS or other network device can comprise/be in data storage arrays that can include drive array controllers configured to manage read and write access to groups of hard disk drives, solid state drives, etc. Drive array controllers, alone or in conjunction with NDS component(s) (server device(s)), also can be configured to manage backup or redundant copies of data stored in data storage/memory unit (DR(s)) to protect against DR/drive failure(s), data failures, etc., e.g., failures that prevent NDS component(s) from accessing parts/units of memory/data storage. As discussed elsewhere, DR(s) can include any suitable form of data repository(ies), including any suitable database(s), such as a structured query language (SQL) database as are known. Various types of data structures can store information (e.g., analytical data) in such a database, including but not limited to tables, arrays, lists, trees, and tuples. Furthermore, databases or other DR(s) in data storage can be monolithic or distributed across multiple physical devices.

In aspects, the NDS memory unit(s) comprises a cloud-based memory comprising an internet-scale file system (e.g., Google File System (GFS)). Examples of cloud-based storage systems having these and other features discussed here include Amazon Simple Storage Service (S3), Nirvanix Cloud Storage, OpenStack Swift, and Windows Azure Binary Large Object (Blob) storage. An NDS-MEMU can comprise a file management system/layer, which defines some or most of the architecture for other data management levels in the Memory Unit (e.g., a Hadoop Distributed File System (HDFS) or similar system with capability to partition and replicate data sets to nodes where they are more likely to be consumed by applications directed by Mapping Functions (mappers)). Such file systems typically comprise a configurable data set replication function, detectably or significantly minimizing the impact of component/device failure in the NDS-MEMU hardware architecture or operating system/software. In aspects, U/Fs of NDS memory unit(s) also can comprise a data management system (DMS), which can include a DBMS. In aspects, U/Fs of the NDS memory unit(s) comprise an execution tool to DoS distribute computational load among components of the memory and which can act as an API for the memory and can include or relate to other U/Fs, such as data inspection, data auditing, or data visualization functions directed to NDS memory. NDS processor(s) also typically comprises a query system for information extraction from the DR.

In aspects, some of the data in the DR of the NDS, e.g., an appreciable amount of data or a material amount of data in the DR, is stored using a non-relational model(s) (e.g., document, graph, key value, and other models that are used to provide efficient storage and access to nontabular data sets). In aspects, some, most, or generally all the data in the DR can be characterized as being stored in a NoSQL DR, e.g., MongoDB, Hadoop HBase, Apache Cassandra, Couchbase, or the like. In aspects, a DR employs both NoSQL and batch processing-optimized Hadoop functions/structures. E.g., HBase (a NoSQL DR structure) can be employed on top of HDFS, to provide low latency functions in Hadoop. Other file systems that can effectively perform similar memory and memory-related functions in a distributed, multi-server environment, can be employed. Typically, a distributed memory system will comprise a master node that stores a data and file/data system chunk servers that store the actual chunks in multiple nodes, typically in a replicated and failure-resistant/fault tolerant manner. A data management system (DMS) typically also will comprise checksum functions or similar functions to check for data integrity and correction/alerting mechanisms associated therewith. Other tools that can be used in the DMS can comprise a MapReduce framework or similar parallel, distributed, and cluster-based data management algorithms, such as Hadoop, Apache Spark, etc. In aspects, the NDS Input Unit can concurrently process data from ≥~250 nodes/streams, ≥~500 nodes/streams, ≥~1000 nodes/streams, ≥~5000 nodes/streams, or ≥~10,000 nodes/streams. In aspects, such processing is associated with ingestion latency of ≤~40 sec, ≤~30 sec, ≤~10 sec, ≤~5 sec, or ≤~1 second (e.g., ≤~0.33 second, ≤~0.1 second, or ≤~0.05 sec), including performance of initial transformations.

As discussed elsewhere, in aspects data ingestion paradigms can employ extract, transform, and load (ETL) procedure(s) in which data is taken from the source, manipulated to fit the properties of a destination system or the needs of the business, then added to that system. In other aspects, as discussed elsewhere, transformations or data ingestion structure requirements are minimal (e.g., requiring less than ~10, less than ~9, or less than ~8 transformations/structures) and the NDS analytical functions primarily operate based on transformations applied to DR stored data, such that data ingestion is primarily, generally only, or substantially only, based on an extract, load, and transform (ELT) method.

Data repositories (“DRs”) can be classified based on how data is primarily, substantially only, or only received (ingested), stored, and accessed/utilized (consumed) in/from the DR. Examples of known DRs include databases, data warehouses, and data lakes. Typically, each DR in a device or NDS's memory can be distinguished based on characteristics in these other ways from other DRs in the overall memory of the device/NDS. E.g., skilled persons can distinguish a database DR from a data lake DR based on, i.a., the structure of the DR, the type of data stored in the DR, etc.

Typically, here, a database means a DR comprising a relational dataset comprising ≥about 5, typically ≥7, ≥8, or ≥about 10 (e.g., ≥~12, ≥15, or ≥~20) interrelated data points comprising attributes and values organized into ≥~3, ≥5, ≥10, ≥50, or ≥~100 records, and can and typically will include higher level structures such as tables, stored queries, etc. Databases typically store data in tables, columns, and rows (records and fields in tables). Databases are typically controlled by database management systems (DBMS), with relational database management systems (RDBMs) and object-oriented databases being common. Most databases employed in NDS memory typically are hierarchical (non-flat). Examples of databases (DBs) that can be employed in aspects include Microsoft SQL, Oracle, Microsoft Access, and Redshift databases. In aspects, NDSs comprise ≥1 and in aspects≥2 relational databases, which collect assembled datasets comprising MA-D, analytical data, output, etc., in database hierarchies, as illustrated elsewhere. Thus, in cases, an NDS comprises multiple query-able DRs, e.g., ≥1 DL/EDL DR and ≥1 relational database (RDB) DR. NDSs also can comprise other memory components, such as an active memory component (e.g., a network RAM), separate instructions for initial analyses, etc., which can be used to perform analyses on data (typically restricted to a limited set of Step(s)/Functions, such as ≤50, ≤30, ≤20, ≤12, ≤8, or ≤5 relative set S(s)/F(s)) performed on incoming data prior to or during ingestion into DRs.

A “data warehouse” typically is distinguished from a database by scale, time of storage, sources of information (data warehouses draw from several sources and integrate such data), structure, and function (e.g., optimization for processing of information rather than just recording transactions). Data warehouses receive related data from several sources and, like databases, apply structure (schema), require a set structure, or both, before storing such data, which can be for long periods of time. Components of data warehouses can be characterized as data marts.

Both databases and data warehouses can be characterized as schema-on-write/recording systems, requiring that data be ingested in a certain format, transformed to a certain format, or both, prior to storage in the DR. In aspects, some or most of the DR content of an NDS, some or most of the DRs of an NDS, or both, are not schema-on-write/recording systems. In aspects, an NDS primary memory comprises a mixture of schema-on-write/recording and non-schema-on-write/recording DRs.

In aspects, some, most, or generally all of the primary NDS memory unit is structured as a data lake (“DL”), an enhanced data lake (“EDL”) (discussed below), or a combination thereof. Uncontradicted, a reference to a DL or an EDL provides implicit support for an aspect comprising the corresponding type of memory structure (although there are aspects of an EDL that are distinct from a true DL). In aspects, a DL/EDL can comprise structured portions (data storage zones adapted/configured to retain or at least assigned to retain structured records, such as in a database) or the NDS-MEMU can comprise a physically or functionally separate structured DR portion, which can comprise, e.g., database(s), a data warehouse, or a data mart (e.g., as a component of memory otherwise having a DL/EDL structure). In aspects, at least part of the NDS-memory, or most, generally all, or all of the NDS memory, is organized in a cloud data warehouse, such as Amazon Redshift, Google BigQuery, Snowflake, and Microsoft Azure SQL Data Warehouse.

a. Data Lakes

In aspects, an NDS memory unit (e.g., the primary NDS-MEMU), is, comprises, primarily comprises, consists essentially of, or generally consists of a DR comprising a data lake DR, an EDL DR, or a system that comprises elements of both systems.

In this disclosure, a data lake (“DL”), is a memory structure that accepts data in forms that are unacceptable to a data warehouse, database, or similar schema-on-write/record DR. Data in DLs is/are typically organized or organized in about 2-4, 2-5, 2-6, or about 2-7 levels of data relationships (e.g., attribute and value; attribute-value-data type; attribute-value-data type-source; or attribute-value-data type-source-time series) in records only on a schema-on-read basis (on the application of a schema to data in the DL). A DL/DLLR typically can be characterized as employing a primarily, generally only, or solely extract-load-transform (ELT) data handling methodology. In aspects, a DL can store data in native format (i.e., raw data and unstructured data). In a basic/true DL, no data silos/sorting exists (heterogenous data in terms of structure, type, etc., are all maintained in an integrated DR). A DL typically receives data from inputs, such as a data pipeline, and stores the data immediately without regard to structure of the data, with the data being accessed by DL access/application platforms (e.g., Apache Hadoop, Apache Spark, a Relational Database Management System (RDBMS)/SQL, or a CT) (e.g., applying Hudi or Parquet data formats).

Data in a DL structure can be contained in one or more electronic data storage devices or structures that hold the stored data, which can be dedicated devices, cloud based/on demand/distributed memory resources, or a CT. In a true DL, data is stored and maintained in an unmodified format from how the data was received by the DL (other than, e.g., an identifier metadata tag applied in some DLs), at least until application of a schema, operation of a query, or both, or other later consumption, use, or interpretation by applications, the NDS, other network devices, or network users. In aspects, DL/EDL resides on a cloud infrastructure (e.g., a private cloud, or a public cloud that offers infrastructure-as-a-service). In aspects, the NDS-memory comprises, or primarily, generally, or is entirely composed of data lake servers, each data lake server is regarded as a data lake server node, and all the data lake server nodes are connected to one another to form a mesh topology structure. An example of a programmable, automatically scalable, cloud-based system for DL or EDL cloud memory applications is the Microsoft Azure Data Lake Service, which can dynamically allocate or de-allocate memory resources in a parallel and distributed manner, and which provides applications such as Hadoop or portions thereof (e.g., YARN), Spark, and SQL-like query engine(s) (SCOPE/U-SQL). Other applications applicable to DL/DLLR systems known in the art include Hive, Map Reduce, HBase, Storm, Kafka, and R-Server. Other alternative DL management systems in the art include IBM® Watson™ or DeepDive™ systems.

In aspects, data in a DL/EDL of NDS(s) comprises a sufficient amount of associated metadata, has undergone a sufficient amount of processing or improvement, or both, such that the data is identifiable as belonging to or having been generated by a specific device (e.g., a specific MA), specific type of MA (e.g., and ECMO or a heart pump), a specific Entity, a specific group of MAs or Entities, a specific HCP, or other such specifically identifiable source or characteristic. In certain further aspects, data in a DL/EDL of NDS(s) comprises sufficient identifying information to be capable of being tied to a specific physiological state, condition, patient, or other such specifically identifiable source or characteristic.

In aspects, the DL/EDL can store≥about 1 trillion, ≥2 trillion, ≥3 trillion, ≥5 trillion, ≥10 trillion, ≥about 20 trillion, or ≥about 50 trillion files (with file sizes of up to 1 petabyte). In aspects, the DL/EDL comprises ≥~20 petabytes, ≥50 petabytes, ≥100 petabytes, ≥250 petabytes, ≥500 petabytes, or ≥~1000 petabytes of capacity (e.g., about 1-500 petabytes, about 2-400 petabytes, about 3-300 petabytes, or 5-250 petabytes). In aspects, average or median data latency in a DL/DLLR or latency for most, generally all, or substantially all data transfers is ≤~10 minutes, ≤~5 minutes, ≤~3 minutes, ≤~2 minutes, ≤~−1 minute, ≤40 sec, ≤30 sec, ≤15 sec, ≤10 sec, ≤5 sec, or ≤~3 sec (e.g., about 2-2,000 sec; 3-1,500 sec; 3-1,200 sec; 3-900 sec; 4-400 sec; 4-240 sec; 3-300 seconds; or about 5-300 sec). In aspects, the average operationality of the DL/EDL or the primary, general, or substantial level of operability over periods of 1 month, 1 quarter, or 1 year, is ≥~98%, ≥99%, ≥99.5%, or ≥~99.9% (e.g., as reflected in a memory vendor SLA measurement or other similar measure).

In aspects, latency in terms of ingestion, most applications, or both, relating to the DR, is measured in see or minutes (e.g., ≤~200 sec, ≤150 sec, ≤90 sec, ≤60 sec, ≤40 sec, ≤30 sec, ≤20 sec, ≤10 sec, ≤5 sec, or ≤~1 second (e.g., K about 0.5 second, ≤0.1 second, or ≤about 0.01 second) in most cases, generally all cases, substantially all cases, or on average.

In aspects, NDS memory also comprises a data warehouse component, wherein a DL or EDL is employed as an initial storage/staging area. In such aspects, data flow into NDS-memory goes from the network inputs (e.g., primarily MAs)/data pipeline, into the DL or EDL, and then some of the DL/EDL stored data is relayed to the data warehouse (typically less than most, e.g., less than about 33%, ≤~25%, ≤15%, <10%, or ≤~5% of received data in any relevant period (month, quarter, or year)). In aspects, a hybrid data warehouse/data lake platform or management tool, such as the commercially available SNOWFLAKE™ memory management application makes up a part of the NDS-memory. In aspects, NDS memory comprises a massively parallel analytic database/DR, which, in aspects, can support near real-time results to complex SQL queries (interactive query capabilities), but which typically filters for or transforms data to highly structured data prior to storage in such a DR.

In aspects, a DL/EDL comprises another memory structure, such as a graph database, which stores event-related data or data trends (e.g., medical procedure event data, patient symptom data, patient outcome data, condition progression data, medical complication data, adverse event data, device performance events, patient response event data, etc.). However, in other aspects, the NDS memory lacks a graph database, reserving the memory for other memory architecture and detectably or significantly (DoS) reducing complexity of data ingestion.

In aspects, the memory unit comprises data compression capabilities, which, in aspects, DoS enhance the data storage capability of the memory unit. In aspects, data compression functions are automatically or on-demand applied after ingestion (and similar decompression employed when compressed data is accessed/consumed). In aspects, compression provides a ≥~2-to-1, ≥~3-to-1, ≥~5-to-1, or ≥~10-to-1 improvement in capacity.

DLs in NDSs of the invention typically are subject to query applications (e.g., using query/search components of any of the above-described platforms) with resulting data being optionally subjected to schemas to organize resulting data for presentation, further analysis, or both, as discussed elsewhere. Aspects of data lake technology, applications, etc., which may be adaptable to DL/EDL applications are described in, e.g., US20200380169, US20180373781, US20190370263, US20200210896, US20200193057, U.S. Ser. No. 10/846,307, U.S. Ser. No. 10/706,045, U.S. Ser. No. 10/572,494, U.S. Ser. No. 10/545,960, U.S. Ser. No. 10/795,895, WO2018236341, CN111221526, CN111460236, CN111221887, CN111221785, and IN919CHE2015A. Additional aspects related to the application of a somewhat enhanced DL (in which time series metadata and other unspecified enhancements to facilitate query applications can be applied) are described in US20180082036.

b. EDLs

In aspects, NDS memory unit(s), comprises, mostly/primarily comprises, consists essentially of, or generally consists of enhanced data lake (“EDL”) DR(s). An “EDL” typically differs from a “true DL” in (1) organizing data stored in the EDL in the DR into governance zones, structured DR zones, or both; (2) imposing minimum structure requirements on some, most, generally all, or all incoming and stored data; or (3) both (1) and (2). In aspects, an EDL DR is distinguished from a true/typical DL in that in ingestion of data into the EDL, the ingestion process(es)/function(s)/unit(s)/engine(s) (a) employ(s) one or more data improvement processes to data prior to storage in the EDL DR (e.g., data review and correction processes); (b) imposes new metadata tags on/to the incoming data, imposes one or more structural requirements on/to incoming Records (records), or both, e.g., such that records stored in the EDL comprise, e.g., ≥~7-8 data attributes; (c) characterizes data and targets/locates records/data into the EDL into one of a plurality of data governance zones subject to different policies; or (d) any combination thereof. Despite such imposition of enhanced data structure on EDL data, an EDL also can comprise one or more DL characteristics including (a) ability to receive unstructured/semi-unstructured heterogeneous data; (b) storage of different types of data in a single integrated collection; (c) primarily, generally, or only schema-on-read data organization; or (d) any combination thereof.

“Ingestion” is understood in the art as the process(es) of and associated with receiving and storing data/records into a memory system/device, such as a DR. Attributes that can be imposed on data/records in an EDL ingestion process can include, e.g., the type of data (e.g., video, text, etc.), feature and associated attribute data (e.g., MA type feature/attribute data), time series data, or query response tags/elements. In aspects, an EDL ingestion process requires that most, generally all, or all MA-D is associated with one, some, most, or all of (1) MA-D source data, (2) event-associated (log) data, (3) cached vs. RT MA-data characteristics, (4) alarm data, (5) MA operating status data, (6) MA-D type data, (7) privacy/RR-requirement indicators, and (8) MA Entity owner data. Imposing such requirements on data during an EDL ingestion process distinguishes the EDL from a true DL and can significantly enhance the performance of an NDS comprising an EDL DR, without imposing so much of a demand on the NDS processor(s)/system(s) involved in the ingestion process to significantly or detectably unacceptably impair/delay ingestion of SMAD, demand on query processes, or both.

In aspects, data in the EDL is stored in two or more distinct zones of the EDL, which are governed by different data management protocols/rules (policies) and comprise data having different identified characteristics applied on intake, after initial intake (e.g., based on a regularly running general schema application program applied at ingestion or based on the operation of on-demand applications such as queries, NDS analytical functions, etc., applied on raw data), or a combination thereof (CT). In aspects, one element of some, most, generally all, or all EDL data governance zone definitions is the presence of PHI. In aspects, one element of the data zone governance zone definition is whether the data has been subject to curation, scoring, or a combination thereof. Data can be tagged for subjecting to policies or policies can be applied to data that comply with certain standards (e.g., using if/then logic or related logical structures).

In aspects, EDL data that is relayed from the NDS as output (e.g., NDS-AD) is stored in another governance zone, subject to different policies, including application of tags/identifiers for data records comprising PHI (e.g., tags that work with other components to block access to or sending of such data in an unredacted or unmodified form to Commercial/commercial user class-associated device(s)/interface(s)). In aspects, part of an EDL is structured as or associated with an element of the NDS-MEMU that is structured as a database or a data warehouse, e.g., as a data mart component of the EDL, in which a material amount, most, generally all, or substantially all the structured data is NDS-AD or a subset thereof or data related thereto (e.g., responses to schema applications in response to queries or otherwise applied on unstructured/semi-structured data in the EDL or other part of the DR).

In facets, the NDS can comprises engine(s)/component(s)/system(s) for data grouping and segregation capabilities. In aspects, NDS can comprise a plurality of data sets or data capable of being characterized as identifiable as belonging to a particular subset of data. For example, in aspects, an NDS can comprise (a) data for MAs in a particular country, (b) nation-specific data governance rules, and (c) system blueprint data shared with one or more other NDSs. According to embodiments, the NDS-MEMU comprises separate governance zones that manage storage of, use of, and access to (a) SMAD, cache data, or both; (b) curated data, scored data, or both; (c) system testing data; and (d) outgoing data. Such function(s) can be performed by, e.g., application of metatag(s) on records/data, identification of record(s)/data matching certain characteristics (e.g., by if/then structure analysis, etc.), or a combination thereof.

In aspects, an EDL accepts only data comprising certain set types of attribute and value/feature-like relationships or stores only data comprising certain set types of attribute and value/feature-like relationships in certain data governance zones. In aspects, the NDS monitors if the NDS is receiving and storing data having certain attribute-and-feature like relationships into the EDL and alerts administrators is such data is not being received. In aspects, an EDL accepts only data comprising certain data structures (e.g., only accepts semi-unstructured data sets, such as JSON data or other semi-unstructured data formats, discussed elsewhere) or stores only data having such structures in certain zones. In aspects, an EDL accepts several data structure type(s), but segregates data in the EDL, e.g., into different governance/content zones of the EDL, based on, i.a., data structure.

In aspects, data structure requirements or transformation processes are applied to only certain types of data streams, and other data streams/input(s) are processed in a less regulated matter (like or more like a traditional DL). For example, in aspects, structure requirements, transformations, or both are placed on some, most, generally all, or all of one, some, most, generally all, or all MA-D data/streams, but other forms of input are not subject to such structure requirements or transformations. E.g., in aspects, the NDS can receive input through emails, other messages, note applications, web page data, etc., or from external data repositories/applications (e.g., CMSS(s)), which data can comprise unstructured data or semi-structured/structured data that has a structure that is subject to changes not under current or previous control of the NDS owner. Video data, image data, audio data, or combinations of such data also can be received by a DL/EDL in raw format, in a format that requires conformity to certain standards, or both.

Ingestion of data into an EDL can comprise, e.g., receiving data stream(s) (e.g., comprising torrential data streams) and applying transformation(s) or imposing structure requirement(s) on such incoming data, prior to storage (during ingestion/pre-ingestion), and typically imposing additional structures/schemas on stored EDL data prior to some, most, generally all, or all applications/consumption of such stored EDL data (post ingestion). Data transformation, as discussed elsewhere, can include application of data improvement unit (DIU) to e.g., initially harmonize data, clean data, validate data, etc. Data transformation step(s)/function(s) can also comprise application of metadata; curation, e.g., into governance zones based on data content/metatags; or a CT. Data transformation can comprise application of encryption (e.g., applying SSL on data in motion or applying HSM-backed keys to “at rest” data in the EDL).

Access to EDL data can require authentication, e.g., multi-factor authentication, and the EDL management functions can include role-based access controls (e.g., via POSIX-based access controls). EDL management function(s) can further comprise auditing access or configuration changes to EDL data (or to other aspects of the NDS or to the NDS generally).

Data comprising personally identifiable information (PII), PHI, or both, can be identified in the data intake process and the DIU can encrypt such data or other confidential information using, e.g., tokenization with format preserving encryption (FPE). In aspects, some, most, generally all, or all metadata applied to incoming data is automatically generated from data input into the EDL based on preprogrammed rules.

As with a DL in general, an EDL, can be subject to querying and schema application (e.g., applied by an analytical unit function), which applies additional structure to the data through a schema, typically comprising ≥~7, ≥10, ≥12, ≥15, or ≥~20 associated data elements for each record identified in the data, wherein query-identified records can number in the, e.g., 100s; 1000s; 10000s; 100000s; or e.g., 1000000s. EDL management functions can further include messaging functions, logging functions, reporting functions, export functions, crawler functions, machine learning modules, or any CT. In aspects, the EDL functions include one or more natural language query functions also to query functions based on particular attribute/field relationships or other data entries/types. A data improvement unit (DIU) can apply processes on incoming or stored data that normalizes, enriches, or tags data streams or stored data. In aspects, DIU processes applied to incoming data/streams do not detectably or significantly (“DoS”) impact latency.

In aspects, an NDS/MAC-DMS processing unit transforms MA-D, imposes requirements on MA-D, or both, such that substantially all MA-D datasets stored in the enhanced data lake comprise medical apparatus source-identification information, MA-D type information, and one or more physiological parameter datasets, each thereof optionally presented in a preprogrammed MAC-DMS-recognizable standard format and the enhanced data lake comprises different data governance zones that are based on the source or content of MA-D datasets, analytical data, or both, stored therein, as described above.

### 2. NDS Processor(s)

An NDS comprises a processor unit(s)/system(s)/component(s) (sometimes called an NDS-PROCU or NDS processor), which are device(s)/system(s) that can execute (read) NDS CEIs stored in NDS memory. As with NDS memory, an NDS can comprise multiple processor units, which can be physically or functionally separate from each other. Such separate processing device(s)/system(s) can form a discrete/inter-operating processor or an NDS can comprise multiple processing unit(s) that at least some, most, or all of the time operate separately from each other. For example, in aspects an NDS comprises (1) first processor(s) associated with a Streaming Data Processor (SDP) unit/engine/system (aka, an SDP or SDE) which, e.g., performs initial analysis tasks on incoming data and separate second (primary) NDS processor(s) that process(es) post-ingestion NDS DR data (e.g., performing NDS DR queries, generating NDS-AD, and managing NDS output, etc.).

Although an SDP is often described as a “processor,” here and in the art, an SDP, in aspects, can lack any separate physical processor device(s)/component(s) (e.g., an SDP can comprise engine(s) that work with separate processor(s), such as a main/overall system processing unit). Thus, an SDP can be alternatively described as an engine (a streaming data engine (“SDE”)) or a Streaming Data Unit (“SDU”). In aspects, an SDP does include separate physical processor component(s) from other processing unit(s) of the NDS. Each such different aspect is implicitly provided by any disclosure of an SDP.

An NDS processor can have any suitable features/component(s) for performing the functions of the NDS or applicable NDS component in accordance with the characteristics/aspects described here. Given the role of the NDS in receiving large amounts of data from multiple MAs, typically in different MA groups (and in cases from different types of MAs, different patient types, different treatment protocols, etc.), to analyze such data, and to provide such data to HCPs and other users of MAs as well as other system components, an NDS processor (a) can comprise significant physical processing capability which goes well beyond the typical processing capability of any single laptop/desktop general purpose computer; (b) can comprise engine(s)/unit(s)/component(s) that employ specialized data management methods to ensure prompt handling, analysis, and relay of data; or (c) can comprise both (a) and (b).

In aspects, the NDS processor comprises a workflow architecture that can be characterized as comprising massively parallel processes/very large workflow (e.g., as such terms are understood in the art in connection with leading cloud-based systems, such as Microsoft Azure systems). E.g., in aspects, the number of cores/processors in a multi-core MA is ≥~10, e.g., ≥~20, ≥~50, ≥~100, ≥~200, ≥~500, or ≥~1,000, such as ≥~2,000, ≥5,000, ≥10,000, ≥12,500, or ≥~15,000 cores (e.g., ~1,000-20,000 cores, 1,000-15,000 cores, 3,000-18,000 cores, 2,000-16,000 cores, or ~2,500-15,000 cores). An NDS processor in aspects is primary, generally only, or entirely composed of cloud-based processor capabilities/functions. In aspects, the NDS processor operates based on a scalable, massively parallel, and distributed processor architecture.

Massively parallel process (MPP) functions/methods can be achieved by use of large system step functions, such as Amazon Web Services (AWS) Step Functions. Microsoft Azure services also include MPP functions. In aspects, an NDS will comprise an architecture comprising (always, on average, or at peak operational capacity)≥~100, ≥~150, ≥~200, ≥~500, ≥~1000, ≥~2000, ≥~5000, or ≥~10,000 processor/memory combination(s)) enabling some, most, generally all, or substantially all analytical functions performed by the NDS processor(s) to complete in e.g., ≤~30, ≤~10, or ≤~5 sec. MPP architectures usable in NDS processor(s) typically comprise interconnect data pathways. In aspects, an NDS processor has a grid computing MPP architecture, computer cluster MPP architecture, or both.

In aspects, one, some, most, or all NDS processor(s) can be characterized as being “highly available” or comprising “highly available” workflow(s). In aspects, an NDS or component of an NDS exhibits≥~97%, ≥~98%, ≥99% availability (accessibility and standard/optimal operability) over a period (e.g., per quarter, year, 3-year period, 5-year period, etc.), and in more particular aspects, a highly available NDS exhibits≥~99.8%, ≥~99.9%, ≥~99.95%, or ≥~99.999% availability. High availability can be achieved by any suitable method(s) including normal means, message queues, lambda retry functions (e.g., in AWS), or a CT. General resources/structures for high availability comprise application of component redundancy, component monitoring and management, failover (node/process substitution/routing), use of distributed replicated volume(s), load balancing, or a combination thereof.

In aspects, NDS processor(s), e.g., a MPP NDS processor, exhibits≥~100 GB/sec bandwidth (e.g., ≥~150 GB/sec, ≥200 GB/sec, or ≥~250 GB/sec bandwidth). In aspects, the NDS processor exhibits≥~2 GHz, ≥2.5 GHz, ≥3 GHz, ≥3.33 GHz, or ≥~3.5 GHz processing. In aspects, the NDS process can perform/process≥~50,000 transactions/events per second, ≥75,000 transactions/events per second, or ≥~100,000 transactions/events per second. In aspects, the NDS processor operates on a ≥~0.5 petaFLOPS, ≥~1 petaFLOPS, ≥~2 petaFLOPS, ≥~3 petaFLOPS, ≥~5 petaFLOPS, ≥~10 petaFLOPS, or ≥~25 petaFLOPS, basis most of the time, generally always, substantially all the time, or on average (e.g., between about 0.5-12.5 petaFLOPS, e.g., 1-15 petaFLOPS, 1-20 petaFLOPS, 2-50 petaFLOPS, 2-80 petaFLOPS, or between about 5-100 or 10-120 petaFLOPS, etc.).

In aspects, an NDS processor comprises partitioning functions, processor capability scaling functions, processor resource reservation functions, checkpointing functions, data queue-based processing functions, error recovery functions (e.g., for redundant processor error functions), or a CT, which detectably or significantly enhance performance of the processor.

Parallel processing systems (including MPP systems); related methods, functions, and data structures; and other related principles, many of which are adaptable to aspects, are described in U.S. Pat. Nos. 5,485,627, 8,799,284, 8,583,896, U.S. Ser. No. 10/802,929, U.S. Pat. Nos. 5,404,562, 4,727,474, 5,765,181, 9,239,741, U.S. Ser. No. 10/339,235, U.S. Pat. Nos. 8,903,841, 5,230,079, 4,380,046, 7,716,336, 9,569,493, U.S. Ser. No. 10/147,103, U.S. Pat. Nos. 5,799,149, 6,185,693, 8,903,841, 5,566,321, US20030110230, US20080034157, US20170270165, US20090031104, US20030195938, U.S. Ser. No. 10/078,565, US20170180272, US20130111188, US20200167362, U.S. Pat. Nos. 5,881,227, 5,103,393, 8,799,284, US20170270165, US20200364226, US20150120368, U.S. Ser. No. 10/372,696, U.S. Ser. No. 10/565,199, U.S. Pat. Nos. 6,098,178, 5,103,393, 5,511,221, 6,957,318, 5,146,608, US20100287557, U.S. Ser. No. 10/303,654, U.S. Pat. Nos. 9,910,821, 9,697,170, 5,390,298, 5,008,815, 5,872,987, 5,253,308, 8,108,718, 6,185,693, 9,448,966, EP0456201, EP0381671, CN103778212, CN103237045, KR101632253, KR1020140063279, KR1020170056773, and KR101083052.

Parallel processing can comprise an architecture of or perform distributed parallel processing, non-distributed parallel processing, or both. “Distributed processing” typically means processing performed on physically separate but networked machines. Non-distributed parallel processing can be performed on interconnected and co-located cores. Parallel processing systems can comprise systems classifiable as clusters, grids, cloud, or a combination thereof. In aspects, the NDS processor comprises heterogenous software, heterogenous hardware, or both, or operates via a heterogenous network (e.g., in terms of components, layers, communication protocols, and other aspects of topology). In aspects, the NDS processor, NDS memory, or both, are dynamic systems (variable over time), in terms of software, hardware, or both.

NDSs, networks, or both, can include or use routers, which can comprise networking/communication equipment configured to provide internal and external communications. Routers can include packet-switching and/or routing device(s)/unit(s) (including switches and/or gateways) configured to provide network communications between NDS component(s) (server processing unit(s)/device(s)) and NDS memory (data storage) via local cluster network, network communications between NDS/NDS component(s) (e.g., server cluster) and other devices, or both, via communication links across the network. Routers can be selected for capability to handle, configured for, or both, to handle the data demands of network, data storage, latency, availability, and throughput of NDS/network and other factors that may contribute to the cost, speed, fault-tolerance, resiliency, efficiency, and/or other design goals of the network/NDS architecture. Router(s) of the NDS/network can comprise security capabilities, such as VPN, firewall, etc. and can also include multiprotocol label switching capabilities. Such routers are known and include, e.g., Cisco Catalyst 8000V routers, Cisco Catalyst 9000 routers, etc. In aspects, routers have scalability capabilities that can be modified in response to instructions from the NDS. In aspects, routers are coupled with LAN switches with similar capabilities. In aspects, some, most, generally all, essentially all, or all routing, switching, and similar functions are performed by software defined wide-area network components/units (SD-WAN appliances) (which can be on-demand components).

In a distributed computing environment, such as may be present in certain NDSs, program modules or subroutines can be located in local or remote memory storage devices. Programs or program modules used in a distributed environment can be distributed electronically over the Internet or over other networks (including wireless networks). In specific facets, DR(s) are stored in whole or in part and processor function(s) are employed via a cloud platform such as Microsoft Azure or AWS. Distributed processor system(s)/component(s) that can make up some, most, generally all, or all parts of an NDS processor can use distributed memory, whereby, e.g., processors can be endowed with chunks of the memory space and communicate via message-passing or similar methods. Each processor can in such aspects have direct access to its local memory and indirect access to nonlocal or remote chunks of memory.

In aspects, NDS engine(s)/component(s)/unit(s)/system(s) are mostly, generally, essentially, or entirely based in public cloud networks/remote server devices (e.g., integrated/linked server cluster(s)) that can be used for outsourced computation, data storage, communication, and service hosting operations. These server(s) may be virtualized (i.e., server(s) may be or comprise virtual machines). Examples of public cloud networks include Amazon Web Services (AWS) and Microsoft Azure Services. NDS can comprise a network management platform and server cluster(s) supporting public cloud networks, providing load balancing, redundancy, high availability, scalability, etc.

In aspects, NDS can comprise virtual machines/servers (emulations comprising memory, processing, and communication resources), which can be embodied by computer device(s), server cluster(s), etc., which typically are managed by a centralized server device, application, etc., which acts as an NDS controller, and can be accessed selectively by authorized NDS administrators. Virtual machine systems through Microsoft, VMWare, and the like are known in the art and can be adapted to such applications.

In aspects, hardware of an NDS processor can comprise specialized node(s) for select analytical functions, which can comprise GPUs, specialized data retrieval/mining units, etc., which can in aspects comprise non-cloud, dedicated hardware units, but which can interface with cloud-based memory/processor components. In aspects, the processor function comprises FPGA(s) that DoS lower processing bottlenecks and DoS/DOS accelerate one or more functions (e.g., search operations). In aspects, some, most, or generally all the processing components comprise multicore, multithreaded, or GPU processors. In aspects, the processor exhibits between about 2.5-25, 2.5-15, 2.5-12, or between about 2.5-10 (e.g., ~1-10, ~2-12, ~1-5, ~2-6, or ~2-10) teraFLOPS of precision. In aspects, the NDS processor/system comprises ≥~100 GB/s of memory bandwidth, such as ≥~125, ≥150, ≥175, or ≥~200 GB/s of bandwidth (e.g., between about 50-250 GB/s bandwidth, such as ~100 Gb/s InfiniBand). In aspects, the NDS processor comprises, primarily comprises, or generally comprises cores with ≥~100, ≥200, ≥300, ≥350, ≥400, or ≥~500 GB RAM. In aspects, the NDS processor comprises, primarily comprises, or generally comprises cores with baseclock speeds of ≥~2 GHz, ≥2.5 GHz, ≥2.75 GHz, ≥3 GHz, ≥3.2 GHz, ≥3.4 GHz, ≥3.5 GHz, ≥3.6 GHz, ≥3.7 GHz, ≥3.8 GHz, or ≥~4 GHz. In aspects, MPI latency in the NDS processor is ≤~5 sec, ≤2 sec, ≤1 second, ≤0.25 sec, ≤0.1 sec, ≤0.01 sec, or ≤~0.005 sec on average, mostly, or generally. In aspects, node connections comprise Gigabit switches, e.g., switches supporting≥~25, ≥35, ≥40, ≥~50 Gpbs, ≥about 75, ≥about 85, or ≥about 100 Gbps link speeds.

In aspects, processor unit function(s)/engine(s) can comprise data scheduler(s) (e.g., a Mesos, YARN, or Sparrow data scheduler). In aspects, a data scheduler of an NDS processor has a response time of ≤~10 sec, ≤~5 sec, ≤~2 sec, or ≤~1 second (e.g., between ~0.5-7.5 sec, ~0.25-5 seconds, or ~1-10 seconds). In aspects, a DMS of an NDS processor comprises a HULL (High-bandwidth Ultra-Low Latency) architecture. In aspects, processor unit function(s)/unit(s)/engine(s)/system(s) include a streaming data processor (also referred to as an SDP, SDE, or stream processor), which can, in aspects, also be classified as a component of an NDS input unit/system. In aspects, the stream processor can perform stream processing functions such as map, filter, joins, and aggregation functions, and other data transformation functions (e.g., as available in Kafka Streams). An SDP, primary processor, or both, can, e.g., comprise engine(s) for assembling time series and other appropriate collected data from MA-Ds. In aspects, reassembly of cache data and associated time series RT MA-D is performed mostly or entirely outside of an SDP (e.g., in the primary processor or a specialized processor/engine), to reduce data processing burden on an SDP/SDE. Reassembly of cache RT MA-D is discussed elsewhere.

Interconnection of components can be facilitated via Ethernet, fiber optic cable, Wi-Fi, or another suitable connection/topology/method. Processing functions include mapping functions for CEI and other data, scheduling functions, for the performance of tasks (including, e.g., ingestion of data, as discussed elsewhere), or both. network interface(s) may also support communication over one or more non-Ethernet media, such as coaxial cables or power lines, or over wide-area media, such as Synchronous Optical networking (SONET) or digital subscriber line (DSL) technologies. Processing functions also can include virtual machine monitors/controls, APIs, etc., for user control of the NDS memory system/unit. Other functions which can be executed by the NDS processor are separately described herein (e.g., input functions, memory functions, analytical functions, and relay functions) (for sake of clarifying processes such functions are often associated with particular units/steps herein, such as an analytical unit, input unit, etc.). Related processing functions that can be implemented by NDS processor(s) are also discussed elsewhere (e.g., use of NoSQL or Hadoop for data ingestion and management in the NDS DR). In aspects, processing is managed through parallel processing in most, generally all, or substantially all cases. In aspects, batch processing is employed by NDS processor(s) for certain processes. Such data processing methods are further discussed elsewhere. In aspects, NDS processing comprises bulk synchronous parallel (BSP) processing component(s)/function(s)/engine(s). Other feature(s)/component(s) of an NDS (and where appropriate networks) used to provide the structure/function of systems/networks described here can also or alternatively include, e.g., virtual switch(es), virtual bridge(s) (e.g., to NICs), virtual adapter(s), NAT service component(s)/system(s)/engine(s), router(s)/router table(s), DNS/CSP system(s), subnet system(s), traffic monitors/managers, traffic filter(s)/firewalls, etc.

### 3. NDS Security Engine(s)/Unit(s)

NDSs can comprise security unit(s) (system(s)/component(s)/engine(s)) comprising security component(s), which can be hardware components, software components/Engines, or a combination thereof (CT). The NDS security unit (also referred to as NDS-SECURU or “NDS security”) can be or comprise any combination of elements that provide one or more data security functions at a network level.

In aspects, NDS security comprises firewall(s). In aspects, the NDS firewall function comprises one or more packet-filtering firewalls (e.g., stateless packet-filter firewalls). In aspects such firewalls comprise protocols for checking packet data against access control list(s), and a protocol for dropping/blocking unauthorized relayed packets, passing authorized packets, or both. In aspects, NDS firewall(s) employ Stateful Packet Inspection (SPI) (aka, dynamic packet filtering). In aspects, the NDS firewall(s) comprise proxy server firewalls (aka, application-level gateways), which, i.a., mask network IP addresses, limit data traffic types, or both. In aspects, NDS firewall(s) comprise circuit-level gateway(s) (ensuring, i.a., safe connections). In aspects, an NDS security unit comprises a deep packet inspection firewall, which analyzes some, most, generally all, substantially all, or all the payload content of some, most, generally all, substantially all, or all received packets, TCP handshakes, or both. In aspects, an NDS security firewall performs both surface and deep packet inspection, e.g., in an ordered fashion, based on analysis of the packets, or both. In aspects, the firewall function(s) also perform antivirus scanning/protection functions, spam filtering functions, application control functions, or a combination thereof. In aspects, the NDS security unit employs cloud firewall(s) (aka, firewall-as-a-service (FWaaS) functions), as made available via, e.g., Microsoft Azure Services, which are scalable, typically in association with the scalability of other NDS elements, e.g., processing capability or memory capacity, or automatically or on-demand in response to increased traffic load independently of other expansion(s) of NDS capabilities. Firewall(s) at the NDS level also or otherwise can be web application firewalls, which can be hardware or software based.

In other aspects, NDS security can comprise data encryption capabilities, anti-virus functions, authentication capabilities, data exclusion/redaction capabilities, or any CT, etc., aspects of which are discussed elsewhere herein (e.g., in respect of methods, NDS memory, or MAs).

### 4. NDS Input Engine(s)/Component(s)/Unit(s)

NDSs comprise at least one NDS Input Unit (NDS-INPU or NDS input). The NDS Input Unit typically includes a combination of hardware component(s) and Engine(s) that handle receipt of data from external sources and the initial processing of such incoming data leading to memory component/system data storage (ingestion).

In aspects, an NDS input unit or component thereof/alternative thereto (e.g., an SDP/SDE) can perform evaluation(s)/analytical procedure(s) on data received from some, most, generally all, or all MAs, other inputs (e.g., ONDIs), or both. In aspects, such “initial analysis” steps are performed primarily, generally only, or only on MA-D. Such initial analyses processes can be, e.g., distinct from other NDS analytical processes (e.g., performed by an analytical unit, on DR data, or both). In one aspect, the NDS input unit can in initial analysis determine if MA-D received from MA(s) is NRT/RT-MAD or cache data, SUMAD, or both. Such functions can be performed by known techniques, such as, e.g., data time synchronization and time series data matching/analysis. In aspects, an initial analysis can include analyzing MA-D for preprogrammed patterns, indicators, etc., that require immediate action based on incoming data, prior to even full data ingestion. In aspects, such preprogrammed patterns are programmable, are modified by machine learning processes, or both. In aspects, the NDS input unit can work with or comprise a controller system/engine/unit that sends control instructions to MAs, ONDIs, or both. In other aspects, an NDS generally, substantially only, or only analyzes data during or after ingestion into NDS memory, rather than performing an initial analysis. In some such aspects, the NDS also can comprise a stream processor that receives SMAD.

The NDS input unit (NDS-INPU) can also comprise a buffer unit/function, which maintains any excess data received through incoming data sources/streams until capacity for initial analysis, initial transformation, and other aspects of ingestion are available. The use of the buffer, at or in excess of one or more thresholds, can provide signal(s) to a scalable system for evaluating scaling or scaling resources to accommodate incoming data load to avoid significant increases in latency over periods (e.g., a day, week, or month).

The NDS input unit can, or can most of the time, generally all the time, or substantially all the time simultaneously receive/process multiple data streams, e.g., streams of TCP packets, e.g., via multiple processing system(s)/function(s), discussed below and elsewhere. Such an NDS input unit can comprise or be a Streaming Data Processor (SDP/SDE), e.g., as discussed further below and elsewhere.

Input received by the NDS input unit can be in any suitable format (e.g., text/alphanumeric data, tabular data, video data, audio data, image data, or any suitable combination thereof). In aspects, some input, e.g., an appreciable amount, a material amount, or other amount (in terms of file numbers, data size, or both), which typically is less than half of the input, is in or can be in the form of unstructured data (e.g., emails, web pages, etc.). In aspects, most, generally all, or substantially all input received by an NDS has a semi-structured format (e.g., CSV, JSON, or Avro data format). In aspects, most, generally all, or substantially all input of data type(s), e.g., MA-D, is in JSON format.

Input can be processed by batch processing, real time/streaming processes, or both. Aspects of batch and real time/streaming data are discussed elsewhere and below. An NDS input unit typically can perform both processes, e.g., applying batch processing on cached data uploads and real time/stream(ing) (RT/S) processing on MA-D most of the time or generally when an MA is online (in substantially continuous communication with the NDS). Batch processing also can be applied to, e.g., event/log data from network components, including MAs. In aspects, an NDS comprises a unit that is, at least in part, specially designed for the analysis of cache data (a cache MA-D processing unit). A cache MA-D processing unit/engine can be a part of a broader component of the MA, such as the MA processing unit. In aspects, a cache MA-D engine/processor comprises coded functions, routines, and the like, which specifically analyze cache MA-D. In aspects, a cache data processor/engine/system is at least partially discrete from other processor(s)/engine(s)/unit(s) of an NDS. In aspects, such a cache processor/engine relays to network device(s) information concerning whether the processor/NDS has received, analyzed, or utilized cache information or not (e.g., by providing status information, alarms, or the like at an MA that relayed cache data to the NDS).

In aspects, an NDS/MAC-DMS processing unit evaluates whether received cache MA-D of a medical apparatus is combinable with received streaming MA-D of the same medical apparatus and if the MAC-DMS processing unit determines that the cache MA-D and streaming MA-D are combinable, combining the streaming MA-D and cache MA-D to form a mixed MA-D data set, wherein the MA-D analyzed by the analytical unit comprises the mixed MA-D data set. Combining of cache MA-D and relevant streaming MA-D typically comprises evaluating the time component of one, typically two, streaming MA-D datasets, and the endpoints of a cache MA-D dataset to determine if there is sufficient proximity in the timepoints/endpoints to combine the cache and streaming datasets. Such an evaluation can comprise generating a putative match, evaluating the quality/readability/analyzability of the putative match (combined cache/streaming MA-D). In this or other respects, the NDS processor (or components thereof) assess(es) the usability of cache data for analytical processes, the suitability of storing cache MA-D in NDS memory, or both.

Analysis of and, if suitable, combination of data/records by an NDS, such as cache data and RT-MA-D from a particular MA, can be performed by any suitable data linkage and assembly methods. A number of such methods are known, and, accordingly, are only briefly described here. In aspects, most, generally all, or all MA-D relayed from MAs (cache data and RT-MA-D) will comprise time information and device-identifying information (which can be considered an entity identifier). In aspects, processor(s) of an NDS, such as a cache data processor, comprises engine(s) that identify common entity identifier information, time of capture/transmission information, and uses such information, i.a., as a/the basis for determining to combine cache data with MA-D. In aspects, MA-D comprise ≥2 or ≥3 identifiers that NDS component(s)/system(s)/engine(s) are preprogrammed to identify, compare, and analyze similarity/matches of in such assessments. In aspects where there is no gap in time between RT MA-D and cache data, a join/merge process can be used to reassemble data from an MA-D

In aspects, an NDS comprises engine(s)/CEI for evaluating where data does not match based on data/record identifier(s), but where data is expected/known to be related/similar; where there are gaps in a time series of data; or both. Such methods can draw upon/incorporate record/data linkage/matching methods/functions known or in tools available in the art.

In aspects, engine(s) involved in data/record linkage determination employ deterministic algorithms (e.g., rejecting matches unless 1, 2, 3, or more entity identification data markers are present, time series information matches, or both). In aspects, such engine(s)/system(s) or other NDS component(s) also or alternatively employ probabilistic data/record linking strategies (e.g., evaluating (1) the discriminatory power of each identifier and (2) the likelihood that two records are a true match based on whether they agree or disagree on the various identifiers). The weight assigned to agreement or disagreement on each identifier can be assessed as a likelihood ratio, comparing the probability that true matches agree on the identifier (e.g., an “m-probability”) to the probability that false matches randomly agree on the identifier (e.g., a “u-probability”). The EM algorithm, for example, is an iterative approach to estimating m- and u-probabilities. See, e.g., Dusetzina S B et al. 2014 Sep. 4, An Overview of Record Linkage Methods. Available from:ncbi.nlm.nih.gov/books/NBK253312/.

Such function(s)/steps(s) can comprise, e.g., binary/pairwise supervised classification, clustering processes, probabilistic data linkage processes, etc. Such processes can comprise transivisity/nontransitivity check functions/processes. In aspects, such processes/engine(s)/function(s) comprise collective entity resolution techniques.

In aspects, such function(s)/engine(s) comprise protocols/algorithms for the protection of confidential information, e.g., PHI, in a network comprising multiple patients, IEs, or classes of users (such as commercial class users). In such aspects, match data may be relayed to some, most, or all client devices (e.g., MAs), but the details of the underlying data are not relayed. Encryption methods, such as Bloom filters, or other encryption techniques described elsewhere, also or alternatively are employed in such processes to protect confidential information.

In aspects, engine(s)/component(s)/system(s) engaged in data/record evaluation and linking use step(s)/engine(s)/algorithm(s) for parsing/blocking records that are more likely to contain matches into smaller sets, whereupon different approaches are taken to measure similarity. In aspects, a smaller/parsed data set is subject to cleansing or elimination prior to further potential linking analysis in most, generally all, or all cases. Typically, identifying similar records in different sets may indicate a link across the data sets, which facilitates cleansing, knowledge discovery, or reverse engineering, which can contribute to master data aggregation. Thus, e.g., engine(s) engaged in such processes can apply blocking, similarity scoring, and approximate matching step(s)/function(s)/code as known in the art, considering, e.g., data type and using such information to determine basis/bases for comparison/evaluation (e.g., simple distance function for numeric data, string comparison rules to identify “distance”, and the like).

In aspects, engine(s)/component(s) engaged in record matching in this or other contexts (e.g., in query processes) employ NLP engine(s)/algorithm(s) (e.g., Term Frequency, Inverse Document Frequency (or tf-idf)). NLP methods are also discussed elsewhere. TIF and similar algorithms splits text into ‘chunks’ (or ngrams), counts the occurrence of each chunk for a given sample and then applies a weighting to this based on how rare the chunk is across all the samples of a data set. Such algorithms are incorporated into ngram functions of programming languages/platforms available or adaptable to systems of the invention. In aspects, close matches also or alternatively are found through cosine similarity methods.

In aspects, the NDS can draw upon an internal library or other sources of information (e.g., third party databases, IE databases, or EMR/EHR information) to fill in gaps. In aspects, gaps are filled in from related MA-D from the MA, similar MA, or MA IE owner.

In aspects, fuzzy matching methods, known in the art, are used in comparisons/data linking. Such methods are known. E.g., in Python Pandas package (Pandas) provide tools for evaluation and merge of matching data (merge_asof). Pandas also can be used to work with time series data, generally, e.g., a pandas.DataFrame object can contain several quantities, each of which can be extracted as an individual pandas. The Python fuzzymatcher library provides an interface to link two pandas DataFrames together using probabilistic record linkage. The Python Record Linkage Toolkit provides a set of functions to automate record linkage and perform data deduplication (employing blocks of data, multiple algorithms for measuring string similarity, ranking of matches using scoring algorithms, use of supervised machine learning in the process, etc.). Similar tools/methods are known or adaptable to NDS processes. In aspects, data/record linking/evaluation and related functions, such as query functions, also take into account schema matching of records/data sets.

Once a match is determined to exist, engine(s) will employ merge, join, or similar functions/operations/steps to bring together/reassemble the relevant data/records (e.g., matching cache data and RT-MA-D from an MA). Such functions are well known in the art and available in common programming languages and commercial systems. E.g., Python Pandas include a high-performance, in-memory join and merge operations and merge and join statements/applications are available in SQL (e.g., using a SAS DATA step). In systems such as Azure/Power BI merge functions are available, as are merge functions that form part of Google BigQuery using SQL functions available through Google Cloud and join operators in Amazon Redshift. Joins can be, e.g., inner joints, right outer joints, left outer joins, full outer joins, cross joins, or any other suitable type of join depending on the data to be linked.

In aspects, an NDS or NDS component, such as a cache processor/engine, can perform a method or comprise an engine that performs a function comprising the steps of START (1) IF an MA goes offline, COLLECT expected related cache data and RT-MA-D from the MA (e.g., within 60, 30, 20, 10, 5, 2, or 1 minutes in time based on time series data); (2) COMPARE the RT-MA-D and cache data; (3) IF cache data EQUALS or is SUFFICIENTLY SIMILAR to RT-MA-D in type and time series (based on comparison standard(s)); (a) join/merge cache data/RT-MA-D; ELSE reject merge/join; (4) optionally relay result to MA/OND; and (5) END.

In aspects, most, generally all, or substantially all the input of the NDS is processed under RT/S processing. In aspects, batch processing tasks are performed using a Hadoop/MapReduce framework or similar framework (e.g., that can perform map and reduce functions, such as split, map, partition, shuffle, sort, and reduce functions), or a framework designed for both batch and RT/S processing, as discussed elsewhere. In either case, but particularly in batch processing, the NDS input unit can comprising a mapper function that takes attribute/value pairs (or key/value pairs) in incoming data, splits such data from any associated incoming file/chunk/stream data, and outputs a key-value pair or multiple key-value pairs for storage (filtering or demultiplexing functions) and optionally combines such output data through a combiner function, performs partitioning functions, performs reducer functions, performs shuffling functions, or any combination thereof. In aspects, batch processing can also be employed in analytical functions on stored data (e.g., performed by the Analytical Unit of the NDS processor). In aspects, some, most, generally all, or all analytical functions performed on stored data are performed through batch processing methods. In aspects, RT/S data delivered to the NDS is processed, i.a., by a system that is designed specifically for the processing of streaming data, such as Apache Storm or a similar system (e.g., a system with capability of processing≥~1,000,000 records per second per node), Apache Kafka, or the like. In aspects, an NDS-INPU comprises 2, 3, or more of such systems working together on various parts of receipt, analysis, and ingestion of RT/S data (e.g., a combination of Kafka and Storm).

Definitions of “real time processing” vary in the art. In aspects, real time (“RT”) processing means processing substantially only or only within a time such that in generally all or substantially all cases the NDS can intake/receive and at least initially analyze streaming MA-D before/during ingestion, or fully analyze automatic functions after ingestion, e.g., such that (1) most, generally all, or all of pre-determined time critical data are analyzed, and (2) (a) alert/alarm functions are delivered, (b) MA device control instructions delivered, (c) MA therapeutic instructions are displayed on applicable MAs, or (d) any combination thereof occurs. In aspects, a system, in operation, provides therapeutic MA control or treatment-related information output prior to any deleterious treatment or diagnostic/monitoring effect in most, generally all, or substantially all cases, or such that the number of output deliveries/applications that would be classified as late are not significant.

RT/S data ingestion typically comprises (mostly, generally, or only comprises) ingestion before the next stream is ingested to not significantly increase the amount of buffer data a significant amount of time. In aspects, the timing of receipt of RT/S data in the NDS does not substantially or significantly differ from the time of initial analysis completion. In aspects, the timing of RT/S data receipt, completion of initial analysis, data ingestion, or all three processes are about the same.

In aspects, “RT processes” mean processes performed in ≤~5 seconds, ≤~3 seconds, ≤~2 seconds, ≤~1 second, or ≤~0.5 seconds (e.g., ≤~0.25 seconds, ≤~0.2 seconds, or ≤~0.1 sec, such as between about 0.1-2.5 sec, 0.01-2 sec, 0.05-2 sec, 0.05-1 sec, 0.005-1.5, 0.001-1 sec, or between about 0.0001-1 sec). In aspects, some RT/S data is not stored by the NDS memory, other than in a buffer or in temporary memory for initial RT analysis. In aspects, most, generally all, or substantially all RT/S data is ingested and stored in the NDS DR. methods to enable stream processing can comprise employing approximation methods for data aggregation/compaction, using random read/write methods (with respect to NDS memory), and micro-processing of stream portions (e.g., single files, few records, etc., per process cycle).

In aspects, NDS input can comprise email input(s). E.g., NDS can comprise e-mail stream monitoring U/F(s) (methods comprising e-mail stream monitoring step(s)). Email input processing Function(s) can comprise protocols for recognition of attachments, email text, or both. Email and related text messaging formats, web pages, etc., can be used to provide substantive subject data, system data, network data, network component data (e.g., MA-D), or requests to the NDS or components of the network. In aspects, natural language processing (“NLP”) and other processing tools/methods can be employed to infer email or other unstructured text message content or intent (e.g., request for NDS services). E.g., an NDS input unit can employ subject matter classification (SMC) algorithm(s) that can ascertain the context of unstructured input data. In aspects, ranking function(s), enrichment function(s), etc.), can be performed on Input to facilitate initial processing of the input (e.g., application of a data queue, initial analysis, etc.). In aspects, initial input data transformation functions are achieved through large system step functions, e.g., Amazon Web Services (AWS) Step Functions or corresponding functions in Microsoft Azure.

In RT/S data processing, NDS input unit(s) can comprise a system/component(s)/function(s) that can be characterized as a stream/streaming processing engine (“SPE”) or stream processing unit (aka, a stream processor or SDP). The SPE can, i.a., identify cases and events in streams of MA-D, e.g., in critical care cases, such as surgical and intensive care unit (ICU) cases/settings. In aspects, an SPE can identify MA type, specific MA, medical procedure used, timing of the medical data, nature of the MA-D (data type), collection type of the MA-D (e.g., RT/S or cached MA-CD), MA status data, MA sensor or MA-associated sensor data (e.g., vital sign data), etc. (e.g., through data transformation or data input requirements). E.g., a time series of MA-D can comprise status data indicating the start of a procedure with a particular MA, end of a procedure with a particular MA, or a change in patient condition that triggers one or more alarms/alerts based on the NDS input unit screening of the initially received data. RT/S data originating from the same MA, same MA-type, same patient, same HCP, same Entity, same location, or ACT, can be tagged or otherwise identified as being related by the NDS input unit. NDS input unit-applied tags can be used to simultaneously analyze related data streams or combine analysis of related data streams to identify clinical cases that meet or exceed certain thresholds/criteria, triggering prioritized actions by the NDS in terms of, e.g., alerts/alarms, control of MA, display of MA protocol information, etc. For example, an MA status data stream, a user input data stream, device setting data stream, and operational data streams can in aspects be used to identify when the device is used and how it is used in clinical case(s). This information may help to distinguish between MAs in maintenance applications vs. in clinical use, clinical trial use, research use, or a combination thereof. In networks comprising multiple types of MAs, MAs providing multiple applications, or both, the input data can be used to identify MA-D streams as being associated with a particular subject, a particular HCP or HCP team, another class of MA users, a particular Entity, or a combination thereof. In aspects, RT/S MA-D or other MA-D input can be combined with information from other network/system components, such as MA owner system information, to infer other relationships that may be important in initial analysis. E.g., MA owner systems can comprise, e.g., scheduling of procedures employing MAs, which when linked with incoming MA data, can indicate how/when MAs are being used in an Entity, and such information can be relayed back to the Entity, to a research class of users, or to commercial class users (providing redaction/exclusion or other protection of PHI consistent with RRs). Typically, such non-critical analyses are, however, applied on stored data, rather than on input (pre-ingestion data).

In aspects, stream processing units, such as a streaming data processor (SDP), use scoreboarding (dynamically scheduling a pipeline so that the instructions can execute out of order when there are no conflicts and processing resources are available) as well as or as an alternative to other types of message queuing or prioritization methods described elsewhere.

Aspects can comprise causing a streaming data processing unit to (a) receive relayed streaming MA-D, (b) perform an initial analysis on the relayed streaming MA-D, and (c) if the initial analysis identifies one or more preprogrammed conditions in the streaming relayed MA-D, to perform one or more of a pre-programmed limited set of initial functions, which initial functions comprise relaying instructions for controlling the operation of one or more medical apparatuses, one or more other network devices, or both. Such a function also can be written/described as START (1) WHILE MA is operational; (a) REPEAT until MA and NDS are not in secure and stable communication; (I) RECEIVE streaming MA-D at Streaming Data Processor; (II) PERFORM initial analysis on streamed MA-D; (III) IF≥1 condition in initial analysis (e.g., indication of patient danger, device failure, etc.); (A) PERFORM initial analysis output function(s) (e.g., control of device relay of alarm, etc., @MA(s), ONDI(s), or both); (B) STORE streaming MA-D; (IV) ELSE; (A) Store streaming MA-D; (2) END WHILE; END.

In aspects, other classes of confidential data, such as personally identifiable information/PHI protected under data RRs, e.g., US HIPAA, EU GDPR, and California CCPA/CRPA RRs, are identified and handled separately in the NDS and in NDS output (e.g., by data curation, selectively relay/display etc.). In aspects, data from research class MAs and other research class ONDs can comprise data subject to other data rules, such as blinded/anonymized data rules, randomized data rules, etc.

As indicated, the NDS input unit can comprise or work with a controller that causes output(s) based on initial analysis performed on SMAD, cache data, or both. E.g., streaming analytic function outputs can comprise alarm/alert algorithms, control algorithms, event detection algorithms, or predictive algorithms. These algorithms may be predefined, or in other embodiments may be created via automated learning. Non-limiting examples of the streaming analytics algorithms may include early detection of deterioration of a patient's health status, detection of complications in an operating room (OR) or ICU, prediction of a medical device needing maintenance, prediction of complications in cases which may require future special care, or prediction of scheduled delays. In aspects, streams of MA-D can indicate the way MA(s) are being used and from initial analysis of data indicating the state of operation of the MA, an event such as a complication during a surgery may be detected and appropriate actions/alarms triggered. In embodiments, streaming analytics U/F(s) can be used to predict a change in patient location, which may be associated with, e.g., a gap in RT/S data flow from the MA-D (and subsequent upload of cached MA-CD).

In aspects, a DMS (e.g., a data processing engine), which can effectively handle both RT/S and batch processing can form or be a component of the NDS Input Unit. A stream processing engine/SDP can typically “tap” incoming data streams in a “noninvasive” manner (without impacting the content of the data streams), and, in aspects, without significantly impacting ingestion latency. An example of such a system is the Spring XD System (Apache). In aspects, the NDS Input Unit comprises or accesses a unit/engine/function (U/F) that can perform multiple iterative data transformation or analyses steps prior to ingestion or concurrently with ingestion, such as in Apache Spark.

In aspects, some, most, or generally NDS input unit/engine functions are performed on data maintained in one or more temporary memory unit(s). E.g., in aspects, methods of the invention comprise collection multiple components of a stream, multiple streams, or both, in a temporary memory and performing one or more NDS input unit functions on such temporarily held data (e.g., analysis for data triggering an immediate alert/alarm, change in MA therapeutic operating parameters, or both).

In aspects, NDS input unit(s) or other components of the NDS (e.g., an NDS relay unit/NDS-RELAYU) will comprise/perform U/Fs for management of data serialization/deserialization (e.g., comprising data serialization libraries, schemas, etc.). E.g., such U/Fs typically can convert complex data structures into a byte stream for storage, transfer, and distribution on physical devices, or for storage (e.g., in an EDL). In aspects, serialization/deserialization U/Fs can convert data in certain formats into other formats that are preferred or to which the system restricts certain input data. E.g., BSON, YAML, or MessagePack data may be converted to JSON data (or the reverse) by a serialization/deserialization function of an NDS. Examples of known technologies/frameworks for facilitating serialization include, e.g., Apache Thrift, Google Protocol Buffers, Apache Avro, and Apache Flume.

In aspects, an NDS input unit performs immediate in-process/in-memory data functions on MA data or other inbound messages (telemetry). In aspects, an NDS input unit performs such data collection and analysis on a limited time repeating cycle basis and/or delays action on such data for a limited time period (e.g., every 5-30 seconds, such as every about 10 seconds or a delay of about 5-30 seconds, such as about 10-20 or about 10-25 seconds). Such functions include immediate analysis of certain data leading to activation of device control functions, MA/ONDI alarms, and the like.

In aspects, an NDS input unit comprises a data validation rule process based on the NDS input unit receiving≥10 packets of MA-D, e.g., ≥about 11, ≥about 12, ≥about 15, ≥about 20, ≥about 25, ≥about 50, ≥about 75, or ≥about 100 or more, such as ≥about 250, ≥about 500, ≥about 750, ≥about 1000, or even more packets of MA-D from the MA every about 1 second, about 2 seconds, about 3 sec, about 4 sec, about 5 sec, about 10 sec, or, e.g., about every 0.1 sec or about every 0.5 seconds (packet size varying per system/network properties, e.g., being about 1-100 kb, about 1.25-75 kb, or about 1.5-65 kb). In aspects, if such a data validation rule is violated, CEI in the NDS input unit or other aspect of the NDS can trigger actions, such as alert/alarm functions, which are discussed elsewhere.

### 5. Data Handlers (Event Hubs, IoT Hubs, Etc.)

NDSs can comprise data handler(s) that form part of an input unit, are separate from an input unit, or both. Data handlers typically are engines/units or methods/functions that perform data analytics functions, routing functions, event handling functions, or a combination of any thereof, and are at least partially distinct from the input unit and relay unit of the NDS. Data handlers typically receive telemetry (data/messages) from multiple input sources (e.g., devices) (in aspects from several input sources simultaneously, in cases thousands or even million messages per second) and further relay such data to other Unit(s) or outputs, often two or more outputs simultaneously. Examples of data handlers include analytics engines and event handlers. Analytics engines, such as streaming analytics engines, event handlers, and similar systems/units, are known. In aspects, data handler(s) include cloud-based Unit(s)/component(s).

In aspects, an NDS comprises one or more event hub data handler units. Event hubs typically are/include Units that process data on a high scale but low profile (e.g., without advanced sequencing functions, delivery guarantees, and the like), which operate with relatively low latency and high reliability. An input for an event hub can be referred to as an event publisher. Event hubs and similar components of the NDS can use various data relay protocols, such as HTTP/AMQP protocols. In aspects, event hub(s) comprise partition(s) (e.g., 2-32 partitions) (ordered sequences that keep events in the event hub). The partitions (partitioned consumer model) enable multiple applications to process the stream concurrently allowing for more speed of processing and control of processing speed. In aspects, an event hub processes data only in a uni-directional manner (not relaying messages to the event publisher, but relaying data from event publishers to event consumers (i.e., downstream units)). Event Hubs can act as a “front door” for an event pipeline, and such event hubs are often called an “event ingestor.”

In aspects, data handler(s) of an NDS comprise internet-of-thing (“IoT”) hub(s). An IoT hub of an NDS typically can perform bi-directional data communications, as well as functions relating to device control, device authentication, device authorization, protocol translation, and combinations. In aspects, an IoT hub performs command and control functions over connected devices, such as MAs. E.g., IoT hubs can comprise preprogrammed instructions for control over devices in response to certain conditions (e.g., certain physiological measures in a patient detected by device(s)). In aspects, an IoT hub can handle device error reporting, e.g., checking failed connection attempts per device, comprise disconnection/disabling a connected device, or both.

In aspects, a data handler performs one or more data functions on a time basis, such as a recurring 1-minute, 2-minute, 3-minute, 5-minute, 6-minute, or 10-minute data collection cycle/window (e.g., a 30 second-600 second data collection window, such as a 2-8 minute data collection window).

Apache Kafka ecosystems comprise known event hubs that can be used in an NDS or that can be used as a model for an NDS event hub. Azure systems include both Azure Event Hubs and IoT Hubs. Big data analytics services of Azure including Databricks, Stream Analytics, ADLS, and HDInsight can read and process the data from the hub. Data handlers also can include event grids, which are units more focused on event processing. An NDS can comprise any suitable one or more of such types of hubs or an equivalent thereof in terms of functions performed/capabilities.

In aspects, data handler(s) of the NDS perform both data analytics and event handling functions. E.g., an analytics engine, such as Azure Stream Analytics, acts as both a real-time analytics and complex event-processing engine that processes streaming data from multiple sources simultaneously. In aspects, the data handler identifies data patterns, data relationships, or both, from data from a number of input sources, including MA-D, NDS-AD, and other inputs. In aspects, analytical functions of such data handlers can initiate actions, workflows (such as creating alerts), feeding information to a reporting tool, storing transformed data for later use, or a combination. Analytics processors typically perform jobs including input, query, and output elements (e.g., receiving data from an event hub or IoT hub, performing SQL query language-based queries or user-defined functions (UDFs) to filter, sort, aggregate, and join streaming data, and relaying data to other components/Units, e.g., Azure Functions, Service Bus Topics or Queues to trigger communications or custom workflows downstream or data in data repositories (for example, a DL/EL, Azure Synapse Analytics, etc.), optionally to train a machine learning model based on historical data or perform batch analytics. Elements of SDPs, e.g., Apache tools such as Kafka, Spark, Storm, and Flink, can be used to perform such functions. Amazon tools, such as Kinesis Streams, Kinesis, and Firehose, can provide similar services.

### 6. Analytical Engine(s)/Unit(s)/Function(s)

NDSs typically comprise component(s) that perform analyses on MA-D, which can be characterized as NDS analytical unit(s)/engine(s)/system(s) (sometimes referred to as NDS-ANALU or similarly an NDS analytical/analysis unit). An NDS analytical unit typically is composed of unit(s)/engine(s) of the NDS and associated memory/processor resources that analyze data in NDS memory, such as in an NDS DR. An NDS can comprise any suitable number of analytical unit(s)/engine(s) of any suitable type(s). In aspects, an NDS comprises at least one analytical unit that primarily, generally, or only analyzes DR data (post-ingestion). In aspects, an NDS comprises an analytical unit that analyzes data pre-ingestion (e.g., as part of an input unit, such as an SDP, as discussed above and below). Data generated by NDS analysis is called NDS-analytical data (“NDS-AD”). In aspects, NDS-AD can be delivered to network components, e.g., MAs or other network/NDS components, and can be the basis of even further analytical functions/methods, resulting in higher levels of NDS-AD, control actions performed by controller unit(s) of the NDS (e.g., control of MA device functions), or both. NDS-AD can comprise organized or structured “raw” MA-D or other input in the NDS DR, data generated by the application of analytical processes/algorithms on stored data, or both. In aspects, the analytical function applies schema(s) on stored data in NDS memory. E.g., an NDS analytical unit can comprise instructions for application of ≥~2, ≥5, ≥10, ≥20, or ≥~50 different schemas that are applied to NDS-AD in delivering data to MAs/ONDIs, performing functions, or a CT. Most processes of the analytical unit can be characterized as CPU/processor bound processes (generating input/output (I/O) requests/operations infrequently), as opposed to I/O-bound processes employed by, e.g., the NDS Input Unit (NDS-INPU) or NDS Relay Unit (NDS-RELAYU). The NDS analytical unit engine(s)/function(s) typically will generate/result in one or more forms of output that are delivered to users of network devices/interfaces. Such output can include formatting and delivering data that is displayed on a device or interface, machine control data for automatic operation of a device, alarm/alert instructions, or a combination thereof.

In aspects, one or more NDS analytical units/engine(s) are fixed analytical units that are not adjustable during operation. In aspects, one or more analytical units are adjustable in operation, or even automatically adjustable in operation. In aspects, machine learning is used to shape a fixed functioning analytical unit. In aspects, machine learning is used to drive/shape the functioning of an analytical unit in operation (e.g., supervised or unsupervised machine learning can be applied to or form a part of the processing of the NDS analytical unit, such as the prediction of physiological states in a subject, best courses of treatment, etc.).

According to aspects, code/CEI that can be characterized as being part of the NDS analytical unit/engine comprises one or more alert/alarm conditions associated with sensor data, and means for detecting the presence of the same, wherein an NDS analytical unit determines if one or more alarm conditions are triggered by, e.g., MA-D, analysis, and the NDS causes an alarm to register in the MA, to be delivered to a device/interface associated with a user associated with the MA/subject, or both, based on the sensor data and permitted user options.

In aspects, the MA-D comprises information about the operating state of the MA-D and an NDS analytical unit evaluates the operating state information in the MA-D in performing one or more analyses.

In aspects, the MAs relay packets comprising time-tagging, e.g., identifiers associated with MA-D indicating a time or sequence of data group. In aspects, the MAs relay packets of data comprising such sequencing information, and the NDS analytical unit comprises a function for analyzing time component(s)/attribute(s) of cache data. In aspects, upon analyzing the time component of cache data (MA-CD), the NDS-ANALU can re-sequence MA-CD that is received in nonchronological order. In aspects, such a resequencing renders MA-CD chronologically ordered.

In facets, an NDS analytical unit comprises a function for identifying past MA-D, current/near current MA-D, and predicted MA-D (NDS-AD) (e.g., tagging data with an applicable time stamp/code). In aspects, the NDS analytical unit performs≥1 predictive function based on the analysis and relays the results of the predictive function to the MAs, other network devices/interfaces (ONDIs), or both. E.g., current data can be subjected to initial analysis, whereas data time stamped as past relevant windows may be rejected or passed directly to DR ingestion. Data creation time, relay time, modification time, or any CT, can also be considered in performing functions on DR data, as exemplified elsewhere.

In embodiments, an NDS analytical unit comprises CEI for monitoring if an event arises and re-performing an analysis upon the occurrence of an event, updating the analysis, and reporting the updated analysis to one or more MAs, or one or more NDS- or network-associated users, e.g., one or more other network devices/interfaces (ONDIs). In aspects, the NDS-analytical unit comprises CEI for monitoring for data patterns meeting pre-established alert conditions and, when data patterns meet such pre-established alert conditions, an NDS analytical unit comprises CEI for signaling that notification of MA(s), ONDI(s), or a CT is required. For example, such alert conditions may be identified in MA-D patterns, NDS/network operation patterns, communication patterns, or any functional element within the systems described herein with which data is associated that is accessible by an NDS analytical unit.

In certain embodiments, an NDS analytical unit performs≥1 function/engine that is regulated as software as a medical device (SAMD). In aspects, an NDS analysis performs≥1 SAMD functions and ≥1 non-SAMD function(s) (NSAMD function(s)). In aspects, the NDS delivers output of the ≥1 SAMD function (or ≥SAMD and ≥1 NSAMD functions) to MAs that differ i.a. in accordance with CEIs that reflect applicable regulatory status of the SAMD and NSAMD functions. In aspects, the ≥1 SAMD comprises providing diagnostic instructions or therapeutic instructions to a health care provider. According to some facets, ≥1 SAMD can change operating conditions of MAs in response to one or more conditions. In aspects, NSAMD functions do not perform such tasks.

In cases, output application(s) are regulated as software as a medical device (SaMD/SAMD) by one or more regulatory authorities (e.g., US FDA), and operation of the NDS or the method comprises applying one or more data transformations, data curation processes, data validation checks, or a combination of any or all thereof, to ensure that the one or more SaMD applications comply with one or more regulatory requirements recorded in processor readable instructions stored in the MAC-DMS memory unit. In aspects, ≥1 output applications in such NDSs are not regulated as SaMDs and the operation of the NDS/method sorts SaMD output(s) from non-SaMD output(s) to ensure compliance with regulatory requirement(s).

In several functions/methods performed by an NDS analytical unit, one or more data transformation(s) occur, as discussed elsewhere. Structured data transformations can be performed in, e.g., SQL. Semi-unstructured data transformations can comprise application of a schema to the data based on the execution of one or more queries.

At least one NDS analytical unit (e.g., a primary NDS analytical unit which performs most of the NDS analytical functions), can comprise query (search and matching) units(s)/function(s), e.g., allowing users, processes, or both to perform queries on DR data. Query units can, in aspects, use alphanumeric data, metadata (tags, links, references, etc.), or both, in combination with matched-record ranking methods, as described elsewhere and in, e.g., U.S. Ser. No. 10/572,221, U.S. Pat. Nos. 6,651,054, 5,826,260, U.S. Ser. No. 10/229,166, U.S. Ser. No. 10/282,472, U.S. Pat. No. 7,970,791. Matching/ranking methods relating to query “hits” (results) can involve User input, User preferences, or both (examples of such methods are described in, e.g., U.S. Ser. No. 10/387,512 and U.S. Pat. No. 7,996,392). Ranking typically will comprise evaluation of multiple ranking factor(s), e.g., keyword meaning, context, etc., matching Record content quality, etc., which are often ascribed weighting rules for analysis by ranking/matching algorithm(s)/Functions(s). Query process(es)/unit(s) can in some aspects/cases use synonym generation methods to expand queries. Relevant principles and methods are described in, e.g., U.S. Pat. Nos. 7,636,714, 8,392,413, U.S. Ser. No. 10/546,012, US20100082657, US20100313258, US20160253418, U.S. Pat. Nos. 9,361,362, 9,489,370, 8,832,092, and 8,812,541. In aspects, a query comprises search/query element(s) that are generated from combination of terms (term combination) in a source or from source(s), according to frequent combination detection, system rules, or other suitable method(s). Examples of combined search term methods are described in, e.g., US20100138411 and US20110184725. In aspects, a query is generated or records are matched through, i.a., decomposition of data sets into data set elements and comparison is made on an element-by-element basis (e.g., based on the comparison of key/value to key/value combinations). An NDS analytical unit or a component thereof/related unit, such as an NDS data improvement unit (DIU), or both, can employ tokenization methods at various levels (e.g., a single term/delimiter level, a bigram level, a trigram level, or higher Ngram level, or a CT, often in combination with testing such different level(s) against expected syntax, common expressions, meaning/NER, or a CT) (e.g., identifying “New York” as a particular stage/city by bigram tokenization, versus evaluating the data as “New” (possibly disregarded) and “York” at an individual token interpretation level). NLP processes performed by NLP U/F(s) can interpret or be performed to interpret heterogenous forms of input, e.g., unstructured input. NLP processes can be combined with the use of specialized SN(s), e.g., SN(s) trained on relevant terms and meanings, synonyms, delimiters and other grammatical/syntax, or formatting rules, and the like. Overlaps in various query-related S(s)/F(s) can exist. E.g., NLP processes typical involve tokenization in the forms of word recognition, sentence segmentation, field segmentation (e.g., in tabular data), or paragraph segmentation. NLP processes typically further comprise part of speech recognition (and part of speech tagging), tense recognition/tagging, and the like. NLP processes also typically comprise application of lemmatization processes to find basic word forms that connect read terms in Input/DS(s), and evaluate word relationships (e.g., by dependency parsing (e.g., shallow parsing/“chunking”), phrase identification, word sense disambiguation, natural language generation, coreference resolution, sentiment classification, and Named Entity Recognition (NER)). NER processes for specific terms can require the inclusion of a specific corpus/corpora. NLP processes can comprise analysis based on sentence/phrase/expression morphology or syntax, and NER processes can draw on factors such semantics or pragmatics. Exemplary NLP processes include, e.g., NLTK, WordNet, BERT (sub-word token) model, and spaCy library resources in Python. The use of subword token method(s) in NLP can aid in detectably reducing out of vocabulary (OOV) interpretation problem(s). NLP processes also can be employed in Input processes, e.g., receipt of instructions, responses to questions, etc., made by an NDS input unit. Other NLP processes and processes relating to matching of data (e.g., in data linkage) are provided elsewhere in this disclosure.

Query processes can include stemming and related methods, e.g., in matching, synonym generation, or both. Stemming can include suffix removal, prefix removal, or both. Several well-known stemming algorithms are known and available, including the Porter Stemmer, the Lovins Stemmer, the Dawson Stemmer, the Krovetz Stemmer, the Xerox Stemmer, N-gram Stemmer, the Lancaster Stemmer, and the Snowball Stemmer. Lemmatization tools/resources and principles are known (e.g., the WordNetLemmatizer function of nltk.stem and the Lemmatizer Class in the spaCy library in Python provide NLP lemmatization functions for common English terms). Development of specialized thesauruses useful for query functions, matching functions, or both are known (see, e.g., US20100198821). String matching/stemming steps that can be used in query/matching processes include applying fuzzy (approximate) string matching methods, which are known and can be performed using available tools/methods e.g., methods that use Levenshtein Distance methods, e.g., methods employing functions of the Python FuzzyWuzzy or fuzzymatcher package/library (setting, e.g., minimum ratios for compared strings). String stemming methods suitable for performance of stemming step(s) incorporation in stemming Functions also are known. In aspects, stemming step(s) include use of a preprogrammed lookup table. In aspects, stemming step(s) include application of preprogrammed prefix stripping rules, suffix stripping rules, lemmatization algorithms, stem database matching algorithms, or a combination thereof (e.g., affix stripping). In aspects, stemming step(s) comprise application of trained/trainable probabilistic stochastic algorithm(s). In aspects, stemming, matching, or both used in method/NDS comprise rules/inputs trained for, comprising, or otherwise factoring analysis/use of particular information, e.g., particular MA-D (e.g., a vital sign data). In aspects, stemming step(s)/Unit(s), matching step(s)/Unit(s), or both, comprise an ML component/method (e.g., ML applied to a stochastic algorithm trained by supervised learning or supervised and semi-supervised learning). In aspects, the matching or stemming functions are also applied to non-string inputs, e.g., images, values, and the like. Any part of this disclosure described in terms of string matching will be understood as providing simultaneous support for application of such other methods or a broader class of data matching/stemming comprising 2 or more of such different types of data. Matching is often performed based on data points/data sets e.g., in key/value pairs, field/record pairs, another schema, or a combination thereof. Examples of search/matching technologies and principles that may be adaptable to S(s)/F(s) of method/NDS are described in, e.g., U.S. Ser. No. 10/572,221, U.S. Ser. No. 10/452,764, U.S. Pat. Nos. 8,145,654, 6,625,615, and US20160306868, and references cited therein. Search S(s)/F(s) can comprise the use of metadata, e.g., search “tags” (e.g., as exemplified by US20140039877, U.S. Pat. No. 7,844,587, US20080270451, U.S. Pat. No. 9,305,100, and US20200097595). Examples of search/matching technologies and principles that may be adaptable to S(s)/F(s) of method/NDS are described in, e.g., U.S. Ser. No. 10/572,221, U.S. Ser. No. 10/452,764, U.S. Pat. Nos. 8,145,654, 6,625,615, and US20160306868, and references cited therein. Search S(s)/F(s) of methods/NDS of the invention can also comprise the use of metadata, e.g., search “tags” (e.g., as using methods described in US20140039877, U.S. Pat. No. 7,844,587, US20080270451, U.S. Pat. No. 9,305,100, and US20200097595 or the like).

In aspects, an NDS analytical unit comprises a task-scheduling/process scheduling Engine/Function (task scheduler or process scheduler). The analytical unit can use or primarily use long-term or medium-term scheduling methods, whereas an NDS input unit or NDS relay unit (NDS-RELAYU) often, primarily, generally, or only use task scheduling unit(s)/function(s) (U(s)/F(s)) that are based on short term (in-memory) scheduling processes. A task processor U/F can comprise a dispatcher component/function, which provides control to the NDS processor/analytical unit for the applicable process, if needed (e.g., in a short-term process). In aspects, task processors comprise context switches/controls, e.g., in response to data events. Task processors can apply scheduling based on any one or more methods including FIFO, priority scheduling, shortest time remaining scheduling, capacity scheduling, Round-robin scheduling, Multilevel queue scheduling, fair scheduling, delay scheduling, dynamic proportional scheduling, or Resource-aware adaptive scheduling (RAS). Examples of such approaches are described in, e.g., Seethalakshmi et al., J Inform Tech Softw Eng 2018, 8:2, DOI: 10.4172/2175-7866.1000226. Task schedules can, in aspects employ threading models (e.g., kernel-level threading).

Task scheduling processes can be components of other frameworks used for design structure matrix (DSM) task management, which can be employed by Unit(s)/Function(s) of an NDS processor, e.g., an example of which is the Multi-Objective Scheduling Algorithm of Many Task in Hadoop (MOMTH). Hadoop, Spark, Storm, and Mesos are very well-known frameworks comprising multiple algorithms for scheduling of short tasks, large jobs, interactive jobs, large/batch jobs, and guaranteed-capacity production jobs. See, e.g., Mbarka Soualhia et al. Task Scheduling in Big Data Platforms: A Systematic Literature Review, Journal of Systems and Software, Volume 134, 2017, Pages 170-189, doi.org/10.1016/j.jss.2017.09.001.Other relevant methods/principles are described in Paul Krzyzanowski “Process Scheduling: Who gets to run next?” at https://www.cs.rutgers.edu/~pxk/416/notes/07-scheduling.html (2014-02-19). and Tong Li; Dan Baumberger; Scott Hahn. “Efficient and Scalable Multiprocessor Fair Scheduling Using Distributed Weighted Round-Robin” http://happyli.org/tongli/papers/dwrr.pdf.

### 7. Data Improvement Engine(s/Unit(s)

In aspects, the NDS comprises units that can be characterized as data improvement unit(s) (DIU(s)) or perform corresponding function(s). In aspects, the NDS comprises a DIU that is a component of the NDS input unit (or such units in an NDS comprise overlapping functions). In aspects, the NDS comprises a DIU operates after initial ingestion of data. In aspects, data ingestion can be a staged process. E.g., in aspects, data input is subject to very limited transformation or structure requirements/screening, but after initial ingestion such data is further subject to DIU processes resulting in a second level transformation of the data, which second level transformed data is stored in the NDS DR in a second ingestion process. In aspects, second level ingested data is subject to NDS processes, such as NDS-ANALU process(es), and the resulting NDS-analytical data (NDS-AD) is subjected to storage, typically in a more structured portion of the DR given the application of schema(s) to such NDS-AD. An NDS-DIU can perform function(s) for improving the quality or usability of input, e.g., harmonizing disparate data, evaluating and validating/rejecting input, etc. Such analytical processes can include automatically performed processes (e.g., machine learning processes applied on DR data) or on-demand (manually triggered) processes, e.g., performing data query(ies) on DR data.

In aspects, an NDS comprises a DIU comprising or operating as a data harmonization unit (NDS-DHU), which can evaluate the characteristics of received data, and apply functions which harmonize disparate data, e.g., data from heterogeneous MAs within the network of MAs, SUMAD and unstructured or structured MA-D, or a combination thereof, rendering such data capable of being joined/compared/analyzed in aggregate. In aspects, an NDS also or alternatively comprises an NDS-DHU which can evaluate if received data is suitable for use (e.g., is approved for immediate use) by an NDS analytical unit or if such data requires modification by application of one or more functions for harmonizing the data prior to use. In facets, an NDS-DHU can evaluate if MA-D in NDS memory is approved for use by an NDS analytical unit. According to further embodiments, the NDS-DHU can determine how any such MA-D approved for use is used by NDS analytical unit(s).

In aspects, initially received MA-D is subject to data cleaning, data harmonization/formatting (blending), or both, in an NDS. Such functionality can comprise, e.g., an assessment of validity of initial MA-D received from each MA or most or substantially all MAs. Data validity assessment(s) can comprise, e.g., measuring the degree to which detected CI data (e.g., values and attributes) conforms to expected attributes/values based on established rules/constraints or archetypes. In aspects, an NDS analytical unit or also or alternatively an NDS-DHU can rank data with lower validity scores lower in analysis, exclude data determined to be invalid/irrelevant, alert a user to invalid data, present valid data, or e.g., report both retained and excluded data. In aspects, the DIU applies one or more tags or other form(s) of metadata to data (e.g., applying one or more nonhierarchical data set(s)/point(s) that can be used to process the associated data downstream in NDS processes). An NDS-DIU can also comprise U(s)/F(s) for carrying out data classification (e.g., grouping data into subject-oriented data sets for ease of processing at one or more data levels, e.g., all MA-D versus other inputs, all MA-D of each type of MA-D, all MA-D associated with each type of patient/procedure employed by such MAs, etc.). A DIU can also perform data categorization S(s)/F(s), e.g., grouping data based on data type or data type and data classification, which can be, e.g., used in storage location or properties or as a basis for additional metadata application/tagging. In aspects, an NDS-DIU employs contextual processing methods in performing data analysis, transformations, or both, and in aspects the NDS-DIU's application of metadata is context dependent. In aspects, applications of context metadata DoS facilitate data processing applications, e.g., data mining. Context can be applied in, e.g., synonym generation for queries, interpretation of records (e.g., interpreting “ha” in input associated with a cardiovascular MA as likely meaning “heart attack,” versus “hyaluronic acid” in aspects relating to dermatological devices). Examples of available systems for performing DIU/data ingestion operations include, e.g., Apache Nifi, Elastic Logstash, etc. In data ingestion, an NDS-DIU can, e.g., categorize data, prioritize data, or both, enabling application of queue processing. In aspects, an NDS-DIU can use metadata, semantic libraries, or master data as the basis of data integration links, which may include dynamic data record links (e.g., between semi-unstructured and unstructured/structured data, e.g., between SUMAD associated with MAs and query response data associated with the MAs). In aspects, the DIU employs NLP methods for translating inputs from multiple natural languages (e.g., English, Chinese, Japanese, German, French, Spanish, Arabic, Hindi, Korean, and a CT). E.g., in aspects inputs from ≥about 2, ≥3, ≥5, ≥10, ≥15, or ≥about 20 natural languages (NLs), NL dialects (e.g., Mandarin and Cantonese), or a CT, can be provided to the NDS, generated by the NDS, or both (e.g., the NDS can relay output in Spanish and English, in Japanese and English, or German and French).

In aspects, an NDS-DIU performs one or more data cleansing Functions. Data cleansing units/functions/methods can detect data issues (e.g., redundant, incomplete, incorrect, inaccurate, or irrelevant data) and either remove or correct problematic data. Common errors that can be detected or corrected by data cleaning methods include spatial errors, duplication errors/redundancy, inconsistency errors, or formatting errors. In aspects, a data cleaning Function (DCF) detects or corrects classification or nomenclature errors. In aspects, the NDS uses a set of definitions or rules to determine inconsistency errors and other types of data corruption errors (e.g., in aspects the method comprises development, maintenance, and application of a dictionary, glossary, or authority file). In aspects, DCFs comprise the use of strict validation rule(s), fuzzy validation rule(s), or both. In aspects, DCFs comprise cross-checking 1 or more data set feature(s) (DSF(s)) or Records with DSF(s) with Records or DSF(s) that have been validated. In aspects, DCFs detect and address structural errors, e.g., incorrect attributes, mislabeling, and the like. A variety of data cleaning methods/algorithms are known and can be applied or adapted for such steps/Functions. Examples of known tools for performing such functions include Duplicate Count Strategy++ (DCS++), Dedup, Progressive Sorted Neighborhood method (PSNM), and the Innovative Windows (InnWin) method. Known data cleansing tools that may be applicable or that comprise applicable methods/approaches include IBM InfoSphere services/products, OpenRefine, Winpure, Trifacta Wrangler, DataMatch components of Data Ladder, Quadient Data Cleaner, and Salesforce's Cloudingo. In aspects, methods of the invention comprise using MLMs in DCF(s). In aspects, DCF(s) comprise rules or inputs trained for or otherwise comprising/factoring in analysis/use of MA-D or other expected network device data (e.g., CMSS data).

In aspects, an NDS-DIU harmonizes data from various MA-D or EMR/EHR-related messaging standards (e.g., Health Level 7 International (HL7 V2.x/v3), Clinical Document Architecture/Continuity Of Care Document (CDA/CCD), American Society for Testing Materials (ASTM), Digital Imaging and Communications in Medicine (DICOM), etc.) and various standards or protocols (e.g., cross-enterprise document sharing (XDS.A/B) cross-enterprise document media interchange (XDM) cross-enterprise document reliable interchange (XDR), patient identifier cross-referencing/patient demographics query (PIX/PDQ) patient administration management (PAM), query for existing data (QED), national counsel for prescription drug programs (NCPDP), etc.), to enable harmonization of such heterogenous inputs. E.g., an NDS-DIU can convert some, most, generally all, or all input into a consistent or compatible format for use within NDS (e.g., ISO/IEEE 11073-10101 nomenclature and its extensions). In aspects, the DIU normalizes streams of incoming time series data or other input by, e.g., converting units of measure in the data (time, volume, size, etc.). In aspects, non-uniform data can be harmonized (e.g., converting mole percentages to weight percentages, converting volumes to weights, inches to centimeters, volume or time units to alternative volume or time units, etc.).

According to facets, data, groups of data, or sets of data, e.g., ~2 or more, ~3 or more, ~5 or more, ~10 or more, ~25 or more, ~50 or more, ~100 or more, ~500 or more, ~1000 or more, ~5000 or more, or even ~10,000 or more pieces, groups, or sets of MA-D are subjected to an assessment of uniformity, an evaluation of types of values, units of values, etc., in, e.g., the NDS-DIU to determine, e.g., the types of values present, units of such values, etc. In aspects, integrity can be used to describe analysis of combinations of 2 or more of accepted (e.g., immediately accepted or harmonized) data, consistent data, accurate entry, complete entry. In aspects, data cleaning follows an evaluation of integrity.

According to aspects, an NDS DIU typically performs data auditing to assess NDS input unit/incoming data anomalies, contradictions, other errors, etc. using rules, constraints, statistical analysis, or any combination thereof.

In aspects, an NDS DIU can apply one or more constraints to evaluate acceptable versus unacceptable data or data acceptable as-received data versus data requiring modification prior to acceptance and use. In aspects, one or more constraints can comprise but may not be limited to, e.g., range constraints (e.g., expectation of values of a particular attribute falling within a numerical range or order of magnitude (e.g., a volume or rate), or nominal values falling within a certain set (e.g., units that match an expected physiological measurement)). Constraints can also be applied to dates, times, and other values per rules (usually maximum and minimum expected values). In aspects, NDS input can be subject to mandatory constraints (e.g., required entries), unique constraints (information that particularly identifies/associates the CI with one or more items of information), or both. In aspects, regular expression pattern recognition is commonly employed in the recognition of such input. In aspects, cross-field validation methods can be used, e.g., across data received as a set from an MA or type of MA or MA meeting a particular set of conditions, cross referencing data from one field to another, such 2 or more data fields representing percentages. In aspects, some, such as ≥~5%, ≥~10%, ≥~15%, ≥25%, or ≥33% of input, is in an unstructured format, is not subject to constraints or content rules/requirements, or both.

According to embodiments, an NDS-DIU or other NDS component can perform a completeness evaluation/assessment, particularly where mandatory constraint rules for information are employed. In aspects, an assessment threshold will determine if and how information, e.g., MA-D or analysis, is utilized. In alternative aspects, an assessment score will evaluate how such data is used, or if such data is transformed or excluded.

In aspects, an NDS-DIU, NDS input unit, or other aspect of an NDS will assess consistency between input data of one or more data types/sources, or within source(s) of input over time (e.g., over the course of a single patient treatment or assessment period). Consistency measures can be performed by, e.g., matching against rules, between sources (e.g., between sources of similar MA-D), or both. In aspects, consistency analysis is performed using measures (which can be reported to a user, employed by algorithms with cutoff functions, etc.). In aspects, a DIU makes consistency determinations and acts (modifies data) based on identified inconsistencies or other detected data issues, e.g., alerting a user of an issue or deciding to omit or amend data according to established rules implemented via the execution of CEI. According to some embodiments, machine learning (ML) can be applied to DIU processes to make enhanced evaluations or consistency determinations, e.g., enhanced comparative or predictive evaluations. ML processes are generally described elsewhere and, in the art.

In aspects, an NDS-DIU standardizes some, most, generally all, or all input of one, some, most, generally all, or all input types. Standardization comprises ensuring that internalized data is expressed in a form that the NDS recognizes or allows (e.g., certain semi-unstructured data formats or data formats/records recognized as comprising minimal data requirements) (e.g., through serialization and other standardization methods described elsewhere here). As an example of the standardization process, if the NDS requires gender be expressed as male, female, and other, but an external entity expresses gender as M, F, and O, and the standardization process translates the M, F, and O into male, female, and other.

An NDS-DIU can perform step(s)/function(s) on input/data to “master” the data. Mastering data refers to a process in which the various ways in which data belonging to a particular individual, device, Entity, or the like can be attributed to that individual, device, Entity, etc. As an example, mastering the data can include recognizing that data attributed to Jonathan Jones and Jon Jones belong to the same person. Mastering functions can comprise storing master data in a series of mastered identities or maps. Mastered identities or maps storage can be accessed during a real-time or batch/analytical patient-specific data analysis to provide the applicable algorithms with synonyms for the referenced person, device, entity, etc.

Input data (input) and ingested data can include both structured and unstructured data, and the various consumption models can include consumption models e.g., data discovery consumption models, data analytics consumption models, scientific applications consumption models and data reporting consumption models, among others. In some implementations, one or more late-stage fit-for-purpose transformers can, in aspects, be employed for data cleansing, data matching, Frame of Reference (FoR) conversion, model mapping, and data aggregation for data analytics, among others. Moreover, in some implementations, these transformers can be configured to work with data in original format within a data repository e.g., a data lake. In aspects, transformers can be configured as plug-ins.

An NDS-DIU typically will comprise duplicate (redundancy) detection and elimination function(s) to minimize record size, enhance efficiency, etc., provided that such function(s) are typically balanced against preprogrammed standard(s) of redundancy as a protection against component failure in a distributed system. Such function(s) can be applied≥2 times in the process of a method, e.g., when data is linked to master data.

### 8. Data Monitors/Dashboards

Systems/NDSs can comprise one or mor data monitor(s)/dashboard(s). A data monitor/dashboard generated by/included in an NDS can comprise a combination of engine(s) that collect specific data in the NDS/network and generate schemas/representation(s) that are adapted/configured to be displayed on network device(s) or other user-accessible interface(s) (e.g., on a variety of web pages accessible from a variety of devices). Monitors/dashboards can provide navigable access to actionable analytics to users, such as concerning the current performance of the NDS/network or improvements in operation of the NDS/network after modifications thereof. In aspects, a system/NDS comprises a dashboard/monitor that provides data visualization functions based on data being ingested into data repository(ies) of the NDS, such as one or more databases, a data lake, or both. In aspects, a dashboard/monitor provides data visualization functions for analytical data (NDS-AD), query-ready data, and the like. In aspects, an NDS comprises at least 2, at least 3, at least 4, or more data monitor units/dashboards. Examples of data dashboards/monitors are known in the art that can be applied to streaming data entering into a system and flowing through the system. For example, data monitors/dashboards based on Apache Kafka are known in the art. One such live monitoring dashboard is Redash (which integrates with other SQL dashboards like Tableau, Grafana, and Apache Superset). Microsoft Power BI dashboards are another example of a type of data monitor known in the art. Power BI dashboards can be based on push datasets, streaming datasets, or a PubSub streaming dataset. A dashboard, such as a Power BI dashboard, can be an output of a stream analytics processor, streaming data processor, or event handler, or can be a process upstream of ingestion into a data repository, such as a database. In aspects, the NDS comprises a dashboard that monitors MA functions, e.g., MA power state/functions, on a repeating basis (e.g., every 1-20 minutes, e.g., every 2-15 minutes). In aspects, monitors are coordinated with functions that provide for analysis of data patterns over time, upon events, and the like. For example, analytical functions can comprise monitoring for events over time/conditions, such as alarms, and providing feedback, reports, or functions based on the same. E.g., for a part of a network, alarm data can be collected over time, and thereby identify patterns based on events/conditions or patterns (e.g., if more alarms occur in a time such as weekends vs. weekdays, the system can identify such patterns). In aspects, through data monitors or otherwise, the NDS can identify issues in patients, devices, or a combination, which are not readily determinable through ordinary medical diagnosis, NDS analysis, and the like.

In cases, operation of an NDS results in generation of representation(s) concerning the quality of data stored in an RDB, entering an RDB, or both, and relaying the representation to the graphical user interface of one or more ONDs in the network (e.g., to ONDs that are associated with one or more NDS administrator users). In aspects, an NDS can comprise ≥2 RDBs and the NDS can comprise multiple units for generating such representations. E.g., in aspects, an NDS comprises a second RDB and operation generates a representation concerning the quality of data stored in the second relational database, entering the second relational database, or both, and relaying such a representation to the graphical user interface of one or more ONDs in the network (e.g., to one or more ONDs associated with one or more NDS administrator users). In aspects, operation of the NDS further results in applying one or more modifications to one or more instructions in the MACMDS memory unit in the event the data quality represented by any such graphical representations (data monitors/dashboards, and the like) concerning data quality entering into or contained in RDBs, e.g., ≥2 RDBs.

### 9. Relational Databases

In aspects, an NDS/system comprises one or more databases, such as one or more relational databases (“RDBs”) in addition to one or more other data repositories, such as one or more data lake(s)/enhanced data lake(s) (DL(s)/EDL(s)). Aspects relating to databases/characteristics of such DRs are described elsewhere. In aspects, an NDS comprises at least two structured databases in addition to at least one DL/EDL. In one example, an NDS comprises a database that stores NDS operational data comprising one or more elements of NDS performance (e.g., generation of NDS-AD, ingestion of data into data repositories, receipt of data from the network, etc.). In one example, an NDS also includes a database comprising NDS-AD, MA-data, or both, in aspects in a query-able format (in aspects the data is mostly, substantially completely, essentially completely, or completely distinct from any other relational DB in the NDS). In aspects, the NDS comprises a unit/function that filters/removes unimportant data/messages prior to storage in a relational database.

In aspects, DR(s) of an NDS comprise (in addition to an EDL), a 1^{st} relational database and operation of the NDS (1) generates a first structured dataset data from analytical output data and (II) stores the first structured dataset data in the 1^{st} relational database, and (2) (a) the DR(s) comprise a 2^{nd} relational database and operation comprises (3) performing one or more analytical functions on data in the EDL to obtain analytical data, (II) generating 2nd structured dataset data from such analytical data optionally in combination with data contained in the 1st relational database, and (III) storing the 2^{nd} structured dataset data in the 2nd RDB.

By combining initial analysis memory (e.g., system/network RAM), DL/EDL, and ≥1 or ≥2 RDBs, NDSs of the invention offer a variety of data functions, providing a broader range of more effective functions, such as near real time analysis of potential life preserving/lifesaving data in system/network RAM; rapid generation of data (e.g., DOS faster generation of data) for nearly current output applications, such as prediction of physiological data through use of data either in system/network RAM or in an EDL; rapid queries of semi-unstructured data in an EDL; and efficient (e.g., DOS more efficient) higher order analysis of data stored in RDB(s).

### 10. NDS Relay En-Ine(s)/Component(s)/Units

An NDS will have the capability of relaying data to some, most, generally all, or all network devices, typically including some, most, generally all, or all MAs in the network, and often also including other network devices/interfaces (ONDIs), e.g., research organization/class MAs and other devices, clinical support organization/class devices, or commercial class devices/interfaces, etc. network communication can be performed through any suitable means of communication, e.g., direct physical communication facilitated by cables, wireless communication (e.g., Bluetooth communication), or, e.g., communication via the internet, or a CT. In aspects, such communication is mostly, generally, or only conducted as secure communication, as discussed elsewhere or as otherwise known in the art. The physical components, engine(s), or both, which are involved in the transmission of data from the NDS to other network devices/interfaces can be characterized as the Relay Unit (NDS-RELAYU or NDS relay).

As with other “unit” constructions, elements of the NDS Relay Unit can overlap with elements described as forming/forming part of the analytical unit (NDS-ANALU), NDS processor (NDS-PROCU), NDS memory (NDS-MEMU), etc., such that the “unit” construct is sometimes best understood as a convenient framework for discussing functions of step(s)/function(s), rather than as always corresponding to a discrete/unique set of components/features or engine(s). Similar approaches to terms are used in the art.

An NDS relay typically securely relays information over the internet, e.g., via encrypted data, by use of VPNs, or through other data protection standards/methods. In aspects, secure information is communicated/transmitted over the internet to one or more MAs either individually or as a group or groups (e.g., to a central server or node for an IE, which then relays data to MAs in the IEs local network). In facets, information transmitted by an NDS-RELAYU is received by an MA input unit, which comprises functions for identifying NDS relayed communications and blocking other unauthorized inputs. In aspects, an NDS-RELAYU communicates, e.g., transmits/relays, information securely to each MA that is specific to the MA, specific to the patient associated with that MA, or both, and which is specifically identified and of one or more specific types that is configured to be acceptable to an MA security unit. The NDS relay can transmit/relay NDS-AD (e.g., predicted physiological data derived from an application of MLM(s) to MA-D) to MAs, ONDIs, or both. Other data transmitted from the NDS-RELAYU can comprise NDS availability information, software/OS update readiness information or components, or a CT. In aspects, data transmitted from the NDS relay to an MA-INPU mostly consists of or consists essentially of biometric predictive data, provision of instructions or control of the MA, NDS status, MA software version status, or a combination thereof. In certain facets, the data permitted to be transmitted from the NDS-RELAYU/DDRU comprises software version status information. In aspects, the NDS allows for the updating of some, most, or all MA software over the internet. In aspects, the NDS does not allow, e.g., the NDS prevents, updating of some, most, or all MA software over the internet. An NDS relay unit typically will comprise a network interface controller, network adapter, such as a server level NIC adapter/controller/card known in the art. Given the capacity of NDSs, such NIC components can comprise, e.g., Gigabit Ethernet NIC component(s), “smartNICs” (e.g., NICs with processing capabilities, such as comprising field programmable gate arrays (FPGAs), providing the server-level NIC with offload capacity independent of other processing functions of the NIC, such as are used in leading cloud computing platforms, such as AWS and Azure cloud services). Such an architecture allows the NDS to perform more efficiently functions such as load balancing, data encryption, or deep packet inspection, which are known in the art in connection with leading cloud processing/storage services. In aspects, an NDS NIC system comprises multiqueue NIC(s), employ NIC partitioning (NPAR, also known as port partitioning) (e.g., employing known SR-IOV virtualization), TCP offload engine technology, or a combination of any or all thereof. In alternative aspects, an NDS also comprises/only comprises a virtual network interface, such as Google Virtual NIC (gVNIC) or Oracle Cloud Infrastructure.

In aspects, an NDS relay unit can comprise an NDS output processing system that can filter data, analyze data, or both filter and analyze relevant data. In aspects, such functionality can be automatically applied functionality, e.g., automatic data filtering, automatic data analysis, or both, e.g., which may be facilitated by suitable programming. In certain embodiments, the NDS relay unit/processor can automatically filter MA-D, analysis (e.g., results from one or more analysis(es)), or both, for delivery to each MA and or alternatively to one or more other network devices.

In aspects, an NDS data relay unit can securely relay information (output) comprising MA-D, analysis (e.g., results from analysis(es)), output applications, or a combination thereof, via the internet (e.g., through secure internet communications), to one or more other network devices/interfaces (ONDIs). In aspects, information relayed to ONDIs comprises information from a plurality of MAs (e.g., any number of MAs) associated with any number of IEs, or is data derived from MA-D received from several MAs associated with 2 or more IEs, e.g., about 3 or more, about 4 or more, or about 5, about 10, about 15, about 20, about 25, about 30, about 35, about 40, or about 50 or more IEs.

In aspects, NDS output includes output applications. Output applications can include instructions for control over aspect(s) of MA operation, ONDI operation, or both. In aspects, output comprises relaying data in schemas or graphical representations. In aspects, output comprises all or part of a software application executed on an MA/OND client device/machine. Such an NDS facilitated/implemented software application can comprise graphically displaying data on a client OND/MA graphical display unit, various data tailored for the class of user associated with the relevant client device (e.g., an aggregate of MA statuses, usages, etc., in the case of a commercial class user, devoid of PHI; patient/device-specific data in the case of an HCP, comprising PHI; and research relevant information in the case of a research class user). Output applications can run on MAs/ONDIs through accessing data (e.g., graphical representations and other data) from an NDS, often in combination with stored elements (e.g., part or all of the graphical representation of the application) stored/generated on a local device. In aspects, applications run on MAs or ONDIs are model-view-controller (MVC) applications (dividing functionality into model, view, and controller functions, as known, to isolate representations of information from the manner in which the information is presented to the user, thereby allowing for efficient code reuse and parallel development). In aspects, such applications are web-based, and offer create, read, update, delete (CRUD) capabilities. In aspects, such capabilities allow new applications to be built on a common application infrastructure (e.g., in the case of applications relayed to NDS administrator class users). An output application can represent a OND/MA GUI in various ways (e.g., generating representation of a GUI using a combination of HTML and JAVASCRIPT including client device-executable code, NDS executable code, or both). The NDS can transmit or otherwise provide such representation(s) (in aspects≥2, ≥3, ≥5 or more depending on conditions, such as patient state, device state, etc.) to a client device for the client device to display on a screen/GUI according to its locally defined look and feel parameters, preprogrammed NDS parameters, or both. Alternatively, a representation of a GUI may take other forms, such as an intermediate form (e.g., JAVA byte-code) that a client device can use to directly generate graphical output therefrom. User interaction with GUI elements, such as buttons, menus, tabs, sliders, checkboxes, toggles, etc. may be referred to as “selection”, “activation”, or “actuation” thereof. These terms may be used regardless of whether the GUI elements are interacted with by way of keyboard, pointing device, touchscreen, or another mechanism. In aspects, components of the NDS organize received data (including MA-D) into web page or web application representations for display on ONDIs. Such a representation may take the form of a markup language, such as the hypertext markup language (HTML), the extensible markup language (XML), etc. In aspects, NDS components/units execute various types of computerized scripting languages, e.g., Perl, Python, PUP, Active Server Pages (ASP), JavaScript, etc., facilitating, i.a., delivery/presentation of web pages to network client devices and MA/OND interaction with such web pages/representations. Languages such as Java also can facilitate generation of web pages on network client devices, provide web application functionality, or both. In aspects, an NDS relay unit delivers outputs (display representations or other data, output applications, or both) via secure internet communication to one or more medical apparatuses, one or more other network devices, or both. Output can comprise (a)analytical data output; (b) one or more output applications comprising instructions that control the operation of (I) one or more medical apparatus functions in one or more of the medical apparatuses of the data network, (II) one or more other network device functions in one or more of the other network devices of the data network, or (III) both (I) and (II); or (c) a combination of (a) and (b).

An NDS relay unit can use any suitable form for reporting analysis/output can be used including reporting by email or other communication method (e.g., SMS/text), reporting by HTML/XML file report, or reporting within a search/operation application that accesses Function(s) of a method/NDS and displays results via a GUI on a networked device (e.g., a smartphone) or a web-accessible interface. In reporting results, S(s)/F(s) typically will comprise reference to confidentiality rule(s), algorithm(s), and the like, e.g., for the protection of PHI, typically drawing on authorization/access level information (e.g., generated by an AM), to ensure that PHI or other confidential information is not inappropriately released from DR(s) of the NDS to users not authorized to view such data based on data ownership, Regulatory Requirements (e.g., GDPR, HIPAA, etc.), or both.

In one aspect, an NDS relay unit delivers MA-D, NDS-AD, or both to a plurality of GUI schemes/interfaces based ≥ in part on user roles (classes). In aspects, such user classes comprise research team member/organization (research class users/ONDIs), commercial employee/agent (commercial class or business purpose users/ONDIs), and administrator class users/organizations (administrator class ONDIs). In aspects, some, most, generally all, or all of the commercial/BP users/ONDIs, administrator ONDIs, or both are associated with the entity(ies) that own or manage the NDS. Research class users/ONDIs can be associated with the NDS owner/operator, independent entities (IEs), or both. In aspects, some, most, generally all, or all of the research class ONDIs/users are associated with IEs (entities independent of the NDS owner/manager entity(ies)). Another possible class of users/ONDIs is a clinical or medical support team (clinical support users/class/ONDIs), which in aspects is associated with the NDS owner/manager. Users and ONDIs can be associated with IEs that are permitted to use the NDS (e.g., customers of the NDS owner, e.g., where the NDS owner is, e.g., a company that makes and sells MAs on (i.e., that use) the NDS). ONDIs and user classes are further discussed elsewhere.

Networks and NDS(s) can comprise any one or more wired or wireless medium/media that conveys data between point(s)/node(s)/component(s) of the network components, NDS components, or both. In aspects, wired or wireless medium can include, for example, a metallic conductor link, a radio frequency (RF) communication link, an Infrared (IR) communication link, an optical communication link, or the like, without limitation. The RF communication link can include, for example, Wi-Fi, WiMAX, IEEE 802.11, DECT, 3G, 4G or 5G cellular standards, Bluetooth, or the like. A communication(s) link can include, e.g., a voice-over-Internet-Protocol (VoIP) line, a cellular network link, an Internet protocol link, or the like. The Internet protocol can include an application layer (e.g., BGP, DHCP, DNS, FTP, HTTP, IMAP, LDAP, MGCP, NNTP, NTP, POP, ONC/RPC, RTP, RTSP, RIP, SIP, SMTP, SNMP, SSH, Telnet, TLS/SSL, XMPP, or the like), a transport layer (e.g., TCP, UDP, DCCP, SCTP, RSVP, or the like), an Internet layer (e.g., IPv4, IPv6, ICMP, ICMPv6, ECN, IGMP, IPsec, or the like), and a link layer (e.g., ARP, NDP, OSPF, Tunnels (L2TP), PPP, MAC (Ethernet, DSL, ISDN, FDDI, or the like), or the like).

An NDS-RELAYU can comprise Unit(s)/Engine(s) that are included in the Input Unit including, e.g., data tagging/curating functions, serialization/deserialization functions, and short term (in-memory) analytical functions. An NDS relay unit will typically employ multiple (e.g., massive) parallel processor structure(s) and framework(s), e.g., those described elsewhere in connection with other U/F(s). An NDS relay unit can, in aspects, also employ distributed processing and processing functions, e.g., queuing, task scheduling, etc., as well as employ data filtering and data structures/schemas for presentation of data (such functions also can be performed by the analytical unit in the generation of NDS-AD).

In aspects, output applications comprise medical apparatus-specific instructions for controlling (a) one or more therapeutic tasks performed by a particular medical apparatus (e.g., causing an MA to change conditions that result in a typical patient in a change of physiological conditions or other change of physical conditions through MA operation so as to treat the symptom(s) or cause(s) of a disorder), (b) one or more preventative tasks performed by a particular medical apparatus (to cause similar changes in operation of an MA that change patient conditions to reduce the risk of developing, reduce the severity upon onset, delay onset, prevent developing, etc.), or (c) both one or more therapeutic tasks and one or more preventative tasks performed by the particular medical apparatus; (2) the particular medical apparatus receives, interprets, and executes the one or more output applications; and (3) the particular medical apparatus changes one or more conditions of patient care based on the received one or more output applications (e.g., as described above).

In aspects, generation of output application(s) comprise(s) (1) generating, for display on a graphical user interface of one or more medical apparatuses, a first representation comprising (I) recommended medical instructions, (II) analytical data relevant to a medical apparatus, patient, or both, which analytical data comprises patient protected health information, or (III) both (I) and (II), (2) relaying the first representation to the one or more medical apparatuses for display on one or more graphical user interfaces thereof, (3) generating, for display on a graphical user interface of one or more other network devices, a second representation comprising analytical data that is devoid of any patient protected health information, and (4) relaying the second representation to one or more other network devices for display on one or more graphical user interfaces thereof, wherein steps (1)-(4) are performed within less than 5 minutes, 2 minutes, 1 minute, 0.5 minute, 0.25 minute, 0.1 minute, less than 2 seconds, or ≤1 second. C. Other Network Devices/Interfaces and Components

In addition to MAs and the NDS, a network can comprise other devices (ONDs) and an NDS can deliver data to other network device interfaces (ONDIs), which, in either case typically can receive, send, or both send and receive data to the NDS. ONDIs can be associated with other classes of users, besides the HCP operators of MAs, MA administrators or MA local/IE network administrators, and NDS administrators. ONDIs can be grouped based on user classes into, e.g., RNDIs (research ONDIs), BPNDIs (business purpose/commercial ONDIs), and ANDIs (administrative ONDIs).

In facets, ONDIs comprise NDS owner/operator (SO) support system(s) (SOSS(s)), and the NDS delivers MA-D, NDS analysis, or both, to NDS owner/operator-associated network access devices (SOANADs). Such support system(s) can comprise (a) NDS analyst/administrator devices/interfaces, (b) a clinical support class of devices/interfaces (CONDIs/CSONDIs), or both, which are often both operated by persons associated with the NDS owner/manager. NDS analysts/administrators can be engaged in NDS maintenance, NDS construction, NDS management, NDS analysis, etc., and access the NDS through administrative ONDIs (ANDIs/AONDIs). Additional classes of ONDIs/users include commercial/business purpose (BP) users/ONDIs, which users and ONDIs also can be associated with the NDS owner/manager. Professionals that operate BP-ONDIs can include MA salespersons and commercial management. Regulatory requirements (“RRs”) may not permit persons from accessing PHI and, accordingly, NDS rules for delivery of data to BP-ONDIs restrict such ONDIs from receiving or accessing such data. In aspects, ONDIs are actual hardware devices (“ONDs”), such as smart phones, tablets, personal computers, and the like.

Still other ONDIs/users belong to research class persons/organizations and such users are associated with research ONDIs (RONDIs/RNDIs). Some, most, generally all, or all research class users can, in aspects, can also have research MAs that are part of the network. Research MAs can include MAs that are of an experimental nature (new MAs, proposed improved MAs, etc.), on-market MAs being operated in a clinical trial or other experiment, or both, which can be present along with or as an alternative to other types of ONDIs associated with researchers (e.g., research class user computers, cell phones, etc.). An NDS can comprise specialized Unit(s) for receiving input from some, most, generally all, or all ONDIs, relaying NDS-AD and other data to such ONDI devices/interfaces, or both. In aspects, one or more ONDI in a network can comprise, e.g., ≥1 sub-collections of ONDIs based on sub-networks, user classes, or both (e.g., a class of researchers associated with a particular IE or study; a class of BP-ONDIs associated with sales of a particular type of MA; etc.) and relevant data will include tags or other identifiers for such sub-classes of users/data sources.

In aspects, NDS administrators/analysts, research class users, or both can be engaged in, i.a., machine learning management, e.g., in supervised machine learning methods. Clinical support users typically are users that provide monitoring support for subjects being treated by MAs, e.g., by providing a second level of support to users in observing MA-D, receiving alerts/alarms based on such MA-D, and notifying the IE owner of the MA-D, relevant HCPs, etc., of any identified and unresolved issues, typically according to standards, protocols, etc. Other owner-associated devices/interfaces can be associated with BP/commercial class users (e.g., sales professionals, medical affairs professionals, etc.). Owner-associated users, particularly BP/commercial users, usually cannot access PHI and, accordingly, the NDS in such aspects typically employs filters, curation methods, etc. to ensure that such Owner-associated users do not receive PHI. Data tagging and matching methods can be employed in such function(s)/engine(s).

In aspects, a network can comprise a commercial class device/interface network, e.g., one or more sales representatives, sales managers, or corporate-level personnel overseeing sales, a salesforce, or sales training, which typically includes users associated with the NDS owner (e.g., salespeople of MAs in the network). According to one facet, users comprise sales representatives that sell or lease MAs on behalf of the NDS owner/operator (SO). In aspects, communication to such networks can facilitate training, aid in informative communication between a sales representative and an independent entity housing one or more MAs, or can, e.g., facilitate or aid in the group of data to support the training of a sales team, e.g., for example provide data on NDS functionality, NDS alerts, aggregated NDS data (e.g., regarding NDS performance, functionality, or the like), or NDS usage. In some embodiments, a NDS or SOSS can combine MA-D, analysis (NDS-Ad), or both, with data from a customer relationship management (CRM) system. Data from a CRM support system (CRMSS) can be directly inputted to the NDS, can be delivered to commercial class devices/interfaces in combination with NDS-AD, etc.

In facets, ONDIs comprise a research platform (RP) that delivers MA-D, analysis, or both, to network access devices (NADs) associated with scientific researcher (SR)-associated network access devices (SRANADs). In aspects, some, most, generally all, or all the SR users are not associated with the SO through employment or similar affiliation (e.g., by being employed by 1 or more IEs). In aspects, the NADs are associated with 1 or more SRANADs, wherein the SRs are associated with the SO. In aspects, research class users comprise both SO-associated and non-SO/IE-associated users, and the NDS comprises data tagging or other data identification means for linking the research user association with research user-associated MAs or other inputs or outputs (e.g., interfaces). In aspects, communication to such a platform can facilitate NDS training, aid in further NDS development efforts, alert developers to NDS errors, provide developers information related to NDS usage levels, demand, and data flow patterns, etc. Also or alternatively, in aspects communication to such a platform can facilitate SO-related, SO-sponsored, or SO-independent medical research, support SO-related, SO-sponsored, or SO-independent clinical studies, or support other such clinical-data group-related endeavors. In aspects, data shared with such an ONDI is controlled and or governed by established data control mechanisms related to pre-established rules (e.g., rules established by the SO, by ≥1 individual SR(s) or groups of SR(s), rules established by patient consent, contract, agreement, scientific protocol, or the like, or, for example, established by health care compliance rules (e.g., government imposed privacy rules e.g., HIPAA rules, GDPR rules, etc.). In aspects, the NDS comprises a repository of such rules or access to such rules and a system for monitoring or regularly updating any such applicable PII/PHI rules.

In aspects, one or more other user classes/groups can comprise a clinical support group associated with clinical support devices/interfaces, which typically can deliver or receive certain types of data to/from an NDS. In aspects, communication to such groups facilitate improved patient care, e.g., in making clinical support staff capable of participating in communication between the NDS and primary care giver(s) co-located with the MA(s), MAG owners, etc. In aspects, communication to clinical support groups can allow such clinical support groups to, e.g., support data interpretation, interpret NDS indicator(s) e.g., alarm(s), etc.

In aspects, the NDS comprises a functional module/engine/unit that can read data directly from an electronic health/medical record (EHR/EMR), write MA-D, NDS analysis, or both, directly to an electronic health record (EHR), or a combination thereof. In aspects, the NDS limits access to EMR/EHR data to HCPs through encryption, data curation, governance zone applications, etc. The terms EMR and EHR provide implicit support for one another here as alternative aspects.

In exemplary aspects, a network comprises ≥3, ≥5, ≥10, ≥20, ≥30, ≥50, ≥100, ≥200, or ≥500 other network devices (ONDs), each OND (I) comprising (A) a processing unit, (B) a memory unit, and (C) a remote controllable graphical user interface, and (II) being associated with a user assigned to at least one class of users, wherein classes of users comprise (A) health care providers authorized to access patient protected health information and (B) commercial users that are subject to restrictions on the receipt of patient protected health information. CEI in the NDS provides for sequestration, redaction, encryption, etc., of PHI, preventing such data from being delivered to commercial class user-associated ONDIs. In aspects, NDS CEI includes instructions for using non-patient-specific aggregate data, etc., so that commercial class users can receive useful and relevant information concerning MAs of interest to the commercial class user(s), such as aggregate usage data, performance data, and the like.

According to facets, MA-D comprises location information and the NDS relay/DDRU simultaneously relays information to ≥2 classes of ONDIs based on facility, metropolitan area, state/region, country, or IE. In some embodiments, MA input unit(s) indirectly receives input derived from one or more research teams and IEs using MAs in clinical research application(s).

In aspects, an NDS processor can automatically filter MA-D, analysis (e.g., results from 1 or more analysis(es)), or both. In aspects the NDS processor can filter data according to 1 or more established or pre-defined rules or sets of rules, e.g., rules or sets of rules relating to confidentiality (e.g., rules established by one or more individual facility(ies), rules established by contract or agreement, or the like) or, for example, health care compliance rules (e.g., government-imposed privacy rules e.g., HIPAA rules, HI-TECH Act rules, GDPR, etc.), such filtration usable for ensuring ONDIs receive only data appropriate to its role. ONDIs can comprise any suitable hardware device(s), software application(s), or both, for receiving output from the NDS. Each class of ONDI user typically will receive data according to one or more schemas and rule sets that are specifically tailored to the class of ONDI user in a Regulatory Requirement-compliant manner. In other aspects, such users can interface with the NDS via web-based interfaces, retrievable on any suitable device, and from several devices supporting web browser applications (e.g., mobile phones, tablets, or laptop computers), and from several such devices with adherence to appropriate log-in/security processes. Accordingly, ONDI processes can be implemented in hardware, firmware, software, or a combination thereof (CT). Hardware in ONDI devices can comprise, e.g., one or more application specific integrated circuits (ASICs), digital signal processors (DSPs), neural network processors (NNPs), digital signal processing devices (DSPDs), programmable logic devices (PLDs), field programmable gate arrays (FPGAs), processors, controllers, micro-controllers, microprocessors, electronic devices, or other electronic units/circuits/components suitable for performing relevant steps/functions. Firmware/software components can comprise, e.g., microcode, procedures, functions, and so on that perform S(s)/F(s). CEI, e.g., program code, can reside in any suitable PTRCRM, e.g., a NTRCRM, executed by processors. Computer(s) that can be ONDI devices (e.g., mobile phones, laptops, servers), networks, and accessible processor functions (e.g., in a distributed computing environment) can comprise processing function(s) and data memory and can optionally supplement one or both with cloud-based resources or NDS resources. In aspects, ONDI computers or computer systems can include a CPU, a ROM, a RAM, a HD, and I/O device(s). I/O devices can include, e.g., a keyboard, monitor, printer, electronic pointing device (for example, mouse, trackball, stylus, touch pad, etc.), or the like. ROM, RAM, and HD are NTCRM memories known for storing computer-executable instructions executable by the CPU or capable of being compiled or interpreted to be executable by the CPU. PTRCRM or NTCRM can include volatile and non-volatile computer memories and storage devices e.g., random-access memories, read-only memories, hard drives, data cartridges, direct access storage device arrays, magnetic tapes, floppy diskettes, flash memory drives, optical data storage devices, compact-disc read-only memories, and other appropriate computer memories and data storage devices. Examples of NTCRM include register memory, processor cache, and non-power-dependent memory media such as ROM, hard drive(s), and ACT. Either type of CRM can refer to, e.g., a data cartridge, a data backup magnetic tape, a floppy diskette, a flash memory drive, an optical data storage drive, a CD-ROM, ROM, RAM, HD, etc. ONDI devices can store, operate, or store and operate an operating system (OS) utilized to control the operation of device(s). OSs are known in the art and include LINUX, WINDOWS, Apple iOS, android, UNIX, SOLARIS, and other suitable platforms.

### D. System Operation Methods and Related Methods

The invention provides, i.a., methods for collecting information from a connected network of medical devices, comprising accessing a network of separately located medical apparatuses (MAs) (typically in ≥2 MAGs owned by different IEs) and collecting the data communicated from the MAs in a medical apparatus network data management system (NDS) that forms a network (e.g., a SDWAN) with the MAs, where such data is primarily, generally, or substantially collected as SMAD, but in which the NDS also receives locally stored/cached MA-D collected and relayed by MAs under condition(s) (e.g., where the MAs and NDS fail to communicate due to one or both being offline). In aspects, MA-D received by the NDS is stored in a DL or EDL DR. In aspects, a network comprises one or more classes of other network devices/interfaces (ONDIs) associated with users in the relevant classes, e.g., a research class, a clinical support class, a business purpose/commercial class, or a combination thereof. In aspects, an NDS simultaneously transmits NDS-AD to most, generally all, or all such other ONDIs regularly or upon event, but such ONDI displayed data is differentiated from each other by schemas/rules that limit and curate data in such displays (e.g., withholding PHI from commercial class users). The MAs, NDS, and other network components/NDS components engaged in such methods can have the components, perform any of the inventions, and exhibit any of the characteristics described elsewhere in connection with such elements. In general, any of the above-described NDS components can be related to steps of method. However, to further clarify aspects of the invention, certain aspects of method steps or operational characteristics of certain elements of NDS/method are described in the following sections.

As also will be clear from the preceding sections of this disclosure, there are several steps that can be involved in the operation of the NDS, at both the MA (device) level, NDS (system/MAC-DMS) level, and ONDI level. At the MA level, such steps include sensing of subject data and analysis thereof and transmission of such data (directly or in an MZMA potentially indirectly) to the NDS and receiving data/instructions from the NDS. At the NDS level, the steps typically include (a) data input/intake (e.g., by an SDP), (b) determination of initial analysis of intake data (e.g., by components of the SDP), (c) performance of initial actions based on initial analysis, (d) ingestion of data into the NDS DR (e.g., an EDL), (e) analysis of DR data (automatically, on-demand, or both), and (f) performance of actions based on NDS-AD of DR data (e.g., changing ONDI displays, changing MA displays, changing MA controls, or a CT).

### I. System Input/Intake Methods and Characteristics

Methods of the invention can comprise receiving input from MAs, typically numerous MAs (e.g., ≥~100, ≥1000, ≥5000, or ≥~10000 MAs), which can be associated with several different IEs (e.g., ≥~3, ≥5, ≥10, ≥20, ≥50, or ≥~100 IEs), a similar number of different locations (+/−~33% of any such values), and comprise different MA types (e.g., ECMO and heart pump devices), different patient types, or any CT. Input to an NDS is typically processed by highly available parallel processing. In aspects, input is processed by distributed massively parallel processors, which in aspects comprise, mostly are, generally are, or entirely are scalable cloud-based processors. In aspects, method include an initial analysis step performed at or soon after intake, such as in a separate memory/processor from the primary memory/processor of the NDS (e.g., in an SDP), resulting in immediate action(s) or outputs in some cases. In aspects, the input method comprises application of data queuing processes, minimal transformation, or structure screening for most or generally all RT/S input data, or both, after NDS DR ingestion. In aspects, data is at least initially ingested into, and mostly, generally, or substantially maintained in a DR comprising a DL or EDL structure. Other particular aspects of method are discussed below, which can be applied to or combined with these and other aspects of this disclosure.

### 1. System Data Security

In aspects, methods comprise application of data transformation steps for ensuring confidentiality of confidential information in DR(s), e.g., PHI, for protecting data transmitted from the NDS, for protecting the NDS from unauthorized/malicious access, or a CT. Such steps can comprise application of one or more encryption, firewall protection (including, e.g., web application firewall(s)), or both.

In aspects, Secure Sockets Layer (SSL) protocols can be employed to protect sensitive/confidential information (SI), which should be maintained as confidential based on contractual, proprietary, or Regulatory Reasons. In aspects, secure channel Java applet transmission also can be used to protect SI. Data encryption algorithms also applicable to such data protection methods include, e.g., RSA Data Security RC4, Data Encryption Standard (DES), and Triple DES (3DES). In aspects, S(s)/F(s) comprise SI access, use, or transfer monitoring, e.g., by employing Database activity monitoring (DAM) software/tools. Examples of other commercially available data security tools that can be applied to an NDS (or that corresponding components can be incorporated into an NDS) include Sophos Intercept X for Server, IBM Security Guardium, Oracle Audit Vault and Database Firewall, Imperva Data Security, Trend Micro ServerProtect, and SQL Secure. In aspects, firewall Functions comprise a list of authorized commands, which can vary, e.g., with level of User/Entity authorization. Firewall and authorization features, e.g., IP address evaluation, etc., can also overlap, as can reporting of unusual information, attacks on the NDS, and the like. In aspects, SI-containing Engine(s), DR(s), or both, are segmented from other DS(s)/Unit(s) by data curation rules/methods. Typically, each type of DS is identified with 1 or more identifiers, including type identifier(s), which aid in segmentation and protection of SI. Typically, an NDS will comprise a confidential information module (CIM), which can comprise or consist of a key management system, which stores access keys, policies, protocols, monitoring method(s), or a CT. Most, or all NDS information can be stored encrypted at rest. Regional storage tags/rules, etc., also can be applied to limit/control or validate information access. In aspects, part(s), most, or all components of an NDS reside on a secure network without ability or with a limited ability to receive internet-based external (incoming) communication(s). A NDS can in aspects comprise several isolated sub-systems, but which, in aspects share or sometimes share message queues. In aspects, data is extracted from an NDS/method only by an API call. Other examples of methods/technologies for the protection of SI in networks/systems, e.g., encryption, firewalls, and the like, which may be adaptable to NDSs/methods, are described in, e.g., U.S. Ser. No. 10/601,593, WO2001037152, U.S. Pat. No. 8,010,791, WO2013064565, U.S. Pat. No. 6,148,342, U.S. Ser. No. 10/581,605, US20180300497, U.S. Pat. Nos. 9,436,841, 6,148,342, and references cited therein. Methods of confidential information protection also are found elsewhere.

A firewall employed by method/NDS can in aspects provide security by selectively granting or denying access to the private network. A firewall can be/comprise/employ a NAT, a proxy, other device(s)/function(s), or a combination of 2 or more of such or similar system(s)/component(s)/engine(s). Such engine(s)/device(s) can block or restrict unauthorized incoming data and unauthorized incoming requests from devices on a private network. Firewalls can isolate devices “behind” them from a public network, and thereby provide security against unsolicited connections. Firewalls can also restrict the way computers inside can access outside public sites, e.g., those on the Internet. One technique for establishing a firewall is to maintain a list of “authorized” addresses. Address information contained in a data packet from a remote device can be examined by applicable security methods/units, e.g., to determine whether the originating source is on the list. Only packets coming from authorized addresses can pass. An NDS can also comprise an encryption function/engine, which can comprise any logic, business rules, functions, or operations for handling the processing of any security related protocol, e.g., SSL or TLS, or any function related thereto. An encryption Unit, e.g., encrypts and decrypts network packets, or any portion thereof, communicated via the appliance, establish SSL or TLS connections on behalf of the client, or both. In aspects, an encryption Unit/Function uses a tunneling protocol to provide a VPN between MAs/ONDIs and a networked NDS.

An application firewall can, in aspects, inspect or analyze any network communication in accordance with the rules or polices of the NDS/CIM to identify any confidential information in any field of the network packet. In embodiments, an application firewall identifies in the network communication PHI/confidential information and take policy action on such network communication (e.g., rewrite, remove, or mask the confidential information, block the message, etc.).

In aspects, proxy server(s) are included in the NDS/network architecture (e.g., placed behind NDS firewall(s)). In cases, proxy server(s) can initiate communication sessions with network client devices (MAs, ONDs, etc.) “through” firewall(s), as known in the art. In such aspects, firewall(s) might not have to be specifically configured to support incoming/outgoing sessions/communications, thereby DOS reducing risks of loss of communication for proxy server managed communication(s).

In aspects, methods also comprise steps for ensuring data in MAs is secure from unauthorized physical tampering, malicious internet tampering/access, or a CT. Such steps can include application of anti-tampering software, firewall security, authentication, and other physical anti-tampering mechanisms, discussed elsewhere. In aspects, method(s) comprise the use of MZMAs, in which one or more zones are relatively restricted, e.g., in not having direct internet interaction (e.g., connection or communication), or in having extremely limited permitted internet data exchanges (e.g., ≤~10, ≤7, ≤5, or ≤~3 types of permitted transactions, which may be, e.g., limited to restricted zone component control, zone/MA operating system update (but typically only with pull demand from a local user), or both).

### 2. Status and Operational Mode Data

In aspects, an NDS can detect mode(s) of operation of MAs or other MA-associated status information (e.g., device malfunction). MA operation mode detection can be performed by receipt of status data transmitted from the MA, from contextual data analysis, or both. E.g., in aspects the NDS can detect MAs in non-clinical operational modes and use such capabilities for, e.g., research and development or scalability testing. In aspects, maintenance of the network/NDS comprises performance of capability testing with multiple test MAs or MA simulators/simulations providing non-operational status signals to the NDS and evaluating the capability of the NDS to operate with such test MAs/simulators or with such test MAs/simulators added to the current network data load. MA modes detected also can include the application state of MAs, e.g., MAs engaged in clinical research, basic/fundamental MA/network research, or both. MA modes can in aspects also include MAs in local MA repair/maintenance modes.

### 3. Data Transformation and Data Characteristics

Steps of methods of the invention can include one or more data transformation processes, which can result in control of devices, display of instructions on device/interface displays, or other data displays or data-controlled applications (e.g., alarms/alerts or reports). Data transformation can be employed at the data ingestion layer, data collector/collection layer (where data is moved from storage to the data pipeline for processing/analysis), data improvement layer (e.g., where steps include data cleaning, data harmonization/formatting, or a CT), data processing/analytics layer (e.g., an NDS analytical unit (NDS-ANALU)), data query layer (where query processes and other intensive analytic applications take place—e.g., through use of application e.g., Apache Hive), or data visualization/application layers.

Methods of the invention also can include processing of data having various different characteristics including, as mentioned, both RT/S and cached data, as well as data in different formats, e.g., unstructured data, semi-unstructured data, structured data, or a combination thereof. Analytical steps of methods can also generate and relay data in a similar variety of formats.

“Structured data” typically means organized data, e.g., data mapped to pre-defined fields in a structured arrangement, e.g., data organized in columns/rows or fields/records. Structured data will typically include ≥about 5, ≥6, or ≥about 7 data relationships, and often include ≥~2, ≥3, or ≥~4 levels of data or types of data or data transformations or specialized data (e.g., several columns (attributes), rows (values), and tabs; tables with records, fields, primary keys, and queries; etc.). Structured data typically comprises relational data. Structured data is typically rigid/fixed given its structure. Unstructured data typically means data that cannot be contained in such a highly structured or organized format (e.g., row-column database), does not have an associated data model, or both. Examples of unstructured data records include web pages, text messages, images, social media content, documents, recordings, etc. Semi-Structured Data is data having a limited amount of defining or consistent characteristics/relationships, but that does not comprise as complex a structure or conform to a structure as rigid as is expected with structured data. Semi-structured data can be characterized by simplicity of relationships, heterogeneity of data content, or both. An example of a semi-structured data record is an email, which has a limited number of structure (value/attribute) elements (sender name, recipient name, transmission date, and subject), but which typically mostly is unstructured (in the primary content of the message). Other examples can include XML, EDI, CSV, ORC, Parquet, TSV, HTML, and OEM documents/objects (noting, however, that some CSV/TSV delimited structures may be classified/classifiable as structured data). Unstructured data can be transformed to semi-structured data by application of metadata (e.g., in the case of video or image data, which may include metadata e.g., date taken, location taken, photographer, or a CT, but which primarily is unstructured data). Semi-structured data attributes are often not ordered consistently. In steps/methods input comprises, primarily comprises, or generally consists of semi-unstructured data, e.g., SUMAD. In aspects, steps of the method comprise receiving some, most, or generally all input of at least some types, e.g., MA-D in a particular semi-unstructured format, e.g., a JSON format. In aspects, serialization/deserialization steps are performed with respect to similar or certain other formats to provide more data in the target format (e.g., JSON or Avro). In aspects, JSON formatted data input can comprise separated lists (e.g., through inclusion of backet separators). In aspects, most, generally all, or substantially all JSON data for each JSON or similar semi-unstructured input file exhibits generally no or substantially no data repetition. In certain methods, the semi-unstructured data inputs in the method(s) are limited to a set of sources, expected data values, etc. In aspects, NDS resources seek to identify data not fitting such expected key/value and type relationships in the input. In aspects, policies prevent the addition of other types of inputs without SO approval and NDS modification. These limitations can ensure that queries based on the semi-unstructured data in the NDS DR are effective, despite the semi-unstructured nature of the collected data, while the semi-unstructured nature of input ensures that input file sizes are small and simple, in aspects DoS decreasing ingestion time. methods can comprise application of MLMs to SUMAD, e.g., text analysis models e.g., key feature extraction, sentiment analysis, context analysis, etc. In aspects, methods include the use of a system that is adapted to process unstructured and semi-structured data, e.g., MongoDB or Aparavi. In aspects, methods comprise employing a system that can process unstructured, structured, and semi-unstructured data concurrently, e.g., the Aster Centerprise System, or Microsoft Azure data management systems.

Methods can comprise application of metadata to data as part of data transformation steps. Metadata typically is “data about data” or data that provide(s) information about a file, record, data set, chunk, stream, packet, or other data structure. Metadata can include source information (e.g., device identifier, device-Entity association, device-location relationship, device-type identifier, subject identifier, subject type, treatment method, associated HCP(s)/users, or a CT). Metadata also or otherwise can comprise structural metadata (e.g., data types, data relationships, attributes, hierarchies, expected ranges/types/units, Levels, etc.), administrative metadata (e.g., permissions, creation date, update date, update instructions, Administrator contact information, etc.), or reference metadata (e.g., how data was obtained—e.g., RT/S MA-D, cached MA-CD, NDS-AD, —or how data was transformed, e.g., application of a schema, result of a query, output of an application, modification by data improvement, e.g., data cleaning, result of MLM, or any CT, etc.). Metadata can include, for example, an access control descriptor, legal definition descriptor, and a summary and aggregated definition, which can be used to control access or drive search heuristics to data in a data lake or other data repository. In addition, implementations can automatically recalibrate, refine, correct, or recalculate data based on substantially continuous tracking of lineage information of an object, provenance of the system, transformation of the process or the version of specific transformations applied to the data.

In aspects, most, generally all, or substantially all data of one or more types (or data input overall) received by the NDS as input is streaming MA data (SMAD), or real time data (RT/S data). “Streaming data” typically refers to data delivered in data streams, which are typically unbound (of unknown or unlimited size), continuously updating (at least while the source is operating and online), data sets. Methods can comprise receiving, optionally analyzing, optionally acting on, and ingesting stream data through use of a streaming data processor (SDP) and related methods for processing data streams, e.g., application of stream partitions. In aspects, methods comprise generation of ordered, replayable, and fault-tolerant sequence of immutable data records, in the generation of stream partitions, where a data record is defined as a key-value pair. Processor topology refers to the computational logic of aspects of data processing. In stream processing applications, a topology can comprise, e.g., a graph of stream processors (nodes or stream processors) that are connected by streams (edges). Each node typically is used to transform or ingest data. Standard operations e.g., map or filter, joins, and aggregations are examples of Functions performed by stream processors, e.g., as part of an Input Function/Unit. Streams can be stateless, but methods can comprise partitioning streams into stateful data records (allowing for application of join, aggregate, and window functions, and for fault-tolerance methods). Unit(s)/Function(s) (timestamp extractors) can apply timestamps to every stream-derived data record. An aggregation operation can take an input stream or table, and yields a new data structure, e.g., a table by combining multiple input records into a single output record. A join operation can merge≥2 input streams or tables based on the keys of their data records and yields a new stream/table. Stream processing methods typically include RT monitoring functions, which comprise status/threat reports/monitoring data/output, trend detection, event detection, etc. In aspects, methods comprise application of methods to ensure that mostly all or generally all data is processed in order. In aspects, methods comprise F(s)/S(s) for ensuring out-of-order partitions are provided with sufficient time to be re-associated with correct partitions. Such methods can include providing for 1 or more data cycles, in which collections of data are generated from partitions, prior to 1 or more levels of analysis (e.g., by Engine(s)/component(s) that can be classified as data stream aggregators). E.g., in one aspect, an initial data collection cycle of ≥~5 seconds, ≥7 seconds, or ≥~8 seconds, is employed, in which partition data is collected, prior to initial analytical methods are employed on the first level assembled data collection, and, in further aspects, a longer period, e.g., of ≥~1 minutes, ≥2 minutes, ≥3 minutes, or ≥~5 minutes, is employed, for assembling a larger, second level data set comprising several collections of the first level, initial datasets, prior to authorizing consumption of such data by one or more applications, e.g., an MLM application. In aspects, first level data sets are continuously evaluated in the construction of a second level data set, and, if an error is detected and cannot be corrected during the second data cycle, the second data cycle can be re-started, without losing some or all the data that would otherwise be collected in the original second cycle window/period. Such methods also exhibit the use of a combination of processes that can be characterized as streaming/RT ingestion with batch processing on collected RT/S data. In aspects, a method comprises collecting streaming MA-D (“SMAD”) in a 1^{st} data aggregation collected over a first data cycle time and that is subjected to an 1st analysis, and a 2^{nd} aggregation that is collected for a 2nd data cycle time and which comprises a collection of 1st aggregation data and which further is subjected to a 2^{nd} analysis, wherein the data cycle time for collecting the second aggregation is ≥5 times as long, ≥10 times as long. or ≥25 times as long as the first data cycle time; the first analysis and second analysis are different; the second analysis is more data intensive, processing intensive, or both than the first process; and the method further comprises evaluating each first aggregation for suitability for inclusion in the second aggregation and re-setting any second data cycle when a first aggregation is determined to not be suitable before the completion of a second aggregation. In aspects, such methods are mostly, generally, or only performed on SMAD-derived data after ingestion into the NDS DR.

Streaming queries performed as part of an initial analysis performed by an SDP can be processed, e.g., using a micro-batch processing engine, which processes data streams as a series of small batch jobs thereby achieving end-to-end latencies as low as between about 1-500, 1-250, or about 1-100 milliseconds and exactly-once fault-tolerance guarantees. Event Hubs, IoT Hub, Azure Data Lake Storage Gen2 and Blob storage are supported as data stream input sources. Kafka Streams and similar frameworks are designed to handle streaming data input and related processes. Event Hubs are used to collect event streams from multiple devices and services, e.g., a network. Blob storage can be used as an input source for ingesting bulk data as a stream, e.g., log files, relayed from MAs. Azure Stream Analytics, a cloud-based service for ingesting high-velocity data streaming from devices, sensors, applications, Web sites, and other data sources and analyzing that data in real time, can be used to perform some, most, or generally all streaming data input or analytical processes in methods. In aspects, such a platform or other methods analyze incoming data for anomalies or information of interest (e.g., streaming data indicating a chance in patient state, which may signal an emergency or that a change in application is required to prevent development of a life-threatening condition, which can trigger the NDS to send alerts/alarms or therapeutic component instructions or control data).

In aspects, a streaming analytics service/Unit (an SDP or component thereof) operates at about 1 MB/sec-about 50 MB/sec of throughput. In aspects, the streaming data flow(s) also are received, initially processed (initially transformed, analyzed, curated, or a CT), and stored, and, in aspects, applied in 1 or more respects, in a period that would be considered RT, as discussed elsewhere here. In aspects, NDS capabilities and processes are such that data ingestion, transformation, application, or relay processes, or any combination thereof, are associated low latencies (e.g., between ~0.0001-100, 0.0001-50, 0.0001-10, or between ~0.0001-1 seconds, or other measure provided herein) are achieved and maintained in most, generally all, or substantially all operational periods, on average, or both, which also or alternatively can characterize the process as RT. In aspects, the streaming characterization is generally always or substantially always supported by substantially continuous data flow from most, generally all, or substantially all network devices. In aspects, an RT system can be characterized as a “soft” RT system with a latency in, e.g., ~0.5-10 sec, or a “hard” RT system with average/most latencies in the range of ~0.001-0.1 or ~0.001-0.1 sec, can be used some, most, or all of the time.

Methods of the invention can comprise performing at least some data transformation, at least some data analytics, at least some data applications, or a CT, on the RT/S data, prior to such data being committed to a NDS DR (ingested). In addition to other methods described here, stream processing methods can use multiple computational units, e.g., the floating-point unit on a graphics processing unit or field-programmable gate arrays (FPGAs), without explicitly managing allocation, synchronization, or communication among those units. In aspects, a series of operations (kernel functions) is applied to each element in the stream. Kernel functions are usually pipelined, and optimal local on-chip memory reuse is attempted, to minimize the loss in bandwidth, associated with external memory interaction. Uniform streaming, where a kernel function is applied to all elements in the stream, is typical. Stream processing hardware can use scoreboarding, for example, to initiate a direct memory access (DMA) when dependencies become known. The elimination of manual DMA management reduces software complexity, and an associated elimination for hardware cached I/O can reduce the data area expanse involved with service by specialized computational units e.g., arithmetic logic units. A stream processor is usually equipped with a fast and efficient memory bus (e.g., crossbar switches or multi-buses, e.g., a 128-bit or 256-bit crossbar switch matrix (4 or 2 segments)). Pipelining is a very widespread and heavily used practice on stream processors, with GPUs featuring pipelines exceeding 200 stages, which can be adapted/employed in methods. The “cost” for switching settings is dependent on the setting being modified. To avoid costs/burdens at various levels of the pipeline, many techniques may be deployed e.g., “tber shaders” and “texture atlases,” which can be adapted to methods. Stream processing data can be held/processed by systems/components called event stream processors (ESP) that are able to ingest data streams and process them with a small response time and no data loss. Streaming event data can be generated by beacons, which can send, e.g., MA location data, to an NDS.

Non-commercial examples of stream programming languages/frameworks/systems include Ateji PX Free Edition, enables a simple expression of stream programming, the Actor model, and the MapReduce algorithm on JVM Auto-Pipe, from the Stream Based Supercomputing Lab at Washington University in St. Louis, ACOTES Programming Model: language from Polytechnic University of Catalonia, Brook language from Stanford, RaftLib—open source C++ stream processing template library originally from the Stream Based Supercomputing Lab at Washington University in St. Louis, StreamIt from MIT, and WaveScript Functional stream processing, also from MIT. Commercial implementations include AccelerEyes' Jacket, a commercialization of a GPU engine for MATLAB, IBM Spade—Stream Processing Application Declarative Engine (See: B. Gedik, et al. SPADE: the system S declarative stream processing engine. ACM SIGMOD 2008.), RapidMind, a commercialization of Sh (acquired by Intel), CUDA (Compute Unified Device Architecture) from Nvidia, Apache Kafka, Apache Storm, Apache Apex, Apache Spark Streaming, Apache Flink, Apache Flume, Amazon Web Services—Kinesis, Kinesis Streams, and Amazon Firehose, Google Cloud—Dataflow, IBM Streams and IBM Streaming Analytics, Oracle Stream Analytics, Oracle GoldenGate, and Microsoft Azure—Stream Analytics and its components/related applications (e.g., PowerBI, Trill steam processor, etc.).

In aspects, methods can comprise remotely managing one or more aspects of the operation of the NDS. E.g., methods can comprise remotely supervising scaling, data transformation, data ingestion rules, queries or other applications, or supervision of MLMs, or relay of data to other network devices. A cloud based NDS or NDS component can comprise a plurality of Virtual Machines (vAppliances) e.g., but not limited to a Bridge Router (BR-RTR, Router, Firewall, and DHCP-DNS (DDNS) across multiple virtual local area networks (VLANs) and potentially across data centers for scale, coupled through Compute node (C-N) nodes (aka servers). Cloud system virtual networks can comprise, e.g., software defined routers, Demilitarized Zone (DMZ) Firewalls, or both. A queue function/method in an RT/S data ingestion process can receive data packets/streams via a redundant system of messaging servers, the packets including instructions for/requests for information from NDS, data for analysis, or both, which the que function can process or relay to a controlling processor (controller), which can, e.g., determine capacity to process the packet, maintain the packet in the buffer until capacity is available, and deliver the packets to NDS/network components when ready for processing. Steps can include, e.g., checking other components, e.g., memory, processor, router, firewall, etc., for load/capacity, in determining availability. In aspects, a stream processor/message broker or other component of the NDS-INPU can generate a data stream constructed from data segments obtained from multiple respective sources (e.g., from 2 or more MAs associated with a subject). In aspects, similar processes are performed at an analytical level, e.g., in reconstructing MA-D from both cached MA-CD and before/after RT/S MA-D. Streaming analytical functions can include, e.g., selecting stream(s) corresponding to a parameter, parameters, or profile, indicative of a condition requiring immediate NDS action, e.g., based on comparison of stream data in-memory to such models, and relaying alert/alarm or device control instructions based on such event detection determination(s). Streaming analytics processes can also evaluate MA performance against similar parameters and assist in determination if an MA is operating properly, is being operated properly, or both, and take corresponding action. Such methods can comprise noninvasive extraction of 1 or more data features from data stream(s). Ingested data is stored in the NDS DR, which can comprise a DL (e.g., a cloud based scalable DL, e.g., Amazon's D3 DL), or an EDL. In aspects, a stream/streaming processor initial intake process can generally, substantially, or entirely be automatic, in generally, substantially, or all cases of operation. In aspects, initial queries/analytics/functions can operate on a substantially continuous basis in stream processor frameworks. In aspects, methods include the use of a live data mart, which is used to aggregate streaming data for query or other applications in real time or near real time. In aspects, streaming processor frameworks can comprise connectors for data conversion/translation. Stream processor clusters can store temporary data locally and transfer global data into and out of a stream register file. To enable multiple stream processors to be interconnected, the stream register file also can, in aspects, connect to a network interface, allowing entire data streams to be transferred from the stream register file of 1 processor to the stream register file of another processor through the Input Unit/NDS. By using software pipelining, each cluster can also process multiple stream elements simultaneously. The combination of data parallelism, instruction-level parallelism, and software pipelining can assist a stream processor to utilize large numbers of arithmetic units for media processing applications. In aspects, an NDS comprises vector processors (and possibly further vector memory/registers) along with or in place of stream processor(s). In aspects, near real time (NRT) processes may be understood as a degree of responsiveness that is sufficiently immediate relative to current events, or as sufficiently low latency or as a capability to keep-up with current events in a reasonably meaningful way relative to such events, but which is slower than RT processing in the applicable context. Additional methods, frameworks, applications, principles, and strategies/architectures applicable to processing of RT/S data are described in, e.g., US20150032879, U.S. Ser. No. 10/672,204, AU2015252037, U.S. Pat. Nos. 8,880,524, 9,270,937, 6,195,701, 7,512,829, 7,627,685, 7,827,299, 8,271,666, 8,689,313, and 9,158,775.

### II. Analytical Processing of MA-Data and Other Data

Processing of data by an NDS processor, such as MA-D, can be performed at varying levels, each level subjected to one or more processes which differ from any other one or more levels. E.g., raw incoming MA-D can be subjected to initial data improvement/enhancement, tagging, or curation, as described elsewhere, in a first level, and, in a second level, such initially transformed MA-D can be subjected to one or more analytical processes by an analytical unit of an NDS processor unit (e.g., an MLM).

An NDS analytical unit/analytics service can extract desired data from NDS DR, e.g., NDS DL/EDL DR and then implement the algorithm or algorithms that have been determined to be pertinent to fulfilling the on-demand real-time patient-specific data analysis request. To provide the processing power and speed needed to perform many algorithms on a large data set efficiently, the analytics service can utilize a computing cluster comprising multiple servers which provide the processing capability required to execute 1 or more algorithms. A cluster can be defined as a type of parallel and distributed system, which consists of a collection of inter-connected stand-alone computers working together as a single integrated computing resource. The computing platform of the NDS (the analytical unit(s)) can also include a data integration services module for processing, transforming, and validating data that is received from external entities e.g., MAs, HCPs, CMRSs, etc., prior to initial or secondary ingestion in the NDS DR. Parallel processing of data-intensive applications typically involves partitioning or subdividing the data into multiple segments which can be processed independently using the same executable application program in parallel on an appropriate computing platform, then reassembling the results to produce the completed output data. Data-parallelism can apply computation independently to each data item of a set of data, which allows the degree of parallelism to be scaled with the volume of data. In aspects, some component of the NDS employs a shared-nothing architecture, which ensures there is no single point of failure in at least some of the NDS environment (e.g., wherein each node operates independently of the others, so if one machine fails others keep running). In aspects, part of the NDS memory operates as a massively parallel analytic DR, e.g., a database employing columnar architecture(s).

Parallel processing in an NDS can operate mostly, generally, essentially, or only on a MIMD (Multiple Instruction Multiple Data) basis. In aspects, part of the processor operates on a SIMD (Single Instruction Multiple Data), e.g., in a vector processor. In aspects, the NDS/methods exhibit/comprise task parallelism, typically comprising independent branches running on separate threads and pipelines with many independent modules. In aspects, the NDS/network also exhibits pipeline-parallelism (e.g., typically in which pipelines are partitioned into subnetworks operating on different tasks, e.g., on streams). In aspects, an NDS also exhibits/is configured to apply data-parallelism, in which data elements are broken down and processed in parallel (and typically re-merged into a result/dataset).

In aspects, an NDS can ingest and initially process most, generally all, substantially all, or all MA-D received by the NDS within 1 second, e.g., within ≤~0.75 sec, ≤~0.5 sec, ≤~0.25 sec, ≤~0.15 sec, ≤~0.1 sec, or even less, e.g., within hundredths, thousandths, or millionths of a second, e.g., within time periods not humanly possible. In aspects, MA-D is critical care medical data. In aspects, an at least initial analysis of SMAD, e.g., for presence of alarm/alert conditions, is performed in ≤~2 minutes, ≤1 min, ≤0.5 min, ≤0.25 min, ≤~5 sec from receipt. In aspects, initial analysis is performed before or concurrently with ingestion of SMAD into the NDS DR.

### 1. Machine Learning

In aspects, the NDS-ANALU comprises executing CEI(s) for performing one or more machine learning (ML) methods on set(s) of data, e.g., data related to a particular condition, physiological parameter, or a CT. In aspects, development of ML method(s) comprises the steps of applying feature learning method(s), feature engineering method(s), or both to function(s) to develop ML method(s); applying supervised or semi-supervised learning/refinement of such ML method-implemented functions; applying reinforced learning, unsupervised learning to enhance such function(s); and eventually allowing function(s) or aspects of function(s) to be governed by the trained model. In aspects, machine-learned implemented functions can, e.g., characterize medical conditions. In aspects, machine-learned implemented functions can identify patterns or relationships and, e.g., unexpected data anomalies. In aspects, machine-learned implemented functions can also be used to predict matches, differences, events, etc.

In aspects, an NDS machine language module (an NDS-MLM) analyzes MA-D from homogenous or heterogenous types of MAs and optionally further performs or recommends the performance of one or more functions of the NDS based on the analysis of such MA-D. According to facets, machine learning (ML) can be applied to all data received or processed by one or more units of an NDS. In aspects, the NDS builds predictive functionality according to increased learning accomplished by the NDS-MLM over time. In aspects, the NDS-MLM can analyze and compare MA-D received from homogeneous MAs and, in analyzing what such data has been associated with in the past.

A variety of known ML algorithms/models are known that can be employed in such approaches, e.g., data classification methods, Naive Bayes classification (or Bayesian network methods), decision tree methods, decision rule methods, regression methods (e.g., logistic regression, lasso regression, SVM regression, ridge regression, or linear regression), random forest methods, support vector machine methods, and neural network methods, which are often employed in supervised ML methods. In aspects, ML models comprise method(s) often used in unsupervised or reinforced ML methods e.g., k-means (or variants thereof, e.g., K means++)/nearest neighbor analytical models, e.g., k-nearest neighbor analysis; other clustering methods (e.g., partitional clustering, mean shift clustering, density based clustering (e.g., DBSCAN methods), or hierarchical clustering (e.g., agglomerative clustering)); and multi-dimensional mapping methods, e.g., self-organizing mapping methods; and affinity mapping (e.g., for detection of events or prediction of events). In aspects, reinforced learning methods are applied e.g., artificial neural network methods. In aspects, ML methods include ML methods for decomplicating data e.g., decomposition methods, e.g., single value decomposition methods, dimensionality reduction methods (e.g., principal component analysis (PCA), Singular value decomposition (SVD), or TSNE), or a combination thereof. In aspects, ML methods employ model-free methods, e.g., in the context of reinforced learning, e.g., a Q-Leaning method. In aspects, MLIFs comprise model-agnostic methods, e.g., Partial Dependence Plot (PDP) methods, ICE methods, ALE plot methods, LIME methods, and the like. Other ML models that can be employed include partial dependence plot methods include, e.g., Generalized Linear Models (GLMs), Generalized Additive Models (GAMs) and the like. ML-implemented function(s) can comprise, e.g., deep learning methods, shallow learning methods, or a combination thereof.

In aspects, ML/AI applications (AIAs) applicable to step(s)/function(s) (S(s)/F(s)) of NDSs/methods can be characterized on the level of supervision of the MLM/AI application. In one aspect, MLM(s) is/are supervised learning (SL) MLM(s). In aspects, MLM(s) are semi-supervised learning MLM(s). In aspects, MLM(s) are unsupervised MLM(s). In aspects, MLM(s) are rewarded MLM(s). In aspects, an NDS/method comprises 2, 3, or all 4 of such types of MLMs. In aspects, SMGA or all MLM(s) of a method/NDS progress from one form of MLM to one or more other forms of MLM (typically less supervised form of MLM, e.g., by progressing from a SL MLM to an SSL MLM or an unsupervised MLM. In aspects, a MLM comprises key/feature recognition S(s)/F(s) based on training datasets relevant to the S(s)/F(s) performed by the method/NDS (e.g., data transformation e.g., tokenization, phrase/sentence/field segmentation, data structure recognition, or a CT), data cleaning, generation of queries (e.g., synonym recognition/application, stemming/truncation, lemmatization, metadata factoring, or a CT), determination of query matches/hits, deciding to enrich DS(s), and enriching of dataset(s)/DS(s)). Specific AIAs/MLMs, e.g., Naïve Bayes, Nearest Neighbor, Decision Tree, and related methods are described elsewhere and known. Conditional random fields (CRF) methodology also can be used in combination with training on relevant data sets in MLM feature engineering/identification step(s) of an MLM. Classification processes can comprise application of a Multinomial Naive Bayes (MNB) classification type algorithm. MLM processes also can comprise use of other clustering algorithms, e.g., mean-shift clustering, Gaussian mixture models, or DBSCAN. MLMs can be dynamically updated over time through feeding of updated training data, User feedback, Administrator input/supervision, or ACT. Training is typically directed to/performed on/using extracted or pre-identified Feature(s)/Element(s). In aspects, PHI/PII or other confidential information is removed from training information or replaced with altered data that is similar to/based on confidential/secret information (SI) data, redacted data, or a combination thereof (CT).

Machine learning models can be implemented and deployed using a machine learning framework known in the art, e.g., a TensorFlow framework, a Microsoft Cognitive Toolkit framework, an Apache Singa framework, or an Apache MXNet framework, or an equivalent thereof or a machine learning framework that performs similar or improved ML functions.

MLM training data in aspects can comprise focused/specialized corpus data (e.g., one or more models of MA sensor data from one or more types of MAs, one or more types of patients, or both) or interface or interact with other processes/resources that also are often characterized as MLM methods/resources, e.g., semantic networks (SN(s)), natural language processors (NLP(s)), or both. Supervised learning (SL) and semi-supervised learning (SSL) MLMs can comprise generation of a confidence score and assessment of the confidence score for the MLM against a threshold (e.g., an auto-tuned threshold), wherein failure to meet the confidence score threshold will route the test to an administrator for real-time review of the ML output. Subsequent administrator-performed or -managed tests/analyses, etc., can be fed back to the applicable Unit/Function to continue NDS processing and also fed back to the ML training set for inclusion or specific training/modification of the MLM. To facilitate ML/AI method(s)/function(s), an NDS can comprise neural network processor(s), or distributed processor(s) capable of being configured as a neural network, or capable of executing software to model or simulate neural networks, which may be used to implement/enhance machine learning.

MLM(s) can be trained by feature engineering (FE) and feature learning (FL) processes against training data, early application data, or both. Some, most, GA, or all MLM(s) of an NDS/method at least initially operate or operate on an SL or SSL basis. In aspects, in initial stages of MLM functioning, a higher level of human (Administrator) involvement can be typical to ensure improvement of the MLM function to comparable or detectably or significantly (DoS) better than average/all human performance. In aspects, the application of MLM(s) results in DoS improvement of performance of the Function with increased use of the Function; DoS improvement of human only performance (if even possible within relevant periods/accuracy), human programmed Function only performance, or both; or DoS improve any or all thereof.

An MA-D collection can be used as training data in a closed-loop fashion whereby the engine becomes continuously or iteratively trained using machine learning techniques in a supervised or unsupervised fashion. In aspects, if there is any inconsistency between the engine findings and Administrator (or research user/HCP user) or other relevant findings (including physician adjustments, confirmation, or rejections), a message can be sent to the MLM recording the event, providing for learning by MLMs, e.g., a ML medical data review system. This can, in aspects, eventually occur in an unsupervised fashion with engines providing feedback to other engines, still creating a closed loop learning scenario. In such cases, the first, second, and even third result may be provided from engines (or engines of engines, e.g., a master MLM that is applied to multiple MLMs or MLM(s) and other analytical processes) and not humans. In aspects, ML interpretations and corrected/validated findings can be captured as a combined dataset/cohort. Such data/training cohorts can be used by ML engines retrospectively to learn, and these data can be injected into the medical data review process by the medical data review system to further verify the performance of HCPs/devices or other non-ML analytical processes, further improve training data, and to develop new ML engines/MLMs.

In aspects, MLMs are used to predict patient conditions in one or more sensed conditions in a relevant period (e.g., the next day, in about 0.25 day, ~1 hour, ~0.5 hour, ~0.25 hour, ~0.1 hour, or ~0.05 hour (~3 min)). In aspects, a prediction MLM is provided specific subject sensor data related to the prediction as well as access to performance data in NDS memory or in the network, which can include data from research class operations or operations of other MAs under similar conditions. Research class devices can comprise research class MAs, which can be considered ONDs as they are not MAs in typical clinical use (though research MAs can be engaged in clinical trials for the development of new MAs, new indications, etc.). In aspects, the MLM is provided access to EMR data. In aspects, MLMs prediction data is relayed by the NDS/Relay Unit, and then back to the MA that the actual subject data was provided, to associated HCPs, etc. E.g., in aspects the prediction is one or more critical care conditions, e.g., cardiovascular conditions, e.g., those described in the Abiomed, Inc. patent references cited herein. In aspects, the MLM data is used for output applications, e.g., control of MA cardiovascular treatment tasks, pulmonary treatment tasks, or both, in relevant MAs. In aspects, the MLM DoS improves on the functioning of one, some, most, generally all, or all aspects of NDS or MA operation.

### 2. Timing of Data Cycles and Data Processing

Methods of the invention can comprise collecting/aggregating data, e.g., mostly or entirely RT/S MA-data, for initial analyses, post-ingestion/later applications (e.g., by U/F(s) of the Analytical Unit), or both. A period of collection of data used to form a collection/aggregate can be considered a collection/aggregation cycle, which is sometimes also simply described as a data cycle. In aspects, data collections for performance of functions (e.g., receipt of data)/initial applications can be used to form larger data collections for NDS analytical unit applications. In aspects, the time to form (i.e., a data cycle of) subsequent/secondary data collection(s) is substantially longer than the data cycle required to form initial data collection(s). E.g., in aspects, a secondary data collection cycle can be ≥5 times, ≥about 10 times, ≥about 20 times, or ≥~50, ≥about ~75, or ≥about ~100 times as long as the initial data cycle. In aspects, a secondary data cycle is ≥about 150, ≥about 250, ≥about 500, or even ≥about 1,000 times as long as an initial data cycle. In aspects, an initial data cycle (e.g., the time to process receipt of a data stream) is subjected to limited data analysis, which can be performed at an NDS input unit level, as described elsewhere herein (e.g., by a streaming processor application). In aspects, functions performed on the initial data cycle data include evaluation of the usability of the initial cycle data as a component of a secondary cycle data. In aspects, methods include rejecting and optionally restarting a secondary cycle data collection in which an initial cycle is rejected by the NDS based on preprogrammed standards. E.g., an NDS analytical unit can complete an MA-D analysis, e.g., application of MLM(s) to MA-D, ≤~10 min, ≤~5 min, ≤~2 min, or less than ~1 minute, and a secondary data cycle is correspondingly timed. In one aspect, the one or more functions of the NDS analytical unit are based on processing a collection of MA-D over a period, typically a period that is significantly longer than the period at which the NDS receives MA-D (an initial data cycle). In aspects such a period is ≥30, ≥40, or ≥60 sec of MA-D per MA-D collection iteration/cycle, e.g., ≥about 1 minute, ≥about 70 sec, ≥about 80 sec, ≥about 90 sec, ≥about 100 sec, ≥about 110 sec, ≥about 2 minutes, ≥about 2.5 minutes, ≥about 3 minutes, ≥about 3.5 minutes, ≥about 4 minutes, ≥about 4.5 minutes, ≥about 5 minutes or more of MA-D per iteration. In aspects, an initial data cycle is, e.g., ≤~40 seconds (sec), ≤~30 sec, ≤~20 sec, ≤~10 sec, ≤~8 sec, ≤~6 sec, ≤~4 sec, ≤~2 sec, or ≤~1 sec (e.g., 0.5 sec or 0.1 sec) and initial analytical processes are completed by streaming processor(s) or other engine(s)/component(s) in such a period. In aspects, NDSs comprise a set of Unit(s) or perform function(s) that can be similarly classified as a cache data processor, which processes MA-CD when such data is relayed from MAs.

Cache data processing can, in aspects, be performed differently than the handling of RT-SMAD and any initial analysis/ingestion, although one or more of such processes can alternatively comprise performing an initial analysis of cached MA-data (also called “CMAD,” MA-CD, or “cache data”) to evaluate if immediate output applications, e.g., alarms are triggered, based on such an initial analysis of cache data. Functions and related resources for performing such processes can be characterized as a cache data processor/unit. Processing of cached data can, in aspects, be performed using batch processes, for example, as such data can be delivered in a larger data set than a typical stream chunk/partition, is discrete (in start, end, and size), or both.

In aspects, an NDS input unit processes both streaming MA-D (SMAD) and MA-CD/CMAD in an integrated function or set of functions. Readers will understand that while RT/S-MA-D is extensively discussed herein such data can, in aspects, not be delivered on a real time basis, but still be delivered in a streaming manner, and such data can be classified simply as streaming MA-D or “SMAD.”

CMAD/MA-CD relayed/delivered to an NDS can be received in addition to the SMAD by an NDS input unit or a component thereof (e.g., an SDP/SDE or a component of an SDP, such as a specialized unit for analyzing MA-CD that can be characterized as a “cache processor”). An NDS input unit can in aspects distinguish MA-CD from RT/S-MA-D based on characteristic(s) of such MA-D, including relevant time stamp, association of related data, tagging applied at the MA level, or a CT. In aspects, methods of the invention include assembling cache data collections, using MA-CD to reconstruct missing data in the relevant SMAD for MAs (e.g., MAs impacted by periods of being offline), or a combination thereof. A cache processor that performs such steps/functions can be a component of the SDP processor, the primary NDS processor, or can be a functionally independent processor, physically independent processor, or both, with respect to an SDP and primary processor. The same relationship can apply to any memory specifically associated with the operation of the cache processor (e.g., a cache processor can comprise its own functionally/physically distinct memory or simply represent/work on memory in SDP or NDS-MEMU). In aspects, cache data is delivered in batches upon an event or passage of a regular interval. In aspects, a cache processor operates through, i.a., batch processing. In aspects, MA-CD relayed by most, generally all, or all MAs of the network comprises data in addition to location data, such as sensor data, time data, device operating characteristics, patient information, data related to an offline state, or any combination thereof. In aspects, MA-CD is transmitted periodically to the NDS as a back-up to RT/S-MA-D (e.g., permitting auditing of the RT/S-MA-D), is permitted on event(s) (e.g., detection of an offline MA state), or both. MAs can relay MA-CD concurrently with RT/S-MA-D or in batches in between periods of RT/S-MA-D transmission. An NDS will be programmed with settings to understand and adapt to the mode of transmission of MA-CD/CMAD from MAs.

In exemplary aspects, methods of the invention can comprise causing an NDS/MAC-DMS processing unit to automatically (1) collect MA-D for a data collection period to form a data collection, (2) evaluate if the data collection is suitable for analysis according to one or more preprogrammed standards/rules, etc., and (3) if the data collection is suitable for analysis, adding the data collection to a data aggregation; (4) repeating steps (1)-(3), until at least 2, 4, 5, 6, 10, 12, 20, 25, 50, 80, or 100 instances of data collection are added to the data aggregation so as to form a complete data aggregation, wherein (5) if any instance of data collection is determined to not be suitable before the complete data aggregation is formed, the method comprises discarding the incomplete data aggregation and re-initiating the method and (6) if a complete data aggregation is formed, the method further comprises performing one or more analytical functions on data comprising the complete data aggregation.

### 3. Scaling of System Resources

In aspects, function(s)/part(s) of an NDS are automatically scalable, e.g., in response to preprogrammed threshold(s) of demand, e.g., the number of MAs in the network; the amount of data transmitted from MAs on a basis (e.g., average amount per day, per week, per month, per quarter, or per year); the amount of analyses performed by the NDS, other operational functions of the NDS (e.g., security functions); the amount of data relayed from the NDS; the amount of data stored in the NDS; the timing requirements of data uptake, data processing, or data delivery in the network; or any combination thereof. In e.g., aspects, the NDS is mostly, generally only, or entirely “contained” in, or otherwise comprises, an automatically scalable cloud-based computer system comprising one or more special purpose units (e.g., specialized memory, specialized processing functions, or both), as described herein. Scalable resources typically include input resources (e.g., SDP resources), but can also include DR resources, processing resources, and the like. Thus, in aspects, the NDS comprises on-demand or automatically responsive expandable components.

In aspects, the NDS/method comprises a scalability testing function/step that anticipates scaling demand (e.g., by one or more parts of the NDS meeting or exceeding one or more indicative values/measures or other indicators), tests for the addition of such resources (optionally reserving such resources for meeting the anticipating demand) and automatically adds such resources when needed as determined by the detection of meeting/exceeding a second set of threshold indicators/values. In aspects, resources are added in a scaled/progressive manner, with optional testing of added resources as they are added, after they are added, or both, to ensure that scaling of the NDS is effective (e.g., that there is no detectable or significant reduction in NDS performance). In aspects, resources can be added on an as-needed basis, upon, e.g., reaching a predetermined threshold of demand, e.g., demand on a Unit, combination of Units, or the entirety of an NDS. In aspects, security or safety measures which ensure that an NDS remains operational, e.g., an NDS is rarely, if ever, rendered non-functional, due to lack of resources to address demand, are present within an NDS/method. In aspects, an NDS maintains sufficient resources or maintains access to sufficient resources to maintain operation at level(s) at or exceeding NDS demand ≥99.5%, e.g., ≥about 99.6%, ≥about 99.7%, ≥~99.8%, ≥about 99.9%, or even 100% of the time.

In aspects, methods include performing scalability testing, capability testing, or both types of testing of the NDS/network using actual MAs or simulated models. In aspects, actual scalability/capability test MAs relay status/mode data to the NDS in association with such testing, such that data from such test MAs can be appropriately tagged and curated/segregated from other MA-D (e.g., not being factored into MLMs).

### III. Regulatory Status of NDS-Data Applications

NDS applications can be subjected to different types of Regulations and related requirements (“RRs”) depending on the nature of the applications controlled by NDS applications. E.g., NDS applications can, in aspects, comprise applications that are regulated as software as a medical device (SaMD/SAMD) (i.e., software intended to be used for medical purpose(s) that perform these purposes without being part of a hardware medical device). In aspects, an NDS performs≥2, ≥3, or ≥~5 SaMD applications concurrently, in ≥1 MA types/classifications (e.g., in ≥2 MA types/classifications in the network). In aspects, NDS SaMD applications are diagnostic applications, therapeutic applications (e.g., applications considered as digital therapeutics), or both. Therapeutic applications can refer to applications that are therapeutic of an untreated condition or condition being initially treated, applications related to the maintenance of a condition, or medical tasks/applications that are related to the prevention of development of a condition (e.g., preventing the condition from developing, reducing the likelihood of development, reducing severity on development, or delaying onset of development, etc.) In aspects, most, generally all, or all the SaMD applications conform to IEC 62304 life cycle/design/development standards. In aspects, SaMD applications performed by NDS include a mix of IEC 62304 Class A, Class B, and Class C applications. In aspects, SaMD applications performed by an NDS include a mix of FDA minor concern, moderate concern, and major concern applications. Given the different regulatory nature and applicable RRs associated with possible NDS applications, NDS or NDS relay unit can employ tagging, conformance standards, other data curation/transformation methods (e.g., removal or protection of PHI), or any combination thereof, to ensure proper treatment of NDS applications consistent with applicable RRs. As such, methods can comprise the analysis of applications/output and the application of such data curation step(s). In aspects, most, generally all, or all SaMD applications of an NDS are only used in clinical settings upon Regulatory Authority review and approval. In aspects, NDS applications include ≥2 SaMD applications with different levels of SaMD approval/classification. E.g., in aspects SaMD applications provide HCPs with information to take therapeutic decisions or apply therapeutic-relevant device controls/parameters with non-critical or critical care diagnosis or therapeutic purposes (and is, e.g., regulated in the EU as a Class IIa device or Class III/Class IIb device, respectively, the latter depending on possible consequences of such decisions). In aspects, SaMD applications are directed towards monitoring of physiological processes (and regulated in, e.g., the EU as a Class IIa device or in critical care settings a Class IIb device).

In other aspects, one, some, most, generally all, substantially all, or all NDS data applications are applications not regulated as a SaMID. In aspects, NDS applications include a mixture of SaMD regulated applications and non-SaMD applications. In aspects, one, some, most, generally all, substantially all, or all NDS applications can be classified as CDSS (clinical support decision systems), which typically are not regulated as medical devices. A CDSS typically comprises a knowledge base (here, MA-D from a subject-associated device and from similar device/patient combinations), an inference engine (e.g., analytical U/F(s)), and communication means/channels (e.g., relay of NDS-AD to the MA, other HCP devices/interfaces in the network, or both). A CDS knowledge base contains rules and associations of compiled data which most often take the form of IF-THEN rules. Alert/alarm functions often are considered CDSSs. Recommended possible courses of action, provision of information, etc. also can be classifiable as CDSS applications. In aspects, the NDS employs an expression language e.g., GELLO or CQL (Clinical Quality Language) in the delivery of some or most CDSS applications. CDSS applications also can employ MLMs (where MLM only applications are employed, such AI applications can be characterized as no knowledge CDSSs). ML/non-knowledge-based systems can comprise, e.g., support-vector machines, artificial neural networks, genetic algorithms (using random sets of possible solutions, which are mutated, iterated, etc.), or a combination thereof. CDSS applications, SaID applications, or both, can comprise analysis of actual and delivery of predicted data, e.g., predicted A1c, predicted heart rate, predicted breath/oxygen concentration, or the like. Metadata tags applied by the NDS/NDS relay unit typically include regulatory status information. Delivery methods include screening of MA location to ensure that only approved SaMIDs are employed in a relevant country/jurisdiction. CDSS functions can include alerts/alarms, as well as provision of treatment/diagnostic recommendations, and similar Function(s).

In aspects, methods of the invention comprise applying core components of an NDS, outside of regulated SaID applications, to MAs, HCPs, and other class users, in other countries. Such application can comprise, e.g., making components of the NDS available to users in different countries, but segregating SaID applications to only users in countries in which such SaID applications are approved by applicable Regulatory Authorities. In aspects, a step/method of making core component(s) of an NDS (e.g., some, most, generally all, or all those component(s)/Engine(s) that are separate from Unit(s) that are classified as national or regional Regulatory Authority regulated SaMD applications) comprises sharing such core components between sub-NDSs or NDS portions/components focused on a particular regulatory regime. In aspects, an NDS, part of an NDS, or functions of an NDS are specifically limited to application(s) in a particular country or other jurisdiction (e.g., an international jurisdiction, e.g., the EU, or a regional jurisdiction, e.g., a state or province). In aspects, an NDS can comprise (a) data for MAs, e.g., MAs in a particular country, (b) a plurality of sets of rules, e.g., nation-specific data governance rules, or (c) both. In aspects, an NDS comprises a core architecture that is copied or shared among ≥2 country/jurisdiction-specific NDSs. E.g., in aspects, NDS blueprint data/core components can be shared with one or more other NDSs, either through copying or shared access over the internet, e.g., 2 or more other NDSs, as in, e.g., 3, 4, 5, 6, 7, 8, 9, 10, 15, 20, 25, 30, 35, 40, 45, or about 50 or more NDSs.

### IV. NDS Processes for Updating MA OS(s)/Software

MAs typically comprise operating systems (OSs) or specialized software that coordinates function(s)/engine(s) of the MA relating to data communications between the MA and the NDS. In aspects, the operating system (OSs); MA software (e.g., software relating to MA:NDS communications, application of MA functions such as therapeutic/diagnostic functions); or both, of some, most, generally all, or all MA(s) in a network are subject to update(s) during the operational lifetime of such devices. With respect to MAs, uncontradicted, the terms software and OS provide implicit support for each other here (e.g., an aspect describing updating an MA OS also implicitly discloses a corresponding aspect of updating MA software). In aspects, OS/software updates are triggered at a local/MA level, at an NDS/network level, or both. In aspects, some, most, generally all, or all updates to an OS/software of specific MA, type of MA, class of MAs (e.g., MAs associated with a particular Entity), or a particular zone/processor of an MZMA, require involvement (input, approval, or other action) at both a local/MA level and at an NDS/network level. In aspects, some MA system (OS/software) updates/modifications are only performable at a local level (e.g., in aspects a highly restricted therapeutic component of an MZMA is only updatable at a local level). In aspects, an NDS makes the availability of updates/modifications to MA systems known to users, e.g., through alerts/alarms or other messaging sent to the MA, other network interfaces/devices, or both. In aspects, MA system updates are mostly, generally entirely, or entirely relayed from the NDS over the internet and downloaded to the MA. In aspects, MA OS/software versioning is included in, or is required to be included in, some, most, generally all, or all MA-D streams/data, facilitating targeting of updates and update availability messages by NDS. In aspects, NDS monitoring of MA or zone software/OS can result in an output leading to the physical sending of an update to a local user, e.g., on a USB drive, external hard drive, disk, or other memory device, such that the local user can, if approved, make local modifications to such OS/software. In aspects, zone-specific processors are operated independently, updated independently, or both from other zone-specific processors (or other zone-specific processors and any primary MA processor), which can occur according to the various methods/procedures described above. In aspects processor(s) of an MZMA can be updated according to a method/process/procedure while a second one or more processor(s) of the MZMA can be updated according to a different method/process procedure.

The invention also provides methods for controlling operation of MAs so as to detect information concerning the operation of MAs in a network, e.g., to determine the level of MA performance, problems with MA performance, identify opportunities for improving MA performance, and the like. In such aspects, an NDS/MAC-DMS processor/analytical engine evaluate(s) the performance of the medical apparatuses in the network, the MAC-DMS, or both, in one or more respects. Such evaluations can be performed automatically, on-demand, or conditionally automatically, in an either continuous, repeating/routine/regular, or only on-demand manner. In an exemplary aspect, such a method comprises evaluating the MA-D of one, some, most, or all MAs against MA-D to previously collected MA-D analytical data, other/related standards (e.g., other measures of a physiological condition, such as subject heart rate, etc.), or to the performance of other MAs of the same or different type. In aspects, methods comprise changing at least one parameter of medical apparatus operation, NDS/MAC-DMS operation, or both, based on the evaluation of the medical apparatuses and the NDS/MAC-DMS. MA-D analyzed in such methods can comprise log data relating to device performance. In aspects, such log data is data collected in a restrictive zone of an MZMA, transmitted to a less restrictive zone, and thereafter relayed to the NDS for analysis (a flow of data that is also applicable to other MZMA-related methods). In aspects, data from a restrictive zone of an MZMA is identified based on data tagging (applicable to this and other MZMA-related aspects). In aspects, such methods are performed on an aggregate/collection of data, which can be collected over many minutes, hours, days, weeks, months, or years. In aspects, such log data/MA-D also or alternatively comprise subject-related sensor data. In aspects, such data is plotted against standards, etc., to identify abnormal patterns, outliers, and the like, which can be subject of NDS analysis and outputs. In aspects, such a method DOS increases the chance of identifying MA operation issues as compared to the identifiability of such issues at a single MA or even single MAG level. E.g., an analysis of MA-D comprising log data comprising heat pump-associated aortic pressure (AOP) or left ventricular pressure (LVP) measurements, unexpected sensor data can lead to the determination of a device operational issue (e.g., a problem with heart pump motor operation, such as motor current).

In another aspect, the invention provides methods of treating medical condition(s) of a subject/patient in a population of subjects/patients, comprising causing each operating medical apparatus (MA) of a MA:NDS data network to repeatedly routinely/automatically or conditionally collect sensor data from the subject/patient associated therewith; wherein the NDS analytical engine/function compares MA-D from some, most, or each operating MA of the MA:NDS data network to (I) the previously collected MA-D analytical data, (II) preprogrammed standards, or (III) both (I) and (II); and the method further comprises causing the NDS/MAC-DMS processor to relay one or more outputs via secure internet communication to one or more MAs, one or more ONDs, or both, the one or more outputs comprising (a) analytical data output relevant to the treatment of the patient relayed to (I) the MA associated with the patient, (II) another network device associated with a healthcare provided associated with the patient, or (III) both (I) and (II), (b) one or more output applications comprising instructions that control the operation of (I) one or more MA functions (e.g., therapeutic functions) in one or more of the medical apparatuses of the data network, (II) one or more other network device functions in one or more of the other network devices of the data network (e.g., registering alerts/alarms) that is associated with a healthcare provider associated with the patient, or (III) both (I) and (II). or (c) a combination of (a) and (b).

In aspects, methods comprise diagnosis of disease conditions also or alternatively to the treatment of disease conditions in MAs. In aspects, operation of the MA:NDS network detectably or significantly increases the probability of identifying a type of condition in subjects associated with MAs in the data network. E.g., by application of MLM(s) to MA-D collected by several/numerous MAs of a network, an MLM of an NDS can be trained to detect warning signs of a condition DOS faster than a human can detect such issues, faster than was previously possible in standard-of-care diagnostic devices, or faster than was previously possible with the NDS. This is one of the benefits of collecting large amounts of data via an NDS of the invention and using such a large data pool to generate NDS-AD, such as MLM prediction(s).

### ILLUSTRATED ASPECTS IN THE FIGURES

Additional aspects of the invention are described in this Section with reference to flowchart illustrations/block diagrams of methods. Readers will understand that generally each “block” of flowchart illustrations/block diagrams, and combinations of blocks therein, can be implemented by execution of CEI by processor(s). CEI units/functions reflected in such “blocks” are provided to processor(s) of applicable device(s)/system(s) to produce a machine, system, or both, such that the CEI executed by a processor implement the systems/functions specified in the block(s). Such CEI are stored in CRM (e.g., NTCRM/PTRCRM) that can direct the applicable computer(s) to function in a particular manner according to the CEI, such that the CRM comprising DR(s) (comprising functional data) and CEI(s) comprises an article of manufacture that performs useful real-world operations.

Although aspects exemplified here are described with reference to flow charts/block diagrams, any portion or combination of each block, combination of blocks, functions, etc., can be combined, separated into separate operations, or performed in other orders, as would be suitable in the context of an NDS or method of the invention. References to “modules,” “units,” “steps,” etc. in this disclosure typically are made for convenience of the reader and are not intended to limit implementation of any method or NDS. Any portion or combination of any block, module, etc. can be implemented as computer executable (program) instructions (e.g., software), hardware (e.g., combinatorial logic, Application Specific Integrated Circuits (ASICs), Field-Programmable Gate Arrays (FPGAs), processor(s), or other hardware), firmware, or any combination thereof (CT). In other words, flowcharts, block diagrams, etc. reflected in the Figures illustrate architecture, functionality, and operation of possible implementations of NDSs/methods. Each block in a flowchart/block diagrams may represent a device, component, module, segment, CEI, or portion of CEI, for implementing the specified step(s)/function(s). In aspects, step(s) of methods or arrangement of function(s) described in respect of such blocks may occur in an order different from that set forth in the Figures. E.g., two blocks shown in succession may, in fact, be executed substantially concurrently, or the blocks may sometimes be executed in the reverse order, depending upon the functionality involved. Readers will note that each block of block diagrams or flowchart illustrations, and combinations of blocks in block diagrams/flowchart illustrations, can be implemented by special purpose hardware-based systems that perform the specified functions or acts or carry out combinations of special purpose hardware and computer instructions.

Different programming techniques can be employed e.g., procedural or object-oriented approaches in NDS(s)/method(s). Any particular routine can execute on a single processor, multiple processors, or even in multiple devices, as suitable. Data can be stored in a single storage medium or distributed through multiple storage mediums and may reside in a single database or multiple databases (or other data storage techniques). Thus, although the steps, operations, or computations may be presented in a specific order, this order may be changed in alternative aspects and any of the specifically disclosed routines/workflows provided here can comprise rearrangement, repeating, skipping, combining, or any combination thereof (CT) of one or more step(s)/function(s) to provide the indicated output. In embodiments, to the extent multiple steps are shown as sequential in the Figures, some combination of such steps in alternative embodiments may be performed at the same time. Where suitable, any sequence of operations described herein can be interrupted, suspended, or otherwise controlled by another process, e.g., an operating system, kernel, etc. The routines can operate in an operating system environment or as stand-alone routines. Functions, routines, methods, steps, and operations described herein can be performed in hardware, software, firmware, or any CT, e.g., as described herein.

For these and other reasons, the illustrative description of aspects in the Figures and described here with respect to the Figures should not be used to limit the scope of the invention as provided by other portions of this disclosure or the disclosure read as a whole in view of the art.

Displayed Figure elements are typically identified with the “#” symbol in the following. Where reference to an element is repeated in a Figure description, additional element reference(s) are sometimes omitted. The abbreviation “n.s.” refers to features/steps that are not shown in the Figure.

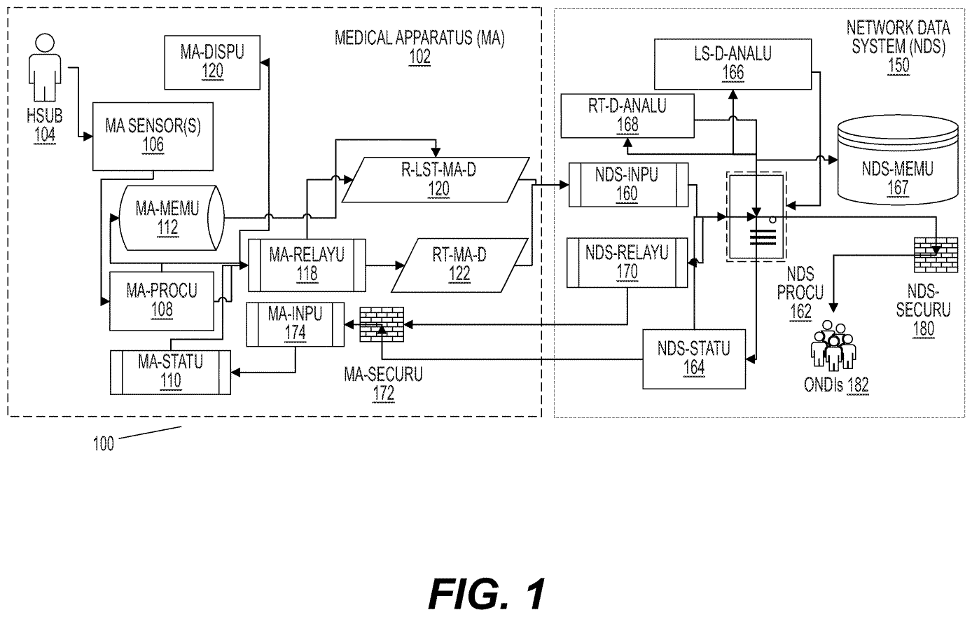

### FIG. 1

FIG. 1 illustrates an exemplary basic relationship #100 between a medical apparatus (“MA”), #102, and a Network Data System (“NDS”), #150. Arrows in this and similar Figures provide a non-limiting illustration of flow of data through a system, network, or network relationship (as shown in this Figure). Lines around components (e.g., MA-MEMU, #112, and MA-DISPU, #120) in the Figure indicate that such components are part of or associated with another shown component/system (here, either the MA or the NDS, as applicable).

MA, #102, can be, for example, a blood pump, e.g., a heart pump, or a cardiopulmonary support system, e.g., a portable extracorporeal membrane oxygenation (ECMO) system, associated with, and usually in therapeutic contact with (often at least partially inserted in) a human subject (HSUB, #104), and comprising electronic/computerized hardware for data collection, storage, processing, transmission, and receipt. MA, #102, comprises sensor(s), #106, which each detect physiological condition(s) in HSUB #104, operating conditions of MA #102, or both. Sensors can include, e.g., flow sensors, pressure sensors, and other sensors associated with medical devices, which collect such data and relay it electronically to the MA processor(s) (MA-PROCU, #108), which is composed of computer processor(s) for processing stored instructions, performing analysis of data, and performing other function(s) as will be described throughout this section. Sensor(s) typically measure physiological states (conditions) (e.g., blood pressure, heart rate, oxygen level, breathing rate, blood agent level, brain function, etc.) as well as other types of sensed data. The MA-PROCU #108 comprises or is associated with component(s) that operate as an MA status unit (MA-STATU, #110) that regularly sends status signal(s)/communication(s) from MA to NDS (e.g., a ping signal or the like), via electronic communication, according to programmed instructions, e.g., the transmission of status signal packets in a secured internet communication transmission, typically over wireless communication (e.g., via Wi-Fi). MA-PROCU #108 records sensor data in a local device memory unit MA-MEMU, #112, which also can contain stored instructions for the functions/modules executed by MA-PROCU. MA-PROCU, #108, also comprises or is associated with component(s) that can constitute a relay unit (MA-RELAYU, #118), which serves to transmit information from MA #102 to NDS #150, e.g., a network interface card/controller (NIC). When online, information relayed by MA-RELAYU comprises locally stored/cache data (R-LST-MA-D also referred to here as MA-CD or L-STR-MA-D, #120) and streaming “real time” MA data (RT-MA-D, #122).

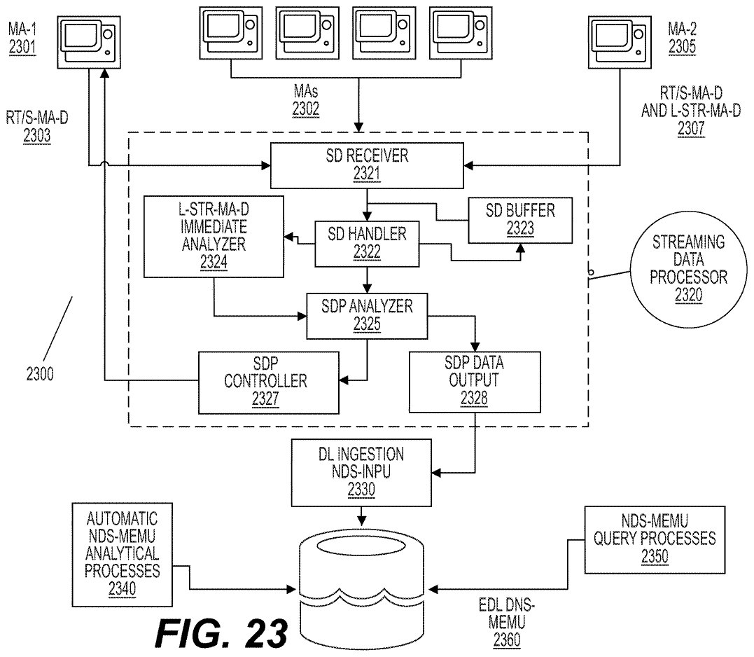

NDS, #150, comprises an input unit (NDS-INPU, #160) that receives, and processes received information relayed from MA, #102. The input unit may represent instructions (protocols/functions) in a virtual server environment for the receipt and processing of MA data or separate physical processor(s), memory, or both (e.g., in the case of an SDP, as, e.g., described in FIG. 23). Received MA data is relayed to the NDS processing unit (NDS-PROCU, #162), which typically will comprise multiple, massively parallel, highly available, virtual/cloud-based, scalable, and distributed processors (reflecting actual processing components, typically in one or more hosting facilities). NDS-PROCU, #162, will typically further comprise or interact with a NDS status unit (NDS-STATU, #164), that transmit status inquiries or receives status signals relayed from MA-STATU, #110, thereby determining status of the networked MA, #102 (status system(s)/component(s) also or alternatively can be contained in an MA). NDS-PROCU can further comprise engines/modules/units/functions for analysis of received data, such as locally stored data analytical unit, LS-D-ANALU, #166, which analyzes locally stored MA data (MA-D) (#120) (aka, cache data, R-LST-MA-D, etc.) and RT-D-ANALU, #168, which analyzes real time/streaming MA-D (#122). Such analytical units (“ANALUs”) can comprise, mostly comprise, consist essentially of, or consist of, e.g., components of memory (e.g., specialized software instructions/engine(s)) in combination with processor(s) that when put in operation form and operate as specialized devices for the handling of such particular types of MA-D. NDS, #150 also comprises an NDS memory unit, NDS-MEMU, #167, which can comprise, primarily comprise, generally consist of, or consist/consist essentially of one or more DR(s), such as a data lake (DL) or enhanced data lake (EDL). The NDS memory unit, i.a., stores received data, analyzed data, and instructions for execution by NDS-PROCU. NDS-PROCU, #162, further comprises or is operatively linked to a NDS data relay unit (NDS-RELAYU, #170), which is responsible for transmission of data to MAs and to other network devices/interfaces (ONDIs, #182) (e.g., system administrators), typically after such data “passes through” (is analyzed/screened by) an NDS security unit (NDS-SECURU, #180), which filters and restricts data relayed from NDS, e.g., to ensure protection of PHI in accordance with regulatory requirements (“RRs”). MA-D received by NDS #150 in such an aspect can be analyzed by one or both of the referenced analytical unit (ANALU) component(s) (#166 and #168) of NDS-PROCU, #162, and the analytical output (NDS-AD) generated by such an analysis can be relayed (1) to the MA, #102, via an MA input unit (MA-INPU, #174) (which can comprise, e.g., an NIC card or similar component/system and related engine(s)), often after passing through a device/MA security unit, such as a device-level firewall or security system, #172, and (2) to other network devices/interfaces (ONDIs, #182). Real time MA data #120 can mostly/typically/essentially be analyzed in a streaming fashion except for when MA #102 is in an offline state, in which case MA-CD/cache data stored during the period that MA #102 is offline is relayed to NDS #150 for processing functions, e.g., reconstruction/harmonization of such stored cache data with RT data received before and after the offline event.

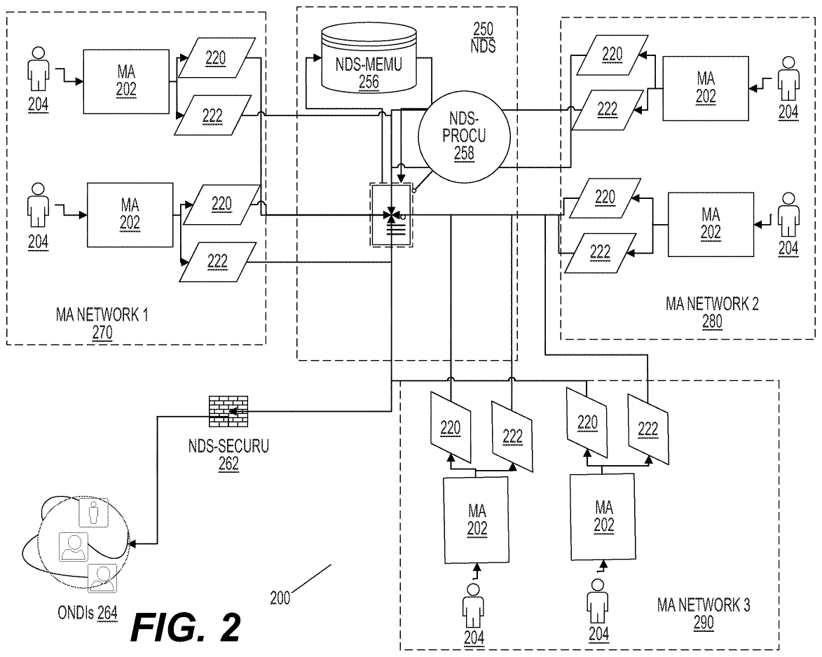

### FIG. 2

FIG. 2 provides an overview of a simplified representation of a network including groups #200 of MAs #202, each MA, #202, being associated with a subject/HSUB, #204, and each MA transmitting real time streaming MA data (RT-MA-D, #220) and, at least sometimes/occasionally, also transmitting locally stored/cache data (L-STR-MA-D/MA-CD, #222), to an NDS, #250. The illustrated NDS comprises a memory unit, NDS-MEMU, #256 and processor, NDS-PROCU, #258, as well as a security unit/engine/system (NDS-SECURU, #262) that ensures compliance with regulatory requirements (RRs) in transmitting data derived from the MAs to ONDIs, #264. In the illustrated exemplary network, MAs are configured into groups/sub-networks (sometimes also called networks), e.g., MA network 1, #270, MA network 2, #280, and MA network 3, #290, with each network comprising multiple MAs (e.g., excluding PHI). Lines around MAs in the Figure aid in defining the MA group/network. Each of such MA networks (which may be called “sub-networks” to avoid confusion with an overall network comprising multiple MA sub-networks/OND subnetworks and an NDS) can be characterized/defined based on owning/controlling entity, region, or other characteristics, e.g., geographic area, device composition, etc. Sub-networks/groups of devices in the overall NDS network can be organized at different levels (e.g., an Independent Entity Sub-network can include regional Sub-networks, hospital Sub-networks, etc.). Sub-networks can comprise entry points/nodes (not shown), which include security systems (e.g., firewalls), etc. MAs in a sub-network can relay sub-network-specific data (e.g., confidential information accessible to the Independent Entity that owns or operates the MAs in the group/sub-network). NDS-PROCU #258 processes data from all associated groups/networks, as well as data stored in NDS-MEMU #256, and delivers analytical results obtained from analysis of such data back to each group/network associated with the MAs therein, but also uses the combined entire network data in performing general analytical processes that can be used in the delivery of data back to MAs/MA users, ONDIs, or both. Such a configuration of systems of the invention allows the system to utilize information from across diverse groups/sub-networks, thereby improving analysis of the overall system (e.g., in terms of machine learning applications), while ensuring that confidential information accessible only some Independent Entities (IEs) are only accessible by the appropriate entities. Such information can be relayed to, e.g., other network devices/interfaces (ONDIs), #264, which can be associated with, e.g., commercial class users, associated with the owner/operator of the NDS, and which provide support/services to two or more IEs associated with different MA groups, after passage through NDS security processes (NDS-SECURU, #262), which ensures that certain information, such as PHI, is not relayed to such ONDIs. In this respect, the NDS (and also or alternatively other devices in the network) are configured to maintain confidentiality of confidential information in the network in at least two different levels of operation by ensuring appropriate identification of data in the network, screening information for such identification, and applying redaction, routing, modification, etc., to protect such information from undesired/inappropriate access/distribution.

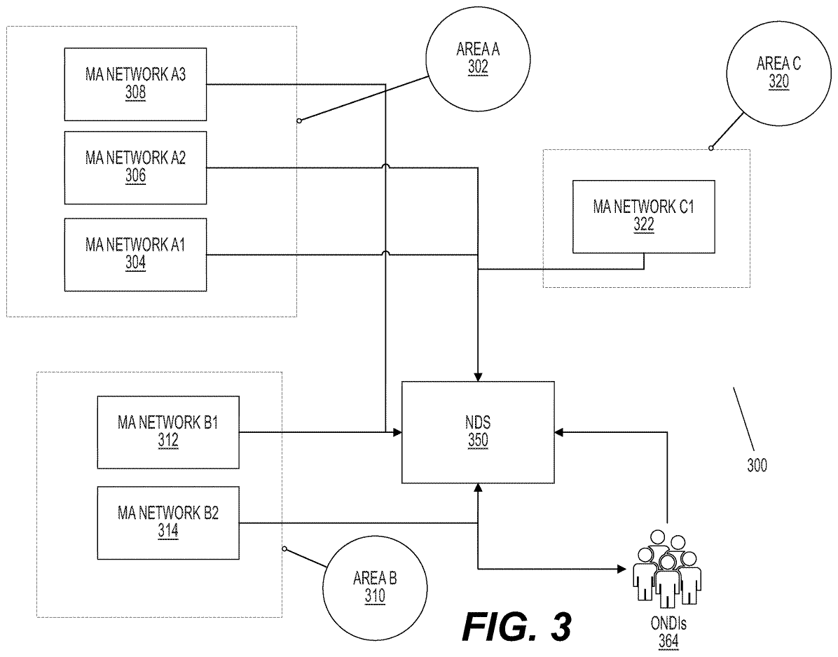

### FIG. 3

FIG. 3 is another simplified diagram of one level of an MA network/group comprising several MA subnetworks associated with different areas (metropolitan regions, states, counties, countries, hospital networks, etc.), reflecting another aspect of operation of systems/networks of the invention. Area A, #302, for example, is shown as comprising three MA groups/networks (MA network/group A1, #304, MA network A2, #306, and MA network A3, #308) (as designated by boxed regions of the Figure), each MA network comprising a plurality of medical devices (e.g., ≥5, ≥10, ≥20, ≥30, ≥40, ≥50, or ≥100 MAs per network), and such MA sub-networks/groups possibly being associated different independent entity (IE) owners (from each other, from the NDS/system owner/operator, or both). Separate Area B, #310 comprises MA group/network (MAG) B1, #312 and MA network/group/sub-network B2, #314, whereas also distinct Area C, #320 comprises only MA network C1, #322. In operation, NDS, #350 receives data from each MA group/network, such data including location information, affiliation information, or both, concerning the MAs in each area/network (e.g., in transmitted packets sent from such MAs, e.g., may be sent in a status signal or other transmission to NDS, #350), thereby permitting NDS #350 to identify the source of data, allowing NDS #350 to return data specific to a particular patient at the MA or MA user level, but also to provide MA network/group or area-level analysis to other network components (e.g., ONDIs, #364), e.g., network/Area/NDS administrators or support staff, e.g., medical science liaisons, salespeople, researchers, NDS/system analysts, or clinical support personnel, studying or observing performance at a network/area level or performing comparative studies between networks or areas. The identification of such area of client devices in the network permits the NDS to perform MA group-level analyses, assists with relay of confidential information to only appropriate recipient devices, or allows NDS to only relay applications that are approved under regulatory requirements in the particular group/area (or comply with requirements of IEs that own/operate MA groups). In such aspects, NDS and network devices are configured to provide data that enables the rapid identification of MA-D as being associated with such different levels of origin (particular MA/patient, group, region, etc.), permitting NDS to perform analysis at such differing levels or provide outputs to users based on such levels, concurrently.

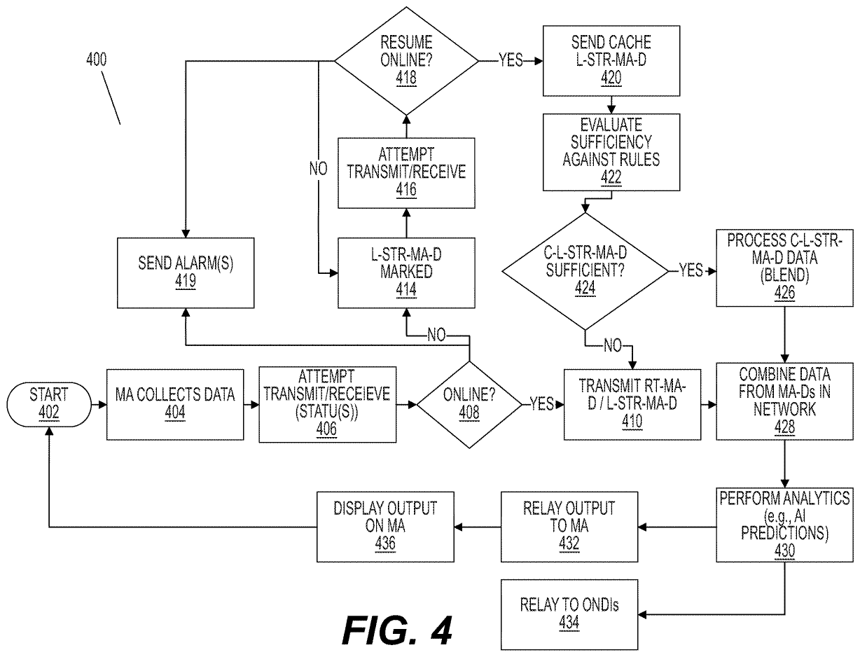

### FIG. 4

FIG. 4 provides an overview of exemplary processes that can be performed by systems (NDSs) in dealing with the issue of potential MA offline periods. At the start, #402, of the depicted exemplary process #400, MA(s) collect(s) data from subjects, #404, e.g., through MA sensor(s). MA(s) in the network regularly attempt(s) to transmit or receive status information from NDS via a status unit (MA/NDS STATU(s) #406) (e.g., periodically transmitting status signals when in one or more operational states/modes). If MA is online, #408, MA transmits RT-MA-D (and possibly also MA-CD), #410. If an MA is not online, MA is adapted to automatically mark (identify/record) the start of the offline state. And MA either continues or initiates collection of MA-CD/cache data #414, storing such cache data in MA memory. In the offline state (or regularly at essentially all times in operation), MA continues to transmit/receive status signals to NDS, #416. If MA is not restored to an online state in a preprogrammed time, #418, MA may through a controller (n.s.) transmit one or more alarms, #419, to 1 or more user(s). If MA resumes an online state, #418, MA transmits cached MA-CD (cache L-STR-MA-D), #420, to NDS. NDS-PROCU or a component thereof, evaluates sufficiency of the cached MA-CD against one or more sufficiency standards/rules or algorithms/protocols, #422, to make a sufficiency determination concerning the use of the cached L-STR-MA-D/MA-CD in ongoing analytical processes associated with the MA-D, network, area, etc., #424. If the data is not sufficient, MA-D may reinitiate transmission of RT-MA-D (possibly also in combination with L-STR-MA-D/MA-CD), #410. If the stored data is deemed sufficient the NDS-PROCU will process the cached-MA-CD (aka, C-MA-CD) (e.g., combining/blending such data with other network stored data (N-STR-D), #426). Such blended data can then be further harmonized/blended with data of other MAs in the network, #428, and the NDS-PROCU will use such data to perform one or more analytical processes, e.g., AI/ML processes, #430, the results of which will be relayed back to MA(s) #432, and displayed in each case on an MA display #436, and data derived from such analysis can also or alternatively be sent to ONDIs #434. Typically, such a process will reinitiate in a repeating manner during MA operation.

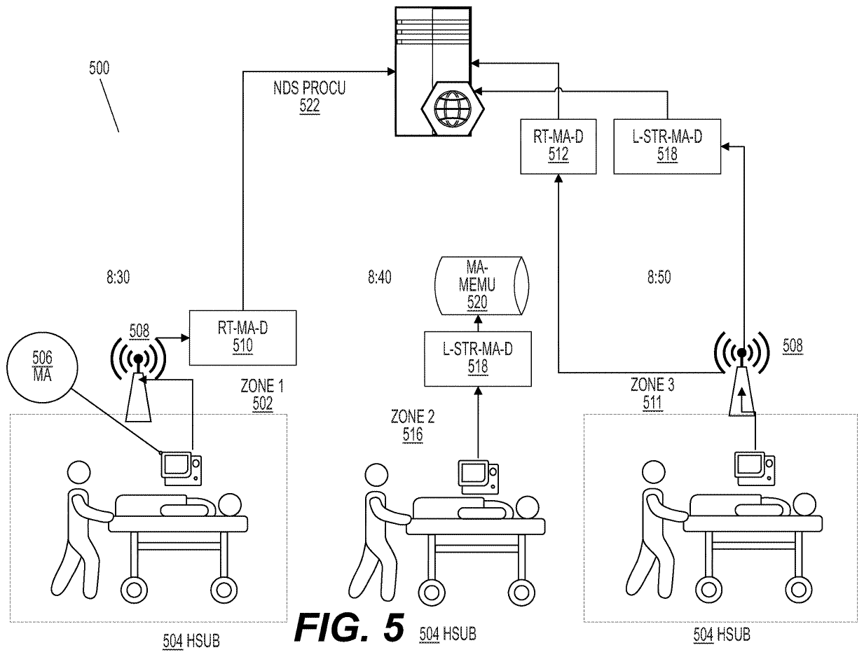

### FIG. 5

FIG. 5 illustrates a scenario #500 that further illustrates the collection and possible processing of cache data/MA-CD/L-STR-MA-D, like FIG. 4, but here with reference to exemplary physical components of parts of the network and locations of a portable MA in operation. A human subject/patient (HSUB), #504, is diagnosed/treated with MA #506, while being transported through Zone 1 #502. MA relays real time streaming MA-D (RT-MA-D #510) to the NDS processor (NDS-PROCU #522), via wireless network point #508 at a first time (8:30). However, at a second time (10 minutes later/8:40), when subject is in Zone 2, #516, which lacks wireless communication capabilities, MA begins to collect tagged/marked L-STR-MA-D (aka, MA-CD or cache data), #518, and stores it in apparatus memory (MA-MEMU, #520). When the subject reaches the location identified as Zone 3, #511 (at 8:50), which is again associated with a wireless relay, #508, MA delivers both new RT-MA-D #512 and the L-STR-MA-D #518 stored during the period when MA was in Zone 2. Additional data such as known or anticipated cause of network communication error can also be relayed, sensed, or predicted, if sufficiently determined/detected by MA/NDS. As temporary offline states are possible either in such transport settings or when there are network or device issues/disruptions, the capability of an NDA/MA network to effectively store, relay, and process such cache data as well as real time MA-D greatly enhances the effectiveness of applications on MA data, resulting in better data, better NDS operation, and better patient care.

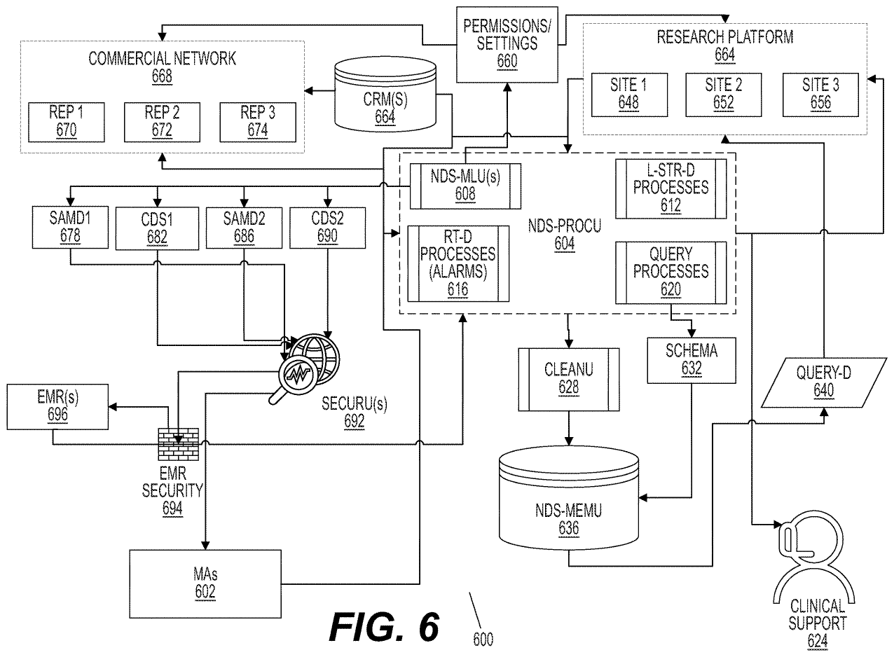

### FIG. 6

FIG. 6 illustrates an aspect in which at least part of an MA/NDS network, #600, which in addition to comprising MA network #602 and NDS comprising, i.a., an NDS processor (NDS-PROCU, #604 and NDS memory, NDS-MEMU, #636), further comprises other network devices/interfaces (ONDIs), exemplified in the illustrated system #600 as a commercial network/group/component, #668, a research group/platform, #644, and a clinical support component/group, #624.

While each of these other network/NDS components, like MA network #602, utilize either MA-D, NDS-AD, or both, concurrently/simultaneously, each such other network/NDS component also typically receives different aspects of such data due to Regulatory Requirements, confidentiality concerns, and different data needs/roles of users in such networks/NDSs. Accordingly, NDS processor (NDS-PROCU #604), not only receives MA-D from MA network #602, applies data improvement functions to such data (e.g., cleaning, harmonizing, etc., #628), and stores such data in memory (NDS-MEMU, #636), but also accesses programmed instructions (engine(s)) in PTRCRM of NDS-MEMU #636 through included processing components that employ such instructions/code to efficiently tag, sort, clean, and analyze MA-D, generate NDS-AD, and to effectively and securely and concurrently distribute NDS-AD and related controls/instructions (applications) to such various components of the network, within a limited time window (e.g., <10 minutes, <5 minutes, <2 minutes, <1 minute, <30 seconds, or <20 seconds of each other) while ensuring compliance with Regulatory Requirements (RRs) and other rules (e.g., NDS-PROCU #604 can comprise output function(s) that ensure output of only data targeted for/delivered to various network components/devices is appropriately delivered to only authorized/appropriate other network component(s)).

Commercial network #668 comprises a network of devices or interfaces (e.g., web pages accessible over the internet on any number of suitable devices) that are accessible by professionals working in commercial roles (represented by, e.g., Rep 1, Rep 2, and Rep 3, #670, #672, and #674, respectively), supporting commercial roles, or interfacing with commercial roles within a healthcare organization that manages/owns the network #600, such as salespeople, medical science liaisons, marketing professionals, business intelligence professionals, etc. Company that owns/manages the system, network, or both, typically also is a company that manufacture(s)/distribute(s) MA(s), sells NDS operation services, or both. In aspects, some, most, generally all, or all professionals accessing devices/interfaces in such a commercial network are in a commercial sales and marketing role for either the network manager or an organization that otherwise can access the NDS/system. Regulatory requirements (RRs) for such professionals can be significantly different from other users of such a system (e.g., HCPs treating a patient with an MA), such as with respect to access to PHI. Accordingly, NDS-PROCU #604 components, e.g., machine learning components (NDS-MLU #608), are adapted/configured to apply different rules in aspects to output provided to commercial network #668 than is provided to, e.g., MAs #602. Specifically, users of commercial network/group, #668, can receive data display(s) structured user role, function, and applicable RRs/other requirements, which is significantly different in content or form from, e.g., displays of data provided to HCPs using and accessing MAs, #602. E.g., Rep 1, #670, may support several hospitals in a hospital network and, accordingly, will receive a display of data on the performance of MAs generally in each hospital, with individual device data limited to high level aspects of performance (usage, errors, status, etc.), without revealing PHI. Rep 2, #672, can receive similar information for a distinct, same, or overlapping network of sites, entities, or networks. The devices of the commercial network can further interact with independent data sources, e.g., Customer Relationship Management (CRM) database #664 (e.g., a Salesforce™ CRM database), which can both provide data directly to NDS-PROCU #604, but which also can provide data to commercial network user interfaces/devices, where such data can be, in aspects, blended by local components of such interfaces/devices, NDS, or both, to provide a combined display of information to such users (e.g., Entity information and sales information from the CRM in combination with usage and clinical outcome information in NDS-AD).

HCPs may receive data from one or more NDS-PROCU functions that are regulated by a Regulatory Authority, e.g., through software as a medical device regulation. E.g., a first software as a medical device application, SAMD1, #678, performed by the NDS processor/engine(s), may provide therapeutic directives to HCPs, whereas a second application, SAMD2 #686 can directly control one or more operational functions/parameters of MAs. Other NDS-PROCU functions employed at an MA level can include clinical decision support (CDS) systems (“CDSSs”). E.g., a first CDSS, CDS1, #682, can provide possible treatment options based on detected MA conditions, received by NDS-PROCU as RT-MA-D, analyzed by both RT-D processes #616 and machine learning unit(s) (NDS-MLU, #608), the latter drawing data from many MAs in the network #602, historic MA data stored in NDS-MEMU #636, or both. The relationship to these components of the NDS to the overall NDS processor, #604 (or primary NDS processor) is reinforced by the box surrounding such components. A second exemplary CDSS, CDS2 #690, comprises an analytical support function that draws from both MA- and other data stored in patient EMR(s) #696 (and optionally NDS sends output to EMR(s)). Exemplary support functions can include providing warnings when MA setting changes appear inconsistent with treatment state based on the CDSS, providing recommended MA settings or other courses of action, or providing alarms based on RT-D processes, #616. EMR data is delivered to NDS-PROCU #604 in a highly secured manner by passage through an EMR Security Unit #694, which can comprise 1 or more firewall(s)/filter(s), e.g., performing packet scanning to identify data packets with acceptable elements while also scanning out potential malicious code/viruses, encryption, and, accordingly, NDS-PROCU #604 will typically comprise function(s) for the recognition of EMR data and routing of such EMR data to avoid access to users of the NDS other than authorized HCP(s). NDS-PROCU #604 can apply, e.g., tagging of data, routed to CDSS vs. SAMD processes, both to provide only such data needed for the individual CDSS/SAMD process and in view of the different Regulatory Authority status of such processes. For example, security or accuracy of different SAMD functions (#678, #686) typically will be demonstrated, validated, and maintained/audited at a level (by the NDS, users, or both), that is above (more intensive/restrictive) what will be required for CDSS processes (#682, #690), especially for SAMD processes that directly control aspects of MA processing. Additional security unit(s) (SECURU(s)) #692, can be applied at the network level, MA level, or both, e.g., firewalls/filters, to ensure only appropriately tagged MA data is delivered for each MA and to each MA function.

Research platform/group/network, #664, similarly typically comprises several users, user organizations, or both. As exemplified here, Research platform #664 comprises a plurality of networks of clinical research sites/devices (here illustratively shown as Site 1, #648, Site 2, #652, Site 3, #656), which typically represent devices/systems/groups managed by different independent entities (e.g., different clinical research organizations, university hospitals, or other organizations involved in clinical research) or even different networks within an entity (e.g., a team working on a particular device or clinical study). Data accessible to research platform #664 can be subject to significantly different RRs than commercial network #668. Modifications to data delivery, access, etc., to support compliance with RRs and other requirements (e.g., contractual requirements) can be governed by, e.g., permissions/settings and applying filters/firewalls (or, permissions/settings) #660 that are applied to, i.a., output of NDS-PROCU, #604. Research platform users also may have access to some, most, or generally all NDS-PROCU functions, e.g., data repository query processes #620, which can allow for querying data stored in NDS-MEMU #636, and which are inaccessible to, e.g., users in the Commercial network #668. Query processes can comprise applying one or more schemas, #632, to data contained in part(s) of the NDS-MEMU #636, which stored NDS data can be, e.g., semi-unstructured data stored in a data lake (DL) format or EDL format, as discussed/exemplified elsewhere, resulting in query response data (Query-D, #640) that is delivered to research platform user(s). Additionally, unlike commercial network #668 users in many aspects, research platform users can in aspects directly provide data to NDS-PROCU, e.g., clinical research data, modeling data, research analytical data, etc. Thus, for example, some NDS analytical processes can use data from research platform, e.g., in the development of machine learning models, where research platform data is a source, the main source, or the only source of information for information for such models. Such models may be limited to certain applications given the research nature of data from the research platform. E.g., such data might be used to in CDSSs, but not be suitable for use in a SAMD function.

Clinical support network/group/component, #624, typically comprises other devices/interfaces (ONDIs) accessible by professionals engaged in monitoring status of MA patients in one or more therapeutic setting(s), e.g., in a professional clinic (MAs #602), research platform setting #644, or both. With respect to users in clinical support component/team, #624, access data from NDS-PROCU #604 can be subject to different rules than other class of users (e.g., commercial users), such users provided with different displays of MA/NDS data (MA-D, NDS-AD, or both), and be accordingly provided different profiles/tools/interfaces than users in either commercial network #668 or research platform #644 (though the application of different schemas on output) (but possibly with more overlap with interfaces delivered to HCPs accessing similar data at MAs, ONDIs, or both). Clinical support personnel can be trained to provide additional support when receiving an alarm based on RT-D processes #616 (data analysis functions performed on streaming MA-D), based on MA status information, based on processes performed on locally stored MA-D uploaded after offline event (#612), or both. In aspects, clinical support personnel are associated with (employed or contracted by) the NDS owner/operator. NDS owner/operator employees and contractors can be subject to legal requirements that provide assurance of confidentiality and compliance with applicable RRs.

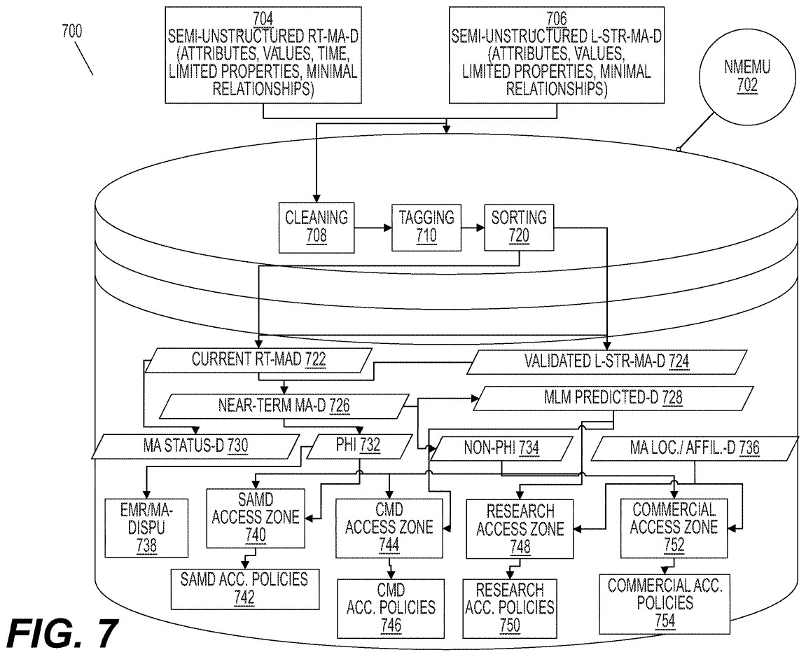

### FIG. 7

FIG. 7 illustrates data management processes and collections of data #700 associated with and mostly taking place/being contained in, or otherwise being associated with, at least part of an exemplary NDS/system memory unit (NMEMU, #702). NMEMU receives both streaming real time medical apparatus data (RT-MA-D) #704 and data locally stored in medical apparatus memory (LSTR-M-AD, #706, aka, cache data/MA-CD). RT-MA-D typically is delivered in a real time (or near real time) manner, in a streaming format, or both. MA-CD is typically delivered through batch data delivery, either periodically, upon an event (e.g., an offline event, as described above), or both, or can be combined with RT-MA-D and sent in a streaming manner once streaming is available (e.g., once an MA is restored to online status). RT-MA-D typically is delivered as semi-unstructured data (SUMAD), unstructured data, or often a combination thereof (e.g., status information, #730, which can be stored as a discrete data collection and analyzed as such, may be unstructured, but attributes and value pairs e.g., location: location information, device type: heart pump, etc. typically are delivered as SUMAD). MA-CD also can be delivered as SUMAD, structured MA-D, or both. SUMAD typically has relatively minimal properties and limited data relationships (e.g., no more than one level of feature-attribute relationships in a limited number of records) allowing for relatively rapid relay and data intake/ingestion facilitating data processing from many inputting MAs transmitting data to NDS processor(s) (n.s.).

To promote data integrity and usability, such a method can further include a step of cleaning incoming data, #708, applying metadata (tagging), #710, and simultaneously or thereafter sorting data for further processing, #720. Harmonization of data from different sources (stored and RT/S data, from different types of MAs, etc.) and other data improvement processes are detailed elsewhere herein. Systems/methods for performing such applications are provided elsewhere. Such steps and certain data collections, described below, are included within the database drawing, to reflect that most, generally all, or all thereof are carried out/contained in the NDS, mostly in the NDS memory unit (NMEMU, #702).

An NDS can, i.a., transform, analyze, and sort MA-D and NDS-AD into a variety of functional categories that impact application of data, access to data, and other characteristics. Timing is one characteristic that can be used in such characterization. E.g., the NDS/method can separately identify and apply RT-MA-D that is deemed “current,” #722, according to preprogrammed rules that sorts RT/streaming data to blocks delivered within a relevant near-term period/“window” (e.g., a period of less than about 5, 3, 2, or 1 about minute(s), or, e.g., ≤about 40 sec, ≤30 sec, ≤20 sec, ≤15 sec, ≤10 sec, ≤5 sec, ≤2 sec, or ≤about 1 second). Near-Term MA-D, #726, can represent a different category of MA-D, associated with a different period defining the group, which may encompass, overlap, or be distinguished from RT-MA-D at this level/step in the NDS/method (process). Categorizing different periods of data into collections can allow the system to perform time-related analyses on such data, often at >2, >3, or more timepoints. E.g., an NDS can perform an analysis on recently received/ingested data (e.g., automatically) and a separate analysis on data that has been stored for only a limited period (e.g., building up a sufficient amount of data to meet a minimum standard for performing a particular application) or over longer time periods (hours, days, weeks, months, quarter years/quarters, or years).

Relatively raw MA data, e.g., RT-MA-D and MA-CD, received by NDS, can be subjected to further processing by the NDS, e.g., in sorting, and the NDS can generate analysis/AD based on such data or on AD. Further, e.g., MA-CD (cache data) (also sometimes shown in the Figures as “L-STR-MA-D” standing for “locally stored MA-D”) can be subjected to validation analysis through validation rules (e.g., minimum data time, minimum data quality, etc.) and thereafter characterized as validated MA-CD, #724. Machine learning model/module (MLM) processes can, e.g., generate physiological parameter or other type of prediction data, #728.

An NDS also can characterize/block data based on confidentiality rules. E.g., PHI, #732, can be separately blocked/separated/categorized/tagged from non-PHI, #734, which facilitates efficient delivery of data to, e.g., users/components) that can access or receive PHI (e.g., an EMR, HCP user, etc.) from those that may not be able to, e.g., users of a commercial network component. Other levels of confidentiality also are typically applied to NDS data, e.g., data accessible by certain user Entities, etc. Such sorting can be associated with location/Affiliation (Entity) data, MA LOC/AFFIL.-D, #736, which also can facilitate analysis at a location, Entity, or group level. NDS-AD based on location/affiliation can also be presented in the form of summary information or non-PHI, accessible by users of a commercial network component. In this and other respects, readers will recognize that such data groupings can overlap others described above (e.g., RT-MA-D will typically comprise PHI).

Combinations of such characteristics also can be used to characterize/form other zones in the network memory #702, and such zones can be subject to rules/governance policies. E.g., SAMD Access Zone, #740, defines a collection of data accessible by SAMD(s), which is governed by SAMD Access Policies, #742, and CMD Access Zone, #744, similarly defines a collection of data managed by the NDS according to preprogrammed CMD Access Policies, #746. As noted elsewhere, SAMD policies may be more stringent than CMD policies due to, i.a., RRs. In a similar manner, additional zones of data including Research Access Zone, #748, managed according to Research Access Policies, #750, and Commercial Access Zone, #752, managed according to Commercial Access (Acc.) Policies, #754, can be defined. The policies of such zones can reflect the various levels of access and kinds of data that users associated with such “zones” can generate and access under RRs and other system rules. Another combination of data can comprise data derived from or directed to delivery to EMR(s), MA-DISPU(s), or both #738, which can include various individual records comprising PHI.

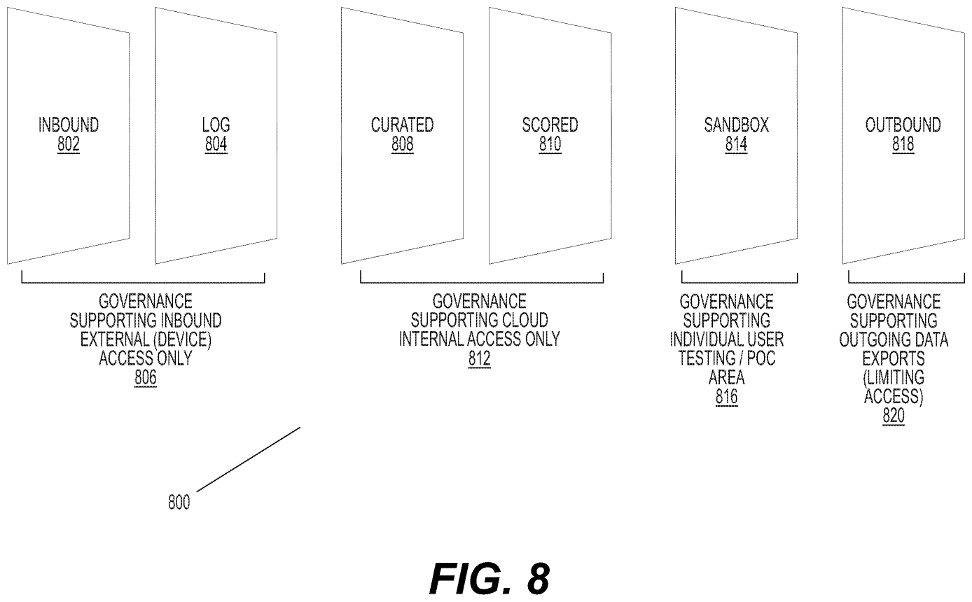

### FIG. 8

FIG. 8 is schematic describing the inclusion/application of different data zones to an NDS data repository/memory unit, #800. A first data zone is composed of inbound MA-D #802, e.g., RT MA-D, and MA log data, #804. Log data can include any data regarding any preprogrammed event occurrence (e.g., transmission of MA-CD upon an offline event, device alarm registration, etc.), but also can comprise status information, timestamped data related to actions serviced by applications or users or both, actions performed by the MA or an MA user (a HCP), decisions taken by applications, actions initiated by applications, runtime characteristics of applications, and any other type of log data. This zone can be characterized by inbound external (MA) access only (#806), preserving the nature of the raw MA-D in this zone (such a zone as part of a memory unit can be considered a general aspect combinable with any other aspect). A second zone, supporting network (NDS/cloud processor unit) (internal) access only includes, e.g., curated data (#808) and scored (#810) data, which can be, at least in part, derived from data stored in the first zone. Curated and scored data (#808 and #810) may be subject to governance policies supporting, e.g., cloud/NDS internal access and data segregation (#812). A third zone, which serves as a sandbox, #814, can be accessible for individual user testing applications, proof of concept testing, etc., #816. A fourth zone comprises outbound data, #818, and is subject to governance policies supporting data exports, #820, e.g., limiting access to other data contained in the overall data unit, delivery of appropriate data to appropriate user(s), provision of application(s) at MAs, etc. Data is sorted into zones through metatags and the like and engine(s) that implement such data sorting/curation, storage, and retrieval are provided elsewhere. The inclusion of such data governance zones, and delivery of data into such zones after initial ingestion, as part of, e.g., an enhanced data lake, can detectably or significantly speed up both data ingestion and subsequent on-demand or conditionally automatic/automatic applications performed on data in zone(s).

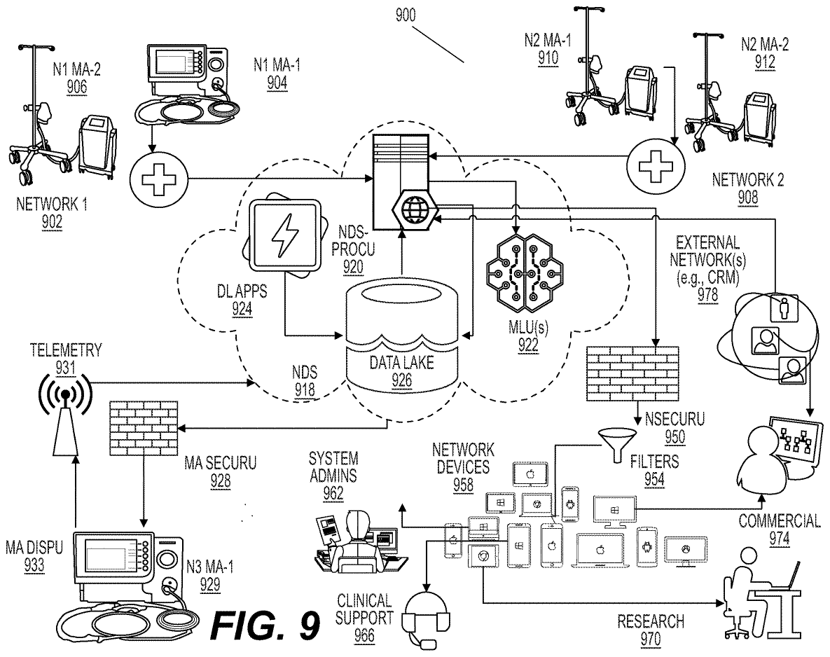

### FIG. 9

FIG. 9 is a representation showing various possible parts of an exemplary network #900 including MA groups and an NDS, #918, with focus on examples of the types of physical device components that can operate within such a network.

The exemplified network representation comprises (and thus the included NDS, #918, has access to receive data from and transmit data to) several different MA groups, comprising, in at least some cases, multiple types of different MAs, which generate different types of MA sensor data. To simplify the description only a few MA groups are illustrated, but, in many aspects, many more MA groups could be associated with the NDS (e.g., ≥about 5, ≥10, ≥15, ≥20, ≥25, ≥35, ≥50, or ≥about 100 groups comprising larger numbers of devices, e.g., ≥about 10, ≥15,≥25, ≥50, ≥100, ≥150, ≥200, or ≥about 250 each, consisting of ≥3, ≥4, ≥5, ≥7, or ≥about 10 types of MAs). Illustrated here, a first network/group of MAs, #902, comprises MA(s) of a first type of MA (N1 MA-1, #904), e.g., a component for monitoring and controlling patient data related to a heart pump, and one or more MAs of a second type of MA (N1 MA-2, #906), e.g., an extracorporeal membrane oxygenation (ECMO) system. A second MA group (Network 2, #908) comprises other unit(s)/group(s) of the first and second types of MAs (N2 MA-1, #910 and N2 MA-2, #912). A third exemplary MA group comprises another type 1 device (N3 MA-1, #929). All three MA groups transmit data to NDS, #918. To illustrate aspects, N3 MA-1, #929, is shown as being associated with a telemetry function, #931, e.g., a Wi-Fi transmitter or other wireless protocol data transmitter which relays information to NDS, #918, and receives NDS-AD, control instructions, etc., back from NDS, once cleared through an MA security unit (MA-SECURU, #928), including displayable information displayed on an MA DISPU, #933, which data display conforms to schema(s)/representation(s) generated by the NDS.

NDS #918 comprises a cloud-based and expandable (demand response scalable) system architecture comprising a processor “unit” (NDS-PROCU, #920), which typically comprises, e.g., an association of massively parallel, distributed, high availability, virtual processors, and a memory unit primarily composed of data lake (DL), #926, which may comprise or be in the form of an enhanced data lake (EDL), as described herein. NDS, #918, also comprises analytical unit(s)/engine(s), that perform functions, such as, e.g., data lake applications, #924 (e.g., query engine, schema application engine, prediction engine(s), etc.), and machine learning units/modules (MLUs, #922), which perform machine learning-based processes or processes that comprise application of machine learning. NDS, #918, also comprises or is associated with a security unit (NSECURU, #950) comprising, e.g., firewall functionality, and is associated with functional filters, #954, which provide for collection and sorting of functional data suitable for use by the various other NDS/network components.

Other components of a network can include a variety of network access/input devices, #958, including laptop computers, tablets, smart phones, etc., which are employed by the various users of the NDS/network. Such users can include system admins, #962, which include users associated with the manager/owner of the system that monitor and attend to system performance issues and play other roles, e.g., supervision of machine learning in supervised machine learning modules. Clinical support, #966, is another other network group/component, which can monitor patient status as a backup function to HCPs. Users in a research component/program, #970, also can be provided some, most, or generally all N-AD, MA-D, or both via other network devices #958. Similarly, users in a commercial group/component, #974, can receive select N-AD (typically not including PHI due to Regulatory Requirements), via Network Devices, and simultaneously can receive data from other external networks, #978, e.g., a customer relationship management database/program, which also or alternatively can relay data directly to NDS such that NDS can generate NDS-AD from combination of such other sources of information along with MA-D or other NDS-AD. From this illustration it can be seen that networks of the invention can comprise a level of inter-operability and complexity that far exceeds anything described previously in the art and that systems of the invention are inventively unique in being adapted/configured to concurrently address inflow of data from and output of data to such various constituent groups of such a complex network, allowing for better management of healthcare-related information at many levels of a healthcare system/operation.

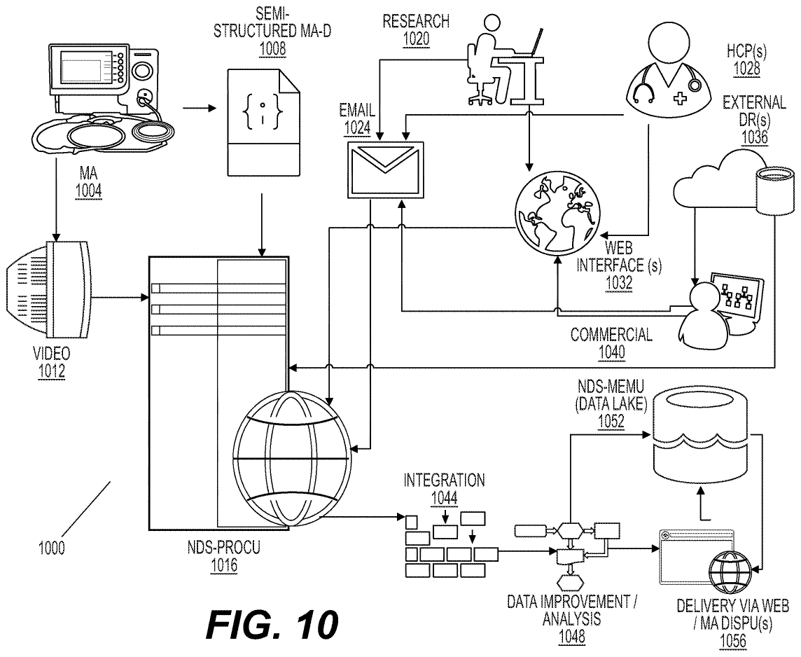

### FIG. 10

FIG. 10 provides another illustration of components of a network, #1000, formed between, i.a., an MA, #1004, NDS (represented by NDS-PROCU, #1016), and other network/NDS components (e.g., NDS-MEMU, #1052), with focus on data flows in such a network. In the illustrated exemplified network, MA, #1004, transmits i.a. video data #1012 (e.g., comprising video recordings of display(s) associated with the MA or subject, the subject, the MA, etc.) and semi-structured MA-D, e.g., JSON formatted semi-structured data #1008, via real time/streaming processes (and, at least in some instances, MA-CD), as well as other status/log data, to NDS processing unit (NDS-PROCU #1016), which can be, e.g., a web/cloud-based scalable, high speed, and highly available processor unit, e.g., any of those such system(s) exemplified elsewhere herein. NDS-PROCU #1016 can receive additional input from ONDIs, e.g., devices associated with users assigned to research unit/group, #1020, HCPs #1028, and commercial unit/group, #1040. Such ONDI users can provide semi-structured data input, structured data input, or both, via, e.g., e-mail, #1024 or web interfaces #1032 (e.g., mobile device apps, web pages, etc.). External data repositories (DR(s))/services, #1036, e.g., a CRM, can both provide data to commercial unit users and also or alternatively directly to NDS-PROCU, #1016 (e.g., in aspects such data is first processed by an NDS processor before being relayed on to system users, such as commercial unit users, but in other aspects such data is also or alternatively combined at a user device/interface level, where such devices receive data directly from such external databases and blend it with NDS-AD, typically according to, i.a., NDS output parameters/controls). NDS-PROCU #1016 can apply various data integration processes #1044 (e.g., data harmonization, validation, cleaning, etc.), data improvement/analysis processes, #1048, e.g., machine learning prediction of expected patient physiological data as a predictive tool for health care management, leading to device display, alerting, or even control of MAs, and then can store such data in a system memory component (NDS-MEMU, #1052), which can comprise, primarily comprise, or consist essentially of a data lake (DL) or enhanced data lake (EDL), and NDS can also deliver NDS-AD via web displays/other internet-accessible interfaces accessible by network/system users or MA DISPU(s), #1056. As can be seen from this illustration, system(s) of the invention are configured/adapted to receive and process a range of data of markedly different types and to use such data in generating analytical information leading to output sent back to devices or other devices/interfaces of the network. The ability of an NDS to process such various inputs allows for more robust data collection and the ability of the NDS to rapidly process such data inputs, by, e.g., inclusion of DL(s), sets such system(s) apart from previously described systems.

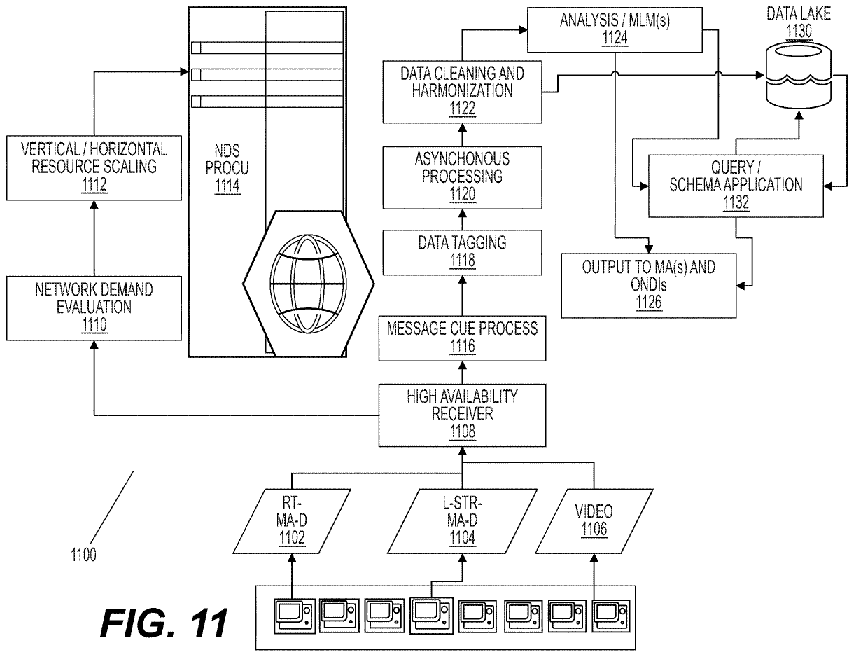

### FIG. 11

FIG. 11 is a flow chart describing steps of exemplary data processing methods #1100 employable in components of a system/network of the invention. In this exemplary method, MAs transmit data, e.g., stream real time data (RT-MA-D, #1102), L-STR-MA-D/MA-CD, #1104, video data, #1106 (e.g., video of MA displays, as described in Abiomed patent documents cited herein), or a combination of some/all thereof, optionally with other types of data, to a high availability receiver, #1108, which can work with or comprises a streaming data processor/engine, and then further to a message queue process, #1116, which applies preprogrammed rules for determining the priority of various data inputs for processing based on the nature of such inputs (e.g., time of transmission/receipt, nature of data, need for data for performance of NDS functions, and size of data/impact on NDS processes).

A network demand evaluation function/step, #1110, concurrently evaluates demand on the NDS in terms of data flows, backlog of data to be processed, etc. If demand exceeds preprogrammed threshold(s), vertical/horizontal resource scaling functionality, #1112, increases NDS resources at either level, e.g., by increasing number of available processors for parallel, distributed processing, that currently make up NDS-PROCU, #1114.

After, before, or during message queueing (#1116), NDS-PROCU can apply other modifications to incoming data, e.g., data tagging, #1118, and data cleaning and harmonization, #1122. NDS-PROCU also can comprise, e.g., asynchronous processing functionality/protocols, #1120, for ensuring that prioritized data is processed first, again, according to factors including data need, data timing, and data impact on NDS functionality, given the need for the NDS to remain highly accessible and to provide very rapid (in part at least near real time or real time) responsiveness in the healthcare setting. Readers will appreciate that such prioritization of certain data types and the inclusion of such asynchronous processing engine(s)/step(s) represent a general aspect of the invention, combinable with any other method/system aspect provided here. Prioritization can be applied based on rules (e.g., with data such as device operability or patient health data taking priority over other data) and effectuated through application of metatags or other data identifiers as described herein or otherwise known in the art. NDS-PROCU also can perform other analysis or apply machine learning module(s) (MLM(s), #1124), such as prediction models for patient physiological parameter(s), outcomes, response(s) to course(s) of treatment, and the like. MA-D and NDS-AD can be stored in the NDS memory unit, which typically comprises a data lake, #1130, that is efficient at receiving semi-unstructured MA-D, again supporting speed of functionality, NDS availability, etc. The NDS can also comprise other on-demand functions, e.g., query functions and schema applications, #1132, in which data in data lake #1130 or other data repository (e.g., an EDL or database) is retrieved, ordered into relational datasets, etc. NDS-AD, obtained through query processes, MLMs, or other analytical processes, can be delivered to ONDIs or MAs, #1126. This overview illustrates some of the various steps performed to enable a system to carry out the various described functions of systems efficiently and effectively in a reliable manner and that distinguish methods/systems of the invention from other previously described methods/systems for processing medical apparatus data.

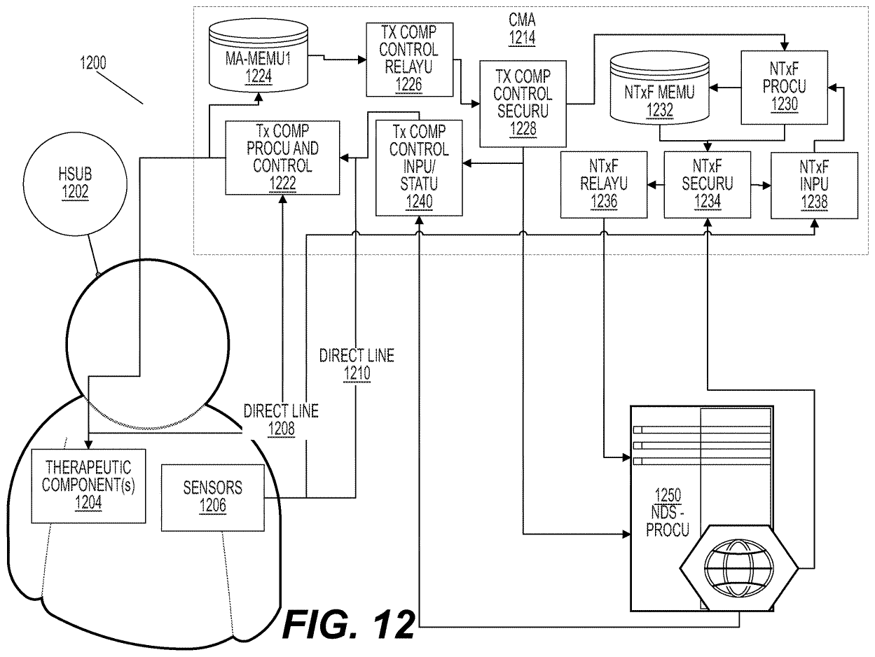

### FIG. 12

FIG. 12 is a simplified illustration of the relationship between components of a specialized type of MA (a multi-zone MA (MZMA) or combined MA (CMA)) that can be in a network with an NDS #1200 in accordance with aspects. A box around processes/components indicates elements that are contained in or otherwise closely functionally associated with the CMA.

In the illustrated example, a patient (HSUB, #1202) is being treated with MA comprising therapeutic component(s), #1204 (e.g., a blood pump, ECMO, or the like), which apply one or more medical treatments to patient/HSUB. Therapeutic application and other patient physiological information obtained from sensors, #1206, or other information related to operation of the therapeutic component/MA is relayed to a data system of the combined MA (CMA, #1214) (aka, an MZMA), typically via direct data transmission line, #1208, or similar communication means, e.g., a local wireless transmission channel, e.g., a secure Bluetooth communication channel. Sensors, #1206, are placed on patient, in patient, or both, and in addition to therapeutic component-relevant measurements can also measure other patient physiological parameters that are not directly tied to the control of therapeutic aspects of MA, and which also communicate with CMA via a separate direct communication line, #1210. The CMA is considered a combined MA (or MZMA) because it comprises a combination of components dedicated primarily or exclusively to the management of therapeutic component (Tx Comp) data and functioning as well as other components dedicated i.a. to the processing of non-Tx functions/data. Such devices are also sometimes referred to as multi-zone MAs (MZMAs), as described elsewhere, particularly when such different zones of components are subject to different levels of network interactivity, modifiability, regulation, etc.

Therapeutic component data is relayed to a therapeutic component (TxComp) processing/control unit (Tx COMP PROCU and Control, #1222), which is part of a therapeutic component data/control zone/component, which can be considered one “side” or part of the CMA. Other components of the therapeutic component “zone” (part or “side”) can include (1) a memory unit, MA-MEMU1, #1224, which stores TxComp MA-D, a TxComp Control RELAYU, #1226, which relays information to the non-therapeutic component “side” or part of the CMA, usually after such data is cleared/screened/authorized by a Tx component security unit (SECURU, #1228), and (2) a Tx component input unit/status unit, #1240, which receives status information from/to NDS-PROCU #1250 or the Non-therapeutic function (NTxF) “side” of the CMA/MZMA.

Non-therapeutic function (NTxF) components can include, e.g., a separate NTxF PROCU, #1230, a separate memory, NTxF MEMU, #1232, a security unit, NTxF SECURU, #1234, and a relay unit, NTxF RELAYU, #1236, and an input unit/receiver, NTxF INPU, #1238. In operation, NTxF INPU #1238 receives non-therapeutic control data from Sensors, #1206, via direct line #1210, and delivers such data to NTxF PROCU, #1230. NTxF PROCU #1230 also receives TxComp data from Tx Comp RELAYU, #1226, via Tx Comp SECURU, #1228, which limits the data that can be relayed from the Tx Comp side of the CMA. A non-limiting flow of data through such steps/functions/components is exemplified by the direction of arrows in the Figure. The second security unit, NTxF SECURU, #1234, can screen data delivered to NTxF INPU #1238 from NDS-PROCU, #1250, e.g., NTxF PROCU updates/patches, etc. Such a direct connection between NDS-PROCU and the Tx Comp components can be limited to status data transmissions only or similar NDS status signals (e.g., the availability of a software update). By limiting the flow of data to the Tx Comp components, the configuration of the CMA/MZMA ensures that such components, which can be engaged in critical life support functions, are protected/isolated from any type of interference relayed over the internet, e.g., a computer virus, hacking, or other harmful/malicious code. Similarly, NTxF RELAYU, #1236, can be responsible for relaying both NTxF data as well as Tx Comp data to NDS-PROCU, #1250, creating a separation of Tx Comp components and NDS with respect to outgoing data flows from the MA to the NDS. CMAs represent an independent aspect of the invention and an important aspect of networks of the invention. Devices adapted to comprising several different components under different levels of network access, security, etc., aids in ensuring that critical life support systems are protected from unauthorized access, tampering, and the like, improving patient safety and confidence in the network/medical apparatuses.

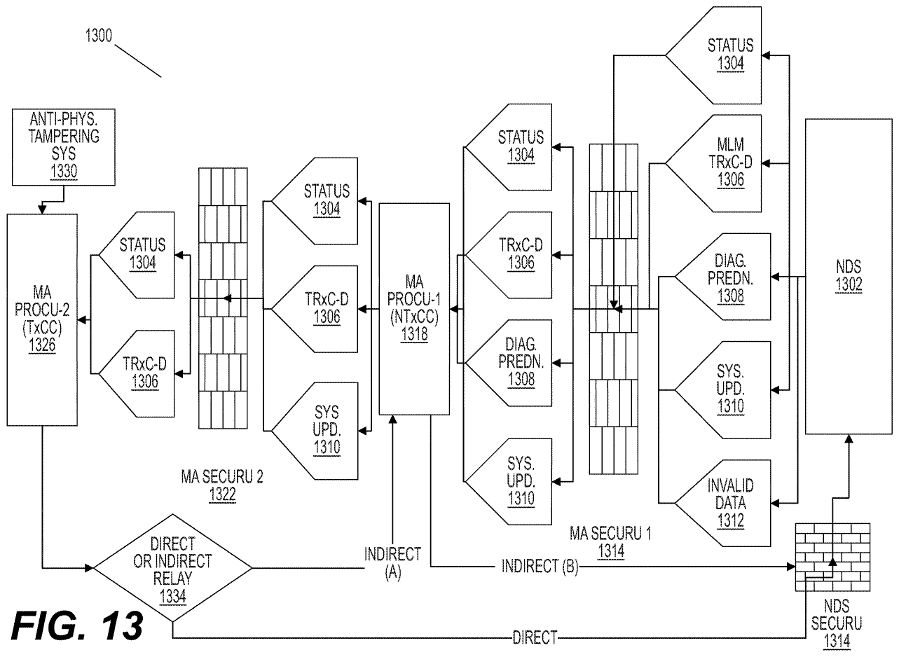

### FIG. 13

FIG. 13 provides an illustration of flow of data #1300 from an NDS into and through a MA device/system similar to the MZMA/CMA device described in FIG. 12. NDS, #1302, transits a variety of data types, here including (1) status query/request, #1304, (2) machine learning module (MLM) therapeutic (TRx) control (C)-data (D) (MLM TRxC-D), #1306; (3) diagnostic prediction (Diag. Predn.) data, #1308, which typically also is derived from application of NDS MLM(s); (4) system update (Sys. Upd.) data, #1310; and (5) invalid data (e.g., data corrupted in transmission/processing), #1312. CMA comprises a first security unit (MA SECURU 1, #1314) that screens such data; blocking the invalid data, #1312, from being relayed into CMA components, but allowing the other four types of data to be acted on by a first MA processor, which works on non-therapeutic control aspects of the CMA (MA PROCU-1 (NTxCC), #1318). MA PROCU-1, #1318, relays diagnostic prediction information to display unit, alarm components, or both (not shown), but does not further transmit diagnostic prediction information, #1308, limiting the data flows to just three data collections, which are received by a second security unit/process, MA SECURU2, #1322, which further blocks system update data, #1310, thereby protecting MA PROCU-2 (which is a part of a therapeutic control component—TxCC—of the MZMA, #1326), from modification via internet-relayed messages. Thus, only, status information, #1304, and data specifically designed to control the operation of the therapeutic control component, #1306, are permitted to flow into MA-PROCU-2 #1326, thereby greatly increasing the security of MA-PROCU-2. CMA/MZMA can also further comprise physical anti-tampering system, #1330, which can detect physical access to components of the therapeutic control unit, resulting in alarms, shutdown, or other steps/results. MA-PROCU-2, #1326, also can relay (#1334) certain data to NDS #1302, either directly “through” (i.e., after screening by) an NDS security unit NDS SECURU #1314, or indirectly (A) through NTxCC #1318, the latter data flow path possibly adding even more security to the operation of the TxCC by limiting direct outflowing communications between the TxCC and the internet.

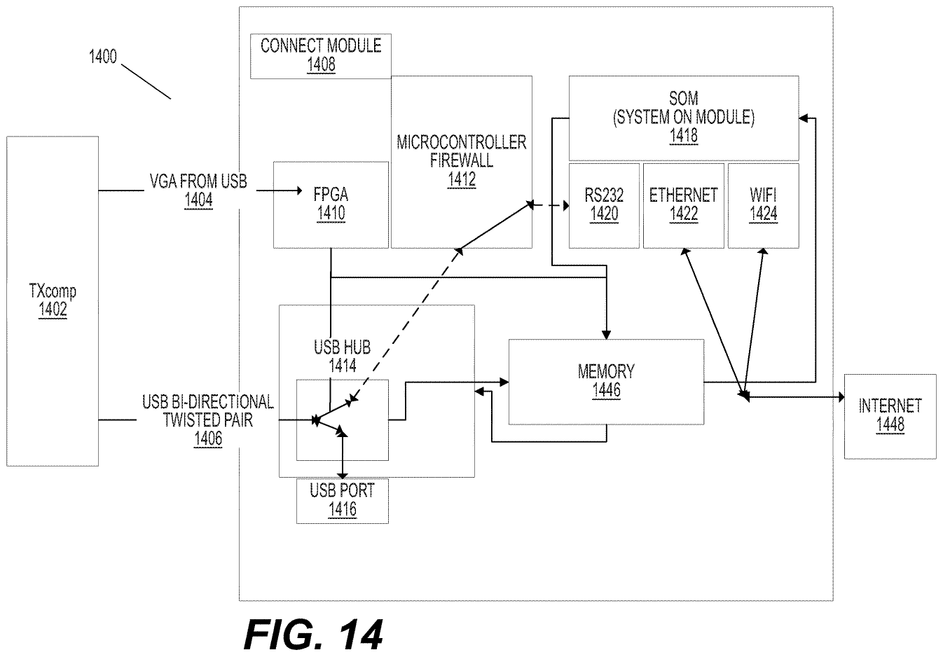

### FIG. 14

FIG. 14 illustrates another exemplary configuration, #1400, of components of a combined medical apparatus (CMA/MZMA). A therapeutic (Tx) component (TXcomp, #1402), can control operation of a medical device, e.g., a heart pump, as described/exemplified elsewhere. TXcomp, #1402 is in communication with a Connect Module, #1408, which records and relays patient condition monitoring functions to an NDS, via an internet connection, #1448, e.g., via ethernet connection #1422 or Wi-Fi connection #1424. A box drawn around several elements indicates that such elements are typically contained in, operate in, or otherwise closely associated with this non-Tx component (the Connect Module). Specifically, data communication between the secure TXcomp component, #1402 and the Connect Module #1408, can relay data via, e.g., (1) a video communication line (video graphic array, VGA, relayed over USB, #1404) that transmits video image data associated with the MA to the Connect Module, which is received by field-programmable gate array (FPGA, #1410) and (2) a second data communication line, over which other data relayed via bi-directional twisted pair USB, #1406, and received by a USB hub, #1414. FPGA #1410 is a component of the processing functionality of the Connect Module and MA, and FPGA #1410 can perform analytical functions on incoming data (e.g., tagging, sorting, validating, cleaning, monitoring for local alarms, etc.). Typically, FPGA #1410 can be dynamically programmed/reprogrammed with a data path that matches the specific workloads of combined device (CMA) function(s). Communications across both channels typically are substantially or completely unidirectional, with, e.g., data only flowing from Tx Comp #1402 to the Connect Module #1408. FGPA #1410 also relays data to USB hub #1414, which is either transmitted to the System on Module (SOM) #1418 for relay to NDS (not shown in this Figure) or to other users with direct access to data for the associated MA. Data can be retrieved locally from the USB hub #1414 via USB port #1416. Both video and other data is relayed to Connect Module Memory #1408, which can be retrieved via applications that relay data to USB hub #1414 for local device access or that can be relayed to system on module, SOM, #1418, for transmission to NDS or other users via internet #1448. SOM #1418 can both receive and transmit data to internet #1448 and can be considered to form both an input/output unit for the MZMA. However, a microcontroller and firewall component, #1412, typically screens incoming data, and limits the data that passes between USB hub #1414 and SOM #1418 in both directions, but particularly in respect of incoming messages (as signified by dashed line) (typically through screening for specialized packets comprising information required to pass through the firewall component). Intercomponent communication (between parts/components of the different zones/parts of the MZMA/CMA) is managed via an RS232 data processing component, #1420, which forms part of the SOM. Components of the system can access the local Connect Module memory #1446 for relaying information to local users (e.g., via USB port #1416) or for relay to NDS via internet #1448, as described elsewhere. An MA/system comprising a combined device system exemplified in this Figure, #1400, can provide the combined device characteristics/functions described elsewhere in connection with other aspects and maintain a high level of security for the therapeutic component, ensuring patient safety and compliance with applicable Regulatory Requirements.

Any of the various systems described herein can be modified by skilled persons. For example, RS232 communication could be replaced with other suitable intercomponent communications standards/equipment; FGPA could be substituted or augmented/coupled with a different integrated/hardware circuit or processor component or coupled with various application-specific integrated circuits (ASICs) or processors (not shown); USB hub and ports can be replaced with other local communication components, hubs, and ports; or any combination thereof.

The exemplified illustrated components of an MZMA reflect how the various principles of the invention, particularly in respect of MZMAs/CMAs, can be put into practice by ordinarily skilled artisans, using known/available components, such as USB ports, FPGAs, and microcontrollers.

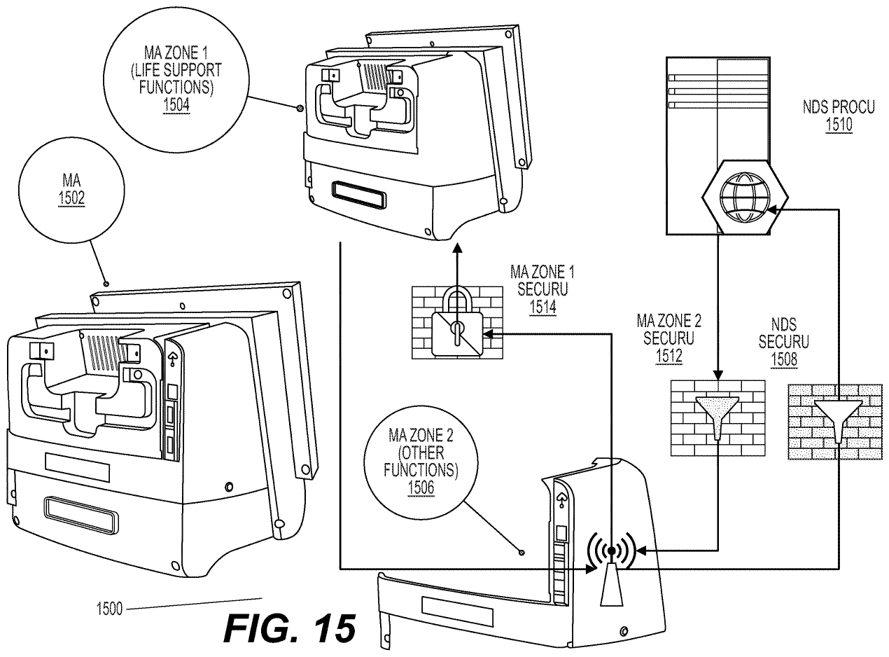

### FIG. 15

FIG. 15 provides another depiction of how mechanical components and data system components of a combined device (a MZMA/CMA) can work together in real world applications. Specifically, a depicted exemplary network connection #1500, comprises a medical apparatus (MA, #1502) and NDS (represented by NDS PROCU, #1510). The combined device component, #1502, is composed of a therapeutic component, MA Zone 1, #1504, which, i.a., controls life support functions and can comprise/provide a local display of data (now shown), and a MA Zone 2, #1506, comprising other elements configured/adapted to perform other functions including receiving diagnostic/patient condition information, performing analytical functions, and relaying information to and receiving information from the internet through, i.a., a wireless transmitter/receiver component. An MA Zone 1 security unit (SECURU, #1514), can be present and utilized to restrict transmission of data from MA Zone 2 #1506 to MA Zone 1 #1504 to only certain, limited forms of data communications meeting format or content requirements. MA Zone 2 #1506 can be associated with a MA Zone 2 security unit (SECURU, #1512), which also screens data at a less restrictive level (e.g., by employing a firewall to block any transmitted data that does not meet data standards preprogrammed into the component that indicate that data is from NDS or other authorized data relay device). Data uploaded from MA Zone 2 #1506 can also be screened before reaching NDS by an NDS security unit (NDS SECURU #1508), which can, e.g., comprise a firewall component/system to ensure that incoming data does not include computer viruses, unauthorized data requests, or other malicious/unauthorized code.

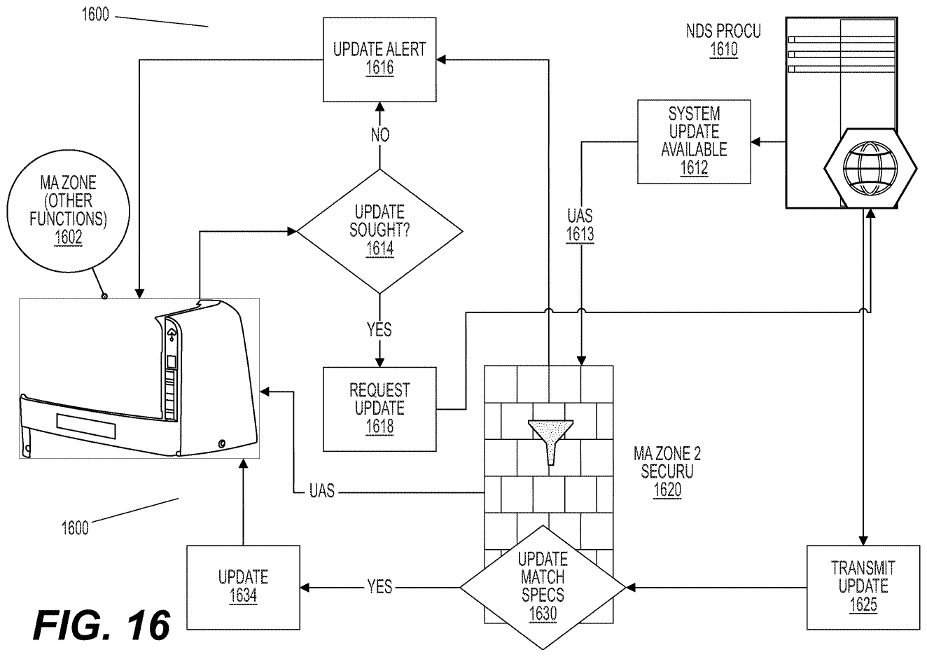

### FIG. 16

FIG. 16 illustrates an exemplary two-step security protocol, #1602, for controlling the updating of software/operating system element(s) of a non-therapeutic component (e.g., Connect Module component) of a combined device (CMA/MZMA), protecting patient safety, aiding in complying with Regulatory Requirements (RRs), or both.

MA Zone 2 #1602, in operation performs other functions than the life support MA control functions handled by associated Tx Comp (n.s.), e.g., collection of patient physiological data, upload and download of NDS data, and local processing and memory storage functions, as described elsewhere. From time-to-time, system updates of the operating system/software of MA Zone 2 #1602 are required/desired, but it can be critical that the system be maintained in a secure state for patient safety and RR compliance, patient safety, confidentiality, etc. NDS processor (NDS PROCU #1610) in such an aspect can relay an update available signal #1613, to MAs, when NDS determines a software update is recommended/necessary, #1612. The update available signal (UAS), #1613, as with other incoming data/messages, will be screened by MA Zone 2 security unit, #1620, and then after determination that the message is authorized allowed to be received by the MA Zone 2 component, #1602. The UAS #1613 can include information about the nature of the update (criticality, impacted functions, update time expected, and impact on systems, etc.), as well as version information, a description of changes, etc.

An authorized user of the MA can determine if a system (e.g., OS) update is approved/sought, #1614. An update may not be sought if, for example, the device is currently in use with a patient; if the device is in a research program, where the local administrator/user(s) wishes to first evaluate the update; or for any other reason. If the update is authorized/sought, #1614, the user can, through the MA, cause the MA to relay a request for an update, #1618, via MA Zone 2 #1602 to NDS PROCU #1610. In aspects, only upon receipt of such a request, or a confirmatory request in response to a system availability signal, will an NDS processor (NDS PROCU #1610) attempt to transmit a software update #1625 to MA Zone 2. The system update data can be screened by MA Zone 2 SECURU #1620. If SECURU, #1620, determines that component(s) of the update data sufficient match expected specifications #1630, per preprogrammed standards, the update is allowed to update the MA Zone 2 software, #1634. Where an update is not sought, #1614, MA Zone 2 or NDS may trigger one or more update alerts #1616 (e.g., special displays, email, or text messages to users/administrators, etc.). Alarms also or alternatively can register on the device, other network devices, etc., in the event there is a delay in updating a device after a period of an update being available, particularly where the update is deemed important to device security, device effectiveness, patient safety, and the like.

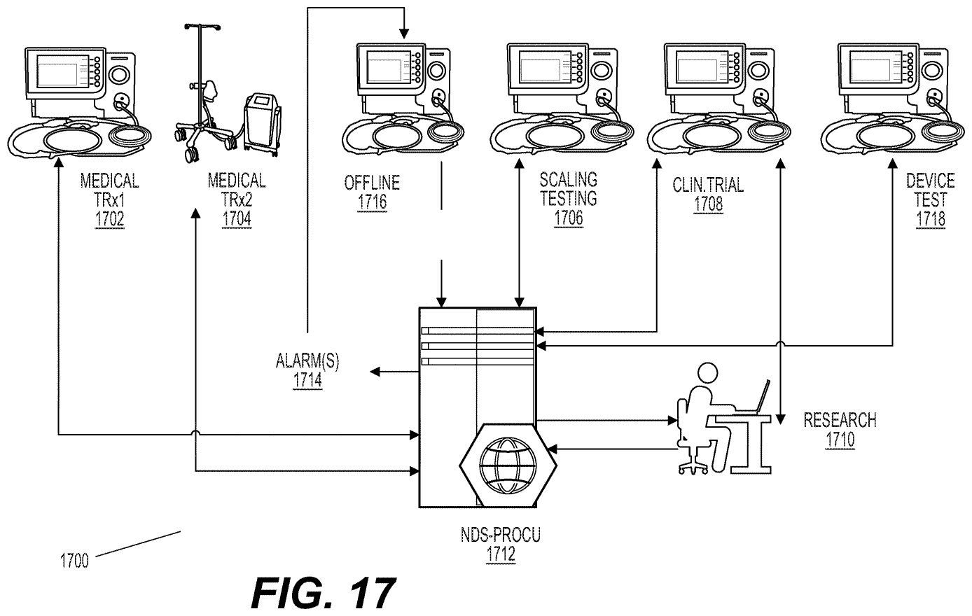

### FIG. 17

FIG. 17 depicts an exemplary portion of a network #1700 comprising several medical apparatuses (MAs), illustrating MAs in communication with an NDS in various operational states, to illustrate how systems of the invention process information from MAs in such different operational states. Specifically, first illustrated MA is in a first medical treatment (TRx1) state, #1702, and second MA is in a second medical treatment state (TRx2), #1704, with relevant information concerning both states being relayed to and received from the NDS processor (NDS-PROCU, #1712) (e.g., different patient physiological measures or device performance measures, different device controls e.g., different SAMD/CDS protocols, or any combination thereof (ACT)). Third MA is shown as in an offline state (#1716). Fourth MA is concurrently engaged in system/network testing, e.g., scalability/scaling testing, #1706, and relaying and receiving information related to such testing to and from NDS-PROCU, #1712. Sixth MA is similarly engaged in local device testing, #1718. Fifth MA is concurrently engaged in a clinical trial, #1708, e.g., the testing of a new application of the type of MA, patient type, disease type, or any combination thereof, and relaying and receiving information from NDS-PROCU #1712 specific to the performance of the clinical trial. A seventh MA is engaged in a research program or research program users can access data from the various other MAs through research group/platform ONDIs, #1710. E.g., a research platform user can engage in testing of an MA, testing NDS implemented functions (e.g., new machine language modules), or any combination thereof, and relaying and receiving data relevant thereto to the NDS or ONDIs. Research component(s) #1710 also can be engaged in the monitoring of the performance of other aspects of the network, e.g., performance one or more of the other MAs described herein.

The NDS processing function (NDS-PROCU #1712) can comprise components for concurrently identifying such different data inputs and outputs (e.g., a processor that identifies the states of the various MA units based on status information relayed from each MA and recognizable by NDS-PROCU) and for ensuring that NDS-AD is concurrently appropriately routed to such MAs. In many aspects, the number of device states, device types, locations, and devices that are specifically identified and concurrently communicated with by NDS-PROCU is ≥~100, ≥~250, ≥~500≥~~1,000, ≥~2,500, ≥~5,000, ≥~10,000, or ≥~25,000, or even more (e.g., ≥~100,000 unique states). Accordingly, systems that accommodate such a network typically will comprise the use of functional data units that provide for the secure and reliable identification of specific device, status, and other information (e.g., entity information, location information, device type, etc.). In aspects, some information is relayed or revalidated only periodically, maintained in NDS memory, or ACT, to reduce incoming data load associated with streaming/real time data transmission to NDS, other data transmission to NDS, and data transmission from NDS to such a sizable group of MAs as well as relay of data to and received data from other network/NDS components or other interfaces that access the NDS, MAs, or both. NDS-PROCU #1712 also simultaneously performs other analytical and control functions, as discussed elsewhere, e.g., evaluation of MA performance in one or more operational states and, if triggered by meeting or exceeding one or more threshold indicator(s), relaying one or more types of alerts or alarms, #1714, to known user interfaces (e.g., mobile devices, email accounts, etc.), MA administrators, MA displays/outputs (e.g., audio outputs), to 1, 2, or more user groups/entities (e.g., clinical monitoring, research, administrators, or HCPs). Data from devices engaged in system/network testing, device testing, etc., are analyzed with respect to such network operational level activities, but such data is specifically separated from data involving human subject applications, to ensure integrity of subject application data.

The ability of an NDS to concurrently receive data from MAs in such states while also processing such data and relaying NDS-AD to such users, allows the NDS to provide substantially improved and diverse functions as compared to medical device management systems known or described in the art e.g., concurrently obtaining treatment from both actual treatment, clinical trials, and research components, and to use such data in the development, refinement, or operation of MLMs employed in SAMD protocols, CDS protocols, or both, while concurrently facilitating round the clock (24/7) operation of the NDS by permitting for NDS/network testing (e.g., scalability testing), device testing, etc., without impacting streams.

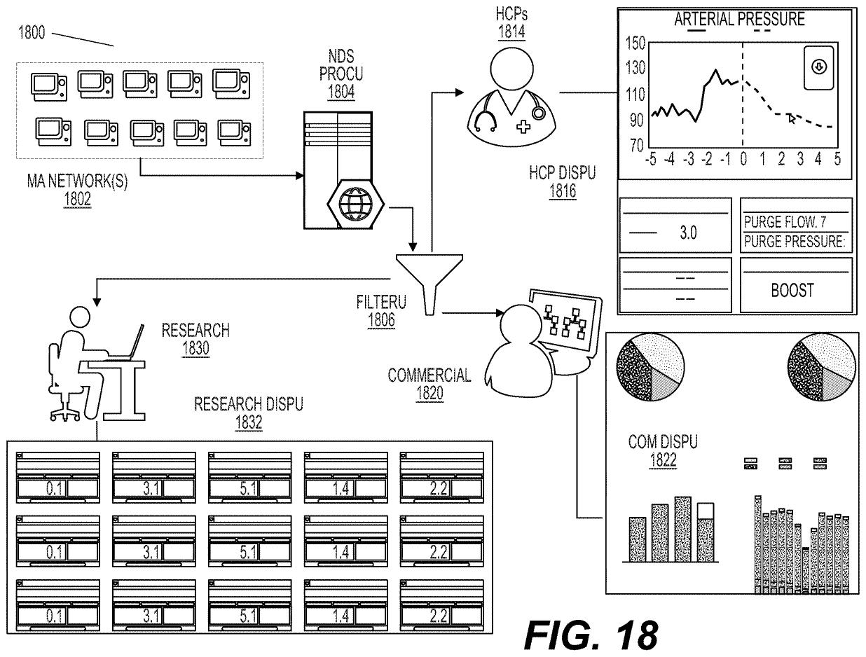

### FIG. 18

FIG. 18 provides a simplified overview of components of a network of the invention, #1800, comprising different user groups, each receiving NDS analytical data (NDS-AD) presented in different specialized data displays according to representations/schemas generated by the NDS. In the illustrated example, an NDS processor (NDS-PROCU, #1804), receives real time/streaming data and other data from MAs in MA network(s), #1802, and applies analytical processes to such incoming MA-D to develop NDS-AD. A filtering/directing unit/engine (FILTERU, #1806), evaluates NDS-AD, and determines recipient and recipient class (user group) to receive NDS-AD, and transmits data that is configured to populate displays used by different user group users in accordance with specialized schemas/representations generated in accordance with preprogrammed instructions/standards. Specifically, health care providers (HCPs, #1814), receive an HCP display on HCP interface/device display units, #1816, which can comprise MA-specific/patient-specific data, e.g., near term actual and predicted physiological parameters (e.g., near term actual and predicted heart rate generated by an MLM processed by NDS PROCU), possibly with other CDSS or SAMD application outputs/functions, such as treatment recommendations, related points for investigation, possible diagnosis, etc. Commercial group users #1820, on the other hand, concurrently receive NDS-AD in a commercial group display configuration, #1822, which typically draws data from both the NDS processing unit and also external commercial database(s), e.g., CRM(s) (not shown) (either directly or indirectly, in the latter case such data being first received by and processed by the NDS prior to relay to the commercial group displays/devices). Commercial group data displayed on commercial display units (COM DISPU, #1822), typically does not include PHI, and usually includes aggregated device data rather than patient/device-specific data. Alternatively, still, research group users #1830, concurrently receive different data displayed via a research group display unit(s), (Research DISPU, #1832), on interfaces/devices, which can also comprise, e.g., performance information concerning several different MAs operating under a variety of conditions, patients diagnosed/treated for various conditions, flows of data in devices, the system, or network, or any combination thereof. The data presented to each of these groups, derived from simultaneously processed NDS-AD, and concurrently delivered and displayed, are, thus, very different in content. For example, research users may receive data on unapproved uses of MAs that would be withheld from HCP displays due to RRs. Similarly, commercial user displays would not receive sensitive patient or IE information that might be displayed on HCP displays. This concept can be applied to additional components, users, etc., reflecting the enhanced and broader performance of an NDS in concurrently handling inputs and delivering outputs to a variety of constituents. In aspects, there is some amount of data overlap analyzed by an NDS coming from two or more such classes, delivered to display(s) of two or more classes, or both. The flexibility/comprehensiveness of such systems further distinguishes systems of the invention from previously described medical device data management systems.

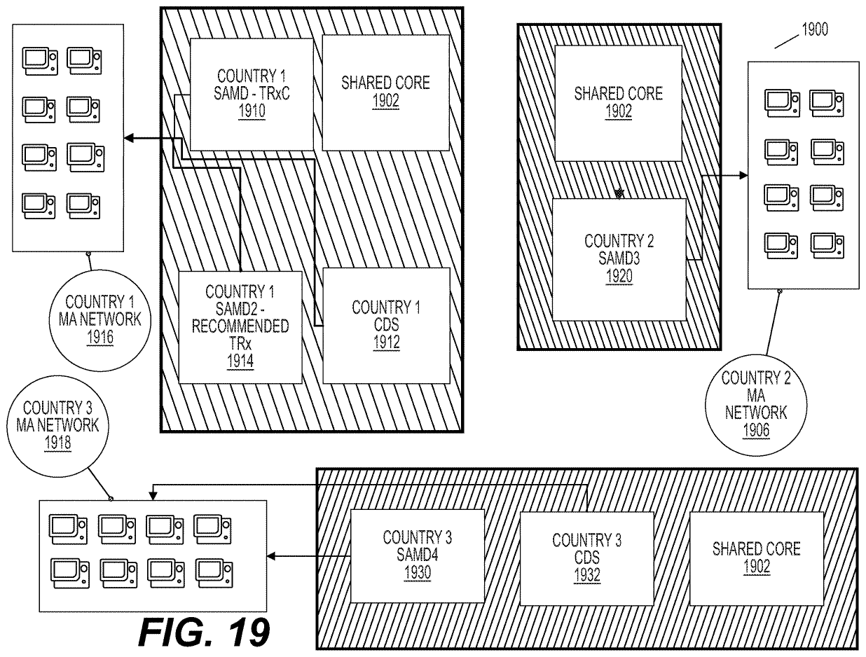

### FIG. 19

FIG. 19 is a simple schematic showing the groupings of data in one or more NDSs comprising overlapping but different NDS/network components, #1900. The illustrated network includes a first country-defined network/group of MAs, #1916, such MAs being located in one country (e.g., the USA), and an NDS or NDS component comprising a shared data core, #1902, which performs at least some base/basic functions of the NDS, e.g., processes for handling real time/streaming data (e.g., messaging queue, prioritization, and NDS scaling components), data cleaning processes, device identification and data association processes, analytical processes (e.g., MLMs), etc. Certain processes performed by NDS, however, such as device-controlling functions related to subject therapy, are subject to certain country-specific regulatory classifications/RRs, e.g., SAMD, #1910 and SAMD2, #1914, and CDS/CDSS, #1912. A second MA network, #1906, of MAs in a second country (e.g., Germany), also delivers data to a local NDS or international NDS, comprising or having access to shared core functions/components #1902, present in/utilized by MAs in country one, but also applies a second country specific SAMD (here, SAMD3, #1920), which is, e.g., an SAMD approved by a Regulatory Authority of the second country. Shaded boxes are included around elements of each MA network to clarify the at least partial separation of some components of such networks. Finally, a third country (e.g., Japan) comprises a third network of MAs, #1918, which also delivers data to an NDS or NDS component, comprising or having access to shared core functions #1902 and comprising country 3-specific functions e.g., SAMD4 (#1930) and CDS (#1932). The different SAMDs (SAMDs 1 (#1910) and 2 (#1914) in country 1, SAMD3 (#1920) in country 2, and SAMD4 (#1930) in country 3, can be subject to different regulatory status, different Regulatory Requirements, and therefore can comprise different data functions or apply such functions on different MA data, but the shared core (#1902) means that many, most, generally all, or substantially all the function components of the NDS are shared or repeated between such different countries. The principle exemplified in this Figure can be applied to more complex systems involving more countries, regions, regulatory requirements, devices, etc., as described in this disclosure. This example illustrates how networks of the invention can be effectively deployed across borders, sharing certain functions, while also complying with local RRs in the provision of output functions.

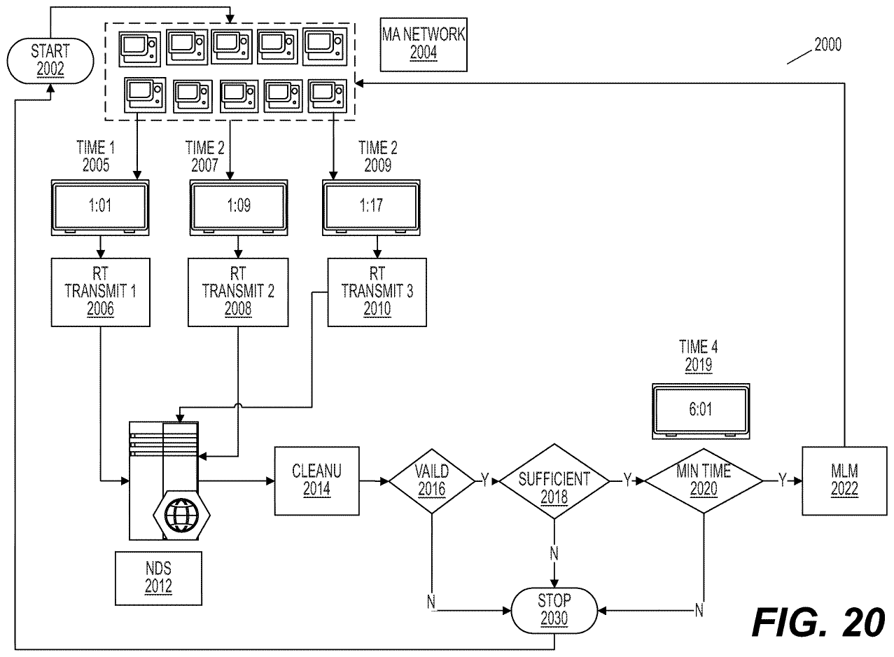

### FIG. 20

FIG. 20 depicts flow of data through a network, #2000, and processing of data over time according to time-dependent data processing rules, reflecting certain aspects of the invention. The illustrated process starts, #2002, by collecting data from MA network, #2004, over time. At time 1, #2005, corresponding to, e.g., 1 minute and 1 second post start (1:01), real time/streaming data is transmitted #2006 to NDS, #2012, and continues to be transmitted during the remainder of a time 1 cycle. At time 2, #2007, a time later (here, eight see later, at 1 minute and nine seconds (1:09)), a new data cycle begins with the transmission of new cycle data #2008 to NDS #2012. At time 3, #2009, eight more seconds later, a third exemplary data cycle begins (at 1:17) with the transmission of another set/collection/unit of RT/streaming data #2010 to NDS #2012. Collection of data in a fixed data cycle sometimes, most of the time, generally always, or always during operation of an MA:NDS network connection, is an aspect of the invention that is applicable to any other aspect described here. The use of collection cycles aids in analysis of MA-D in that, e.g., certain types of data can, in aspects, be expected to repeat (at least in format) in each cycle or each number of cycles according to preprogrammed rules. For example, MAs can be configured to deliver a set amount of physiological sensor data, device performance data, device status data, etc., within a preprogrammed data cycle, which is used as an analytical tool by the NDS in selecting data for analysis. Systems/methods of the invention also can include collection of larger sets of data from smaller cycles or other collections of data over time, over events, etc.

For example, such “cycle data” (data delivered in an expected transmission cycle for a set of streaming data), once received, can be subjected to various data processing step(s)/engine(s), e.g., data cleaning (e.g., by a data cleaning unit/engine, CLEANU, #2014). A collection of cycle data can be assessed for data validity, #2016, according to data validation rules, and if, e.g., enough valid data is present for collection of a larger data cycle (e.g., data is deemed sufficient, #2018) in terms of, e.g., data content and integrity, such data or a collection of such data and other cycle data is or can be maintained in temporary storage for combination with other data collections to form a larger data collection. E.g., temporarily stored cycle data can be evaluated for whether a minimum amount of data is present, #2020 (e.g., as measured by a minimum period with continuous collection of data therein) for the operation of a machine learning module (MLM, #2022). In the exemplified embodiment, about five minutes of collected cycle data (comprising, e.g., about 37-38 cycles worth of RT/streaming data) is collected and maintained with temporary storage/memory until a minimum amount of time passes or data is collected, #2020 (as exemplified here, this occurs at six minutes and 1 second, 6:01, from start of data collection #2019). This second, larger, data collection can then be subjected to processing, e.g., through application of machine learning module (MLM, #2022), to generate NDS-AD, which is relayed back to devices in the MA network, #2004. However, if data is not valid or sufficient for formation of a second collection/aggregation, #2016, #2018, the collection of data being added to the growing collection held in temporary storage may be stopped, #2030, and the process of collecting such a larger collection of data from smaller collections/data cycles started over again. The assessment of data validity can be based on any number of factors including, e.g., matching/linking with other data, completeness of data (e.g., continuity across a time series, expected schema, etc.), and the like (e.g., as determined by regression analysis, probabilistic matching algorithms, and the like). This cessation of data collection during a minimum collection time, rather than running the process over the entire period before making the evaluation, ensures that more evaluations are made, as NDS time is not wasted on a collection of data that ultimately will not be sufficient to meet standards of processing functions, e.g., an MLM, #2022. As noted, the exact times here are exemplary, and such processes can be performed with larger or smaller times, units of data, etc., and with higher order collections of data (e.g., number of first collections forming a larger second collection, and several second collections forming a third collection, etc.).

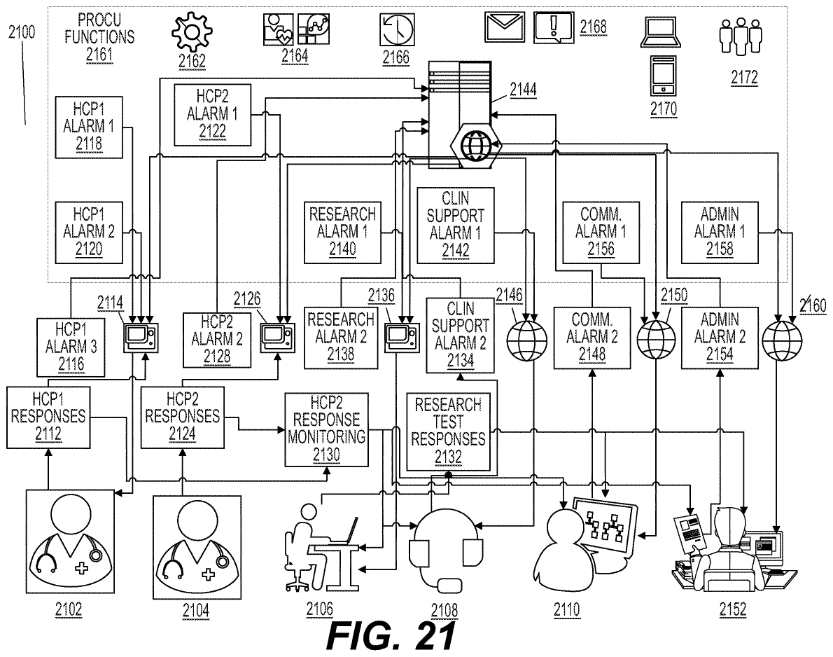

### FIG. 21

FIG. 21 provides another overview of components of a network, #2100, comprising several user groups, an NDS, and MAs, highlighting the ability of such an NDS to provide a variety of alerts/alarms to different users of different groups according to aspects.

A shown, first health care provider (HCP 1, #2102), associated with MA1, #2114, establishes alarm/alert settings, #2162, via setting controls on the MA (e.g., for a first alarm, #2120) or user via another network device/interface (e.g., for a second alarm, #2120) or both, and such settings are relayed to NDS, #2144. NDS also or alternatively applies processor level alarm(s)/alert(s) based on user group, applicable to, e.g., HCP1 (#2118), which settings, alarms, or both, is/are relayed to MA #2114 or other user device(s)/interface(s), or even devices or interfaces associated with other network user(s). HCP1 responses to alert(s)/alarm(s), #2112, can dictate operation of device components, modify NDS operation, or both, and also can be monitored by various user groups, through HCP response monitoring function, #2130, e.g., research unit, #2106, clinical support group, #2108, administrators, #2152, or a combination of any or all thereof. Accordingly, both local and network set alerts/alarms are applied to HCP1, and HCP1 responses can be monitored. Monitoring of responses can provide information concerning, e.g., the effectiveness of alarms/alerts relayed to HCP1, performance of HCP1, or both. HCP2, #2104, associated with MA2 #2126, similarly sets alarm/alert settings, #2128, and the NDS also sets HCP2 relevant alarms #2122, which are sent to MA2 #2126 when certain thresholds are met or exceeded (e.g., physiological parameters e.g., blood flow, heart rate, oxygen level, organ function, etc., which are detected by sensors associated with MAs (not shown)). HCP2 responses, #2124, to alarms/alerts, like HCP1, also can be monitored by HCP monitoring function, #2130, and such information relayed to various user groups, e.g., clinical support, research team, administrators, or any combination thereof (e.g., as described for HCP1). Readers will appreciate that in operation of many networks, ≥10, ≥50, ≥100, ≥150, ≥200, ≥250, ≥500, or ≥~1000 HCPs and MAs can be in a network, each with some level of custom alarm settings, reflecting the level of capability of systems/networks of the invention. Readers will also understand that the above-described and exemplified concept/principle of generally monitoring responses to alarms, analyzing such responses, and changing one or more aspects of NDS operation, MA operation, or both, based on such responses and analysis (e.g., changing alarm condition/nature), is a general aspect of the invention that can be combined with any other aspects described here.

Research team users, #2106, similarly provide local alarm/alert settings, #2138, at MA, #2136, and NDS also or alternatively sets research team NDS level alarm/alert settings, #2140, also displayed at MA, other user devices/interfaces, or both. Research team alarms may vary considerably from HCP alarms. E.g., HCP alarms may be limited to a specific set of alarm/alert settings that have been demonstrated to be effective in clinical practice, whereas research team users can be engaged in studying new alarm/alert settings for new devices, conditions, alarm/alert media, etc. Research group test responses, #2132, can be monitored and analyzed in the process of developing improved alarm/alert settings, protocols, etc., which can be later deployed to HCP devices/HCPs.

Clinical support team/group, #2108, can also set clinical support alarms at a user level, #2134, whereas NDS also or alternatively can have clinical support team/group alarms, #2142, but typically clinical support will receive output and set alarms via web interfaces, #2146, or other devices, rather than at the MA level. NDS #2144 relays MA-specific alarms/alerts to some user groups, but not others, e.g., clinical support.

Commercial group users/devices, #2110, also may sometimes receive alarms/alerts, but for Regulatory Requirement reasons, such alarms/alerts will typically lack any PHI, and it also can be the case that such users will receive information from a subgroup of MAs, e.g., also may be the case with clinical support team(s). Like clinical support, commercial group users can set local alarm settings, #2148, and can have alarms set at an NDS level, #2156. Also, like clinical support, commercial group users will typically not access alarms/alerts at the MA level, but rather will typically input alarm settings via a web interface, #2150, and receive alarms/alerts through various user devices (e.g., mobile phones etc.).

Administrator group users, #2152, which can include professionals monitoring/maintaining the NDS or performing NDS functions, e.g., engaging in supervised machine learning, similarly can set individual alarms/alerts, #2154, typically through a web portal/interface, #2160, and receive administrator class alarms/alerts set at an NDS level, #2158.

Processor unit (PROCU) functions, #2161, including the above-described NDS level alerts/alarms, also can include NDS level alarm/alert settings #2162, which can include, e.g., the type or content of data provide with the alert/alarm, or displayed data, #2164; timing, trigger, or repetition settings, #2166 (including, e.g., repetitions ~3, ~4, or ≥~5 times); communication channel settings, #2168 (e.g., whether alarms/alerts are relayed by, for example, email, text message, device notifications, automated or manual phone calls, log-in alerts, or combination(s) thereof); target devices/interfaces settings #2170 (e.g., mobile phone, workstation, etc. where alarms are presented/registered); and the alarm/alert recipient group settings, #2172, to also receive, be recommended to receive, or be notified of alarms at the user or NDS level for a particular individual, group, device, area, or entity, etc. Processor and NDS functions include the capability to manage such a level of different alarm/alert settings, users, across complex networks with various subnetworks, MAs, etc., through the methods and components discussed in this disclosure (e.g., efficient data tagging, employment of scalable, highly available, and typically massively parallel distributed processing at a cloud processor level). The highly customized alarm systems of the networks of the invention reflect yet another distinguishing characteristic of such a sophisticated/complex system as compared to previously described medical device data management systems.

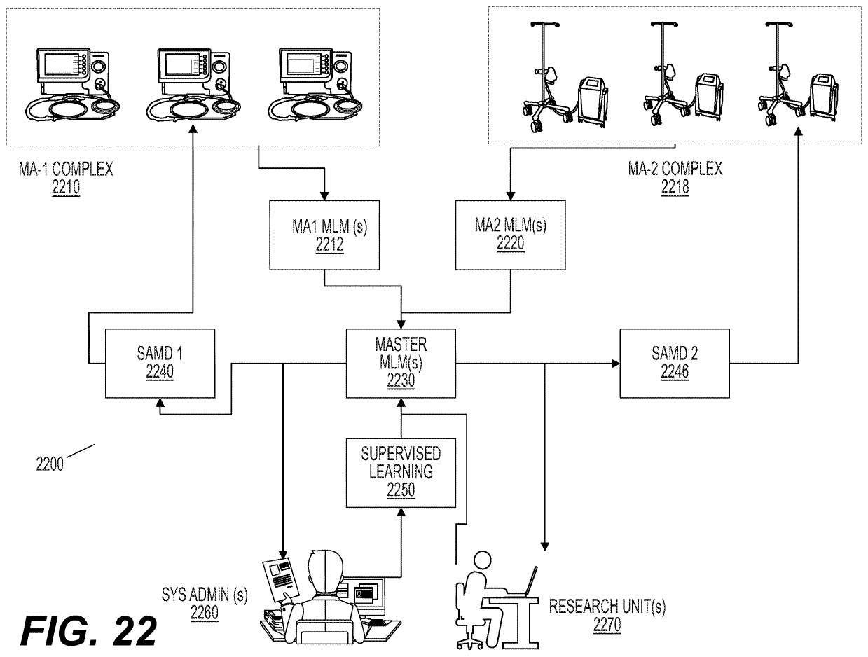

### FIG. 22

FIG. 22 provides a simplified overview of a part of a network #2200 comprising multiple machine learning modules (MLMs) according to another aspect of the invention. Complex(es)/group(s) of a first type of MA (MA-1 Complex, #2210) provide real time/streaming and other data, e.g., MA-CD to NDS, and at least some of such MA-D is processed by MLM(s) that is/are trained to analyze MA-1 type data, MA1 MLM(s) #2212. Similarly, complex(es) of a second type of MA (MA-2 Complex, #2218), deliver real time/streaming or other MA-D to network, some of which data is acted on by MLM(s) focused on MA-2 type data, MA2 MLM(s) #2220. To coordinate such data, particularly where patients are engaged with both MA-1 and MA-2 type devices, the system/NDS includes one or more master machine learning module(s), #2230, which receives either raw data from the multiple MAs, from the MA-specific MLM(s), or both, and analyzes such data to arrive at a combined overview and analysis of the data. Master MLM(s) can comprise supervised learning component(s) or be trained through supervised learning processes, #2250, in which NDS administrator(s), #2260, research unit users, #2270, or both, can receive proposed master MLM outputs/models and MLM adjustments, bases, and the like, and provide feedback/correction or other supervision, where necessary, to either base MLM(s), master MLM(s), or other component(s) of the system/network. Master MLM(s) can override one or more MA-specific data streams, combine the different MA data streams to come up with a diagnosis different than that obtained from device-specific MLM(s), or confirm the conclusion of one or more MA-specific data streams. MLM(s) can provide or act as a supervisory component to different SAMD modules that either control MAs, provide therapeutic directions to HCPs via MAs or specific to the operation of MAs, or a combination thereof (e.g., a first SAMD component, SAMD 1, #2240, can provide medical device applications to device(s) in MA-1 Complex #2210 and a second SAMD component, SAMD 2, #2246, can provide medical device applications to device(s) in MA-2 Complex, #2218). Such principles of using a master machine learning module can be extended to data generated from additional types of MAs; MAs operating under specific patient, Entity, or Regulatory Requirement conditions; or a combination thereof (which data can optionally be first analyzed by more specific/lower level MLMs as exemplified in this Figure). Master MLM(s) can, in aspects, operate a conditional level, wherein such MLM(s) are triggered e.g., by data in one or more MA-specific MLM(s) or MA-D meeting or exceeding preprogrammed thresholds, triggering the review/intervention by master MLM(s). Master MLMs can be generated or improved through supervised learning methods, such as are described elsewhere. Inclusion of a master MLM component in an NDS can provide for DoS better patient outcomes, especially where patients are being treated with multiple MAs, as is often the case with patients in critical health conditions (e.g., multi-system failure or multi-system conditions). Master MLMs can be trained based on, e.g., outcome data associated with therapeutic applications, sensor data, and the like, associated with the various MA and other inputs provided to an NDS. Machine learning methods for clinical diagnosis and related applications/principles and methods are known in the art (see, e.g., US20160012349, WO2021012225, US20210098130, WO2020047171, US20200357515, WO2021044431, US20200402663. US20200111570, WO2018220565, US20210202094, U.S. Ser. No. 10/861,606, U.S. Ser. No. 11/037,070, US20210065898, US20190371464, US20190348178, US20210090738, WO2019246086, as well as other references cited herein. Such methods/principles and systems/components can be combined with or adapted to the aspects, principles, and methods described in connection with this Figure or provided in any other parts of this disclosure. Systems/networks of the invention clearly can provide more robust data sets for better training of medical diagnosis/treatment-related machine learning applications than previously possible based on previously described medical data management systems, thereby improving the performance of many ML systems/methods.

### FIG. 23

FIG. 23 depicts flow of data into, within, and out from a streaming data processor (SDP/SDE) component of an exemplary system/NDS and the selective processing and analysis of streaming data before ingestion into NDS memory.

Data (MA-D) is sent from several MAs, #2302, including MA-1, #2301, which transmits RT/S-MA-D, #2303, and MA-2, #2305, which transmits a data stream comprising both RT/S-MA-D and L-STR-MA-D/MA-CD #2307 (e.g., after an off-line event). Focus is placed on a few MAs herein for simplicity and conciseness, but recognizing that in most cases≥10, ≥50, ≥100, ≥200, ≥500, or ≥1000 MAs of one or more types are transmitting data comprising MA-D simultaneously and concurrently with input(s) from different ONDs, etc.

Most, generally all, or all the MA-D transmitted from the MAs, #2302, including MA-1 and MA-2, #2303 and #2305, respectively, can be in a fixed/known semi-unstructured format to increase speed of processing and ingestion (e.g., most, generally all, or all MA-D can be relayed/presented/contained in JSON file formatting). Streaming Data Processor (“SDP”), #2320, includes a streaming data (“SD”) receiver module/component, #2321, which can be considered a type of Input Unit (or a part of an Input Unit). A box around these and other components in the Figure signifies that these components/functions are part of or are otherwise closely associated with the SDP. SDP can receive multiple streams of data simultaneously from the numerous MAs that network with the NDS. The SDP receiver, #2321, can effectively receive and register several streams simultaneously (e.g., from ≥100, ≥200, ≥500, ≥1000, ≥2500, ≥5000, ≥10,000, ≥25,000, or ≥50,000 stream clients concurrently transmitting data). The SDP receiver can be considered an aspect of or share resources with other elements of SDP memory, such as stream registry file(s). Communication into the SDP can also comprise or utilize various network interfaces, which are known in the art.

Effective stream processing by the SDP can be achieved by any suitable means, examples of which are described elsewhere. E.g., SDP can perform parallel receipt on received streams of data, and typically perform a limited series of operations (e.g., kernel functions), typically to most, generally all, or all the elements in a stream. Most, generally all, substantially all, or all processing conducted in the SDP is performed in SDP hardware and software, with very little or no reference to/interaction with the main NDS memory (e.g., a data lake/EDL primary NDS-MEMU, #2360). In aspects, most, generally all, all of the streaming processor functions are performed uniformly to elements in a stream, at least in a stream of RT/S-MA-D (i.e., such data streams are subjected to uniform stream processing/uniform streaming). In aspects, known compiler components can be employed to automate and optimize in-memory/on-chip processing of stream data in the SDP Unit (in which SDP memory is at least functionally separated from the primary memory of the NDS). At the hardware level, the SDP, #2320 can be equipped with, e.g., a multi-memory bus system (e.g., crossbar switches) (e.g., one or more ≥512 MB cross bard switches), and a multi-processor/cluster processing unit, and memory distinct from the primary system memory (e.g., the EDL), in terms of physical or data rule separation. Besides RT/S-MA-D and L-STR-MA-D/MA-CD, SDP receiver, #2321, also can receive unstructured data (e.g., from email or text message inputs, n.s.). In aspects, most, generally all, or all data inputs from other systems (e.g., networked third party CRMs) are handled separately from inputs to the SDP.

Component(s)/Engine(s) (e.g., kernel(s)) can be characterized as/make up an SD Handler, #2322, which can be a component of an SDP, and which, i.a., analyzes the nature of the incoming data streams, e.g., identifying and transmitting MA-CD to the MA-CD immediate analyzer (which can also be considered a “cache processor”), a component of the SDP that performs immediate analysis on the MA-CD, #2324, determining if there are conditions in the MA-CD that require triggering operation of alarm(s), control over the associated device, or other NDS Action (in this respect this component can also be considered an event processor). In aspects, the MA-CD immediate handler may perform or performed a limited amount of MA-CD and RT-D-MA-D harmonization/reconstruction for this component of the SDP to determine if there is immediate data requiring immediate action in terms of alerts, MA control, or otherwise (i.e., an initial analysis as discussed elsewhere). The SD Handler also can send streaming data to additional SDP memory, such as an SD buffer (e.g., #2323), or queue to handle according to processing/prioritization rules based on component availability, stream load, etc. In aspects, known software systems for receiving and processing data streams suitable that can be considered suitable components of SDP receiver #2321 and handler #2322 (e.g., Kafka, Apache Flink, etc.,) are discussed elsewhere. An SDP Buffer #2323 can be composed of, e.g., a registry file/file system, such as stream registry file(s) of the kind known in the art, and additional local SDP memory.

In SDP memory streaming data that is ready for analysis is analyzed by the SDP Analyzer Unit/Function, #2325, which determines whether RT/S-MA-D includes any data elements that require the triggering of alarms, control of MA(s), which are under the control of an SDP Controller, #2327, which comprises engine(s) for control of network devices based on SDP processes/analysis (e.g., in provision of treatments, in control of displays, e.g., in performing CDSS functions, etc.). The SDP analytical process typically is limited to certain data elements only (e.g., device data, critical sensor data, etc.) and excludes many other elements of data stored in the primary NDS memory, #2360 (e.g., data concerning any associated commercial users would not be analyzed at this level of processing). RT/S-MA-D and MA-CD is thereafter or otherwise transmitted out of the SDP by an output, #2328, to other component(s) of the NDS. In this respect, the SDP Handler can be viewed as an agent that converts streams incoming to the SDP to outgoing streams/other outputs (e.g., local output registry files).

After outputting from the SDP, other components of the NDS memory unit perform data ingestion functions, #2330, typically after data cleaning, harmonization, or both, as described elsewhere herein, resulting in storage of the data in the primary NDS memory, such as a data lake or EDL, #2360. This component of the SDP (among others) can also be considered part of an NDS Input Unit (NDS-INPU). A primary NDS Processor (n.s.), can, after DL/EDL ingestion, perform automatic, on-demand, or conditionally automatic NDS-MEMU analytical functions, #2340, on data stored in the DL/EDL (e.g., machine leaning functions as described elsewhere) and can perform on-demand functions, such as data query processes, #2350. Ingestion functions applied during ingestion, #2330, can include application of metadata, particularly to unstructured data, and segregation of incoming data of different formats and contents into EDL management zones. On-demand query and automatic query processes including only EDL data that was received in an unstructured manner or both unstructured and semi-structured data may be different from those applied to semi-structured data (e.g., the former being based on keyword searching and the latter focused on attribute/value pairs). Both types of query functions can include both types of queries against such different groups of data, generating different NDS-AD. EDL data recognizable/used in such query functions often will include data not analyzed by the SDP, such as non-critical sensor data, non-critical device performance data, Commercial user association, and the like.

By separating resource-intensive analytical processes such as ML processes and query processes from the immediate processes performed in the SDP, exemplary NDSs of the invention can more frequently operate in a real time or near-real-time state, despite the significant amount of incoming streaming data received and processed by the NDS. Use of an efficient primary memory, such as an EDL, further ensures that even second-line processes, such as automatic NDS-MEMU analytical processes can be performed within a matter of minutes from NDS receipt of streaming data.

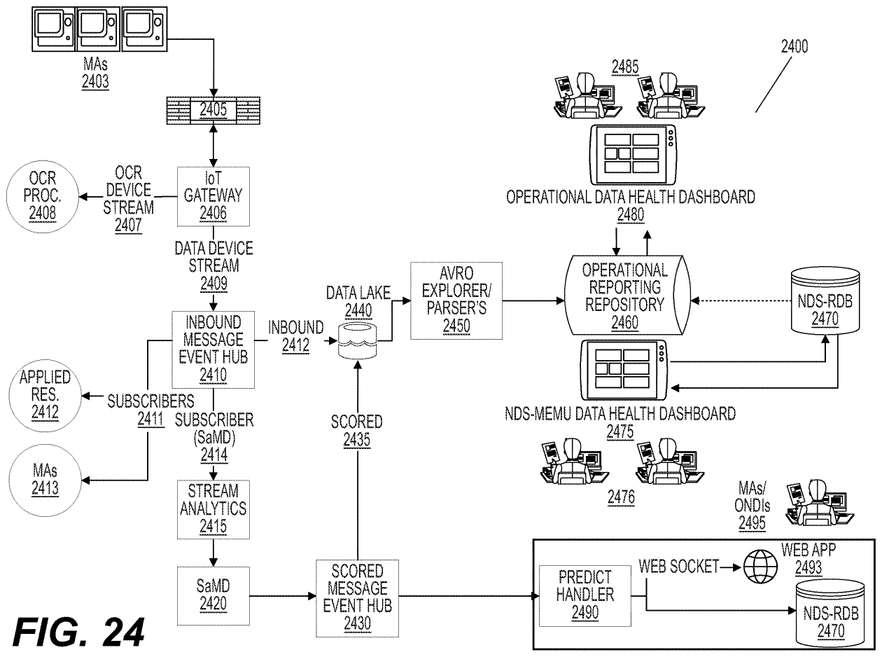

### FIG. 24

FIG. 24 depicts yet another diagram of parts of an exemplary system/NDS of the invention, #2400. Medical apparatuses (MAs) #2403, which may include one or more MZMAs (not shown), single component MAs, or both, relay data to, and receives data from, the NDS. While only a few MAs are shown in this Figure for sake of simplicity, a network could include ≥50, ≥100, or ≥1000 MAs, all relaying data to or receiving data from the NDS, simultaneously, in operation, which can be of different types, statuses, locations, affiliations, etc. (not shown).

Data flow into and out of one or more of the MAs #2403 can be controlled by a device security system or firewall #2405. One or more MAs also may be subject to other anti-tampering protections as discussed elsewhere herein (not shown). In operation, each MA relays data (e.g., in streaming digital alphanumeric format, image format, or both), rapidly to NDS (NDS/MAC-DMS) (e.g., at a rate of (1) ~5-15 digital alphanumeric messages per second (e.g., ~7-12 messages per second, such as 8 messages per second) or (2) 1 image every 10-30 seconds (e.g., every ~20 seconds)). If an MA is operational and securely and stably networked with an NDS/MAC-DMS, such communications are at least substantially continuous. Most/all such communications are typically relayed via secure internet communications.

Incoming MA-data is received by an IoT (internet-of-things) gateway component of the NDS (NDS/MAC-DMS), #2406, which can act as or comprise a streaming data processor (SDP) and can be considered or considered a component of an input unit, as described elsewhere herein. Gateway, #2406, can broadcast/relay digital messages as a data stream, #2409, to an inbound message event hub, #2410. Simultaneously or otherwise concurrently, gateway, #2406, also can relay messages to subscribers (e.g., other engine(s), connected database(s)/system(s), or ONDIs). E.g., as shown, gateway, #2406, also relays image data, #2407, to an OCR processing module (OCR PROC., #2408), which converts OCR-readable elements of image data to alphanumeric data.

Inbound message event hub, #2410, which also exhibits elements/functions of an SDP, can both route and selectively relay data, including MA-data, to subscribers and handle some or all aspects of ingestion into NDS (NDS) memory. In the latter sense, inbound message event hub, #2410, can collect, optionally process, and relay inbound message data to an NDS data lake (DL)/enhanced DL (EDL), #2440, via one of several simultaneous output streams, #2412, typically on a timed or conditional relay basis. Datasets comprising MA-D in such inbound output streams, #2412, typically consist generally, essentially, or entirely of MA-D (i.e., are not significantly modified and do not comprise analytical data). Gateway, #2406 or message hub, #2410, can collect data in periods of 1-10, 2-8, 3-6, 3-7, 2-6, or 4-6 (e.g., about 5) minute intervals, and then repeatedly relay such collected data to the DL/EDL in continuous/repeated batches/collections/units. Inbound message and event hub, #2140, typically will also include capability for compressing data prior to ingestion in the DL/EDL (e.g., in an AVRO format known in the art). Inbound message and event hub, #2410, also can relay messages or pre-ingestion analytical data, commands, and the like, to one or more other subscribers. #2411. E.g., as illustrated, inbound message event hub #2410 simultaneously relays message data to applied research unit users/systems/devices, #2412 and to various other MAs in the network #2413 (e.g., sending device command instructions, device display instructions, and the like). Inbound message event hub #2410 also simultaneously relays SaMD-relevant data, #2414, to an SaMD processor, #2420, after analysis by a stream analytics engine, #2415. As exemplified in this way, message hub, #2410, can selectively direct particular messages/data (or data types) to various functional units of the NDS. E.g., a hub might only relay HCP-associated MA-derived MA-D to an SaMD, and route research user class MA-D to other functional units (not shown).

Stream analytics engine/unit, #2415, can perform various data analytical functions on streaming data relayed to SaMD/SAMD module, #2420, including, e.g., data filtering, data cleaning, data validation, NDS administrator alerting, and the like. SaMD engine, #2420, generates analytical data (NDS-AD), e.g., scored message data, which is relayed, first to a scored message event hub, #2430, which, in turn, relays scored data #2435, to data lake/EDL, #2440, such scored message data being received and stored in a different governance zone of the DL/EDL (not shown) from inbound data stream #2412. Scoring of data as part of a process of data transformation can be applied generally to any aspect of the invention described here, such scoring being applied by, e.g., comparison of data against one or more preprogrammed and programmable data classification or validation rules applied by NDS component(s). Simultaneously/concurrently, scored message event hub, #2430, relays prediction data to a prediction handler unit/engine, #2490, which in turn relays analytical data (NDS-AD) (here comprising, e.g., physiological parameter predictions) to web applications/interfaces, #2493 (e.g., by generating an output formatted for display on a web application interface) and various network MAs and Other Network Devices and Interfaces (ONDIs) (aka, ONDs), #2495. Relayed information also can include output functions, such as instructions for triggering alarms, changing device operation, etc.

Data in DL/EDL, #2440, cab be automatically regularly or otherwise periodically subject to compression by a data compression unit, such as an AVRO Explorer, #2450, or an equivalent thereof, and compressed DL data is then relayed to one or more structured database data repositories, such as a first database (here, an operational reporting repository, #2460, which stores data concerning NDS performance including (1) data received directly the analytical processor or other functional components of the NDS and (2) data obtained or received from a second relational database (NDS-RDB, #2470), which comprises structured data sets generated from output relayed to network components from the NDS/MAC-DMS). Data in the operational reporting repository/first database also is stored in a structured format suitable for inclusion in a relational database (e.g., comprising multiple levels of data in various fields, records, and tables in an ordered hierarchy) and can be used for performing queries, reports, and the like, using more complicated data structures than are stored in other parts of the NDS's data repository, such as in an enhanced data lake, providing another means of ensuring or enhancing NDS operability.

Operational data health dashboard, #2480, can be considered a functional module (FM) comprising or corresponding to engine(s)/unit(s)/system(s) that provides system/NDS analyst #2485 with the ability to provide continuous, regularly recurring, conditionally occurring, or on-demand monitor data quality entering into or in the operational reporting repository. The dashboard can be a schema/representation delivered to various device(s)/interface(s) or can be a dedicated device/NDS component, in either case providing visual indicator(s) of various aspects of data health (gaps in data, delays in data intake/processing, consistency of data, amount of data modification, correlation of data to expected data parameter(s), and the like). Using data visualization dashboards such as this exemplified dashboard, #2480, data quality problems can be determined manually (e.g., through visual inspection), automatically (by comparing data against predetermined standards, scanning for missing/damaged records/datasets, etc., or a combination thereof). NDS analyst users can upon learning of such data issues make changes to aspect(s) of NDS operation to reduce the likelihood, severity, or frequency of such data errors arising. The use of similar dashboard monitoring methods/systems can be applied to any other aspect.

As noted, system-relayed data can be relayed here to a second relational database, NDS-RDB, #2470, which can serve as a basis for network-visible functions/outputs, such as data queries (not shown). Data in and entering NDS-RDB, #2470, is accessible by NDS-memory unit (NDS-MEMU) data health dashboard, #2475, which allows system analysts #2476, and possible other users, such as independent entity network analysts, to evaluate data entering or that is already contained in the second relational database.

This example demonstrates various aspects of the invention, including the processing of data through an NDS, analysis of events early in NDS analytical processes, employment of structured databases as a supplement to a DL/EDL DR, and the use of monitoring dashboard(s) to ensure data quality/data health in operation of the system, all of which reflect additional distinguishing aspects/features of systems/networks of the invention.

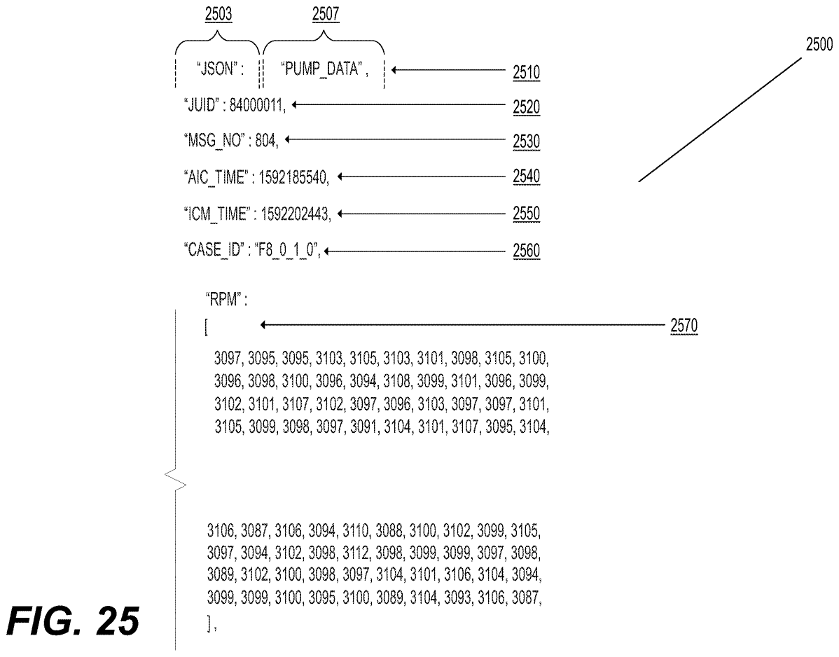

### FIGS. 25, 26, 27, and 28

FIGS. 25-28 are exemplary semi-unstructured datasets of the types that can be relayed by MAs to an NDS and acted on by NDS component(s), such as an SDE, stored in a DR, such as an EDL, and used to generate NDS-AD, which is, in turn, used in the generation of output, such as output applications (not shown in FIGS. 25-28).

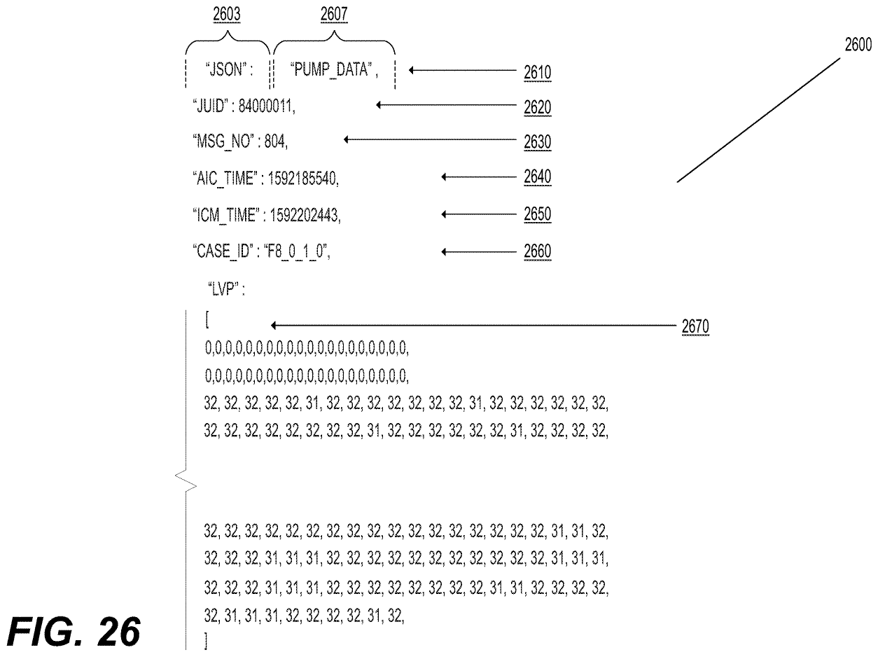

FIG. 25 specifically includes a semi-structured data structure (a JSON record), #2500, that includes several pre-defined attribute entries, #2503, and corresponding value entries, #2507, forming records/fields within the semi-structured data structure. The datasets of FIGS. 26, 27, and 28, are also semi-unstructured JSON-type datasets including attribute/value pairs.

As shown in each of FIGS. 25-28, such datasets can include dataset identification information, usually provided as the first part of the dataset (as shown), which can enable NDS component(s) to associate the dataset with a particular MA and derive MA status, ownership, location, type, etc. In general, methods of the invention can include analysis of and systems can be configured to receive data from MAs that include multiple points of data, contained in multiple different semi-unstructured collections, comprising multiple types of attribute/value pairs, at least some of such pairs including a large number of values for two, three, four, or more attributes, such attributes typically including sensor data, device performance data, or both, as exemplified in these Figures and described in the following paragraphs.

In FIG. 25, for example, a data record, #2600, includes a first line including a value reflecting the type of data record, #2503, “JSON,” indicating that the data record is a JSON formatted record). The first line, #2510, of the data record, #2500, further comprises a second value, #2507 identified as “Pump_data” signaling that the record pertains to pump MA performance/status information. Note, that in neither case in the first line is an explicit attribute field provided, reflecting that, in cases, an attribute can be determined based on, i.a., position in the data record alone.

Second record, #2520, associated with the attribute “JUID,” includes one record-identifying information record/entry/value (“84000011”) that can be used to separate this dataset from other datasets received by the NDS. Third records, #2530, associated with the attribute “MSG NO,” provides another identifier value (“804”), here relating to the specific dataset, distinguishing this dataset (message) from other datasets sent from the same MA or same MA in the same period. Fourth record, #2540, provides a time indicator record for one zone of an associated MZMA from which the MA/dataset is relayed. Fifth record, #2550, provides a second time indicator record for the second zone of the associated MZMA. Sixth record, #2560, associated with the attribute “CASE_ID,” provides a third dataset/source identifier (here the value “F8_0_1_0”).

As can be seen from this exemplary dataset, a SUMAD dataset generated by an MA can comprise multiple identifiers, which can identify datasets (e.g., associated with specific time(s), particular MA(s), or particular function(s) performed by particular MA(s), etc.), and also several time indicators, especially in the case of MZMA(s). Such principles can be modified and applied to any other aspects of the invention. Inclusion of multiple identifiers in a data record, can aid in data security, data analysis, or both.

The second part of the dataset, #2500, shown in FIG. 25, provides several values associated with the “RPM” attribute, #2570. This exemplifies how MA can include in one dataset or one part of a dataset device performance data (here pump rotations per minute (RPM)).

The dataset in FIG. 26, #2600, includes both similar attribute #2603 and value #2607 record layout and similar identifying (sometimes called header) information in the first part of the illustrated dataset (e.g., a PUMP_DATA value, #2610, and other identifiers as described above, #2620, #2630, #2640, #2650, and #2660) and a dataset containing multiple values for the “LVP” attribute, #2670, which reflects MA sensor data (specifically, left ventricular pressure (“LVP”)), measured in a patient associated with the MZMA. A string of data at the start of the value records (including multiple “0” entries) might reflect a non-operational state or other invalid data that might be discarded in a data analysis.

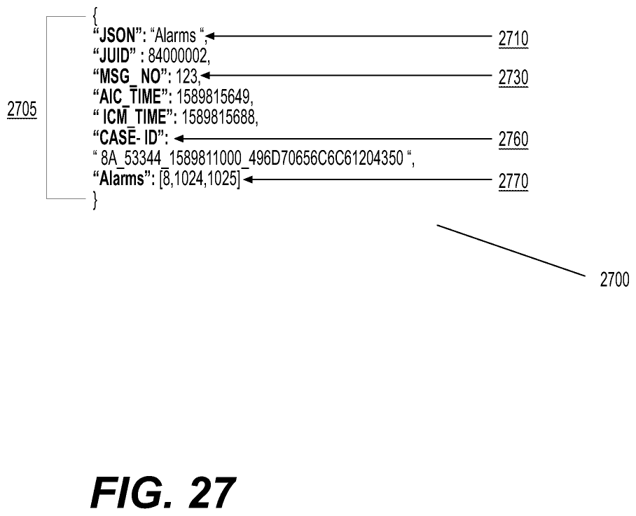

The similar semi-unstructured dataset, #2700, shown in FIG. 27, includes a collection of value/attribute pair records, #2705, including different identifying information and device performance data. Specifically, first records, “JSON” includes the value “Alarms,” #2710, identifying the dataset as including alarm data, providing for, e.g., quick detection by an SDE when the MA-D is received by NDS. Unique message number and case number records, #2730 and #2760, are once again provided, and device performance data here is in the form of data identifying alarms registered in the MA, #2770.

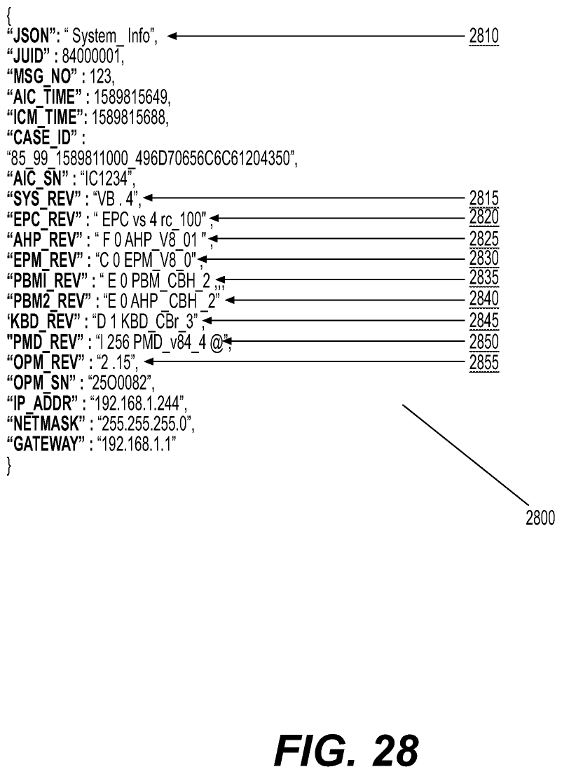

The semi-unstructured dataset, #2800, shown in FIG. 28, includes a first dataset identifier/record, #2810, as well as a variety of system/software status information. E.g., various operating system, software, and hardware versions are provided in records #2815, #2820, #2825, #2830, #2835, #2840, #2845, #2850, and #2855, exemplifying how multiple types of status information records can be included in a SUMAD dataset. Such information can be used by the NDS to determine if the MA requires updating, to understand the performance capabilities of the MA, etc.

In aspects, some or all the above-described types of MA-D are combined into a single dataset relayed to an NDS (e.g., a dataset can include any combination of device status information; device performance information; system, software, or hardware version information; sensor data, along with multiple points of MA/dataset identifiers). The semi-unstructured nature and layout of such data provides for rapid and efficient analysis/processing and storage of the data by NDSs/methods, which is important in the context of providing medical treatment or diagnosis.

### Technical Effects

Skilled persons will recognize that the systems and methods of the invention afford several technical effects, providing tool(s) which have heretofore not been available or solving several problems which have heretofore not been addressed or addressed in a similar or sufficient manner by known systems/methods, by use of the various technical features of this disclosure. Various technical effects are described elsewhere in this disclosure, and a few specific/exemplary technical effects are highlighted/reinforced here.

One exemplary technical effect of the invention is the ability to securely, and in a real-time or near-real time manner, manage medical device data generated by a plurality of separately located medical apparatuses (MAs) (e.g., MAs in a WAN), the plurality of separately located MAs each having an interface with a network data management system (NDS), which could not otherwise be feasibly or timely managed, by a human or group of humans, and from which useful and time-sensitive data can be extracted, compiled processed, and applied or otherwise acted upon, which could also not otherwise be feasibly accomplished by a human or groups of humans, which is addressed herein through technical functions of MAs and NDSs.

In aspects, technical effects of the invention include the concurrent control of the operations of a plurality of networked MAs in a network with an NDS (a DCDMS), such control of operations based on analysis of both SMAD and cache data stored during periods when MAs are offline and performing analytical and control functions based on the combination of such data. Such control of operations can comprise delivery of therapeutic instructions, delivery of predictions, or control of therapeutic components.

In this and other respects, the invention improves the functioning of medical devices/apparatuses in a network as well as the overall functioning of the system itself. The system provides for improved management of multiple devices simultaneously based on evaluation of sensor data taken from the population of devices, other data inputs, and historical collections of collective device data and individual device/patient data. Without the combination of elements of the inventive systems described herein, such medical devices would not operate as accurately or effectively, and other users of the system would not be able to access data from the system in as useful and compliant manner as is possible using systems of the invention.

In aspects, technical effects associated with the invention include the protection of sensitive MA components from unauthorized internet communication intrusion, hacking, etc., which can comprise providing multi-component MAs (multi-zone MAs (MZMAs)), which comprise a restrictive component/zone, which has either significantly restricted internet communications or no direct internet communications. In aspects, control of such a component requires local action in addition to network level action, e.g., local request for a software modification/upgrade when such a modification/upgrade is available.

In aspects, technical effects associated with the invention include the ability to process streaming data inputs from a large number of MAs, often MAs of different types, associated with different subjects with different conditions, operating under different states, etc., but providing real time or near real time analysis of such data, through the use of MPP functions, and employing an enhanced data lake memory structure and ingestion process, as well as data cueing and system scaling according to preprogrammed instructions executed by the processor(s) of the system. In aspects, systems and methods of the invention include performing limited initial analysis on RT/S-MA-D and MA-CD in intake prior to or during NDS DR ingestion and, where appropriate, performing initial control or output actions based on such initial analysis. One way in which this is achieved is through the employment of multiple processing systems and memory components (e.g., an initial SDP comprising an SDP processor and memory and a primary processor unit and primary memory/DR, such as an enhanced data lake).

In aspects, technical effects associated with the invention comprise the concurrent/simultaneous relay of analytical data generated from analysis of MA data to a variety of users in different user classes, including HCPs and commercial/BP class users/devices (ONDIs), wherein the delivered data is tailored to such different users and provides for, e.g., redaction or exclusion of PHI from the data delivered to commercial class users. In aspects, technical effects also or alternatively comprise receipt of input data from research class users, and the use of such data along with clinical MA data in generating some system analytical data, while also segregating such data and analytical data based on such data for compliance with regulatory requirements and for other clinical/business purposes.

Technical functions/benefits of the system(s)/method(s) can, for example, lead to the provision of enhanced patient care, e.g., including predictive functionality or also or alternatively new learning(s), potentially leading to the prevention of otherwise expected adverse medical events or future improvements in care, technical effects which can prove invaluable to healthcare operation(s) and human life.

Such exemplary technical functions of MAs include MAs comprising device physical, transferrable, and reproducible computer readable media (MAPTRCRM) comprising a processing function for executing device computer executable instructions (MACEIs) and a device memory (DM) that in operation records information comprising sensor information over time; a device display unit (MA-DISPU); a device relay unit (MA-RELAYU/DDRU) capable of transmitting real-time (RT-MA-D) or stored (MA-CD) structured, unstructured, or semi-structured device information (MA-D) comprising sensor information to the NDS; a device data input unit (MA-INPU); and a device data security system (MA-SECURU) comprising microcontroller(s) providing data protection functionality including restricting data based on established approved data types.

The inventive systems and methods provided here comprise the use of multiple time-based data collection cycles, different timed actions, and different data governance zones within system memory, to improve the quality of data analysis and the ability of the system to process rapidly incoming streaming data from numerous devices simultaneously.

Exemplary technical functions of NDSs include NDSs comprising a memory unit (NDS-MEMU) comprising an NDS PTRCRM further comprising a searchable data repository (DR) receiving and storing semi-unstructured MA-D (SUMAD), and NDS CEI (NCEI) encoding instructions for functions carried out by the NDS; an NDS processing function (NDS-PROCU) that executes the NCEI; an NDS data input unit (NDS-INPU) that can automatically receive data from MAs in communication with the NDS and distinguish the type(s) of MA-D received from each MA (e.g., RT-MA-D, MA-CD, or both); an NDS analytical unit (NDS-ANALU) that analyzes real-time, stored, and semi-unstructured MA-D to generate an analysis and, further, apply such analysis to the performance of one or more NDS functions; an NDS device data harmonization unit (NDS-DHU) that evaluates if MA-D is approved for use by the NDS-ANALU and determines how such approved MA-D is used by the NDS-ANALU; an NDS output processing system (NDS-PROCU) which can automatically filter MA-D, analysis, or both, for selective delivery to each MA or to other network devices/interfaces (ONDIs) e.g., other clinical, non-clinical, or research components, based on confidentiality and health care compliance rules; an NDS data relay unit (NDS-RELAYU) that securely relays over the internet specific information to specific target locations, e.g., to each MA that is MA-specific, patient-specific, or both, and that is specifically identified and of one or more specific data types that is configured to be adaptable by MA-SECURU(s), as well as relays information comprising MA-D, analysis, or both, to one or more ONDIs wherein the information relayed to ONDIs can comprise information from a number of MAs associated with independent entities (IEs); and an NDS stored data analytical function (NDS-ANALU) that generates stored data analysis (LS-D-ANALU) based on application of one or more schemas to semi-unstructured M-AD.

System(s) and method(s) of the invention can centrally collect, centrally store, and centrally analyze MA-D derived from independently operating MAs of a plurality of IEs, which together form an MA-network, the MA-D comprising a variety of data originating from MAs, and selectively communicates information based upon such data collection and analysis to a plurality of entities, e.g., clinical entities, sales entities, and research entities, wherein such communication is limited based on target recipient profile(s).

Such processes are like processes recognized as comprising patentable subject matter by patenting authorities. E.g., such processes be compared to, for example, the collection and distribution of stock-related data (stock quotes) described in Example 21 of the United States Patent and Trademark Office (USPTO) guidance related to subject matter eligibility available via the USPTO, whereby stock quote alert(s) are formatted into data blocks according to established information formatting protocols or preferences or rules, or address/destination of the data, and wherein data is filtered or analyzed according to set established specifications prior to distribution to an ultimate destination over a communication channel. Other aspects are like, e.g., Example 21 as well in the fact that, as described herein, data can be cached in such a manner that data becomes available (e.g., is transmitted, e.g., in the example stock alerts are provided) when the system is in an online status. Accordingly, aspects described here provide significantly more than simply organizing and comparing data. As is exemplified in the provided USPTO example, the invention comprises use of a transmission component with specific, limited functionality (e.g., microprocessor) and a memory to store rules/settings/preferences, transmitting data and associated alerts from a first location over a data channel to at least a second location, and providing a mechanism for receiving and interpreting the data or alert, even if such data/alert is collected when all aspects of the system are not necessarily online such that the collected data is transmitted once open communication is established.

The combination of memory and processing functions in combination with the data sources, which act upon the data received from the source, provide significantly more than the abstract idea of using data collection techniques to manage, e.g., data. The application of function-specific processors, analytical units, data modification units, memory units, relay units, and other functional modules described here within a single system to receive, transmit, manage, control, and apply medical data from a plurality of units (e.g., comparable to a plurality of video cameras) and to integrate the same to provide meaningfully different new data sets provide, again, significantly more than the abstract idea of data management.

Technical functions of system(s)/method(s) herein describe identification and analysis of aberrant patterns which can direct or inform further processing, thus modifying that which an NDS user would otherwise encounter absent such an NDS or method; further the systems and methods provided here employ corrective action to such aberrant data or patterns in data, e.g., by applying corrections or directing alerts, directing activity specifically according to the identification of or correction of, aberrant data or patterns of data (see, e.g., USPTO guidance, supra, Example 46).

The centralized collection, storage, and analysis of MA-D, which can be generated by MAs associated with a critical life support function, from independently operating MAs within or representing a plurality of IEs, provides additional clinical oversight over that available local to each MA, provides opportunities for supporting clinical research, product development and sales efforts, and further provides mechanisms for improved MA product performance by virtue of the at least semi-remote controllability of aspects of the systems and methods disclosed herein.

Thus, exemplary element(s) of technical features include continuous data transmission by NDS-RELAYU(s)/DDRU(s) to NDSs in data format(s) acceptable to the MA-SECURU(s); NDS functions for identifying past, present, and predicted MA-D, the NDS-ANALU capable of performing predictive function(s) based on the analysis and relaying the results of the predictive function to the MAs, ONDIs, or both; the NDS-ANALU performing function(s) regulated as software as a medical device (SAMD) and non-SAMD functions (NSAMDs), the system delivering output of SAMD and NSAMD functions to MAs in accordance with CEIs that reflect applicable regulatory status of the SAMD and NSAMD functions; SAMD being capable of changing the operating conditions of MA(s) in response to one or more conditions; the NDS-RELAYU delivering MA-D, analysis, or both to a plurality of GUI schemes based at least in part on user roles; CEI comprising one or more alarm conditions associated with sensor data, the NDS-ANALU determining if one or more alarm conditions are triggered by MA-D, analysis, or both, and the NDS causing an alarm to register in the MA, in a user associated network access device (NAD), or both based on sensor data and permitted user options; the MA-SECURU comprising anti-tampering detection functionality; and, e.g., the NDS-MEMU comprising separate governance zones managing storage of, use of, and access to various types of data (e.g., RT-MA-D, MA-CD, or both, curated data, scored data, or both, system testing data, and outgoing data). Physical components, e.g., processors and processing systems are employed along with communication channels and data recognition methods to solve the problem of providing comprehensive and effective medical device data management which integrates, manages, and effectively and proactively utilizes all the various data provided by a plurality of medical devices within complex and modern medical treatment environments. systems and methods provided herein solve the problem of securely managing such vast, varied, and complex medical data in a compliant and effective manner.

Technical effects can include combination(s) of any such above-described effects.

### LISTING OF EXEMPLARY ASPECTS

The following is a non-limiting list of exemplary aspects of the invention, intended to illustrate some embodiments of the invention presented in summary form. Similar to patent claims, aspects described in the paragraphs of this section may make reference to (depend on/from) one or more other paragraphs. Readers will understand that such references mean that the features/characteristics or steps of such referenced aspects are incorporated into/combined with the referring aspect. E.g., if an aspect in a paragraph (e.g., paragraph 501/aspect 2) refers to another aspect by paragraph or provided aspect number (e.g., paragraph 500/aspect 1), it will be understood that both the elements, steps, or characteristics of paragraph 500/aspect 1 in addition to those in paragraph 501/aspect 2.

In another aspect, the invention provides a system/NDS (e.g., a MAC-DMS) that comprise (1) a streaming data processing Unit that automatically and continuously receives and processes MA-D relayed from a number of medical apparatuses that the system has permission to receive MA-D from and that performs an initial analysis on received streaming MA-D to determine if one or more preprogrammed conditions are present in the streaming MA-D and, if such one or more conditions are present, acting as a controller of one or more of the medical apparatuses, one or more other network devices, or both by performing one or more of a preprogrammed limited set of initial functions, which initial functions comprise relaying instructions for controlling the operation of one or more medical apparatuses networked with the system, one or more other network computerized devices networked with the NDS, or both; (2) a system memory component that comprises processor-executable instructions and one or more data repositories, the one or more data repositories comprising an enhanced data lake that automatically stores at least some of the MA-D and further stores at least some of the analytical data separately from, and under different access conditions than, the MA-D and that comprises (a) governance rules for sorting and managing stored data, (b) pre-determined data formatting standards applicable to at least some of the stored data, or (c) both (a) and (b); (3) an analytical engine that performs one or more analytical functions at least partially based on analytical data stored in the enhanced data lake upon request from a user, upon occurrence of a preprogrammed condition, or both; (4) a cache MA-D processing engine that analyzes cache MA-D when received by a medical apparatus and determines if the cache MA-D should be stored in the MAC-DMS memory component, analyzed by the analytical engine, or both; and (5) an analytical data controller that relays one or more outputs via secure internet communication to one or more medical apparatuses, one or more other network computerized devices, or both, the one or more outputs comprising (a) analytical data output; (b) one or more output applications comprising instructions that control the operation of (I) one or more medical apparatus functions in one or more of the networked medical apparatuses, (II) one or more other network device functions in one or more of the other network devices networked with the system, or (III) both (I) and (II) (aspect 1).

Further provided is a system according to aspect 1, whether the system comprises an engine that determines if MA-D of a medical apparatus is combinable with received streaming MA-D of the same medical apparatus, and if the system engine determines that the cache MA-D and streaming MA-D are combinable, combines the streaming MA-D and cache MA-D to form a mixed MA-D data set, wherein the MA-D analyzed by the analytical engine utilizes the mixed MA-D data set in the generation of analytical data or output (aspect 2).

Also provided is a system according to either aspect 1 or aspect 2, wherein (1) the one or more output applications comprise medical apparatus-specific instructions for controlling (a) one or more therapeutic tasks performed by a particular medical apparatus networked with the system, (b) one or more preventative tasks to be performed by the particular medical apparatus, or (c) both one or more therapeutic tasks and one or more preventative tasks to be performed by the particular medical apparatus; and (2) the system causes the particular medical apparatus to receive, interpret, and execute the one or more output applications, thereby changing the conditions of patient care based on the received one or more output applications (aspect 3).

Further provide is a system according to any one or more of any of aspects 1-3 wherein the system (1) generates, for display on a graphical user interface of one or more networked medical apparatuses, a first representation comprising (a) one or more recommended medical instructions, (b) analytical data relevant to a medical apparatus, patient, or both, which analytical data comprises patient protected health information, or (c) both (a) and (b); and (2) relays the first representation to the one or more networked medical apparatuses for display on one or more graphical user interfaces thereof (aspect 4).

Further provided is a system according to aspect 4, wherein the system further (3) generates, for display on a graphical user interface of one or more other network devices, a second representation comprising analytical data that is devoid of any patient protected health information; and (4) relays the second representation to one or more other network devices for display on one or more graphical user interfaces thereof, wherein the system is adapted such that steps (1)-(4) are performed within less than one minute (aspect 5).

Also provided is a system according to any one or more of aspects 1-5, wherein (1) the one or more data repositories further comprise a first relational database, (2) operation of the system (a) generates a first structured dataset data from analytical output data, and (b) stores the first structured dataset data in the first relational database; wherein (3) the one or more data repositories comprises a second relational database, and (4) the system (a) performs one or more analytical functions on data in the enhanced data lake to obtain analytical data, (b) generates a second structured dataset data from such analytical data optionally in combination with data contained in the first relational database, and (c) stores the second structured dataset data in the second relational database (aspect 6).

Also provided is a system according to aspect 6, wherein the system automatically (1) (a) prepares a third representation concerning the quality of data stored in the first relational database, entering the first relational database, or both, and relaying the third representation to the graphical user interface of one or more other network devices in the data network that are associated with one or more system administrator users, (b) prepares a fourth representation concerning the quality of data stored in the second relational database, entering the second relational database, or both, and relaying the fourth representation to the graphical user interface of one or more other network devices in the data network that are associated with one or more system administrator users, or (c) both (I) prepares a third representation concerning the quality of data stored in the first relational database, entering the first relational database, or both, (II) relays the third representation to the graphical user interface of one or more other network devices in the data network that are associated with one or more system administrator users, (III) prepares a fourth representation concerning the quality of data stored in the second relational database, entering the second relational database, or both, and (IV) relays the fourth representation to the graphical user interface of one or more other network devices in the data network that are associated with one or more system administrator users; and (2) applies one or more modifications to one or more instructions in the system memory in the event the data quality represented by the third representation, fourth representation, or both, indicate a lack of quality of first relational database data, second relational database data, or both (aspect 7).

Further provided is a system according to any one or more of aspects 1-7, wherein the system in operation automatically (1) collects MA-D for a data collection period to form a data collection; (2) evaluates if the data collection is suitable for analysis according to one or more preprogrammed standards; and (3) if the data collection is suitable for analysis, adds the data collection to a data aggregation, repeating steps (1)-(3), until at least ten instances of data collection are added to the data aggregation so as to form a complete data aggregation; wherein (4) if any instance of data collection is determined to be unsuitable before the complete data aggregation is formed, the system automatically discards the incomplete data aggregation and re-initiating the data collection; and (5) if a complete data aggregation is formed, the system causes one or more analytical functions to be performed on data comprising the complete data aggregation (aspect 8).

Also provided is a system according to any one or more of aspects 1-8, wherein the system is adapted to identify data from networked medical apparatuses based on association with three or more independent entities, several other network devices based on association with a commercial class of users, or both, and to deliver data selectively to one or more different medical apparatuses, one or more different other network devices, or both, thereby avoiding inadvertent disclosure of confidential information, unauthorized disclosure of personal health information, or both (aspect 9).

Further provided is a system according to any one or more of aspects 1-9, wherein the system is adapted to identify data delivered from at least two different medical apparatuses (e.g., wherein a first type of medical apparatus that performs one or more pulmonary therapeutic tasks and a second type of medical apparatus that performs one or more cardiovascular therapeutic tasks) (aspect 10).

The invention also provides a system according to aspect 10, wherein the system comprises a machine learning module that is automatically applied to MA-D received from both a first type of medical apparatuses and a second type medical apparatuses to generate predicted, patient-specific, physiological parameters associated with the therapeutic tasks being performed by the medical apparatuses, and to deliver such predicted patient-specific physiological parameters to medical apparatuses of one or both of the two different medical apparatus types, to other network devices, or a combination thereof (aspect 11).

Further provided is a system according to any one or more of aspects 1-11,wherein the system is adapted to identify data from research class user-associated medical apparatuses or other network devices and to apply pre-programmed rules, conditions, or both, for combining MA-D obtained from medical apparatuses associated with health care providers with research user class-associated devices to form a mixed data set, and to perform one or more analytical engine functions at least in part based on the mixed data set (aspect 12).

Also provided is a system according to any one or more of aspects 1-12, wherein the system automatically imposes requirements on MA-D, or both, such that substantially all MA-D datasets stored in the enhanced data lake comprise medical apparatus source-identification information, MA-D type information, and one or more physiological parameter datasets, each thereof presented in a preprogrammed system-recognizable standard format and (2) the enhanced data lake is adapted to comprise different data governance zones that are based on the source or content of MA-D datasets, analytical data, or both, stored therein (aspect 13).

Further provided is a system according to one or more of aspects 1-13, wherein the system is adapted to automatically (1) generate and apply one or more output applications that comprise provision of one or more alarms registered at one or more medical apparatuses, one or more other network devices, or a combination thereof; and (2) provide user adjustable triggering parameters, communication parameters, repetition parameters, or a combination of any or all thereof, for the one or more alarms, wherein the system is adapted such that the parameters can be (a) set at a local medical apparatus level, local other network device level, or both, (b) set at a system level based on medical apparatus type, patient type, user type, or a combination of any or all thereof, or (c) set according to both (a) and (b) (aspect 14).

In one aspect, the invention provides a computer-implemented method for controlling operation of medical apparatuses and other devices in a data network comprising (1) providing a data network, the data network comprising (a) at least 5, 10, 20, 50, or 100 remote-controllable, selectively operable, and internet-connected medical apparatuses, some, most, or each of the medical apparatuses comprising (I) one or more sensors that detect one or more patient-associated physiological states of any patient operationally associated with the medical apparatus that convert information regarding such one or more physiological states to processor-readable medical apparatus data (MA-D), the MA-D comprising MA-D that complies with a pre-established semi-structured data format, (II) an output engine that selectively relays data via secure internet data communications, (III) a medical apparatus memory component that selectively stores MA-D and comprises processor-executable instructions for analyzing and relaying MA-D and evaluating the connection between the medical apparatus and the network, and (IV) a medical apparatus processor that executes the instructions in the medical apparatus memory component, (b) a MAC-DMS comprising (I) a MAC-DMS processor that reads computer readable instructions to analyze data and perform functions, (II) a streaming data processing engine, (III) a cache MA-D processing engine, (IV) an analytical engine, and (V) a MAC-DMS memory component that comprises processor-executable instructions and one or more data repositories, the one or more data repositories comprising an enhanced data lake that stores at least some of the MA-D and at least some of the analytical data separately and under different access conditions, and (c) at least 3 other network devices, each other network device (I) comprising (A) a processor, (B) a memory component, and (C) a remote controllable graphical user interface, and (II) being associated with a user assigned to at least one class of users, wherein classes of users comprise (A) health care providers authorized to access patient protected health information and (B) commercial users that are subject to restrictions on the receipt of patient protected health information; (2) causing each operating medical apparatus to repeatedly collect sensor data from the patient; (3) causing each operating medical apparatus to automatically and repeatedly assess whether a secure network connection is available, and (a) when a secure and stable network connection is available, automatically relaying data comprising MA-D in a substantially continuous manner via secure internet data communication to the MAC-DMS, or (b) when a secure and stable network connection is not available, (I) storing MA-D in the medical apparatus memory component as cache MA-D until a secure and stable network connection becomes available and (II) when a secure and stable network connection becomes available, relaying the cache MA-D to the MAC-DMS via secure internet data communication; (4) causing the MAC-DMS processor to automatically use the streaming data processing engine to (a) receive the relayed streaming MA-D, (b) perform an initial analysis on the relayed streaming MA-D, and (c) perform one or more of a preprogrammed limited set of initial functions if the initial analysis identifies one or more preprogrammed conditions in the streaming relayed MA-D, which initial functions comprise relaying instructions for controlling the operation of one or more medical apparatuses, one or more other network devices, or both; (5) causing the MAC-DMS processor to use the cache MA-D processor to (a) receive cache MA-D when cache MA-D is relayed from a medical apparatus to the MAC-DMS, and (b) determine whether the received cache MA-D is suitable for analysis by the analytical engine, storage in at least one of the one or more data repositories, or both; (6) causing the MAC-DMS processor to automatically store at least some of the MA-D in the enhanced data lake; (7) causing the MAC-DMS processor to use the analytical engine to perform one or more analytical functions on MA-D stored in the enhanced data lake upon request from a user, upon occurrence of a preprogrammed condition, or both, and (8) causing the MAC-DMS processor to relay one or more outputs via secure internet communication to one or more medical apparatuses, one or more other network devices, or both, the one or more outputs comprising (a) analytical data output, (b) one or more output applications comprising instructions that control the operation of (I) one or more medical apparatus functions in one or more of the medical apparatuses of the data network, (II) one or more other network device functions in one or more of the other network devices of the data network, or (III) both (I) and (II), or (c) a combination of (a) and (b) of step (8) (aspect 15).

Further provided is a computer-implemented method for controlling operation of medical apparatuses and other devices in a data network comprising (1) providing a data network, the data network comprising (a) at least 10 remote-controllable, selectively operable, and internet-connected medical apparatuses, each of the at least 10 medical apparatuses comprising (I) one or more sensors that detect one or more patient-associated physiological states and convert information regarding such one or more physiological states to processor-readable medical apparatus data (MA-D), the MA-D comprising MA-D that complies with a pre-established semi-structured data format, (II) an output engine that selectively relays data via secure internet data communications, (III) a medical apparatus memory component that selectively stores MA-D and comprises processor-executable instructions for analyzing and relaying MA-D and evaluating the connection between the medical apparatus and the network, and (IV) a medical apparatus processor that executes the instructions in the medical apparatus memory component, (b) a MAC-DMS comprising (I) a MAC-DMS processor that reads computer readable instructions to analyze data and perform functions, (II) a streaming data processing engine, (III) a cache MA-D processing engine, (IV) an analytical engine, and (V) a MAC-DMS memory component that comprises processor-executable instructions and one or more data repositories and (2) causing each operating medical apparatus to automatically and repeatedly assess whether a secure network connection is available, and (a) when a secure and stable network connection is available, automatically relaying data comprising MA-D in a substantially continuous manner via secure internet data communication to the MAC-DMS or (b) when a secure and stable network connection is not available (I) storing MA-D in the medical apparatus memory component as cache MA-D until a secure and stable network connection becomes available and (II) when a secure and stable network connection becomes available relaying the cache MA-D to the MAC-DMS via secure internet data communication; (3) causing the MAC-DMS processor to automatically use the streaming data processing engine to (a) receive the relayed streaming MA-D, (b) perform an initial analysis on the relayed streaming MA-D, and (c) perform one or more of a preprogrammed limited set of initial functions if the initial analysis identifies one or more preprogrammed conditions in the streaming relayed MA-D, which initial functions comprise relaying instructions for controlling the operation of one or more medical apparatuses, one or more other network devices, or both; (4) causing the MAC-DMS processor to use the analytical engine to (a) identify a collection of MA-D representing a period of MAC-DMS operation, medical apparatus operation, or both, to form a first unit of MA-D, (b) analyze the first unit of MA-D to determine if the first unit of MA-D complies with a preprogrammed unit analysis condition, (c) add the current first unit of MA-D to initiate formation of a second unit of MA-D if the first unit of MA-D complies with the preprogrammed first unit analysis condition, and (d) repeat steps (a)-(c) until either (I) the current first unit does not comply with the preprogrammed first unit analysis condition or (II) the second unit of MA-D is complete; (5) performing one or more analytical functions on a second unit of MA-D; and (6) causing the MAC-DMS processor to relay one or more outputs via secure internet communication to one or more medical apparatuses based on the outcome of the one or more analytical functions, the one or more outputs comprising (a) analytical data generated from the second unit, (b) one or more output applications comprising instructions that control the operation of one or more medical apparatus functions in one or more of the medical apparatuses of the data network, or (c) a combination of (a) and (b) (aspect 16).

The invention yet further provides a computer-implemented method for controlling operation of medical apparatuses and other devices in a data network comprising (1) providing a data network, the data network comprising (a) a number (e.g., at least 10) remote-controllable, selectively operable, and internet-connected medical apparatuses, each of the medical apparatuses comprising (I) one or more sensors that detect one or more patient-associated physiological states and convert information regarding such one or more physiological states to processor-readable medical apparatus data (MA-D), the MA-D comprising MA-D that complies with a pre-established semi-structured data format, (II) an output engine that selectively relays data via secure internet data communications, (III) a medical apparatus memory component that selectively stores MA-D and comprises processor-executable instructions for analyzing and relaying MA-D and evaluating the connection between the medical apparatus and the network, and (IV) a medical apparatus processor that executes the instructions in the medical apparatus memory component, (b) a medical apparatus controller and data management system (“MAC-DMS”) comprising (I) a MAC-DMS processor that reads computer readable instructions to analyze data and perform functions, (II) a streaming data processing engine, (III) a cache MA-D processing engine, (IV) an analytical engine, and (V) a MAC-DMS memory component that comprises processor-executable instructions and one or more data repositories, the one or more data repositories comprising an enhanced data lake that stores at least some of the MA-D and at least some of the analytical data separately and under different access conditions, and (c) at least 3 other network devices, each other network device (I) comprising (A) a processor, (B) a memory component, and (C) a remote controllable graphical user interface, and (II) being associated with a user assigned to at least one class of users, wherein classes of users comprise (A) health care providers authorized to access patient protected health information and (B) commercial users that are subject to restrictions on the receipt of patient protected health information, (2) causing each medical apparatus to perform one or more steps for assessing whether a secure network connection is available, and (a) when a secure and stable network connection is available, automatically relaying data comprising MA-D in a substantially continuous manner via secure internet data communication to the MAC-DMS, or (b) when a secure and stable network connection is not available (I) causing the medical apparatus to perform one or more steps for storing MA-D in the medical apparatus memory component as cache MA-D until a secure and stable network connection becomes available and (II) when a secure and stable network connection becomes available, causing the medical apparatus to perform one or more steps for relaying the cache MA-D to the MAC-DMS via secure internet data communication; (3) causing the MAC-DMS processor to automatically use the streaming data processing engine to (a) perform one or more steps for receiving streaming MA-D, (b) perform an initial analysis on the relayed streaming MA-D, and (c) perform one or more steps for initially analyzing the MA-D for presence of a limited set of indicators of one or more urgent conditions if the initial analysis identifies one or more preprogrammed conditions in the streaming relayed MA-D, and, if such one or more urgent conditions are present in the MA-D, causing the streaming data processing engine to perform one or more steps for controlling the operation of one or more medical apparatuses, one or more other network devices, or both; (4) causing the MAC-DMS processor to use the cache MA-D processor to (a) perform one or more steps for receiving cache MA-D when cache MA-D is relayed from a medical apparatus to the MAC-DMS, and (b) perform one or more steps for determining whether the received cache MA-D is suitable for analysis by the analytical engine, storage in at least one of the one or more data repositories, or both; (5) causing the MAC-DMS processor to perform one or more steps for automatically storing at least some of the MA-D in the enhanced data lake; (6) causing the MAC-DMS processor to use the analytical engine to perform one or more steps for analyzing MA-D stored in the enhanced data lake upon request from a user, upon occurrence of a preprogrammed condition, or both; and (7) causing the MAC-DMS processor to perform one or more steps for relaying one or more outputs via secure internet communication to one or more medical apparatuses, one or more other network devices, or both, the one or more outputs comprising (a) analytical data output, (b) one or more output applications comprising instructions that control the operation of (I) one or more medical apparatus functions in one or more of the medical apparatuses of the data network, (II) one or more other network device functions in one or more of the other network devices of the data network, or (III) both (I) and (II), or (c) a combination of (a) and (b) (aspect 17).

The invention also provides methods of treating a medical condition of a patient in a population of patients, the method comprising (1) providing a data network, the data network comprising (a) a number of remote-controllable, selectively operable, and internet-connected medical apparatuses, each of the medical apparatuses (I) being associated with a patient and (II) comprising (A) one or more sensors that detect one or more patient-associated physiological states from the associated patient and convert information regarding such one or more physiological states to processor-readable medical apparatus data (MA-D), the MA-D comprising MA-D that complies with a pre-established semi-structured data format, (B) an output engine that selectively relays data via secure internet data communications, (C) a medical apparatus memory component that selectively stores MA-D and comprises processor-executable instructions for analyzing and relaying MA-D and evaluating the connection between the medical apparatus and the network, and (D) a medical apparatus processor that executes the instructions in the medical apparatus memory component, (b) a MAC-DMS comprising (I) a MAC-DMS processor that reads computer readable instructions to analyze data and perform functions, (II) a streaming data processing engine, (III) a cache MA-D processing engine, (IV) an analytical engine, and (V) a MAC-DMS memory component that comprises (A) processor-executable instructions and (B) one or more data repositories, the one or more data repositories comprising an enhanced data lake that stores (i) at least some of the MA-D, (ii) at least some of the analytical data, or (iii) both (i) and (ii), in any case storing at least some of such data separately and under different access conditions, and (iv) a collection of previously collected MA-D analytical data obtained from substantially similar medical apparatuses associated with similar patients, and (c) at least 3 other network devices, each other network device (I) comprising (A) a processor, (B) a memory component, and (C) a remote controllable graphical user interface, and (II) being associated with a user assigned to at least one class of users, wherein classes of users comprise (A) health care providers authorized to access patient protected health information associated with the patient and (B) commercial users that are subject to restrictions on the receipt of patient protected health information; (2) causing each operating medical apparatus to repeatedly collect sensor data from the patient associated therewith; (3) causing each of the medical apparatuses to automatically and repeatedly assess whether a secure network connection is available and (a) when a secure and stable network connection is available, automatically relaying data comprising MA-D in a substantially continuous manner via secure internet data communication to the MAC-DMS or (b) when a secure and stable network connection is not available (I) storing MA-D in the medical apparatus memory component as cache MA-D until a secure and stable network connection becomes available and (II) when a secure and stable network connection becomes available, relaying the cache MA-D to the MAC-DMS via secure internet data communication; (3) causing the MAC-DMS processor to automatically use the streaming data processing engine to (a) receive the streaming MA-D relayed from the medical apparatuses, (b) perform an initial analysis on the relayed streaming MA-D received from each medical apparatus, and (c) perform one or more of a preprogrammed limited set of initial functions if the initial analysis identifies one or more preprogrammed conditions in the streaming relayed MA-D, which initial functions comprise relaying instructions for controlling the operation of one or more medical apparatuses, one or more other network devices, or both; (4) causing the MAC-DMS processor to use the cache MA-D processor to (a) receive cache MA-D when cache MA-D is relayed from a medical apparatus to the MAC-DMS and (b) determine whether the received cache MA-D is suitable for analysis by the analytical engine, storage in at least one of the one or more data repositories, or both; (5) causing the MAC-DMS processor to automatically store at least some of the MA-D in the enhanced data lake; (6) causing the MAC-DMS processor to use the analytical engine to perform one or more analyses on at least some of the analytical data stored in the enhanced data lake upon request from a user, upon occurrence of a preprogrammed condition, or both, wherein the analytical engine (a) performs an analysis using analytical data generated by MA-D from the medical apparatuses and in performing the analysis and (b) compares MA-D from each medical apparatus to (I) the previously collected MA-D analytical data, (II) preprogrammed standards, or (III) both (I) and (II); and (7) causing the MAC-DMS processor to relay one or more outputs via secure internet communication to one or more medical apparatuses, one or more other network devices, or both, the one or more outputs comprising (a) analytical data output relevant to the treatment of the patient relayed to (I) the medical apparatus associated with the patient, (II) another network device associated with a healthcare provided associated with the patient, or (III) both (I) and (II), (b) one or more output applications comprising instructions that control the operation of (I) one or more medical apparatus functions in one or more of the medical apparatuses of the data network, (II) one or more other network device functions in one or more of the other network devices of the data network that is associated with a healthcare provider associated with the patient, or (III) both (I) and (II). or (c) a combination of (a) and (b) (aspect 18).

The invention also provides a computer-implemented method for controlling operation of medical apparatuses and other devices in a data network comprising (1) providing a data network, the data network comprising (a) a number of remote-controllable, selectively operable, and internet-connected medical apparatuses, each of the at least 10 medical apparatuses comprising (I) one or more patient interactive components comprising (A) one or more physical components that in operation modify one or more aspects of an associated patient's physical condition, (B) one or more sensors that detect one or more physiological states in the associated patient, or (C) both (A) and (B), (II) a component for converting such information into electronic medical apparatus data (MA-D), the MA-D comprising data that complies with a pre-established semi-structured data format, (III) an output engine that selectively relays data via secure internet data communications, (IV) a medical apparatus memory component that selectively stores MA-D and comprises processor-executable instructions for analyzing and relaying MA-D and evaluating the connection between the medical apparatus and the network, and (V) a medical apparatus processor that executes the instructions in the medical apparatus memory component (b) a MAC-DMS comprising (I) a MAC-DMS processor that reads computer readable instructions to analyze data and perform functions, (II) a streaming data processing engine, (III) a cache MA-D processing engine, (IV) an analytical engine, and (V) a MAC-DMS memory component that comprises (A) processor-executable instructions and (B) one or more data repositories, the one or more data repositories comprising an enhanced data lake that stores (i) at least some of the MA-D, (ii) at least some of the analytical data, or (iii) both (i) and (ii), in any case storing at least some of such data separately and under different access conditions, and (iv) a collection of previously collected MA-D analytical data obtained from substantially similar medical apparatuses, and (c) at least 3 other network devices, each other network device (I) comprising (A) a processor, (B) a memory component, and (C) a remote controllable graphical user interface, and (II) being associated with a user assigned to at least one class of users, wherein classes of users comprise (A) health care providers authorized to access patient protected health information and (B) commercial users that are subject to restrictions on the receipt of patient protected health information; (2) causing each operating medical apparatus to repeatedly collect sensor data and to convert such data into MA-D; (3) causing each operating medical apparatus to automatically and repeatedly assess whether a secure network connection is available and (a) when a secure and stable network connection is available, automatically relaying data comprising MA-D in a substantially continuous manner via secure internet data communication to the MAC-DMS or (b) when a secure and stable network connection is not available (I) storing MA-D in the medical apparatus memory component as cache MA-D until a secure and stable network connection becomes available and (II) when a secure and stable network connection becomes available, relaying the cache MA-D to the MAC-DMS via secure internet data communication; (4) causing the MAC-DMS processor to use the cache MA-D processor to (a) receive cache MA-D when cache MA-D is relayed from a medical apparatus to the MAC-DMS and (b) determine whether the received cache MA-D is suitable for analysis by the analytical engine, storage in at least one of the one or more data repositories, or both; (5) causing the MAC-DMS processor to automatically store at least some of the MA-D in the enhanced data lake; (6) causing the MAC-DMS processor to use the analytical engine to automatically evaluate the performance of the medical apparatuses in the network and the MAC-DMS by (a) evaluating the MA-D of each medical apparatus against MA-D from each medical apparatus, (b) generating analytical data from the MA-D from all of the medical apparatuses and evaluating the analytical data by comparing it to the previously collected MA-D analytical data, or (c) performing both (a) and (b); and (7) changing at least one parameter of medical apparatus operation, MAC-DMS operation, or both, based on the evaluation of the medical apparatuses and the MAC-DMS (aspect 19).

Also provided is a method according to any one of aspects 15-19, wherein the method comprises causing the MAC-DMS to evaluate whether received cache MA-D of a medical apparatus is combinable with received streaming MA-D of the same medical apparatus, and if the MAC-DMS determines that the cache MA-D and streaming MA-D are combinable, combining the streaming MA-D and cache MA-D to form a mixed MA-D data set, wherein the MA-D analyzed by the analytical engine comprises the mixed MA-D data set (aspect 20).

Further provided is a method of any one of aspects 15-20, wherein (1) the one or more output applications comprise medical apparatus-specific instructions for controlling (a) one or more therapeutic tasks performed by a particular medical apparatus, (b) one or more preventative tasks performed by a particular medical apparatus, or (c) both one or more therapeutic tasks and one or more preventative tasks performed by the particular medical apparatus; (2) the particular medical apparatus receives, interprets, and executes the one or more output applications; and (3) the particular medical apparatus changes one or more conditions of patient care based on the received one or more output applications (aspect 21).

Additionally provided is a method of any one or more of aspects 15-21, wherein the generation of one or more output applications comprises (1) generating, for display on a graphical user interface of one or more medical apparatuses, a first representation comprising (a) one or more recommended medical instructions, (b) analytical data relevant to a medical apparatus, patient, or both, which analytical data comprises patient protected health information, or (c) both (a) and (b); (2) relaying the first representation to the one or more medical apparatuses for display on one or more graphical user interfaces thereof, (3) generating, for display on a graphical user interface of one or more other network devices, a second representation comprising analytical data that is devoid of any patient protected health information; and (4) relaying the second representation to one or more other network devices for display on one or more graphical user interfaces thereof, wherein steps (1)-(4) are performed within less than one minute (aspect 22).

Further provided is a method of any one or more of aspects 15-22, wherein (1) the one or more data repositories further comprise a first relational database; (2) the method comprises (a) generating first structured dataset data from analytical output data, and (b) storing the first structured dataset data in the first relational database; (3) the one or more data repositories comprises a second relational database, and (4) the method comprises (a) performing one or more analytical functions on data in the enhanced data lake to obtain analytical data, (b) generating second structured dataset data from such analytical data optionally in combination with data contained in the first relational database, and (c) storing the second structured dataset data in the second relational database (aspect 23).

Also provided is a method of aspect 23, wherein the method comprises (1) first (a) preparing a third representation concerning the quality of data stored in the first relational database, entering the first relational database, or both, and relaying the third representation to the graphical user interface of one or more other network devices in the data network that are associated with one or more system administrator users, (b) preparing a fourth representation concerning the quality of data stored in the second relational database, entering the second relational database, or both, and relaying the fourth representation to the graphical user interface of one or more other network devices in the data network that are associated with one or more system administrator users, or (c) preparing (I) a third representation concerning the quality of data stored in the first relational database, entering the first relational database, or both, (II) relaying the third representation to the graphical user interface of one or more other network devices in the data network that are associated with one or more system administrator users, (III) preparing a fourth representation concerning the quality of data stored in the second relational database, entering the second relational database, or both, and (IV) relaying the fourth representation to the graphical user interface of one or more other network devices in the data network that are associated with one or more system administrator users; and (2) thereafter applying one or more modifications to one or more instructions in the MAC-DMS memory in the event the data quality represented by the third representation, fourth representation, or both, indicate a lack of quality of first relational database data, second relational database data, or both (aspect 24).

Additionally provided is a method of any one or more of aspects 15-24, wherein any one or more of the medical apparatuses comprise one or more multi-zone medical apparatuses (MZMAs), each MZMA comprising two or more distinct zones, each zone (1) comprising a separate processor that processes at least some MA-D not processed by the processor of at least one other component; and (2) (a) receiving information from different sensors, (b) performing different therapeutic or preventative medical tasks, (c) receiving information from different sensors and performing different therapeutic or preventative medical tasks, or (d) perform a combination or some or all of (a)-(c), wherein at least one zone in each MZMA is subject to a different level of interaction with one or more other parts of the data network than at least one other zone of the MZMA (aspect 25).

Also provided is a method of aspect 25, wherein at least one zone of at least one of the one or more MZMAs is associated with application of a therapeutic medical task and is not in direct communication with the data network (aspect 26).

Further provided is a method of aspect 25 or aspect 26, wherein at least one zone of the at least one or more MZMAs (1) is associated with application of a therapeutic medical task, a preventative task, or both; (2) is in communication with the MAC-DMS; and (3) only permits a pre-established amount of input from the MAC-DMS, wherein changes to the operating system, software, or approved forms of MAC-DMS input in the at least one zone of the at least one MZMA requires local approval from an authorized operator of the at least one MZMA (aspect 27).

Additionally provided is a method of any one or more of aspects 15-27, wherein the method further comprises causing the MAC-DMS to automatically (1) collect MA-D for a data collection period to form a data collection; (2) evaluate if the data collection is suitable for analysis according to one or more preprogrammed standards; and (3) if the data collection is suitable for analysis, adding the data collection to a data aggregation, repeating steps (1)-(3), until at least ten instances of data collection are added to the data aggregation so as to form a complete data aggregation; wherein (4) if any instance of data collection is determined to be unsuitable before the complete data aggregation is formed, the method comprises discarding the incomplete data aggregation and re-initiating the method; and (5) if a complete data aggregation is formed, the method further comprises performing one or more analytical functions on data comprising the complete data aggregation (aspect 28).

Further provided is a method of any one or more of aspects 15-28, wherein (1) the medical apparatuses of the data network are owned by at least three different independent entities; (2) at least one user of one or more other network devices is a commercial class user and is legally associated with the owner of the MAC-DMS; and (3) the analytical data output provided to one or more other network devices associated with the one or more commercial class users (a) comprises data generated from a collection of medical apparatuses owned by a plurality of independent entities and (b) excludes one or more classes of MAC-DMS-identified independent entity confidential information (aspect 29).

Also provided is a method of any one or more of aspects 15-29, wherein (1) the medical apparatuses comprise at least two different medical apparatus types, the medical apparatus types comprising a first type of medical apparatus that performs one or more pulmonary therapeutic tasks and a second type of medical apparatus that performs one or more cardiovascular therapeutic tasks and (2) the method comprises applying a machine learning module on MA-D received from both the first type of medical apparatuses and the second type medical apparatuses to generate predicted, patient-specific, physiological parameters associated with the therapeutic tasks being performed by the medical apparatuses, and to deliver such predicted patient-specific physiological parameters to medical apparatuses of one or both of the two different medical apparatus types, to other network devices, or a combination thereof (aspect 30).

Further provided is a method of any one or more of aspects 15-30, wherein (1) classes of users comprise one or more research user classes; (2) one or more medical apparatuses in the data network are associated with one or more users in the one or more research user classes; and (3) the method comprises (a) the MAC-DMS receiving input from at least some of the research user-associated medical apparatuses, (b) the MAC-DMS combining MA-D from at least some of the research user-associated medical apparatuses with MA-D obtained from medical apparatuses associated with health care providers to form a mixed data set, and (c) the MAC-DMS performing one or more analytical engine functions at least in part based on the mixed data set (aspect 31).

Further provided is a method according to any one or more of aspects 15-31, wherein (1) the MAC-DMS transforms MA-D, imposes requirements on MA-D, or both, such that substantially all MA-D datasets stored in the enhanced data lake comprise medical apparatus source-identification information, MA-D type information, and one or more physiological parameter datasets, each thereof presented in a preprogrammed MAC-DMS-recognizable standard format and (2) the enhanced data lake comprises different data governance zones that are based on the source or content of MA-D datasets, analytical data, or both, stored therein (aspect 32).

Still further provided is a method of any one or more of aspects 15-32, wherein (1) one or more output applications comprise provision of one or more alarms registered at one or more medical apparatuses, one or more other network devices, or a combination thereof and (2) triggering parameters, communication parameters, repetition parameters, or a combination of any or all thereof, for the one or more alarms, are (a) set at a local medical apparatus level, local other network device level, or both, (b) set at a MAC-DMS level based on medical apparatus type, patient type, user type, or a combination of any or all thereof, or (c) are set according to both (a) and (b) (aspect 33).

Also provided is a method of aspect 33, wherein (1) the output application(s) comprise (a) one or more output applications that are regulated as software as a medical device (SaMD/SAMD) by ≥1 regulatory authorities and (b) one or more output applications that are not regulated as SaMDs; and (2) the method comprises applying one or more data transformations, data curation processes, data validation checks, or a combination of any or all thereof, to ensure that the one or more SaMD applications comply with one or more regulatory requirements recorded in processor readable instructions stored in the MAC-DMS memory (aspect 34).

In an aspect, the invention provides internet-based data network comprising (1) a number of remote-controllable, selectively operable, and internet-connected medical apparatuses, each of the medical apparatuses comprising (a) sensor(s) that detect one or more patient-associated physiological states and convert information regarding such one or more physiological states to processor-readable medical apparatus data (MA-D), the MA-D comprising MA-D that complies with a pre-established semi-structured data format, (b) an output engine that selectively relays data via secure internet data communications, (c) a medical apparatus memory component that selectively stores MA-D and comprises processor-executable instructions for one or more medical apparatus computer implemented data engines, (d) a medical apparatus processor that executes the instructions in the medical apparatus memory component, and (e) a network status engine that (I) automatically repeatedly checks for availability of a secure and stable internet connection when in operation, (II) when a secure and stable internet connection is present, relays at least some of the MA-D via such secure and stable internet connection to one or more intended recipient internet connected systems, (III) when a secure and stable internet connection is not present, causes at least some of the MA-D to be stored in the medical apparatus memory component as cache MA-D until a secure and stable internet connection is established or reestablished and thereafter relays at least some of the cache MA-D to the one or more recipient internet connected devices or systems; and (2) a medical apparatus controller and data management system (“MAC-DMS”) comprising (a) a system processor that reads computer readable instructions to analyze data and perform functions, (b) a streaming data processing engine that automatically and continuously receives and processes MA-D relayed from the medical apparatuses and performs an initial analysis on received streaming MA-D to determine if one or more preprogrammed conditions are present in the streaming MA-D and, if such one or more conditions are present, acts as a controller of one or more of the medical apparatuses, one or more other network devices, or both by performing one or more of a preprogrammed limited set of initial functions, which initial functions comprise relaying instructions for controlling the operation of one or more medical apparatuses, one or more other network computerized devices, or both, (c) a MAC-DMS memory component that comprises processor-executable instructions and one or more data repositories, the one or more data repositories comprising an enhanced data lake that automatically stores at least some of the MA-D and further stores at least some of the analytical data separately from, and under different access conditions than, the MA-D, (d) an analytical engine that performs one or more analytical functions at least partially based on analytical data stored in the enhanced data lake upon request from a user, upon occurrence of a preprogrammed condition, or both, (e) a cache MA-D processing engine that analyzes cache MA-D when received by a medical apparatus and determines if the cache MA-D should be stored in the MAC-DMS memory component, analyzed by the analytical engine, or both, and (f) a network device controller (analytical data controller) that relays one or more outputs via secure internet communication to one or more medical apparatuses, one or more other network computerized devices, or both, the one or more outputs comprising (I) analytical data output; (II) one or more output applications comprising instructions that control the operation of (A) one or more medical apparatus functions in one or more of the medical apparatuses of the data network, (B) one or more other network device functions in one or more of the other network devices of the data network, or (C) both (A) and (B); or (III) a combination of (I) and (II) (aspect 35).

In a further aspect, the invention provides an internet-based data network comprising (1) several (e.g., ≥~10) remote-controllable, selectively operable, and internet-connected medical apparatuses, each of the medical apparatuses comprising (a) one or more sensors that detect one or more patient-associated physiological states and convert information regarding such one or more physiological states to processor-readable medical apparatus data (MA-D), the MA-D comprising MA-D that complies with a pre-established semi-structured data format, (b) an output engine that selectively relays data via secure internet data communications, (c) a medical apparatus memory component that selectively stores MA-D and comprises processor-executable instructions for one or more medical apparatus computer implemented data engines, (d) a medical apparatus processor that executes the instructions in the medical apparatus memory component, (e) a network status engine that (I) automatically repeatedly checks for availability of a secure and stable internet connection when in operation, (II) when a secure and stable internet connection is present, relays at least some of the MA-D via such secure and stable internet connection to one or more intended recipient internet connected systems, (III) when a secure and stable internet connection is not present, causes at least some of the MA-D to be stored in the medical apparatus memory component as cache MA-D until a secure and stable internet connection is established or reestablished and thereafter relays at least some of the cache MA-D to the one or more recipient internet connected devices or systems; and (2) a MAC-DMS comprising (a) a MAC-DMS processor that reads computer readable instructions to analyze data and perform functions, (b) a streaming data processing engine that automatically and continuously receives and processes MA-D relayed from the medical apparatuses and performs an initial analysis on received streaming MA-D to determine if one or more preprogrammed conditions are present in the streaming MA-D and, if such one or more conditions are present, acts as a controller of one or more of the medical apparatuses, one or more other network devices, or both by performing one or more of a preprogrammed limited set of initial functions, which initial functions comprise relaying instructions for controlling the operation of one or more medical apparatuses, one or more other network computerized devices, or both, (c) a MAC-DMS memory component that comprises processor-executable instructions for one or more MAC-DMS-implemented engines and one or more data repositories, the one or more data repositories comprising (I) an enhanced data lake comprising a plurality of data lake management zones each data lake management zone being associated with different data lake management zone access rules, (II) a first relational database, and (III) a second relational database, (d) one or more MA-D memory ingestion engines that analyzes MA-D and records MA-D to one or more data lake management zones based on one more preprogrammed criteria, (e) an analytical engine that performs one or more analytical functions at least partially based on analytical data stored in the enhanced data lake upon request from a user, upon occurrence of a preprogrammed condition, or both, (f) an analytical data memory ingestion engine that analyzes analytical data based on one or more preprogrammed conditions and based on such preprogrammed conditions records (I) analytical data generated directly from MA-D in the first relational database, (II) analytical data generated from data contained in the enhanced data lake in the second relational database, and (III) analytical data in one or more data lake management zones of the enhanced data lake, (g) a data repository inspection engine that collects information concerning data being ingested into the first relational database, data being ingested into the second relational database, or both, and relays such information to one or more graphical user interfaces accessible by one or more system administrators, and (h) a network device controller that relays one or more outputs via secure internet communication to one or more medical apparatuses, one or more other network computerized devices, or both, the one or more outputs comprising (I) analytical data output; (II) one or more output applications comprising instructions that control the operation of (A) one or more medical apparatus functions in one or more of the medical apparatuses of the data network, (B) one or more other network device functions in one or more of the other network devices of the data network, or (C) both (A) and (B); or (III) a combination of (I) and (II) (aspect 36).

In an aspect, the invention provides a network in accordance with aspect 35 or aspect 36, wherein (1) the one or more data repositories further comprise a first relational database; (2) the method comprises (a) generating first structured dataset data from analytical output data, and (b) storing the first structured dataset data in the first relational database; (3) the one or more data repositories comprises a second relational database; and (4) the system (a) performs one or more analytical functions on data in the enhanced data lake to obtain analytical data, (b) generates second structured dataset data from such analytical data optionally in combination with data contained in the first relational database, and (c) stores the second structured dataset data in the second relational database (aspect 37).

In a further aspect, the invention provides a network according to any one or more of aspects 35-37, wherein any one or more of the medical apparatuses comprise one or more multi-zone medical apparatuses (MZMAs), each MZMA comprising two or more distinct zones, each zone (1) comprising a separate processor that processes at least some MA-D not processed by the processor of at least one other component; and (2) being adapted to (a) receive information from different sensors, (b) perform different therapeutic or preventative medical tasks, (c) receive information from different sensors and performing different therapeutic or preventative medical tasks, or (d) perform a combination or some or all of (a)-(c), wherein at least one zone in each MZMA is subject to a different level of interaction with one or more other parts of the data network than at least one other zone of the MZMA (aspect 38).

Further provided is a network according to aspect 38, wherein at least one zone of at least one of the one or more MZMAs is associated with application of a therapeutic medical task and is not in direct communication with the data network (aspect 39).

Also provided is a network according to aspect 38 or aspect 39, wherein at least one zone of the at least one or more MZMAs (1) is associated with application of a therapeutic medical task, a preventative task, or both; (2) is in communication with the MAC-DMS; and (3) only permits a pre-established amount of input from the MAC-DMS; wherein changes to the operating system, software, or approved forms of MAC-DMS input in the at least one zone of the at least one MZMA requires local approval from an authorized operator of the at least one MZMA (aspect 40).

Further provided is a network according to any one or more of aspects 35-40, wherein the MAC-DMS is configured to automatically (1) collect MA-D for a data collection period to form a data collection; (2) evaluate if the data collection is suitable for analysis according to one or more preprogrammed standards; and (3) if the data collection is suitable for analysis, adding the data collection to a data aggregation, repeating steps (1)-(3), until at least ten instances of data collection are added to the data aggregation so as to form a complete data aggregation; wherein (4) if any instance of data collection is determined to be unsuitable before the complete data aggregation is formed, the method comprises discarding the incomplete data aggregation and re-initiating the method; and (5) if a complete data aggregation is formed, the method further comprises performing one or more analytical functions on data comprising the complete data aggregation (aspect 41).

Also provided is a network of any one or more of aspects 35-42, wherein (1) the medical apparatuses comprise at least two different medical apparatus types, the medical apparatus types comprising a first type of medical apparatus that performs one or more pulmonary therapeutic tasks and a second type of medical apparatus that performs one or more cardiovascular therapeutic tasks; and (2) the system is adapted to apply a machine learning module on MA-D received from both the first type of medical apparatuses and the second type medical apparatuses to generate predicted, patient-specific, physiological parameters associated with the therapeutic tasks being performed by the medical apparatuses, and to deliver such predicted patient-specific physiological parameters to medical apparatuses of one or both of the two different medical apparatus types, to other network devices, or a combination thereof (aspect 43).

Further provided is a network according to any one or more of aspects 35-43, wherein (1) (a) other network device(s), or both are associated with one or more research user classes; (b) one or more medical apparatuses in the data network are associated with one or more users in the one or more research user classes; or (c) both (a) and (b); (2) the MAC-DMS is adapted to (a) receive input from at least some of the research user-associated medical apparatuses, (b) combine MA-D from at least some of the research user-associated medical apparatuses with MA-D obtained from medical apparatuses associated with health care providers to form a mixed data set, and (c) perform one or more analytical engine functions at least in part based on the mixed data set (aspect 44).

Also provided is a network according to any one or more of aspects 35-44, wherein (1) the system/MAC-DMS is adapted to generate one or more output applications that cause operation of one or more alarms registered at one or more medical apparatuses, one or more other network devices, or a combination thereof and (2) the system/MAC-DMS is programmable with respect to alarm triggering parameters, communication parameters, repetition parameters, or a combination of any or all thereof, and such parameter(s) can be (a) set at a local medical apparatus level, local other network device level, or both, (b) set at a MAC-DMS level based on medical apparatus type, patient type, user type, or a combination of any or all thereof, or (c) are set according to both (a) and (b) (aspect 45).

Further provided is a network according to any one or more aspects of 35-45, wherein (1) the one or more output applications comprise (a) one or more output applications that are regulated as software as a medical device (SaMD) by one or more regulatory authorities and (b) one or more output applications that are not regulated as SaMDs; and (2) the system/MAC-DMS is adapted to apply one or more data transformations, data curation processes, data validation checks, or a combination of any or all thereof, to ensure that the one or more SaMD applications comply with one or more regulatory requirements recorded in processor readable instructions stored in the MAC-DMS memory (aspect 46).

In another aspect, the invention provides a medical apparatus comprising (1) a high security zone comprising a collection of directly interoperating high security zone components, the high security zone components comprising (a) one or more therapeutic components that in operation apply medical treatment to a patient and are controllable through received electronic control instructions, (b) a high security zone memory component that comprises computer readable media comprising instructions for a plurality of computer-implemented instructions that control operation of one or more high security zone components, (c) a high security zone computer processor component that executes computer-implemented instructions contained in the high security zone memory component to send electronic instructions to the one or more therapeutic components to control operation of the one or more therapeutic components, (d) a high security zone communication component that (I) receives electronic communications from (A) at least one medium security zone component and (B) a local physical input, (II) comprises a security engine that analyzes communications received from the medium zone component to ensure that only communications that comply with one or more validation standards are allowed to control operation of one or more high security zone components, and (III) relays electronic communication to (A) at least one medium security zone component and (B) a local medical apparatus output, (e) a physical anti-tampering system that limits access to the high security zone memory component to only authorized users, and wherein (f) the high security zone lacks any capability for direct internet communications; and (2) a medium security zone comprising a second collection of interoperative components, the medium security zone components comprising (a) one or more sensors that measure one or more physical conditions of (I) performance of one or more therapeutic components in the high security zone, (II) one or more physiological conditions of an associated patient, or (III) both (I) and (II) as medical apparatus data (MA-D), and convert such measurements into electronically transmittable data, (b) a medium security zone memory component that comprises (I) computer readable media comprising instructions for a plurality of computer-implemented instructions that control operation of one or more medium security zone components and (II) a medium security zone component for storage of MA-D, and (c) a medium security zone computer processor component that executes computer-implemented instructions contained in the medium security zone memory component including (I) a network status engine that evaluates if a secure and stable internet collection is available and (A) when such a connection is available (i) relays MA-D to one or more intended internet recipient devices and (ii) receives instructions relayed from one or more remote control devices via secure internet communications, or (B) when such a connection is not available, stores MA-D in the medium zone security memory component as cache data until such a connection is available and then relays the cache data to one or more intended recipient devices, and (II) an inter-apparatus communication engine that receives data from, and sends electronic instructions to, the therapeutic component (aspect 47).

Further provided is a medical apparatus according to aspect 47, wherein the apparatus is configured such that an operating system of the medium zone security computer processor component, an operating system of the high security computer processor component, or both, are only modifiable over the internet in response to a request (pull signal) sent from the medical apparatus to a server comprising software updates for the one or more operating systems (aspect 48).

Also provided is a medical apparatus according to aspect 48, wherein the operating system of the high security computer processor component is configured to only be modified by a user with physical access to the high security computer processor component (aspect 49).

In yet another aspect, the invention provides a data network comprising (1) several separately located and at least partially remotely controllable medical apparatuses (MAs) that are in substantially continuous communication with a network data system (NDS), the MAs being under physical control of a number (e.g., at least 5) separate independent entities (IEs), each MA of the system comprising (a) at least 1 sensor that in operation detects conditions in a patient, (b) device memory comprising physical, transferrable, and reproducible computer readable media (PTRCRM) comprising device computer executable instructions (CEI) and records comprising sensor information collected over time; (c) a device computer processing component for reading CEI in device memory, (d) a device display unit, (e) a device data relay unit that in operation transmits device information (MA-D) that can comprise sensor information from the MA to the NDS via the internet, wherein MA-D relayed by the MA relay unit in operation comprises real time sensor data (RT-MA-D), stored sensor data (MA-CD), or both RT-MA-D and MA-CD, and wherein MA-D comprises both structured and unstructured data, (f) a device data input unit, and (g) optionally a device data security system that optionally comprises at least 1 microcontroller that in operation performs 1 or more data protection functions, e.g., comprising restricting data input to only data that is specifically identified data of an approved data type; (2) a network data system (NDS or system) and (2) a network data management system (NDS) comprising (1) a NDS memory unit comprising an NDS PTRCRM comprising (A) at least one searchable data repository (DR) that receives and stores semi-unstructured MA data (SUMAD) and (B) NDS CEI (NCEI) encoding instructions for functions carried out by the NDS; (2) a NDS processing function that executes the NDS CEI, (3) a NDS data input unit that automatically receives data from MAs in communication with the NDS and determines if MA-D received from each MA is RT-MA-D, MA-CD, or both, (4) a NDS analytical unit that analyzes RT-MA-D, MA-CD, and SUMAD to generate an analysis, and applies the analysis to the performance of 1 or more NDS functions, (5) optionally a NDS device data harmonization unit that evaluates if RT-MA-D in MA-D is approved for use by the NDS analytical unit and determines how approved RT-MA-D is used by the NDS analytical unit, (6) a NDS output processing system that automatically filters MA-D, analysis, or both, for delivery to each MA and other network devices (OND) in the data network, based on confidentiality rules, health care compliance rules, or both, (7) an NDS data relay unit that securely relays over the internet (A) information to each MA that is specific to the MA, associated patient, or both and (B) information comprising MA-D, analysis, or both, to 1 or more other network devices/interfaces (ONDs or ONDIs), wherein the information relayed to ONDIs comprises information from a number of MAs associated with the IEs or derived from MA-D received from a number of MAs associated with 2 or more IEs, and (8) a NDS analytical unit that generates an analysis (analytical data) based on application of 1 or more schemas to the SUMAD (aspect 50).

An aspect is a network according to aspect 50, wherein the NDS comprises an engine/unit for detecting volume of incoming data (demand) and for automatically increasing NDS processor capability in response to the volume of incoming data meeting or exceeding a preprogrammed threshold (aspect 51).

A further aspect is a network according to aspect 50 or aspect 51, wherein the NDS relay unit in operation continuously transmits NDS status information to the MAs in the network in a format that is acceptable to the MA security unit of the MAs (aspect 52).

Another aspect is a network according to any one or more of aspects 50-52, wherein MA CEI in the MAs comprises instruction to store MA-D as MA-CD in the event that the MA does not receive an indication that the NDS is operational until the MA receives an indication that the NDS is operational (aspect 53).

A further aspect is a network according to any one or more of aspects 50-53 wherein the NDS comprises a function for identifying past, present, and predicted MA-D, and the NDS analytical unit performs at least 1 predictive function based on the analysis and relays the results of the predictive function to the MAs, ONDs, or both (aspect 54).

An aspect is a network according to any one or more of aspects 50-54, wherein the NDS analytical unit performs at least one function that is regulated as software as a medical device (SAMD) and at least one other non-SAMD function (NSAMD), and the system delivers output of the at least one SAMD and at least one NSAMD functions to MAs in accordance with CEIs that reflect applicable regulatory status of the SAMD and NSAMD functions (aspect 55).

A further aspect is a network according to aspect 55, wherein the at least one SAMD comprises providing diagnostic instructions or therapeutic instructions to an HCP (aspect 56)

A further aspect is a network according to aspect 55 or aspect 56, wherein the at least one SAMD can change operating conditions of MAs in response to condition(s) (aspect 57).

A further aspect is a network according to any one or more of aspects 50-57, wherein ONDs in the comprise a system owner (SO) support system (SOSS) that delivers MA-D, analysis (NDS-AD), or both, to SO-associated network access devices (SOANADs) (aspect 58).

A further aspect is a network according to aspect 58, wherein the SOSS combines MA-D, NDS-AD, or both, with data from a customer relationship management support system (CRMSS) (aspect 59).

A further aspect is a network according to aspect 59, wherein ONDI/NDS users comprise sales representatives that sell/lease network MAs for the SO (aspect 60).

A further aspect is a network according to any one or more of aspects 50-60, wherein the network comprises one or more research platform(s) (RP(s)) that receives or delivers MA-D, delivers analysis, or a combination thereof, to ONDIs, MAs, or both, associated with scientific researcher (SR)-associated network access devices (SRANADs), wherein the SRs are not associated with the SO (aspect 61).

A further aspect is a network according to any one or more of aspects 50-61, wherein the MA-D comprises information about the operating state of the MA-D and the NDS analytical unit evaluates the operating state information in the MA-D in analytical processes (aspect 62).

A further aspect is a network according to any one or more of aspects 50-62, wherein the MAs comprise 2 or more heterogenous types of MAs, such that the NDS determines the MA device type prior to relaying NDS-analysis or NDS output applications (aspect 63).

A further aspect is a network according to any one or more of aspects 50-63, wherein at least most of the MAs are associated with a critical life support function e.g., supporting or treating the cardiovascular system, lung, brain, or kidney (aspect 64).

A further aspect is a network according to any one or more of aspects 50-64, wherein the NDS comprises a machine learning module (MLM) that analyzes MA-D from heterogenous types of MAs and performs or recommends the performance of one or more functions of the NDS based on the analysis of the heterogenous MA MA-D (aspect 65).

A further aspect is a network according to any one or more of aspects 50-65, wherein the NDS comprises a functional module that can write MA-D, analysis (analytical data output based on analysis of MA-D), or both, directly to an electronic health record (aspect 66).

A further aspect is a network according to any one or more of aspects 50-66, wherein data permitted to be transmitted from the NDS relay unit to the MA input unit/MA consists essentially of biometric predictive data, provision of instructions or control of the MA, NDS status, MA software version status, or a combination thereof (CT) (aspect 67).

A further aspect is a network according to aspect 67, wherein data permitted to be transmitted from the NDS to the MA comprises MA software version status, but the MA, system/NDS, or both, prevents updating MA software over the internet, optionally permitting updating in response to a locally initiated pull command from an MA (aspect 68).

An aspect is a network according to any one or more of aspects 50-68, wherein MA-D comprises location information and the NDS simultaneously relays information to at least 2 classes of ONDIs based on facility, metropolitan area, state/region, country, or IE (aspect 69).

A further aspect is a network according to any one or more of aspects 50-69, wherein the MA receives input from one or more research teams, a plurality of IEs using the MAs in clinical application, or both (aspect 70).

A further aspect is a network according to any one or more of aspects 50-70, wherein the NDS delivers MA-D, analysis, or both to a plurality of GUI schemes (ONDIs), based at least in part on user roles comprising independent research team member, system owner device sales or leasing promotional agent, IE user or administrator, system owner clinical or medical support team personnel, and system owner system analyst (aspect 71).

A further aspect is a network according to any one or more of aspects 50-71, wherein NDS CEI comprises one or more alarm conditions linked to sensor data, the NDS (e.g., the NDS analytical unit) determines if one or more alarm conditions are triggered by MA-D, MA-D analysis (analysis/NDS-AD), or both, and the NDS causes an alarm to register in the MA, in a user associated OND, or both, based on sensor data and user options (aspect 72).

A further aspect is a network per any one or more of aspects 50-72, wherein the MA comprises an anti-tampering detection component(s)/function(s) that, e.g., sends a signal to the NDS, an OND, or both, if a prohibited tampering event occurs (aspect 73).

A further aspect is a network according to any one or more of aspects 50-73, wherein MA-D comprises images of MAs, the MA environment, or both, and non-image sensor data (aspect 74).

A further aspect is a network according to any one or more of aspects 50-74, wherein the NDS memory comprises two or more separate governance zones that manage storage of, use of, and access to (a) RT-MA-D, MA-CD, or both; (b) curated data, scored data, or both; (c) system testing data; (d) outgoing data; or (e) a combination of some or all thereof (aspect 75).

A further aspect is a network according to any one or more of aspects 50-75, wherein the NDS comprises (a) data for MAs in a particular country, (b) nation-specific data governance rules, and (c) system blueprint data shared with other NDS(s) (aspect 76).

A further aspect is a network according to any one or more of aspects 50-76, wherein MAs relay packets comprising sequencing information and the NDS comprises a unit/function for analyzing the time component of MA-CD and resequencing MA-D received in nonchronological order (aspect 77).

A further aspect is a network according to any one or more of aspects 50-77, wherein one or more functions/units of the NDS are based on processing at least about 20, 30, or 60 seconds of MA-D per iteration (e.g., every 20-200 seconds, 30-180 seconds, or 45-90 seconds), the NDS comprises a data validation rule process based on the NDS receiving at least 5, 8, or at least 10 packets of MA-D from the MA every at least 2, 3, or 5 seconds (e.g., every 2-10, 2-6, 3-9, 4-8, or 3-8 seconds), and the NDS reinitiates the iteration upon the occurrence of any validation rule violation (aspect 78).

A further aspect is a network according to any one or more of aspects 50-78, wherein each cycle of MA-D collected by the NDS for each MA in forming a data collection for analysis is at least about 100, at least about 250, at least about 500, or at least about 1,000 times as long as each cycle of transmission of a data packet from the MA to the NDS (aspect 79).

A further aspect is a network according to any one or more of aspects 50-79, wherein the NDS can detect MAs in non-clinical operational modes and use such capabilities for enhanced scalability testing (aspect 80).

In one aspect, the invention provides a network such as that described in any one or more of aspects 50-80, wherein at least some of the MAs of the network are mobile MAs that in operation at least some of the time transmit RT-MA-D to the NDS via a wireless communication protocol, e.g., Wi-Fi, and comprise a capability of detecting if a secure/stable communication channel is available, collecting cache MA-D when a secure/stable wireless communication channel is not available, and relaying the stored cache-data when the communication channel is available again (aspect 81).

A further aspect is a network according to any one or more of aspects 50-81, wherein at least some of, if not all, of the MAs of the network comprise MAs that provide treatment for one or more critical life support systems (e.g., the respiratory system, the cardiovascular system, or the nervous system) (aspect 82).

In one aspect, the invention provides a network such as that described in any one or more of aspects 50-82, wherein at least some MAs of the network comprise at least 2 separate components that are subjected to separate security zones/rules (aspect 83).

In one aspect, the invention provides a network such as described in aspect 83, wherein at least some of the multi-zone MAs (MZMAs) comprise a highly restricted therapeutic application component that provides a critical life support system treatment function, comprises physical anti-tampering protection, and comprises MA CEIs that are only locally modifiable (aspect 84).

In one aspect, the invention provides a network such as that described in aspect 83 or aspect 84, wherein at least some of the MAs of the network comprise a patient monitoring component that comprises a processing unit that can receive system update availability but also comprises MA CEIs that are only modifiable through a pull request to the NDS (aspect 85).

In one aspect, the invention provides a system according to the systems of any one or more of aspects 50-85, wherein the system comprises a streaming data processor that performs a limited initial analysis (e.g., limited by analyzing for a set number of rules/datapoints) on incoming data and through controller(s) of the NDS triggers application(s)/alarms, etc., at MAs, ONDIs, or both, based on the limited initial analysis (aspect 86).

Further provided is a method for the real-time management of a number of medical devices in a device data network comprising (1) accessing a number of separately located and at least partially remotely controllable medical apparatuses (“MA”s) that are in substantially continuous communication with a network data system (NDS), optionally under physical control of at least 5 separate independent entities (IEs), each MA of the system comprising (a) at least 1 sensor that in operation detects conditions in a patient, (b) device memory comprising PTRCRM comprising device computer executable instructions and a device memory (DM) that in operation records information comprising sensor information over time; (c) a device processor that reads/executes the MA CEI, (d) a device display unit, (e) a device data relay unit that in operation transmits device information (MA-D) that can comprise sensor information from the MA to the NDS via the internet, wherein most of the MA-D relayed by the MA in operation comprises real time sensor data (RT-MA-D), stored/cache sensor data (MA-CD), or both RT-MA-D and MA cache data (MA-CD), and wherein MA-D comprises both structured and unstructured data, (f) a device data input unit (MA-INPU), and (g) an optional device data security system, which optionally comprises at least 1 component, e.g., a microcontroller, that performs one or more data protection functions comprising restricting data input to only data that is specifically identified data of an approved data type; and (2) collecting the data communicated from (a) in a medical apparatus network data management system (NDS) that (I) comprises an NDS input unit/engine that automatically receives system-acceptable data relayed to the NDS, typically comprising MA-D relayed from MAs, (II) stores the received data in a NDS memory comprising PTRCRM, (3) comprises an analytical unit/engine analyzes/evaluates the received data, (III) comprises an NDS processor that executes stored instructions/CEI contained in the NDS memory (e.g., based on the analysis of the received MA-D), and (IV) comprises an NDS relay unit/engine that communicates new data, output functions (such as MA control instructions), or both, back to devices of the network (e.g., MAs), wherein the NDS memory comprises one or more query-able/searchable data repositories (e.g., a data lake or an enhanced data lake, which in either case receives and stores semi-unstructured MA data (SUMAD) in a format that at least partially complies with pre-establish standards, thereby increasing the speed of processing of the MA-D (aspect 87).

Another aspect is a method of aspect 87, wherein the NDS comprises an engine/unit that automatically performs an initial analysis on received MA-D and performs one or more functions based on the analysis of the MA-D and instructions for controlling operation of the analytical unit to analyze data stored in the NDS memory after being stored (ingested) therein, wherein the initial analysis comprises less analyzed conditions than the post memory-ingestion analysis, is performed automatically, is performed within a short amount of time of receipt of the data (e.g., <3 minutes, <2 minutes, <1 minute, or <0.5 minute), or a combination of any or all thereof (aspect 88).

An aspect is a method of aspect 87 or aspect 88, wherein the NDS comprises a device data harmonization unit/engine, that evaluates if RT-MA-D in MA-D is in a format approvable for analysis by the NDS analytical unit prior to analyzing the data, optionally modifying non-conforming data if possible prior to analysis (aspect 89).

An aspect is a method according to any one or more of aspects 87-89, wherein the NDS comprises an output filtering/routing engine/unit that automatically tags/filters MA-D, analytical data, output applications, or a combination thereof, for delivery to MA(s) or ONDIs, based on preprogrammed and programmable confidentiality or health care compliance rules, wherein the NDS simultaneously relays information to different MAs, ONDIs, or both based on the most recently received MA-D but without delivering the same information to any of the networked MAs or ONDIs (aspect 90).

An aspect is a method according to aspect 90, wherein the NDS is adapted to recognize MA-D that identifies each authorized MA and ONDI that delivers data to or receives data from the NDS, and an entity associated therewith, thereby facilitating protection of confidential information belonging to the entity from being disclosed to MAs/ONDIs associated with other independent entities also accessing the network (aspect 91).

An aspect is a method according to any one or more of aspects 87-91, wherein the NDS comprises an engine/unit that identifies MA-CD (cache data) relayed from MAs and determines if such MA-CD is to be stored in the NDS memory, used by the analytical unit, combined with RT-MA-D, or a combination of any or all thereof (aspect 92).

An aspect is a method according to any one or more of aspects 87-92, wherein the NDS comprises a unit/engine that automatically screens MA-D for, or causes MA-D to comply with, one or more schemas during data analysis, data storage, or both (aspect 93).

An aspect is a method according to any one or more of aspects 87-93, wherein most, generally all, or all MAs comprise a unit/engine that evaluates the presence of a secure/stable communication channel for relaying MA-D to the NDS and wherein such MAs, if such a channel is not present, collect MA-D as cache MA-D (MA-CD) until such a connection is re-established and thereafter relays such cache MA-D to the NDS (aspect 94).

Another aspect is a method according to aspect 94, wherein the NDS comprises a unit/engine that automatically and regularly/continuously transmits NDS status information to the MAs in the network and the MAs comprise CEI that collects MA-D when such signal is not received and relays such collected cache MA-D whenever the signal is next received (aspect 95).

Another aspect is a method according to any one or more of aspects 87-95, wherein the NDS comprises an engine/unit for identifying past, present, and predicted MA-D, and the NDS-analytical unit performs at least 1 predictive function, based on the analysis of the MA-D, and relays the results of the predictive function to the MAs, ONDs, or both (aspect 96).

A further aspect is a method according to any one or more of aspects 87-96, wherein the NDS performs at least one function that is regulated as software as a medical device (SAMD) and at least one other non-SAMD function (NSAMD), and the NDS/system delivers output of the at least one SAMD and at least 1 NSAMD functions to MAs in accordance with CEIs that reflect applicable regulatory status of the SAMD and NSAMD functions (aspect 97).

In another aspect, the invention provides a method such as that described in aspect 97, wherein the at least one SAMD comprises providing diagnostic instructions or therapeutic instructions to an HCP (aspect 98).

In another aspect, the invention provides a method such as that described in aspect 97 or aspect 98, wherein the at least one SAMD changes operating conditions of MAs in response to condition(s) reflected in the MA-D or analysis (aspect 99).

A further aspect is a method according to any one or more of aspects 87-99, wherein the ONDIs comprise a system owner (SO) support system (SOSS) that delivers MA-D, analysis, or both, to SOANADs (aspect 100).

In an aspect, the invention provides a method such as that described in aspect 100, wherein the SOSS combines the MA-D, analysis, or both, with data from a CRMS (aspect 101).

In an additional aspect, the invention provides a method such as that described in aspect 101, wherein the users comprise sales representatives that sell or lease the device on behalf of the system owner (aspect 102).

In another aspect, the invention provides a method such as that described in any one or more of aspects 87-102, wherein the network comprises a research platform (RP) comprising MAs or ONDs associated scientific researcher (SR)-associated network access devices (SRANADs), wherein SRs are not associated with the SO and data received from the SRANADs is used in at least some analysis functions (aspect 103).

In another aspect, the invention provides a method such as that described in any one or more of aspects 87-103, wherein the MA-D comprises information about the operating state of the MA-D and the NDS evaluates MA operating state information in performing analyses, delivering output, or both (aspect 104).

In another aspect, the invention provides a method such as that described in any one or more of aspects 87-104, wherein the MAs comprise 2 or more heterogenous types of MAs and the NDS determines the type of device each MA prior to performing analysis, relaying output, or both (aspect 105).

A further aspect is a method according to any one or more of aspects 87-105, wherein at least most of the MAs of the network are associated with a critical life support function e.g., the cardiovascular system, lung, brain, or kidney (aspect 106).

In an aspect, the invention provides a method such as that described in any one of aspects 87-106, wherein the NDS comprises a machine learning module (MLM) that analyzes MA-D from heterogenous types of MAs and performs or recommends the performance of one or more functions of the NDS based on the analysis of the heterogenous MA MA-D (aspect 107).

A further aspect is a method according to any one or more of aspects 87-106, wherein the NDS comprises a functional module that can write MA-D, analysis, or both, directly to an EHR/EMR (aspect 108).

A further aspect is a method per any one or more of aspects 87-108, wherein the DM is adapted to only receive input from the MA (aspect 109).

In another aspect, the invention provides a method such as that described in any one or more of aspects 87-109, wherein data permitted to be transmitted from the NDS to at least some MAs consists essentially of biometric predictive data, provision of instructions or control of the MA, NDS status, MA software version status, or a combination thereof (aspect 110).

In one aspect, the invention provides a method such as that described in aspect 110, wherein data permitted to be transmitted from the NDS comprises MD software version status, but the system/NDS, the MA, or both, prevents updating MA software over the internet (e.g., the information resulting in an alarm or indicator that a manual/local update of the MA software is required, a pull signal for updating of the software is called for, etc.) (aspect 111).

A further aspect is a method according to any one or more of aspects 87-111, wherein MA-D comprises location information and the NDS simultaneously relays information to at least 2 classes of ONDIs based on facility, metropolitan area, state/region, country, or independent entity-association (aspect 112).

A further aspect is a method according to any one or more of aspects 87-112, wherein the NDS receives input from one or more research teams, a plurality of IEs using the MAs in clinical application, or both, separately identifies/tags such data, and uses such data in at least some analytical operations (aspect 113).

A further aspect is a system according to any one or more of aspects 87-113, wherein the NDSs delivers MA-D, analysis, or both to a plurality of GUIs in MAs/ONDs comprising a plurality of GUI schemes/display types, based at least in part on user roles, such user roles comprising independent research team member, system owner device sales or leasing promotional agent, IE user or administrator, system owner clinical or medical support team personnel, and system owner system analyst (aspect 114).

In another aspect, the invention provides a method such as that described in any one or more of aspects 87-114, wherein the NDS CEI comprises instructions for causing one or more alarm to be registered at MAs, ONDs, or both, based on the presence of one or more alarm-triggering conditions in MA-D, analysis, or both, wherein such conditions are programmable at an NDS level, a MA/OND level, or both (aspect 115).

In an aspect, provided is a method such as that described in any one or more of aspects 87115, wherein some, most, or all of the MAs comprise an anti-tampering detection function that sends a signal to the NDS if a prohibited tampering occurs in an MA (aspect 116).

In an aspect, the invention provides a method such as that described in any one or more of aspects 87-116, wherein MA-D comprises images of the MA (MA components/screens, etc.), the MA environment, or both, as well as non-image sensor data (aspect 117).

In another aspect, the invention provides a method such as that described in any one or more of aspects 87-117, wherein the NDS memory or components thereof (e.g., a data lake/enhanced data lake) comprises separate governance zones that, e.g., manage storage of, use of, and access to (a) MA-D or both; (b) curated data, scored data, or both; (c) system testing data; (d) outgoing data/output; or (e) a combination of any or all of (a)-(d) (aspect 118).

In an aspect, provided is a method such as that described in any one or more of aspects 87-118, wherein the NDS comprises (a) data for MDs in a country, (b) nation-specific data governance rules, and (c) system operation/blueprint instructions/engine(s) shared with one or more other NDSs, which are optionally concurrently operated, share resources, etc. (aspect 119).

In another aspect, the invention provides a method such as that described in any one or more of aspects 87-119, wherein the MAs relay packets comprising sequencing information and the NDS comprises an engine/unit (performs a function) for analyzing the time component of MA-CD and resequencing MA-CD that is received in nonchronological order (aspect 120).

In another aspect, the invention provides a method such as that described in any one or more of aspects 87-120, wherein one or more NDS functions are based on collection of MA-D for at least 20, 30, 40, 60, 90, 120, or 180 seconds of MA-D per iteration (e.g., 30-300 seconds of data, 40-120 seconds of data, or 25-100 seconds of data) and analysis of such collection(s) to determine suitability for use in an analysis, such as an MLM analysis (aspect 121).

In another aspect, the invention provides a method according to any one or more of aspects 87-121, wherein the NDS comprises a data collection process/engine based on collection of an expected amount of packets of data (e.g., 2-100, 5-100, 5-75, 5-80, 5-50, 5-25, or 5-10 packets of MA-D) received over a set period of time (e.g., every 1-10 seconds, every 2-12 seconds, every 2-8 seconds, every 3-6 seconds, or every ~5 seconds), wherein the NDS optionally analyzes a collection before it is complete for compliance with data sufficiency requirements/content rules, and the NDS reinitiates the data collection upon the occurrence of any validation rule violation (aspect 122).

In another aspect, the invention provides a method such as that described in aspect 121 or aspect 122, wherein a cycle of MA-D collection for analysis of a MA-D collection is at least about 100, at least about 250, at least about 500, or at least about 1,000 times as long as at least one period for the NDS to relay output to the MA, ONDIs, or both or for the MAs to relay MA-D to the NDS (aspect 123).

A further aspect is a method according to any one or more of aspects 87-123, wherein the NDS can detect MAs that are in non-clinical operational modes (aspect 124).

Another aspect is a method according to aspect 124, wherein the method comprises performing network scalability testing on non-clinical operating mode MAs prior to incorporating such MAs into the network (aspect 125).

In an aspect, the invention provides a method as that described in any one or more of aspects 87-125, wherein at least some of the MAs are mobile MAs that in operation at least some of the time transmit RT-MA-D to the NDS by wireless communication protocol (aspect 126).

In another aspect, the invention provides a method as described in any one or more of aspects 87-126, wherein at least some of the MAs comprise at least 2 components that are subjected to separate security zones (aspect 127).

In another aspects, the invention provides a method such as that described in aspect 127, wherein at least some of the MAs comprise a highly restricted therapeutic application component that provides a critical life support system treatment function, comprises physical anti-tampering protection, and comprises MA CEIs that are only locally modifiable (aspect 128).

In an aspect, the invention provides a method as that described in any one or more of aspects 87-128, wherein at least some of the MAs comprise a patient monitoring component comprising a processing unit that can receive system update availability but also comprises MA CEIs that are only modifiable through a pull request delivered to the NDS (aspect 129).

Another facet is a method of performing a method according to any one or more of aspects 87-129, wherein the method comprises performing an initial analysis on intake data received by an engine/unit of the NDS (e.g., a streaming data processor) and causing a control component/unit to deliver one or more actions to MAs, ONDIs, or both, based on the initial analysis, wherein the initial analysis occurs before complete ingestion of MA-D into an NDS memory data repository, such as an enhanced data lake (aspect 130).

A further aspect of the invention is a medical apparatus and control system comprising: (1) access to several (e.g., ≥25, ≥50, or ≥~100) remote controllable and internet connectable medical apparatuses (MAs), the MA(s) (I) comprising subject sensor means for sensing subject data and sensor data conversion means for converting sensor data to internet transmittable data (MA-D) and optionally perform therapeutic task means, (II) means for streaming MA-D in a semi-unstructured via secure internet data communication in a substantially continuous manner when in operation and online, (III) comprising means for collecting cached MA-D in and storing such MA-D in a means for containing electronic data (MA memory), (IV) means for sensing the status of communications between the MA and the network; and (V) means for relaying cached MA-D upon occurrence of preprogrammed condition(s) or event(s); (2) an NDS (e.g., MAC-DMS) comprising (I) means for storing data comprising an enhanced data lake (EDL) architecture and applying an EDL-compatible data input ingestion process, (II) massively parallel processing means for processing streaming and cached MA data and for performing automatic and on-demand secondary analysis on data stored in the MAC-DMS data storing means, (III) analytical processing means for analyzing data in the MAC-DMS data storing means to generate analytical data, instructions for output applications performed on MAs, or both, and (IV) means for relaying analytical data, output applications, or both; and (3) several (e.g., ≥~10, ≥~20, or ≥~100) ONDs/ONDIs, each comprising processor means and display means, each ONDI being associated with a class of user and such classes of users comprising (I) HCPs that interact with MAs and are authorized to access PHI and (II) commercial users that do not directly interact with the MA and are subject to restrictions on the receipt of PHI; wherein output applications control the operation of one or more functions in the MAs and concurrently and separately relay analytical data output based on the analysis of the MA-D to at least some of the ONDs associated with commercial class users (aspect 131).

### Additional Aspects Including Means or Steps for Performing Functions

Some aspects of the invention are described with respect to (1) a function and (2) either a “means” of a system/device(s) or “step” of a method for carrying out the function, with the intent that such means include all recited means or steps and their equivalents in the art. To aid the reader, exemplary description or reference(s) to support for select means/steps for performing functions is provided here, but it will be recognized that additional support for various means/steps for performing functions is provided elsewhere here.

For example, means for sensing subject physiological states (steps for collecting sensor data) are provided in, e.g., paragraphs [0139]-[0144] etc. Means for assessing if an adequate/suitable data connection is available are described in, e.g., paragraph [0160] etc. Data input means/input means/input steps (means for receiving data) are described in, e.g., paragraphs [0254]-[0286](with focus on NDS input units/methods) and paragraph [0169](with focus on MA input unit(s)). Steps/means for relaying/transmitting data are provided in, e.g., paragraphs [0158]-[0168](with focus on MA relay units/methods) and paragraphs [0326]-[0337](with focus on NDS relay units/methods). Data improvement means; means/step for improving data are provided in, e.g., paragraphs [0320][0307]-[0328].

Means/steps for reading/interpreting CEI/code and data (“processing means” or “processing steps”) are provided in, e.g., paragraphs [0153]-[0157](with a focus on MA processors) and paragraphs [0234]-[0250](with a focus on NDS processors). Means/steps for analyzing system data (e.g., MA-D) are provided in, e.g., paragraphs [0293]-[0306] and paragraphs [0369]-[0389] etc. Means/steps for analyzing and combining cache data with RT-MAD are provided in, e.g., paragraphs [0260]-[0271] etc.

Memory means; means/step for storing data are provided in, e.g., paragraph [0152](with focus on MA memory) and paragraphs [0199]-[0233](with focus on NDS memory). EDLs and DLs can be considered means/steps for storing unstructured or semi-unstructured data. Means for recording SUMAD, such as JSON file formats, are provided in, e.g., paragraph [0234] or paragraph [0259] or paragraph [0361] etc. Means for processing data streams (steps for processing streaming data) are provided in, e.g., paragraphs [0278]-[0280], paragraph [0292] or paragraph [0364] or paragraph [0367] etc.

Descriptions/support for other means/steps of functions also are provided herein, such as means/steps for protecting confidentiality of information (e.g., using encryption methods disclosed herein and their equivalents), means for restricting incoming data (e.g., using firewalls described herein and their equivalents), etc.

1-16. (canceled)

17. A video transmission device, comprising:
a video input port configured to couple to a video output port of a controller of a medical apparatus and configured to receive image data via the video output port of the controller of the medical apparatus;
a memory;
first circuitry configured to receive the image data from the video input port and store the image data in the memory, wherein the first circuitry is reprogrammable based on a workload of the controller;
a network interface configured to transmit a signal comprising the image data to a network system and to receive an incoming signal comprising additional data from the network system;
second circuitry configured to:
retrieve the image data from the memory, and
transmit the image data to the network interface; and
a firewall module configured to authenticate the additional data received from the network system.

18. The video transmission device of claim 17, wherein the image data comprises a screen capture of a display of the medical apparatus.

19. The video transmission device of claim 17, wherein the network system comprises one or more cloud-based resources.

20. The video transmission device of claim 17, wherein the additional data comprises an alert that a software update for the controller is available.

21. The video transmission device of claim 20, wherein firewall module is configured to:
determine that the alert is authentic;
transmit the alert to the second circuitry;
receive, from the second circuitry, a request for the software update; and
in response, transmit the request for the software update to the network system.

22. The video transmission device of claim 17, further comprising:
a USB input port configured to couple to a corresponding output port of the controller of the medical apparatus; and
a USB output port configured to couple to a corresponding input port of a device other than the medical apparatus.

23. A medical apparatus comprising:
a controller comprising a video output port; and
a video transmission device coupled to the controller, the video transmission device comprising:
a video input port coupled to the video output port and configured to receive image data via the video output port of the controller,
a memory,
first circuitry configured to receive the image data from the video input port and store the image data in the memory, wherein the first circuitry is reprogrammable based on a workload of the controller,
a network interface configured to transmit a signal comprising the image data to a network system and to receive an incoming signal comprising additional data from the network system,
second circuitry configured to:
retrieve the image data from the memory, and
transmit the image data to the network interface, and
a firewall module configured to authenticate the additional data received from the network system.

24. The medical apparatus of claim 23, further comprising a display, wherein the image data comprises a screen capture of the display.

25. The medical apparatus of claim 23, wherein the network system comprises one or more cloud-based resources.

26. The medical apparatus of claim 23, wherein the controller is configured to control one or more therapeutic functions of the medical apparatus.

27. The medical apparatus of claim 23, wherein the additional data comprises an alert that a software update for the controller is available.

28. The medical apparatus of claim 27, wherein firewall module is configured to:
determine that the alert is authentic;
transmit the alert to the second circuitry;
receive, from the second circuitry, a request for the software update; and
in response, transmit the request for the software update to the network system.

29. The medical apparatus of claim 23, wherein the video transmission device further comprises:
a USB input port configured to couple to a corresponding output port of the controller of the medical apparatus; and
a USB output port configured to couple to a corresponding input port of a device other than the medical apparatus.

30. A system comprising:
a medical apparatus comprising:
a controller comprising a video output port, and
a video transmission device coupled to the controller, the video transmission device comprising:
a video input port coupled to the video output port and configured to receive image data from the controller,
a memory,
first circuitry configured to receive the image data from the video input port and store the image data in the memory, wherein the first circuitry is reprogrammable based on a workload of the controller,
a network interface configured to transmit a signal comprising the image data to a first computing device and to receive an incoming signal comprising additional data from a first computing device,
second circuitry configured to:
retrieve the image data from the memory, and
transmit the image data to the network interface, and
a firewall module configured to authenticate the additional data received from the first computing device; and
the first computing device, wherein the first computing device is configured to:
receive the image data from the medical apparatus, and
transmit the image data to a second computing device.

31. The system of claim 30, wherein the medical apparatus further comprises a display, and the image data comprises a screen capture of the display.

32. The system of claim 30, wherein the first computing device comprises one or more cloud-based resources.

33. The system of claim 30, wherein the controller is configured to control one or more therapeutic functions of the medical apparatus.

34. The system of claim 30, wherein:
the second computing device comprises a display, and
the second computing device is configured to display the image data received from the first computing device.

35. The system of claim 30, wherein the second computing device is a second medical apparatus.

36. The system of claim 30, wherein the medical apparatus comprises a heart pump.
